(19) United States
(12) Patent Application Publication
(10) Pub. No.: US 2026/0355165 A1
(43) Pub. Date: Nov. 12, 2026

(54) 2,3,4,9-TETRAHYDRO-1H-PYRIDO[3,4-B]INDOLE DERIVATIVES AS ESTROGEN RECEPTOR MODULATORS FOR THE TREATMENT OF CANCER

(71) Applicant: Olema Pharmaceuticals, Inc., San Francisco, CA (US)

(72) Inventors: Brian R. Hearn, Moraga, CA (US); Xufeng Cao, Daly City, CA (US); David Yeghikyan, Fremont, CA (US); Rupa S. Shetty, San Francisco, CA (US); Brian Rich, San Francisco, CA (US); Reena Chawla, San Francisco, CA (US); Dirk A. Heerding, Bend, OR (US)

(21) Appl. No.: 19/104,887

(22) PCT Filed: Aug. 18, 2023
(86) PCT No.: PCT/US2023/030599

§ 371 Date: Feb. 19, 2025

Related U.S. Application Data
(60) Provisional application No. 63/399,276, filed on Aug. 19, 2022.

(51) Int. Cl. A61K 31/437; A61K 31/4196; A61K 31/436; A61K 31/451; A61K 31/4745; A61K 31/498; A61K 31/506; A61K 31/519; A61K 31/52; A61K 31/527; A61K 31/5685; A61K 31/585; A61K 31/685; A61K 39/395; C07D 471/04; C07D 519/00

(52) U.S. Cl. CPC A61K 31/437; A61K 31/4196; A61K 31/436; A61K 31/451; A61K 31/4745; A61K 31/498; A61K 31/506; A61K 31/519; A61K 31/52; A61K 31/527; A61K 31/5685; A61K 31/585; A61K 31/685; A61K 39/3955; A61K 39/39566; C07D 471/04; C07D 519/00

(57) ABSTRACT

The present disclosure provides 2,3,4,9-tetrahydro-1H-pyrido[3,4-b]indole derivatives of formula I as estrogen receptor modulators for the treatment of cancer.

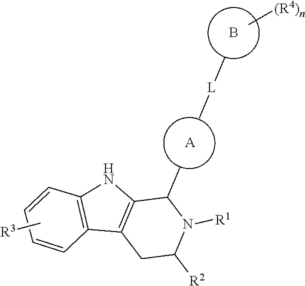

### RELATED APPLICATIONS

This application claims priority to and benefit of U.S. Application No. 63/399,276, filed Aug. 19, 2022, the entire contents of which are hereby incorporated by reference.

### BACKGROUND

The estrogen receptor (ER) plays important roles in various diseases, disorders, and conditions, such as cancers, including breast cancers, menopause-related conditions or symptoms, and osteoporosis. An, K-C. Asian Spine J 10(4); 787-91 (2016 August). About 70% of human breast cancers are hormone dependent and ER-positive. Lumachi, et al., Curr. Med. Chem., 20(5):596-604 (2013). A variety of treatments have been developed to target the estrogen receptor and/or its activities.

### SUMMARY

Selective estrogen receptor modulators (SERMs) and/or degraders (SERDs) are a particularly useful or promising tools for such therapy. For example, an estrogen receptor modulator that acts as an agonist (or partial agonist) in bone tissue may be useful for treating osteoporosis, e.g., in post-menopausal women. Further, an estrogen receptor modulator that acts as an antagonist in breast tissue may be useful for treating breast cancer. In some instances, the same estrogen receptor modulator may be used in both scenarios.

In some embodiments, the present disclosure provides compounds that are estrogen receptor modulators. In some embodiments, provided compounds are estrogen receptor agonists, e.g., as defined herein. In some embodiments, provided compounds are estrogen receptor antagonists, e.g., as defined herein.

Additionally, there remains a need for anti-estrogen agents that can completely inhibit estrogen receptors, including those coded for by both wild-type and mutant versions (e.g., those containing activating mutations) of the gene encoding Estrogen Receptor-alpha (ERα), Estrogen Receptor 1 (ESR1). The estrogen receptor is a tripartite protein comprising two distinct transcriptional activation functions (AF1 and AF2). Complete anti-estrogen activity requires inactivation of both AF1 and AF2. Activating mutations in the gene that codes for estrogen receptor 1 allows for activation of both AF1 and AF2 even in the absence of estrogen.

Many patients develop resistance to certain therapies that target the estrogen receptor (ER) over time. Certain first line therapies for treating ER-associated diseases, disorders, or conditions, are found to exhibit agonistic activity in conjunction with their antagonistic properties. Fulvestrant, in contrast, is the only approved therapy that exhibits complete anti-estrogenic activity, but is not orally bioavailable, and must be administered parenterally.

In some embodiments, the present disclosure provides certain compounds and compositions that are complete estrogen receptor antagonists, and therefore do not suffer from the deficiencies found in previous therapies.

Additionally, in some embodiments, provided compounds may be orally bioavailable.

In some embodiments, the present disclosure provides an estrogen receptor modulator (e.g., an estrogen receptor agonist, an estrogen receptor antagonist, and/or a complete estrogen receptor antagonist) that is a compound of Formula I.

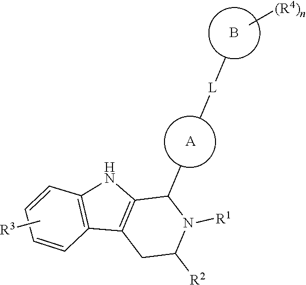

or a pharmaceutically acceptable salt thereof, wherein A, B, L, n, R¹, R², R³, and R⁴ are as defined herein.

In some embodiments, the present disclosure provides methods of treating a disease, disorder, or condition associated with an estrogen receptor. In some embodiments, the present disclosure provides a methods of treating a disease, disorder, or condition associated with a mutation of an estrogen receptor.

In some embodiments, the present disclosure provides methods of treating a cancer. In some embodiments, the present disclosure provides methods of treating a cancer comprising administering a compound of Formula I or a pharmaceutically acceptable salt thereof, optionally in combination with an anti-cancer agent.

In some embodiments, the present disclosure provides methods of preventing recurrence of a cancer. In some embodiments, the present disclosure provides methods of preventing recurrence of a cancer comprising administering a compound of Formula I or a pharmaceutically acceptable salt thereof, optionally in combination with an anti-cancer agent.

In some embodiments, the present disclosure provides methods of treating osteoporosis, e.g., in post-menopausal women. In some embodiments, the present disclosure provides methods of treating osteoporosis comprising administering a compound of Formula I or a pharmaceutically acceptable salt thereof.

In some embodiments, the present disclosure provides methods of treating one or more menopausal symptoms or conditions. In some embodiments, the present disclosure provides methods of treating one or more menopausal symptoms comprising administering a compound of Formula I or a pharmaceutically acceptable salt thereof.

### DETAILED DESCRIPTION OF CERTAIN EMBODIMENTS

The present disclosure provides compounds and compositions useful as estrogen receptor modulators (e.g., estrogen receptor agonists, estrogen receptor antagonists, and/or complete estrogen receptor antagonists). In some embodiments, such compounds include those of the formulae described herein, or a pharmaceutically acceptable salt thereof, wherein each variable is as defined and described herein.

### COMPOUNDS AND DEFINITIONS

Compounds of this disclosure include those described generally above and are further illustrated by the classes, subclasses, and species disclosed herein. As used herein, the following definitions shall apply unless otherwise indicated. For purposes of this disclosure, the chemical elements are identified in accordance with the Periodic Table of Elements, CAS version, Handbook of Chemistry and Physics, 75^{th} Ed. Additionally, general principles of organic chemistry are described in “Organic Chemistry”, Thomas Sorrell, University Science Books, Sausalito: 1999, and “March's Advanced Organic Chemistry”, 5^{th} Ed., Ed.: Smith, M. B. and March, J., John Wiley & Sons, New York: 2001, the entire contents of which are hereby incorporated by reference.

Unless otherwise stated, structures depicted herein are meant to include all stereoisomeric (e.g., enantiomeric or diastereomeric) forms of the structure, as well as all geometric or conformational isomeric forms of the structure. For example, the R and S configurations of each stereocenter are contemplated as part of the disclosure. Therefore, single stereochemical isomers, as well as enantiomeric, diastereomeric, and geometric (or conformational) mixtures of provided compounds are within the scope of the disclosure. For example, in some cases, Table 1 shows one or more stereoisomers of a compound, and unless otherwise indicated, represents each stereoisomer alone and/or as a mixture. Unless otherwise stated, all tautomeric forms of provided compounds are within the scope of the disclosure.

Unless otherwise indicated, structures depicted herein are meant to include compounds that differ only in the presence of one or more isotopically enriched atoms. For example, compounds having the present structures including replacement of hydrogen by deuterium or tritium, or replacement of a carbon by ¹³C- or ¹⁴C-enriched carbon are within the scope of this disclosure.

About or approximately: As used herein, the term “approximately” or “about,” as applied to one or more values of interest, refers to a value that is similar to a stated reference value. In general, those skilled in the art, familiar within the context, will appreciate the relevant degree of variance encompassed by “about” or “approximately” in that context. For example, in some embodiments, the term “approximately” or “about” may encompass a range of values that are within (i.e., ±) 25%, 20%, 19%, 18%, 17%, 16%, 15%, 14%, 13%, 12%, 11%, 10%, 9%, 8%, 7%, 6%, 5%, 4%, 3%, 2%, 1%, or less of the referred value.

Administering: As used herein, the term “administering” or “administration” typically refers to the administration of a composition to a subject to achieve delivery of an agent that is, or is included in, a composition to a target site or a site to be treated. Those of ordinary skill in the art will be aware of a variety of routes that may, in appropriate circumstances, be utilized for administration to a subject, for example a human. For example, in some embodiments, administration may be ocular, oral, parenteral, topical, etc. In some particular embodiments, administration may be bronchial (e.g., by bronchial instillation), buccal, dermal (which may be or comprise, for example, one or more of topical to the dermis, intradermal, interdermal, transdermal, etc.), enteral, intra-arterial, intradermal, intragastric, intramedullary, intramuscular, intranasal, intraperitoneal, intrathecal, intravenous, intraventricular, within a specific organ (e.g., intrahepatic), mucosal, nasal, oral, rectal, subcutaneous, sublingual, topical, tracheal (e.g., by intratracheal instillation), vaginal, vitreal, etc. In some embodiments, administration may be parenteral. In some embodiments, administration may be oral. In some particular embodiments, administration may be intravenous. In some particular embodiments, administration may be subcutaneous. In some embodiments, administration may involve only a single dose. In some embodiments, administration may involve application of a fixed number of doses. In some embodiments, administration may involve dosing that is intermittent (e.g., a plurality of doses separated in time) and/or periodic (e.g., individual doses separated by a common period of time) dosing. In some embodiments, administration may involve continuous dosing (e.g., perfusion) for at least a selected period of time. In some embodiments, administration may comprise a prime-and-boost protocol. A prime-and-boost protocol can include administration of a first dose of a pharmaceutical composition followed by, after an interval of time, administration of a second or subsequent dose of a pharmaceutical composition.

Agonist: As used herein, the term “agonist” generally refers to an agent whose presence or level correlates with elevated level or activity of a target, as compared with that observed absent the agent (or with the agent at a different level). In some embodiments, an agonist is one whose presence or level correlates with a target level or activity that is comparable to or greater than a particular reference level or activity (e.g., that observed under appropriate reference conditions, such as presence of a known agonist, e.g., a positive control). In some embodiments, an agonist may be a direct agonist in that it exerts its influence directly on (e.g., interacts directly with) the target; in some embodiments, an agonist may be an indirect agonist in that it exerts its influence indirectly (e.g., by acting on, such as interacting with, a regulator of the target, or with some other component or entity.

Aliphatic: The term “aliphatic” refers to a straight-chain (i.e., unbranched) or branched, substituted or unsubstituted hydrocarbon chain that is completely saturated or that contains one or more units of unsaturation, or a monocyclic hydrocarbon or bicyclic hydrocarbon that is completely saturated or that contains one or more units of unsaturation, but which is not aromatic (also referred to herein as “cycloaliphatic”), that has a single point or more than one points of attachment to the rest of the molecule. Unless otherwise specified, aliphatic groups contain 1-12 aliphatic carbon atoms. In some embodiments, aliphatic groups contain 1-6 aliphatic carbon atoms (e.g., C₁₋₆). In some embodiments, aliphatic groups contain 1-5 aliphatic carbon atoms (e.g., C₁₋₅). In other embodiments, aliphatic groups contain 1-4 aliphatic carbon atoms (e.g., C₁₋₄). In still other embodiments, aliphatic groups contain 1-3 aliphatic carbon atoms (e.g., C₁₋₃), and in yet other embodiments, aliphatic groups contain 1-2 aliphatic carbon atoms (e.g., C₁₋₂). Suitable aliphatic groups include, but are not limited to, linear or branched, substituted or unsubstituted alkyl, alkenyl, or alkynyl groups and hybrids thereof. A preferred aliphatic group is C₁₋₆ alkyl.

Alkyl: The term “alkyl”, used alone or as part of a larger moiety, refers to a saturated, optionally substituted straight or branched chain hydrocarbon group having (unless otherwise specified) 1-12, 1-10, 1-8, 1-6, 1-4, 1-3, or 1-2 carbon atoms (e.g., C₁₋₁₂, C₁₋₁₀, C₁₋₈, C₁₋₆, C₁₋₄, C₁₋₃, or C₁₋₂). Exemplary alkyl groups include methyl, ethyl, propyl, butyl, pentyl, hexyl, and heptyl.

Alkylene: The term “alkylene” is refers to a bivalent alkyl group. In some embodiments, “alkylene” is a bivalent straight or branched alkyl group. In some embodiments, an “alkylene chain” is a polymethylene group, i.e., —(CH₂)ₙ—, wherein n is a positive integer, e.g., from 1 to 6, from 1 to 4, from 1 to 3, from 1 to 2, or from 2 to 3. An optionally substituted alkylene chain is a polymethylene group in which one or more methylene hydrogen atoms is optionally replaced with a substituent. Suitable substituents include those described below for a substituted aliphatic group and also include those described in the specification herein. It will be appreciated that two substituents of the alkylene group may be taken together to form a ring system. In certain embodiments, two substituents can be taken together to form a 3- to 7-membered ring. The substituents can be on the same or different atoms. The suffix “-ene” when appended to certain groups herein are intended to refer to a bifunctional moiety of said group. For example, “-ene”, when appended to “cyclopropyl” becomes “cyclopropylene” and is intended to refer to a bifunctional cyclopropyl group, e.g.,

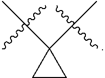 [SMILES: *C1(*)CC1]

Alkenyl: The term “alkenyl”, used alone or as part of a larger moiety, refers to an optionally substituted straight or branched chain or cyclic hydrocarbon group having at least one double bond and having (unless otherwise specified) 2-12, 2-10, 2-8, 2-6, 2-4, or 2-3 carbon atoms (e.g., C₂₋₁₂, C₂₋₁₀, C₂₋₈, C₂₋₆, C₂₋₄, or C₂₋₃). Exemplary alkenyl groups include ethenyl, propenyl, butenyl, pentenyl, hexenyl, and heptenyl. The term “cycloalkenyl” refers to an optionally substituted non-aromatic monocyclic or multicyclic ring system containing at least one carbon-carbon double bond and having about 3 to about 10 carbon atoms. Exemplary monocyclic cycloalkenyl rings include cyclopentenyl, cyclohexenyl, and cycloheptenyl.

Alkynyl: The term “alkynyl”, used alone or as part of a larger moiety, refers to an optionally substituted straight or branched chain hydrocarbon group having at least one triple bond and having (unless otherwise specified) 2-12, 2-10, 2-8, 2-6, 2-4, or 2-3 carbon atoms (e.g., C₂₋₁₂, C₂₋₁₀, C₂₋₈, C₂₋₆, C₂₋₄, or C₂₋₃). Exemplary alkynyl groups include ethynyl, propynyl, butynyl, pentynyl, hexynyl, and heptynyl.

Antagonist: As will be understood by those skilled in the art, the term “antagonist” generally refers to an agent whose presence or level correlates with decreased level or activity of a target, as compared with that observed absent the agent (or with the agent at a different level). In some embodiments, an antagonist is one whose presence or level correlates with a target level or activity that is comparable to or less than a particular reference level or activity (e.g., that observed under appropriate reference conditions, such as presence of a known antagonist, e.g., a positive control). In some embodiments, an antagonist may be a direct antagonist in that it exerts its influence directly on (e.g., interacts directly with) the target; in some embodiments, an antagonist may be an indirect antagonist in that it exerts its influence indirectly (e.g., by acting on, such as interacting with, a regulator of the target, or with some other component or entity.

Aryl: The term “aryl” refers to monocyclic and bicyclic ring systems having a total of six to fourteen ring members (e.g., C₆-C₁₄), wherein at least one ring in the system is aromatic and wherein each ring in the system contains three to seven ring members. In some embodiments, an “aryl” group contains between six and twelve total ring members (e.g., C₆-C₁₂). The term “aryl” may be used interchangeably with the term “aryl ring”. In certain embodiments, “aryl” refers to an aromatic ring system which includes, but not limited to, phenyl, biphenyl, naphthyl, anthracyl and the like, which may bear one or more substituents. Unless otherwise specified, “aryl” groups are hydrocarbons. In some embodiments, an “aryl” ring system is an aromatic ring (e.g., phenyl) that is fused to a non-aromatic ring (e.g., cycloalkyl). Examples of aryl rings include that are fused include

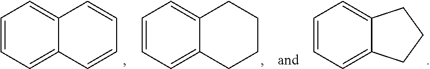

Biological sample: As used herein, the term “biological sample” typically refers to a sample obtained or derived from a biological source (e.g., a tissue or organism or cell culture) of interest, as described herein. In some embodiments, a source of interest comprises an organism, such as an animal or human. In some embodiments, a biological sample is or comprises biological tissue or fluid. In some embodiments, a biological sample may be or comprise bone marrow; blood; blood cells; ascites; tissue or fine needle biopsy samples; cell-containing body fluids; free floating nucleic acids; sputum; saliva; urine; cerebrospinal fluid, peritoneal fluid; pleural fluid; feces; lymph; gynecological fluids; skin swabs; vaginal swabs; oral swabs; nasal swabs; washings or lavages such as a ductal lavages or broncheoalveolar lavages; aspirates; scrapings; bone marrow specimens; tissue biopsy specimens; surgical specimens; feces, other body fluids, secretions, and/or excretions; and/or cells therefrom, etc. In some embodiments, a biological sample is or comprises cells obtained from an individual. In some embodiments, obtained cells are or include cells from an individual from whom the sample is obtained. In some embodiments, a sample is a “primary sample” obtained directly from a source of interest by any appropriate means. For example, in some embodiments, a primary biological sample is obtained by methods selected from the group consisting of biopsy (e.g., fine needle aspiration or tissue biopsy), surgery, collection of body fluid (e.g., blood, lymph, feces etc.), etc. In some embodiments, as will be clear from context, the term “sample” refers to a preparation that is obtained by processing (e.g., by removing one or more components of and/or by adding one or more agents to) a primary sample. For example, filtering using a semi-permeable membrane. Such a “processed sample” may comprise, for example, nucleic acids or proteins extracted from a sample or obtained by subjecting a primary sample to techniques such as amplification or reverse transcription of mRNA, isolation and/or purification of certain components, etc.

Carrier: As used herein, the term “carrier” refers to a diluent, adjuvant, excipient, or vehicle with which a composition is administered. In some exemplary embodiments, carriers can include sterile liquids, such as, for example, water and oils, including oils of petroleum, animal, vegetable or synthetic origin, such as, for example, peanut oil, soybean oil, mineral oil, sesame oil and the like. In some embodiments, carriers are or include one or more solid components.

Combination therapy: As used herein, the term “combination therapy” refers to those situations in which a subject is simultaneously exposed to two or more therapeutic regimens (e.g., two or more therapeutic agents or modality(ies)). In some embodiments, the two or more regimens may be administered simultaneously; in some embodiments, such regimens may be administered sequentially (e.g., all “doses” of a first regimen are administered prior to administration of any doses of a second regimen); in some embodiments, such agents are administered in overlapping dosing regimens. In some embodiments, “administration” of combination therapy may involve administration of one or more agent(s) or modality(ies) to a subject receiving the other agent(s) or modality(ies) in the combination. For clarity, combination therapy does not require that individual agents be administered together in a single composition (or even necessarily at the same time), although in some embodiments, two or more agents, or active moieties thereof, may be administered together in a combination composition, or even in a combination compound (e.g., as part of a single chemical complex or covalent entity).

Comparable: As used herein, the term “comparable” refers to two or more agents, entities, situations, sets of conditions, etc., that may not be identical to one another but that are sufficiently similar to permit comparison therebetween so that one skilled in the art will appreciate that conclusions may reasonably be drawn based on differences or similarities observed. In some embodiments, comparable sets of conditions, circumstances, individuals, or populations are characterized by a plurality of substantially identical features and one or a small number of varied features. Those of ordinary skill in the art will understand, in context, what degree of identity is required in any given circumstance for two or more such agents, entities, situations, sets of conditions, etc. to be considered comparable. For example, those of ordinary skill in the art will appreciate that sets of circumstances, individuals, or populations are comparable to one another when characterized by a sufficient number and type of substantially identical features to warrant a reasonable conclusion that differences in results obtained or phenomena observed under or with different sets of circumstances, individuals, or populations are caused by or indicative of the variation in those features that are varied.

Composition: Those skilled in the art will appreciate that the term “composition” may be used to refer to a discrete physical entity that comprises one or more specified components. In general, unless otherwise specified, a composition may be of any form—e.g., gas, gel, liquid, solid, etc.

Cycloaliphatic: As used herein, the term “cycloaliphatic” refers to a monocyclic C₃₋₈ hydrocarbon or a bicyclic C₅₋₁₀ hydrocarbon that is completely saturated or that contains one or more units of unsaturation, but which is not aromatic, that has a single point or more than one points of attachment to the rest of the molecule.

Cycloalkyl: As used herein, the term “cycloalkyl” refers to an optionally substituted saturated monocyclic or polycyclic ring system of about 3 to about 10 ring carbon atoms. Exemplary monocyclic cycloalkyl rings include cyclopropyl, cyclobutyl, cyclopentyl, cyclohexyl, and cycloheptyl.

Dosageform or unit dosage form: Those skilled in the art will appreciate that the term “dosage form” may be used to refer to a physically discrete unit of an active agent (e.g., a therapeutic or diagnostic agent) for administration to a subject. Typically, each such unit contains a predetermined quantity of active agent. In some embodiments, such quantity is a unit dosage amount (or a whole fraction thereof) appropriate for administration in accordance with a dosing regimen that has been determined to correlate with a desired or beneficial outcome when administered to a relevant population (i.e., with a therapeutic dosing regimen).

Dosing regimen or therapeutic regimen: Those skilled in the art will appreciate that the terms “dosing regimen” and “therapeutic regimen” may be used to refer to a set of unit doses (typically more than one) that are administered individually to a subject, typically separated by periods of time. In some embodiments, a given therapeutic agent has a recommended dosing regimen, which may involve one or more doses. In some embodiments, a dosing regimen comprises a plurality of doses each of which is separated in time from other doses. In some embodiments, individual doses are separated from one another by a time period of the same length; in some embodiments, a dosing regimen comprises a plurality of doses and at least two different time periods separating individual doses. In some embodiments, all doses within a dosing regimen are of the same unit dose amount. In some embodiments, different doses within a dosing regimen are of different amounts. In some embodiments, a dosing regimen comprises a first dose in a first dose amount, followed by one or more additional doses in a second dose amount different from the first dose amount. In some embodiments, a dosing regimen comprises a first dose in a first dose amount, followed by one or more additional doses in a second dose amount same as the first dose amount. In some embodiments, a dosing regimen is correlated with a desired or beneficial outcome when administered across a relevant population (i.e., is a therapeutic dosing regimen).

Excipient: As used herein, the term “excipient” refers to a non-therapeutic agent that may be included in a pharmaceutical composition, for example, to provide or contribute to a desired consistency or stabilizing effect. Suitable pharmaceutical excipients include, for example, starch, glucose, lactose, sucrose, gelatin, malt, rice, flour, chalk, silica gel, sodium stearate, glycerol monostearate, talc, sodium chloride, dried skim milk, glycerol, propylene, glycol, water, ethanol and the like.

Heteroaliphatic: The term “heteroaliphatic” or “heteroaliphatic group”, as used herein, denotes an optionally substituted hydrocarbon moiety having, in addition to carbon atoms, from one to five heteroatoms, that may be straight-chain (i.e., unbranched), branched, or cyclic (“heterocyclic”) and may be completely saturated or may contain one or more units of unsaturation, but which is not aromatic. The term “heteroatom” refers to nitrogen, oxygen, or sulfur, and includes any oxidized form of nitrogen or sulfur, and any quaternized form of a basic nitrogen. The term “nitrogen” also includes a substituted nitrogen. Unless otherwise specified, heteroaliphatic groups contain 1-10 carbon atoms wherein 1-3 carbon atoms are optionally and independently replaced with heteroatoms selected from oxygen, nitrogen, and sulfur. In some embodiments, heteroaliphatic groups contain 1-4 carbon atoms, wherein 1-2 carbon atoms are optionally and independently replaced with heteroatoms selected from oxygen, nitrogen, and sulfur. In yet other embodiments, heteroaliphatic groups contain 1-3 carbon atoms, wherein 1 carbon atom is optionally and independently replaced with a heteroatom selected from oxygen, nitrogen, and sulfur. Suitable heteroaliphatic groups include, but are not limited to, linear or branched, heteroalkyl, heteroalkenyl, and heteroalkynyl groups. For example, a 1- to 10 atom heteroaliphatic group includes the following exemplary groups: —O—CH₃, —CH₂—O—CH₃, —O—CH₂—CH₂—O—CH₂—CH₂—O—CH₃, and the like.

Heteroaryl: The terms “heteroaryl” and “heteroar-”, used alone or as part of a larger moiety, e.g., “heteroaralkyl”, or “heteroaralkoxy”, refer to monocyclic or bicyclic ring groups having 5 to 10 ring atoms (e.g., 5- to 6-membered monocyclic heteroaryl or 9- to 10-membered bicyclic heteroaryl); having 6, 10, or 14 π-electrons shared in a cyclic array; and having, in addition to carbon atoms, from one to five heteroatoms. Heteroaryl groups include, without limitation, thienyl, furanyl, pyrrolyl, imidazolyl, pyrazolyl, triazolyl, tetrazolyl, oxazolyl, isoxazolyl, oxadiazolyl, thiazolyl, isothiazolyl, thiadiazolyl, pyridyl, pyridazinyl, pyrimidinyl, pyrazinyl, indolizinyl, purinyl, naphthyridinyl, pteridinyl, imidazo[1,2-a]pyrimidinyl, imidazo[1,2-a]pyridyl, imidazo[4,5-b]pyridyl, imidazo[4,5-c]pyridyl, pyrrolopyridyl, pyrrolopyrazinyl, thienopyrimidinyl, triazolopyridyl, and benzoisoxazolyl. The terms “heteroaryl” and “heteroar-”, as used herein, also include groups in which a heteroaromatic ring is fused to one or more aryl, cycloaliphatic, or heterocyclyl rings, where the radical or point of attachment is on the heteroaromatic ring (i.e., a bicyclic heteroaryl ring having 1 to 3 heteroatoms). Nonlimiting examples include indolyl, isoindolyl, benzothienyl, benzofuranyl, dibenzofuranyl, indazolyl, benzimidazolyl, benzotriazolyl, benzothiazolyl, benzothiadiazolyl, benzoxazolyl, quinolyl, isoquinolyl, cinnolinyl, phthalazinyl, quinazolinyl, quinoxalinyl, 4H-quinolizinyl, carbazolyl, acridinyl, phenazinyl, phenothiazinyl, phenoxazinyl, tetrahydroquinolinyl, tetrahydroisoquinolinyl, pyrido[2,3-b]-1,4-oxazin-3(4H)-one, 4H-thieno[3,2-b]pyrrole, and benzoisoxazolyl. The term “heteroaryl” may be used interchangeably with the terms “heteroaryl ring”, “heteroaryl group”, or “heteroaromatic”, any of which terms include rings that are optionally substituted.

Heteroatom: The term “heteroatom” as used herein refers to nitrogen, oxygen, or sulfur, and includes any oxidized form of nitrogen or sulfur, and any quaternized form of a basic nitrogen.

Heterocycle: As used herein, the terms “heterocycle”, “heterocyclyl”, “heterocyclic radical”, and “heterocyclic ring” are used interchangeably and refer to a stable 3- to 8-membered monocyclic, a 6- to 10-membered bicyclic, or a 10- to 16-membered polycyclic heterocyclic moiety that is either saturated or partially unsaturated, and having, in addition to carbon atoms, one or more, such as one to four, heteroatoms, as defined above. When used in reference to a ring atom of a heterocycle, the term “nitrogen” includes a substituted nitrogen. As an example, in a saturated or partially unsaturated ring having 0-3 heteroatoms selected from oxygen, sulfur or nitrogen, the nitrogen may be N (as in 3,4-dihydro-2H-pyrrolyl), NH (as in pyrrolidinyl), or NR⁺ (as in N-substituted pyrrolidinyl). A heterocyclic ring can be attached to its pendant group at any heteroatom or carbon atom that results in a stable structure and any of the ring atoms can be optionally substituted. Examples of such saturated or partially unsaturated heterocyclic radicals include, without limitation, azetidinyl, oxetanyl, tetrahydrofuranyl, tetrahydrothienyl, pyrrolidinyl, piperidinyl, decahydroquinolinyl, oxazolidinyl, piperazinyl, dioxanyl, dioxolanyl, diazepinyl, oxazepinyl, thiazepinyl, morpholinyl, and thiamorpholinyl. A heterocyclyl group may be mono-, bi-, tri-, or polycyclic, preferably mono-, bi-, or tricyclic, more preferably mono- or bicyclic. A bicyclic heterocyclic ring also includes groups in which the heterocyclic ring is fused to one or more aryl rings. Exemplary bicyclic heterocyclic groups include indolinyl, isoindolinyl, benzodioxolyl, 1,3-dihydroisobenzofuranyl, 2,3-dihydrobenzofuranyl, and tetrahydroquinolinyl. A bicyclic heterocyclic ring can also be a spirocyclic ring system (e.g., 6- to 11-membered spirocyclic fused heterocyclic ring having, in addition to carbon atoms, one or more heteroatoms as defined above (e.g., one, two, three or four heteroatoms)). A bicyclic heterocyclic ring can also be a bridged ring system (e.g., 7- to 11-membered bridged heterocyclic ring having one, two, or three bridging atoms.

Modulator: The term “modulator,” as used herein, refers to a compound (e.g., a small molecule) that can alter the activity of another molecule (e.g., a protein). For example, in some embodiments, a modulator can cause an increase or decrease in the magnitude of a certain activity of a type of molecule as compared to the magnitude of the activity in the absence of the modulator. For example, a modulator can be an agonist or an antagonist of a particular target, as those terms are defined herein. For example, in some embodiments, a modulator is an agonist. In some embodiments, a modulator is an antagonist.

Oral: The phrases “oral administration” and “administered orally” as used herein have their art-understood meaning referring to administration by mouth of a compound or composition.

Parenteral: The phrases “parenteral administration” and “administered parenterally” as used herein have their art-understood meaning referring to modes of administration other than enteral and topical administration, usually by injection, and include, without limitation, intravenous, intramuscular, intra-arterial, intrathecal, intracapsular, intraorbital, intracardiac, intradermal, intraperitoneal, transtracheal, subcutaneous, subcuticular, intraarticulare, subcapsular, subarachnoid, intraspinal, and intrasternal injection and infusion.

Partially unsaturated: As used herein, the term “partially unsaturated” refers to a ring moiety that includes at least one double or triple bond between ring atoms. The term “partially unsaturated” is intended to encompass rings having multiple sites of unsaturation, but is not intended to include aromatic (e.g., aryl or heteroaryl) moieties, as herein defined.

Patient or subject: As used herein, the term “patient” or “subject” refers to any organism to which a provided composition is or may be administered, e.g., for experimental, diagnostic, prophylactic, cosmetic, and/or therapeutic purposes. Typical patients or subjects include animals (e.g., mammals such as mice, rats, rabbits, non-human primates, and/or humans). In some embodiments, a patient is a human. In some embodiments, a patient or a subject is suffering from or susceptible to one or more disorders or conditions. In some embodiments, a patient or subject displays one or more symptoms of a disorder or condition. In some embodiments, a patient or subject has been diagnosed with one or more disorders or conditions. In some embodiments, a patient or a subject is receiving or has received certain therapy to diagnose and/or to treat a disease, disorder, or condition.

Pharmaceutical composition: As used herein, the term “pharmaceutical composition” refers to an active agent, formulated together with one or more pharmaceutically acceptable carriers. In some embodiments, the active agent is present in unit dose amount appropriate for administration in a therapeutic or dosing regimen that shows a statistically significant probability of achieving a predetermined therapeutic effect when administered to a relevant population. In some embodiments, pharmaceutical compositions may be specially formulated for administration in solid or liquid form, including those adapted for the following: oral administration, for example, drenches (aqueous or non-aqueous solutions or suspensions), tablets, e.g., those targeted for buccal, sublingual, and systemic absorption, boluses, powders, granules, pastes for application to the tongue; parenteral administration, for example, by subcutaneous, intramuscular, intravenous or epidural injection as, for example, a sterile solution or suspension, or sustained-release formulation; topical application, for example, as a cream, ointment, or a controlled-release patch or spray applied to the skin, lungs, or oral cavity; intravaginally or intrarectally, for example, as a pessary, cream, or foam; sublingually; ocularly; transdermally; or nasally, pulmonary, and to other mucosal surfaces.

Pharmaceutically acceptable: As used herein, the phrase “pharmaceutically acceptable” refers to those compounds, materials, compositions, and/or dosage forms which are, within the scope of sound medical judgment, suitable for use in contact with the tissues of human beings and animals without excessive toxicity, irritation, allergic response, or other problem or complication, commensurate with a reasonable benefit/risk ratio.

Pharmaceutically acceptable salt: The term “pharmaceutically acceptable salt”, as used herein, refers to salts of such compounds that are appropriate for use in pharmaceutical contexts, i.e., salts which are, within the scope of sound medical judgment, suitable for use in contact with the tissues of humans and lower animals without undue toxicity, irritation, allergic response and the like, and are commensurate with a reasonable benefit/risk ratio. Pharmaceutically acceptable salts are well known in the art. For example, S. M. Berge, et al. describes pharmaceutically acceptable salts in detail in J. Pharmaceutical Sciences, 66: 1-19 (1977).

Prevent or prevention: As used herein, the terms “prevent” or “prevention”, when used in connection with the occurrence of a disease, disorder, and/or condition, refer to reducing the risk of developing the disease, disorder and/or condition and/or to delaying onset of one or more characteristics or symptoms of the disease, disorder or condition. Prevention may be considered complete when onset of a disease, disorder or condition has been delayed for a predefined period of time.

Substituted or optionally substituted: As described herein, compounds of the invention may contain “optionally substituted” moieties. In general, the term “substituted,” whether preceded by the term “optionally” or not, means that one or more hydrogens of the designated moiety are replaced with a suitable substituent. “Substituted” applies to one or more hydrogens that are either explicit or implicit from the structure (e.g.,

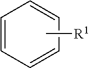

refers to at least

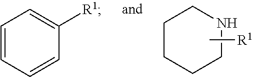

and refers to at least

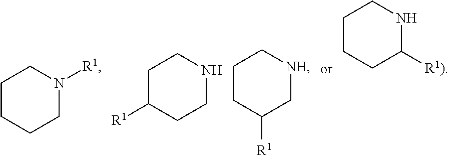

Unless otherwise indicated, an “optionally substituted” group may have a suitable substituent at each substitutable position of the group, and when more than one position in any given structure may be substituted with more than one substituent selected from a specified group, the substituent may be either the same or different at every position. Combinations of substituents envisioned by this invention are preferably those that result in the formation of stable or chemically feasible compounds. The term “stable,” as used herein, refers to compounds that are not substantially altered when subjected to conditions to allow for their production, detection, and, in certain embodiments, their recovery, purification, and use for one or more of the purposes provided herein. Groups described as being “substituted” preferably have between 1 and 4 substituents, more preferably 1 or 2 substituents. Groups described as being “optionally substituted” may be unsubstituted or be “substituted” as described above.

Suitable monovalent substituents on a substitutable carbon atom of an “optionally substituted” group are independently halogen; —(CH₂)₀₋₄R^{◯}; —(CH₂)₀₋₄OR^{◯}; —O(CH₂)₀₋₄R^{◯}, —O—(CH₂)₀₋₄C(O)OR^{◯}; —(CH₂)₀₋₄CH(OR^{◯})₂; —(CH₂)₀₋₄SR^{◯}; —(CH₂)₀₋₄Ph, which may be substituted with R^{◯}; —(CH₂)₀₋₄O(CH₂)₀₋₁Ph which may be substituted with R^{◯}; —CH═CHPh, which may be substituted with R^{◯}; —(CH₂)₀₋₄O(CH₂)₀₋₁-pyridyl which may be substituted with R^{◯}; —NO₂; —CN; —N₃; —(CH₂)₀₋₄N(R^{◯})₂; —(CH₂)₀₋₄N(R^{◯})C(O)R^{◯}; —N(R^{◯})C(S)R^{◯}; —(CH₂)₀₋₄N(R^{◯})C(O)NR^{◯}₂; —N(R^{◯})C(S)NR^{◯}₂; —(CH₂)₀₋₄N(R^{◯})C(O)OR^{◯}; —N(R^{◯})N(R^{◯})C(O)R^{◯}; —N(R^{◯})N(R^{◯})C(O)NR^{◯}₂; —N(R^{◯})N(R^{◯})C(O)OR^{◯}; —(CH₂)₀₋₄C(O)R^{◯}; —C(S)R^{◯}; —(CH₂)₀₋₄C(O)OR^{◯}; —(CH₂)₀₋₄C(O)SR^{◯}; —(CH₂)₀₋₄C(O)OSiR^{◯}₃; —(CH₂)₀₋₄OC(O)R^{◯}; —OC(O)(CH₂)₀₋₄SR^{◯}; —(CH₂)₀₋₄SC(O)R^{◯}; —(CH₂)₀₋₄C(O)NR^{◯}₂; —C(S)NR^{◯}₂; —C(S)SR^{◯}; —SC(S)SR^{◯}, —(CH₂)₀₋₄OC(O)NR^{◯}₂; —C(O)N(OR^{◯})R^{◯}; —C(O)C(O)R^{◯}; —C(O)CH₂C(O)R^{◯}; —C(NOR^{◯})R^{◯}; —(CH₂)₀₋₄SSR^{◯}; —(CH₂)₀₋₄S(O)₂R^{◯}; —(CH₂)₀₋₄S(O)₂OR^{◯}; —(CH₂)₀₋₄OS(O)₂R^{◯}; —S(O)₂NR^{◯}₂; —(CH₂)₀₋₄S(O)R^{◯}; —N(R^{◯})S(O)₂NR^{◯}₂; —N(R^{◯})S(O)₂R^{◯}; —N(OR^{◯})R^{◯}; —C(NH)NR^{◯}₂; —P(O)₂R^{◯}; —P(O)R^{◯}₂; —OP(O)R^{◯}₂; —OP(O)(OR^{◯})₂; —SiR^{◯}₃; —(C₁₋₄ straight or branched alkylene)O—N(R^{◯})₂; or —(C₁₋₄ straight or branched alkylene)C(O)O—N(R^{◯})₂, wherein each R^{◯} may be substituted as defined below and is independently hydrogen, C₁₋₆ aliphatic, —CH₂Ph, —O(CH₂)₀₋₁Ph, —CH₂-(5- to 6-membered heteroaryl ring), or a 3- to 6-membered saturated, partially unsaturated, or aryl ring having 0-4 heteroatoms independently selected from nitrogen, oxygen, or sulfur, or, notwithstanding the definition above, two independent occurrences of R^{◯}, taken together with their intervening atom(s), form a 3- to 12-membered saturated, partially unsaturated, or aryl mono- or bicyclic ring having 0-4 heteroatoms independently selected from nitrogen, oxygen, or sulfur, which may be substituted as defined below.

Suitable monovalent substituents on R^{◯} (or the ring formed by taking two independent occurrences of R^{◯} together with their intervening atoms), are independently halogen, —(CH₂)₀₋₂R^{•}, -(haloR^{•}), —(CH₂)₀₋₂OH, —(CH₂)₀₋₂OR^{•}, —(CH₂)₀₋₂CH(OR^{•})₂, —O(haloR^{•}), —CN, —N₃, —(CH₂)₀₋₂C(O)R^{•}, —(CH₂)₀₋₂C(O)OH, —(CH₂)₀₋₂C(O)OR^{•}, —(CH₂)₀₋₂SR^{•}, —(CH₂)₀₋₂SH, —(CH₂)₀₋₂NH₂, —(CH₂)₀₋₂NHR^{•}, —(CH₂)₀₋₂NR^{•}₂, —NO₂, —SiR^{•}₃, —OSiR^{•}₃, —C(O)SR^{•}, —(C₁₋₄ straight or branched alkylene)C(O)OR^{•}, or —SSR^{•} wherein each R^{•} is unsubstituted or where preceded by “halo” is substituted only with one or more halogens, and is independently selected from C₁₋₄ aliphatic, —CH₂Ph, —O(CH₂)₀₋₁Ph, or a 3- to 6-membered saturated, partially unsaturated, or aryl ring having 0-4 heteroatoms independently selected from nitrogen, oxygen, or sulfur. Suitable divalent substituents on a saturated carbon atom of R^{◯} include ═O and ═S.

Suitable divalent substituents on a saturated carbon atom of an “optionally substituted” group include the following: =0 (“oxo”), ═S, ═NNR*₂, ═NNHC(O)R*, ═NNHC(O)OR*, ═NNHS(O)₂R*, ═NR*, ═NOR*, —O(C(R*₂))₂₋₃O—, or —S(C(R*₂))₂₋₃S—, wherein each independent occurrence of R* is selected from hydrogen, C₁₋₆ aliphatic which may be substituted as defined below, or an unsubstituted 5- to 6-membered saturated, partially unsaturated, or aryl ring having 0-4 heteroatoms independently selected from nitrogen, oxygen, or sulfur. Suitable divalent substituents that are bound to vicinal substitutable carbons of an “optionally substituted” group include: —O(CR*₂)₂₋₃O—, wherein each independent occurrence of R* is selected from hydrogen, C₁₋₆ aliphatic which may be substituted as defined below, or an unsubstituted 5-6-membered saturated, partially unsaturated, or aryl ring having 0-4 heteroatoms independently selected from nitrogen, oxygen, or sulfur.

Suitable substituents on the aliphatic group of R* include halogen, —R^{•}, -(haloR^{•}), —OH, —OR^{•}, —O(haloR^{•}), —CN, —C(O)OH, —C(O)OR^{•}, —NH₂, —NHR^{•}, —NR^{•}₂, or —NO₂, wherein each R^{•} is unsubstituted or where preceded by “halo” is substituted only with one or more halogens, and is independently C₁₋₄ aliphatic, —CH₂Ph, —O(CH₂)₀₋₁Ph, or a 3- to 6-membered saturated, partially unsaturated, or aryl ring having 0-4 heteroatoms independently selected from nitrogen, oxygen, or sulfur.

Suitable substituents on a substitutable nitrogen of an “optionally substituted” group include —R^{†}, —NR^{†}₂, —C(O)R^{†}, —C(O)OR^{†}, —C(O)C(O)R^{†}, —C(O)CH₂C(O)R^{†}, —S(O)₂R^{†}, —S(O)₂NR^{†}₂, —C(S)NR^{†}₂, —C(NH)NR^{†}₂, or —N(R^{†})S(O)₂R^{†}; wherein each R^{†} is independently hydrogen, C₁₋₆ aliphatic which may be substituted as defined below, unsubstituted —OPh, or an unsubstituted 3- to 6-membered saturated, partially unsaturated, or aryl ring having 0-4 heteroatoms independently selected from nitrogen, oxygen, or sulfur, or, notwithstanding the definition above, two independent occurrences of R^{†}, taken together with their intervening atom(s) form an unsubstituted 3- to 12-membered saturated, partially unsaturated, or aryl mono- or bicyclic ring having 0-4 heteroatoms independently selected from nitrogen, oxygen, or sulfur.

Suitable substituents on the aliphatic group of R^{†} are independently halogen, —R^{•}, -(haloR^{•}), —OH, —OR^{•}, —O(haloR^{•}), —CN, —C(O)OH, —C(O)OR^{•}, —NH₂, —NHR^{•}, —NR^{•}₂, or —NO₂, wherein each R^{•} is unsubstituted or where preceded by “halo” is substituted only with one or more halogens, and is independently C₁₋₄ aliphatic, —CH₂Ph, —O(CH₂)₀₋₁Ph, or a 3- to 6-membered saturated, partially unsaturated, or aryl ring having 0-4 heteroatoms independently selected from nitrogen, oxygen, or sulfur.

Small molecule: As used herein, the term “small molecule” means a low molecular weight organic and/or inorganic compound. In general, a “small molecule” is a molecule that is less than about 5 kilodaltons (kD) in size. In some embodiments, a small molecule is less than about 4 kD, 3 kD, about 2 kD, or about 1 kD. In some embodiments, the small molecule is less than about 800 daltons (D), about 600 D, about 500 D, about 400 D, about 300 D, about 200 D, or about 100 D. In some embodiments, a small molecule is less than about 2000 g/mol, less than about 1500 g/mol, less than about 1000 g/mol, less than about 800 g/mol, or less than about 500 g/mol. In some embodiments, a small molecule is not a polymer.

In some embodiments, a small molecule does not include a polymeric moiety. In some embodiments, a small molecule is not and/or does not comprise a protein or polypeptide (e.g., is not an oligopeptide or peptide). In some embodiments, a small molecule is not and/or does not comprise a polynucleotide (e.g., is not an oligonucleotide). In some embodiments, a small molecule is not and/or does not comprise a polysaccharide; for example, in some embodiments, a small molecule is not a glycoprotein, proteoglycan, glycolipid, etc.). In some embodiments, a small molecule is not a lipid.

In some embodiments, a small molecule is a modulating agent (e.g., is an inhibiting agent or an activating agent). In some embodiments, a small molecule is biologically active. In some embodiments, a small molecule is detectable (e.g., comprises at least one detectable moiety). In some embodiments, a small molecule is a therapeutic agent.

Those of ordinary skill in the art, reading the present disclosure, will appreciate that certain small molecule compounds described herein may be provided and/or utilized in any of a variety of forms such as, for example, crystal forms (e.g., polymorphs, solvates, etc), salt forms, protected forms, pro-drug forms, ester forms, isomeric forms (e.g., optical and/or structural isomers), isotopic forms, etc.

Those of ordinary skill in the art will appreciate that certain small molecule compounds have structures that can exist in one or more steroisomeric forms. In some embodiments, such a small molecule may be utilized in accordance with the present disclosure in the form of an individual enantiomer, diastereomer or geometric isomer, or may be in the form of a mixture of stereoisomers; in some embodiments, such a small molecule may be utilized in accordance with the present disclosure in a racemic mixture form.

Those of skill in the art will appreciate that certain small molecule compounds have structures that can exist in one or more tautomeric forms. In some embodiments, such a small molecule may be utilized in accordance with the present disclosure in the form of an individual tautomer, or in a form that interconverts between tautomeric forms.

Those of skill in the art will appreciate that certain small molecule compounds have structures that permit isotopic substitution (e.g., ²H or ³H for H; ¹¹C, ¹³C or ¹⁴C for ¹²C; ¹³N or ¹⁵N for ¹⁴N; ¹⁷O or ¹⁸O for ¹⁶O; ³⁶Cl for ³⁵Cl or ³⁷Cl; ¹⁸F for ¹⁹F; ¹³¹I for ¹²⁷I; etc.). In some embodiments, such a small molecule may be utilized in accordance with the present disclosure in one or more isotopically modified forms, or mixtures thereof.

In some embodiments, reference to a particular small molecule compound may relate to a specific form of that compound. In some embodiments, a particular small molecule compound may be provided and/or utilized in a salt form (e.g., in an acid-addition or base-addition salt form, depending on the compound); in some such embodiments, the salt form may be a pharmaceutically acceptable salt form.

In some embodiments, where a small molecule compound is one that exists or is found in nature, that compound may be provided and/or utilized in accordance in the present disclosure in a form different from that in which it exists or is found in nature. Those of ordinary skill in the art will appreciate that, in some embodiments, a preparation of a particular small molecule compound that contains an absolute or relative amount of the compound, or of a particular form thereof, that is different from the absolute or relative (with respect to another component of the preparation including, for example, another form of the compound) amount of the compound or form that is present in a reference preparation of interest (e.g., in a primary sample from a source of interest such as a biological or environmental source) is distinct from the compound as it exists in the reference preparation or source. Thus, in some embodiments, for example, a preparation of a single stereoisomer of a small molecule compound may be considered to be a different form of the compound than a racemic mixture of the compound; a particular salt of a small molecule compound may be considered to be a different form from another salt form of the compound; a preparation that contains only a form of the compound that contains one conformational isomer ((Z) or (E)) of a double bond may be considered to be a different form of the compound from one that contains the other conformational isomer ((E) or (Z)) of the double bond; a preparation in which one or more atoms is a different isotope than is present in a reference preparation may be considered to be a different form; etc.

Those skilled in the art will further appreciate that, in small molecule structures, the symbol ~, as used herein, refers to a point of attachment between two atoms. Additionally or alternatively, the symbol ~ refers to a point of attachment ring in a spirocyclic manner.

Treat: As used herein, the terms “treat,” “treatment,” or “treating” refer to any method used to partially or completely alleviate, ameliorate, relieve, inhibit, prevent, delay onset of, reduce severity of, and/or reduce incidence of one or more symptoms or features of a disease, disorder, and/or condition. Treatment may be administered to a subject who does not exhibit signs of a disease, disorder, and/or condition. In some embodiments, treatment may be administered to a subject who exhibits only early signs of the disease, disorder, and/or condition, for example, for the purpose of decreasing the risk of developing pathology associated with the disease, disorder, and/or condition.

### Estrogen Receptor Modulators

In some embodiments, the present disclosure provides compounds that are estrogen receptor modulators. In some embodiments, provided compounds are estrogen receptor agonists. As used herein, an “estrogen receptor agonist” refers to a compound or composition that produces an agonistic effect when contacting the estrogen receptor of a subject or biological sample. In some embodiments, an estrogen receptor agonist is characterized by having (i) at least 80% increase in the E2-normalized signal in the AP assay (agonist mode) of Example 209 and (ii) no more than 80% reduction in the E2-normalized signal in the AP assay (antagonist mode) of Example 209. In some embodiments, provided compounds are estrogen receptor antagonists. As used herein, an “estrogen receptor antagonist” refers to a compound or composition that produces an antagonistic effect when contacting the estrogen receptor of a subject or biological sample. In some embodiments, an estrogen receptor antagonist is characterized by having:
1. (i) between 10% and 80% increase in the E2-normalized signal in the AP assay (agonist mode) of Example 209 and (ii) between 10% and 80% reduction in the E2-normalized signal in the AP assay (antagonist mode) of Example 209;
2. (i) a pIC₅₀ greater than 6.4 and at least a 10% reduction in the E2-normalized signal in the AP assay (antagonist mode) of Example 209; and (ii) no more than 10% increase in E2-normalized signal in the AP assay (agonist mode) of Example 209; or
3. (i) a pIC₅₀ greater than 7.5 and at least a 10% reduction in the E2-normalized signal in the AP assay (antagonist mode) of Example 209; and (ii) no more than 10% increase in E2-normalized signal in the AP assay (agonist mode) of Example 209.

In some embodiments, the present disclosure provides compounds that are complete estrogen receptor (ER) antagonists. As used herein, a “complete estrogen receptor antagonist” refers to a compound or composition that produces an antagonistic effect when contacting the estrogen receptor of a subject or biological sample, with minimal agonistic effect (e.g., with no or substantially no agonistic effect). Complete estrogen receptor antagonism is determined according to methods described herein, for example in Example 209. In some embodiments, a complete estrogen receptor antagonist is characterized by having (i) a pIC₅₀ greater than 6.4 and at least a 10% reduction in the E2-normalized signal in the AP assay (antagonist mode) of Example 209; and (ii) no more than 10% increase in E2-normalized signal in the AP assay (agonist mode) of Example 209. In some embodiments, a complete estrogen receptor antagonist is characterized by having (i) a pIC₅₀ greater than 7.5 and at least a 10% reduction in the E2-normalized signal in the AP assay (antagonist mode) of Example 209; and (ii) no more than 10% increase in E2-normalized signal in the AP assay (agonist mode) of Example 209. In some embodiments, a complete estrogen receptor antagonist is an agent (e.g., a small molecule compound) that shows ER antagonism and no or substantially no ER agonism in one or more of ERα protein level assays, MCF-7 cell line assays, Ishikawa cell line assays (measuring wild type ER and certain mutants including mutants lacking AF1 and/or AF2 domains), and rodent uterine weight gain assays. See, generally, WO 2017/059139. Alternatively or additionally, in some embodiments, a complete estrogen receptor antagonist has three characteristics: it (1) inhibits both activating function 1 (AF1) and activating function 2 (AF2), as complete anti-estrogen activity requires inactivation of both AF1 and AF2; (2) promotes ER degradation; and (3) lacks the partial ER agonist activity observed with certain other agents. Without being bound by theory, it is understood that complete inhibition of both AF1 and AF2 is required for complete estrogen receptor activity, activating mutations in the gene that codes for estrogen receptor 1 allows for activation of both AF1 and AF2 even in the absence of estrogen.

In some embodiments, the present disclosure provides a compound of Formula I:

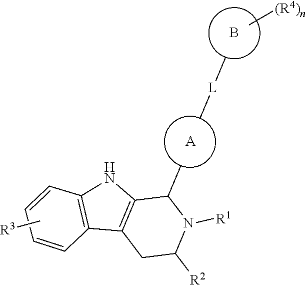

or a pharmaceutically acceptable salt thereof, wherein:
A is an optionally substituted 7- to 10-membered bicyclic heteroaryl comprising 1-4 heteroatoms selected from N, O, and S or an optionally substituted 3- to 6-membered heterocyclyl comprising 1-3 heteroatoms selected from N, O, and S;
L is a covalent bond or an optionally substituted bivalent group selected from —C₁-C₆ aliphatic-, -L^{a}-C₀-C₅ aliphatic-, and —C₁-C₅ aliphatic-L^{a}-, wherein L^{a} is selected from —S—, —SO—, —SO₂—, and —N(R^{a})—;
B is selected from —OH, —CO₂H, C₁-C₆ aliphatic, 3- to 12-membered heterocyclyl comprising 1-3 heteroatoms selected from N, O, and S, and C₃-C₆ cycloaliphatic;
R¹ is selected from hydrogen and optionally substituted C₁-C₆ aliphatic;
R² is selected from hydrogen and optionally substituted C₁-C₆ aliphatic;
R³ is selected from hydrogen, halogen, —CN, —OR^{a}, —C(O)R^{a}, —C(O)₂R^{a}, —OC(O)R^{a}, —C(O)N(R^{a})₂, —OC(O)N(R^{a})₂, —NO₂, —N(R^{a})₂, —N(R^{a})C(O)R^{a}, —N(R^{a})C(O)₂R^{a}, —N(R^{a})S(O)₂R^{a}, —SR^{a}, —S(O)₂R^{a}, —S(O)N(R^{a})₂, —S(O)₂N(R^{a})₂, and an optionally substituted C₁₋₆ aliphatic group;
each R⁴ is independently oxo, halogen, —CN, —OR^{a}, —N(R^{a})₂, —C(O)R^{a}, —OC(O)R^{a}, —C(O)₂R^{a}, —C(O)N(R^{a})₂, —N(R^{a})C(O)R^{a}, or an optionally substituted group selected from C₁-C₆ aliphatic and 3- to 12-membered heterocyclyl comprising 1 to 3 heteroatoms selected from N, O, and S;
each R^{a} is independently selected from hydrogen and optionally substituted C₁-C₆ aliphatic; and
n is 0 to 5.

As defined generally above, A is an optionally substituted 7- to 10-membered bicyclic heteroaryl comprising 1-4 heteroatoms selected from N, O, and S or an optionally substituted 3- to 6-membered heterocyclyl comprising 1-3 heteroatoms selected from N, O, and S. In some embodiments, A is 7- to 10-membered bicyclic heteroaryl comprising 1-4 heteroatoms selected from N, O, and S or 3- to 6-membered heterocyclyl comprising 1-3 heteroatoms selected from N, O, and S, each optionally substituted with halogen, —(CH₂)₀₋₄R^{◯}, or —(CH₂)₀₋₄OR^{◯}.

In some embodiments, A is optionally substituted 7- to 10-membered bicyclic heteroaryl comprising 1-4 heteroatoms selected from N, O, and S. In some embodiments, A is 7- to 10-membered bicyclic heteroaryl comprising 1-4 heteroatoms selected from N, O, and S optionally substituted with halogen, —(CH₂)₀₋₄R^{◯}, or —(CH₂)₀₋₄OR^{◯}. In some embodiments, A is 7- to 10-membered bicyclic heteroaryl comprising 1-4 heteroatoms selected from N, O, and S, optionally substituted with halogen or C₁-C₆ aliphatic. In some embodiments, A is optionally substituted 7-membered bicyclic heteroaryl comprising 1-4 heteroatoms selected from N, O, and S. In some embodiments, A is optionally substituted 8-membered bicyclic heteroaryl comprising 1-4 heteroatoms selected from N, O, and S. In some embodiments, A is optionally substituted 9-membered bicyclic heteroaryl comprising 1-4 heteroatoms selected from N, O, and S. In some embodiments, A is optionally substituted 10-membered bicyclic heteroaryl comprising 1-4 heteroatoms selected from N, O, and S.

In some embodiments, A is

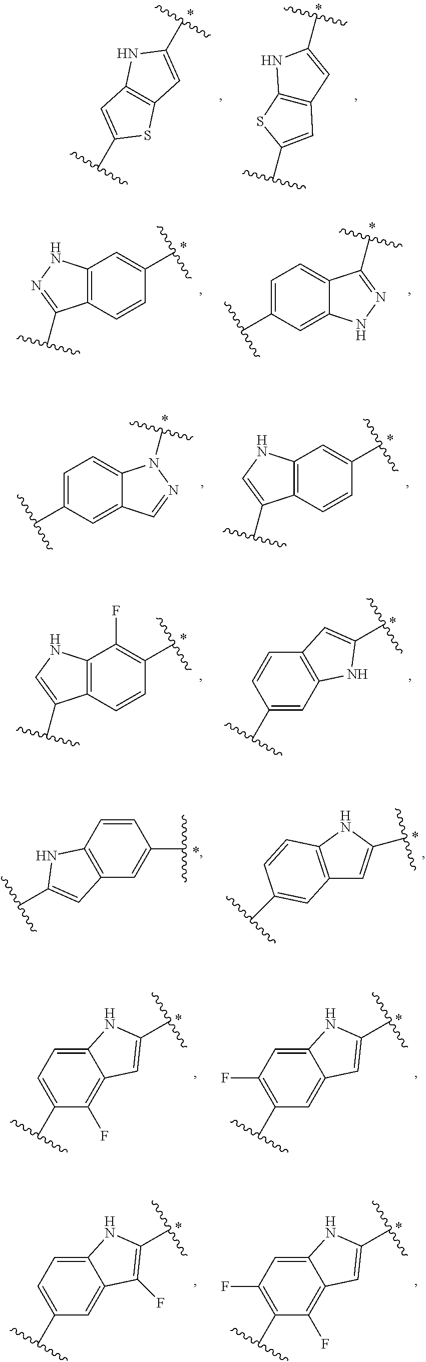

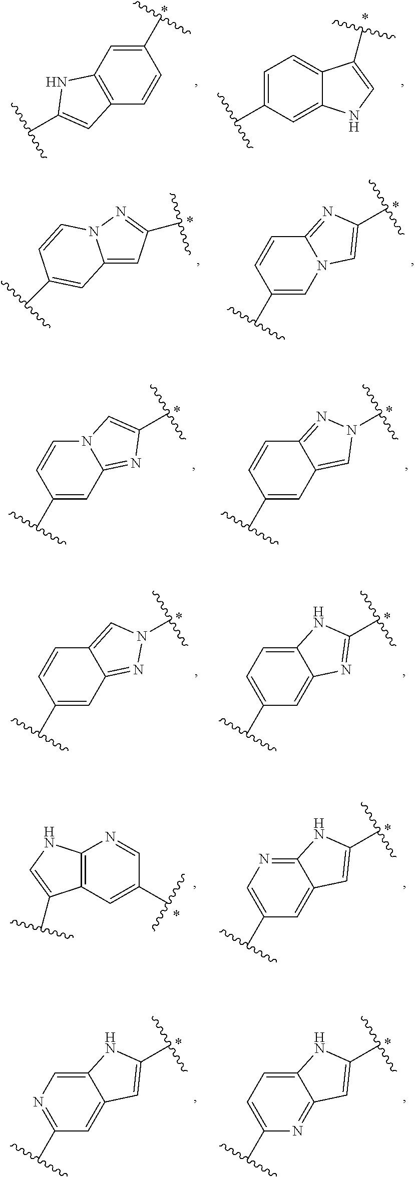

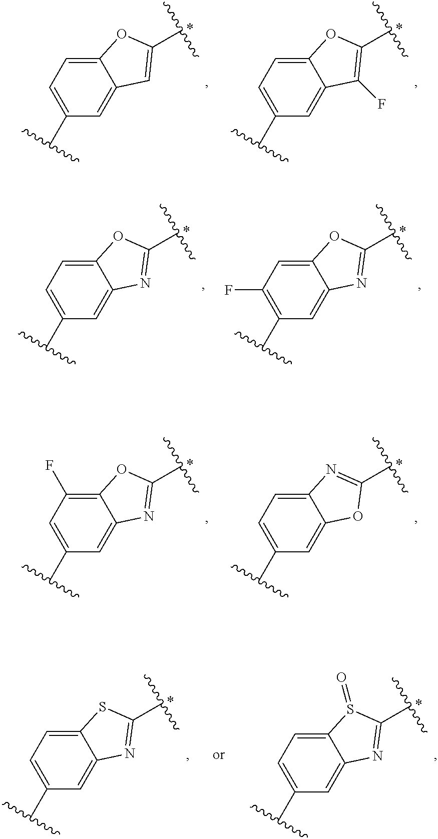

wherein * represents a point of attachment to moiety L.

In some embodiments, A is

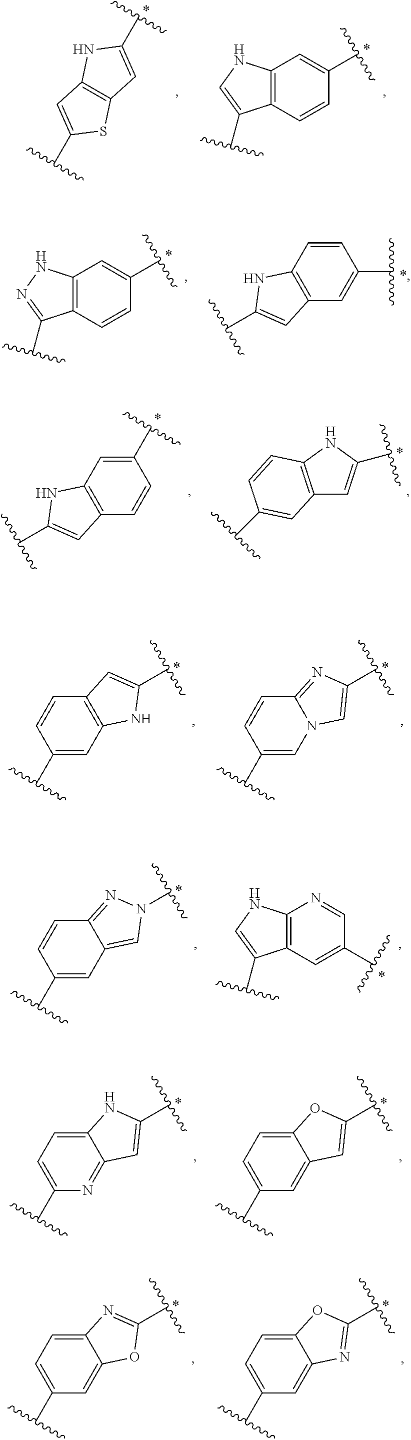

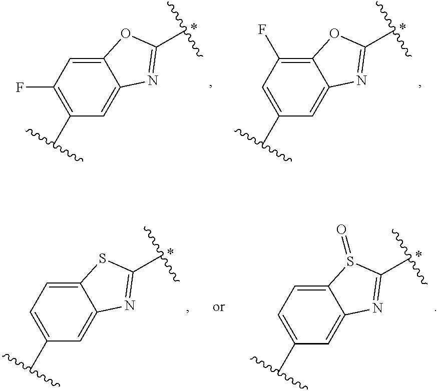 [SMILES: *c1nc2cc(*)c(F)cc2o1 ; *c1cc(F)c2oc(*)nc2c1 ; *c1ccc2sc(*)nc2c1 ; *C1=Nc2cc(*)ccc2S1=O]

In some embodiments, A is

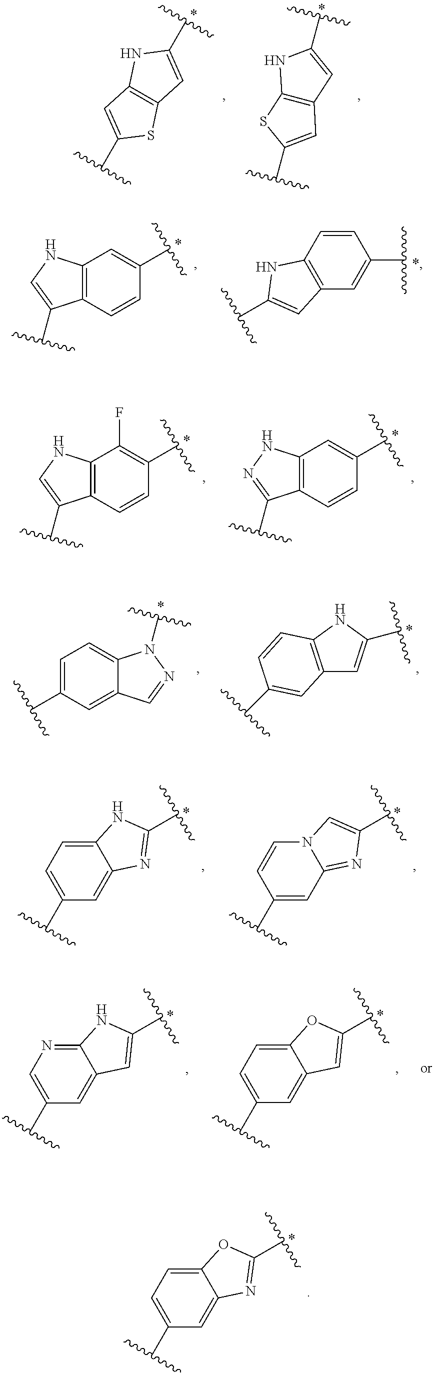

In some embodiments, A is

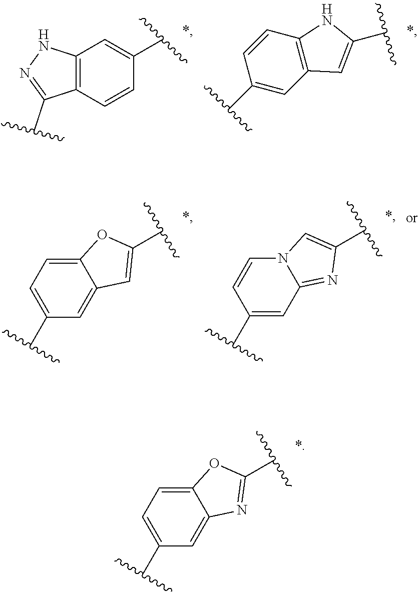 [SMILES: *c1ccc2c(*)n[nH]c2c1 ; *c1ccc2[nH]c(*)cc2c1 ; *c1ccc2oc(*)cc2c1 ; *c1ccn2cc(*)nc2c1 ; *c1ccc2oc(*)nc2c1]

In some embodiments, A is optionally substituted 3- to 6-membered heterocyclyl comprising 1-3 heteroatoms selected from N, O, and S. In some embodiments, A is optionally substituted 4- to 6-membered heterocyclyl comprising 1-3 heteroatoms selected from N, O, and S. In some embodiments, A is 3- to 6-membered heterocyclyl comprising 1-3 heteroatoms selected from N, O, and S optionally substituted with halogen, —(CH₂)₀₋₄R^{◯}, or —(CH₂)₀₋₄OR^{◯}. In some embodiments, A is 3- to 6-membered heterocyclyl comprising 1-3 heteroatoms selected from N, O, and S comprising 1-4 heteroatoms selected from N, O, and S, optionally substituted with halogen or C₁-C₆ aliphatic.

In some embodiments, A is optionally substituted piperidinyl. In some embodiments, A is:

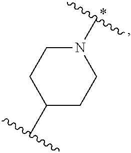 [SMILES: *C1CCN(*)CC1]

wherein * represents a point of attachment to moiety L.

As described generally above, L is a covalent bond or an optionally substituted bivalent group selected from C₁-C₆ aliphatic, -L^{a}-C₀-C₅ aliphatic-, and —C₁-C₅ aliphatic-L^{a}-, wherein L^{a} is selected from —S—, —SO—, —SO₂—, and —N(R^{a})—. It will be appreciated that the point of attachment of L is indicated by the direction in which its definition is written, such that the leftmost atom is attached to A and the rightmost atom is attached to B. For example, when L is —S—CH₂—, L is attached to A via the sulfur atom and to B via the carbon atom.

In some embodiments, L is a covalent bond.

In some embodiments, L is optionally substituted C₁-C₆ aliphatic. In some embodiments, L is —(CH₂)₁₋₆—. In some embodiments, L is —CH₂—, —CH₂—CH₂—, —CH₂—CH₂—, —CH₂—CH₂—CH₂—, —CH₂—CH₂—CH₂—CH₂—, —CH₂—CH₂—CH₂—CH₂—CH₂—, or —CH₂—CH₂—CH₂—CH₂—CH₂—CH₂—. In some embodiments, L is —CH₂—.

In some embodiments, L is optionally substituted -L^{a}-C₀-C₅ aliphatic. In some embodiments, L is —S—C₀-C₅ aliphatic, —SO—C₀-C₅ aliphatic, —SO₂—C₀-C₅ aliphatic, or —N(R^{a})—C₀-C₅ aliphatic. In some embodiments, L is —S—C₀-C₅ aliphatic. In some embodiments, L is —SO—C₀-C₅ aliphatic. In some embodiments, L is —SO₂—C₀-C₅ aliphatic. In some embodiments, L is —N(R^{a})—C₀-C₅ aliphatic. In some embodiments, L is L^{a}. In some embodiments, L is —S—, —SO—, —SO₂—, or —N(R^{a})—. In some embodiments, L is —S—. In some embodiments, L is —SO—. In some embodiments, L is —SO₂—. In some embodiments, L is —N(R^{a})—. In some embodiments, L is —N(H)—. In some embodiments, L is —N(CH₃)—.

In some embodiments, L is selected from a covalent bond, —CH₂—, —CH₂—CH₂—, —CH₂—CH₂—CH₂—, —S—, and —SO₂—. In some embodiments, L is selected from a covalent bond, —CH₂—, —CH₂—CH₂—, —CH₂—CH₂—CH₂—, —S—, and —N(H)—.

As described generally above, B is selected from —OH, —CO₂H, C₁-C₆ aliphatic, 3- to 12-membered heterocyclyl comprising 1-3 heteroatoms selected from N, O, and S, and C₃-C₆ cycloaliphatic. In some embodiments, when B is —OH or —CO₂H, n is 0.

In some embodiments, B is selected from 3- to 12-membered heterocyclyl comprising 1-3 heteroatoms selected from N, O, and S and C₃-C₆ cycloaliphatic. In some embodiments, B is selected from —OH, —CO₂H, C₁-C₆ aliphatic, and 3- to 12-membered heterocyclyl comprising 1-3 heteroatoms selected from N, O, and S. In some embodiments, B is selected from —CO₂H and 3- to 12-membered heterocyclyl comprising 1-3 heteroatoms selected from N, O, and S.

In some embodiments, B is —OH.

In some embodiments, B is —CO₂H.

In some embodiments, B is C₁-C₆ aliphatic. In some embodiments, B is C₁-C₆ alkyl.

In some embodiments, B is 3- to 12-membered heterocyclyl comprising 1-3 heteroatoms selected from N, O, and S. In some embodiments, B is a 4- to 7-membered heterocyclyl comprising 1-3 heteroatoms selected from N, O, and S. In some embodiments, B is a 4- or 7-membered heterocyclyl comprising 1-3 heteroatoms selected from N, O, and S.

In some embodiments, B is 3- to 6-membered heterocyclyl comprising 1-3 heteroatoms selected from N, O, and S. In some embodiments, B is 3-membered heterocyclyl comprising 1 heteroatom selected from N, O, and S. In some embodiments, B is 4-membered heterocyclyl comprising 1-2 heteroatoms selected from N, O, and S. In some embodiments, B is 5-membered heterocyclyl comprising 1-3 heteroatoms selected from N, O, and S. In some embodiments, B is 6-membered heterocyclyl comprising 1-3 heteroatoms selected from N, O, and S. In some embodiments, B is azetidinyl, pyrrolidinyl, piperidinyl, piperazinyl, or morpholinyl. In some embodiments, B is azetidinyl or pyrrolidinyl. In some embodiments, B is azetidinyl. In some embodiments, B is pyrrolidinyl. In some embodiments, B is selected from:

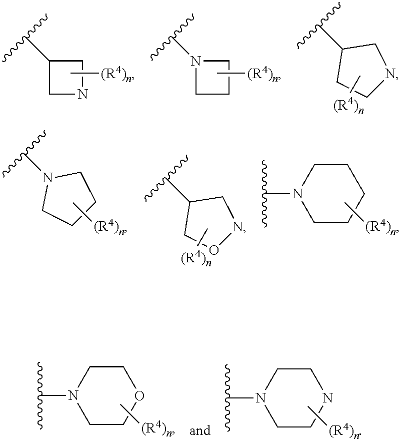

In some embodiments, B is selected from:

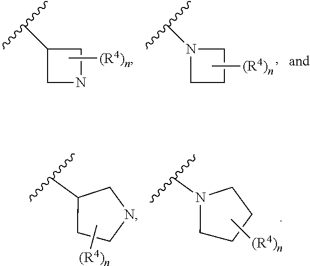

In some embodiments, B is:

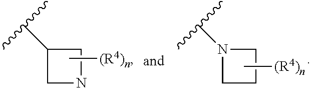

In come embodiments, B is:

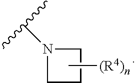

In some embodiments, B is 6- to 12-membered bicyclic fused or spirocyclic heterocyclyl. In some embodiments, B is 6- to 8-membered bicyclic fused or spirocyclic heterocyclyl. In some embodiments, B is 6- to 12-membered bicyclic fused heterocyclyl. In some embodiments, B is 6- to 8-membered bicyclic fused heterocyclyl. In some embodiments, B is 6- to 12-membered bicyclic spirocyclic heterocyclyl. In some embodiments, B is 6- to 8-membered bicyclic spirocyclic heterocyclyl. In some embodiments, B is selected from:

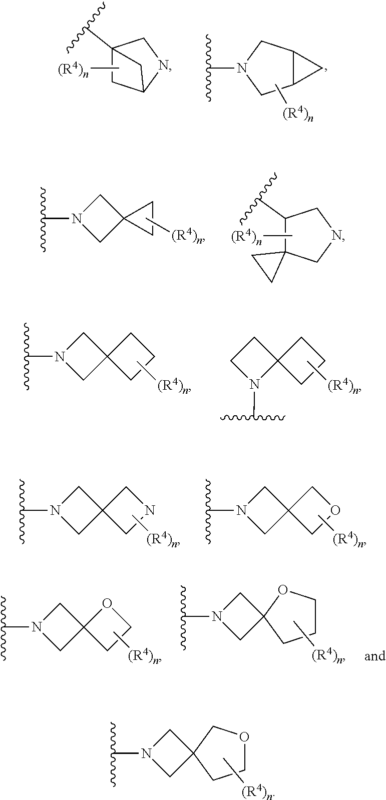

In some embodiments B is selected from:

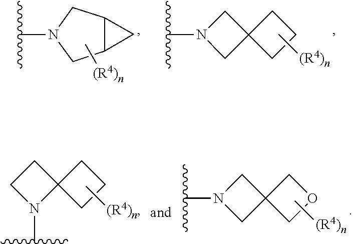

In some embodiments, B is selected from:

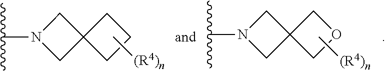

In some embodiments, B is:

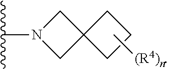

In some embodiments, B is C₃-C₆ cycloaliphatic. In some embodiments, B is cyclopropyl, cyclobutyl, cyclopentyl, or cyclohexyl.

As described generally above, each R⁴ is independently oxo, halogen, —CN, —OR^{a}, —N(R^{a})₂, —C(O)R^{a}, —OC(O)R^{a}, —C(O)OR^{a}, —C(O)N(R^{a})₂, —N(R^{a})C(O)R^{a}, or an optionally substituted group selected from C₁-C₆ aliphatic and 3- to 12-membered heterocyclyl comprising 1 to 3 heteroatoms selected from N, O, and S.

In some embodiments, R⁴ is oxo.

In some embodiments, R⁴ is halogen. In some embodiments, R⁴ is flouro.

In some embodiments, R⁴ is —CN.

In some embodiments, R⁴ is —OR^{a}. In some embodiments, R⁴ is —OH. In some embodiments, R⁴ is —O—C₁-C₆ aliphatic (e.g., —OCH₃).

In some embodiments, R⁴ is —N(R^{a})₂. In some embodiments, R⁴ is —N(H)(R^{a}). In some embodiments, R⁴ is —NH₂. In some embodiments, R⁴ is —N(H)C₁-C₆ aliphatic. In some embodiments, R⁴ is —N(C₁-C₆ aliphatic)₂. In some embodiments, R⁴ is —N(H)CH₃, —N(H)CH₂CH₃, —N(CH₂CH₃)₂, —N(H)CH₂CH₂CH₃, —N(CH₃)₂, —N(CH₃)CH₂CH₃, or —N(CH₃)CH₂CH₂CH₃.

In some embodiments, R⁴ is —C(O)R^{a}. In some embodiments, R⁴ is —C(O)—C₁-C₆ aliphatic optionally substituted with —(CH₂)₀₋₄OR^{◯}. In some embodiments, R⁴ is —C(O)CH₃, —C(O)CH₂OCH₃, or —C(O)CH₂CH₂OCH₃.

In some embodiments, R⁴ is —OC(O)R^{a}. In some embodiments, R⁴ is —OC(O)—C₁-C₆ aliphatic.

In some embodiments, R⁴ is —C(O)₂R^{a}. In some embodiments, R⁴ is —C(O)OH. In some embodiments, R⁴ is —C(O)₂—C₁-C₆ aliphatic.

In some embodiments, R⁴ is —C(O)N(R^{a})₂. In some embodiments, R⁴ is —C(O)N(H)R^{a}. In some embodiments, R⁴ is —C(O)NH₂. In some embodiments, R⁴ is —C(O)N(H)C₁-C₆ aliphatic.

In some embodiments, R⁴ is —N(R^{a})C(O)R^{a}. In some embodiments, R⁴ is —N(H)C(O)R^{a}. In some embodiments, R⁴ is —N(H)C(O)C₁-C₆ aliphatic optionally substituted with —(CH₂)₀₋₄OR^{◯}. In some embodiments, R⁴ is —N(H)C(O)CH₂OH or —N(H)C(O)CH₂OCH₃.

In some embodiments, R⁴ is an optionally substituted C₁-C₆ aliphatic. In some embodiments, R⁴ is C₁-C₆ aliphatic optionally substituted with halogen, —(CH₂)₀₋₄R^{◯}, —(CH₂)₀₋₄OR^{◯}, —O(CH₂)₀₋₄R^{◯}, —CN, —(CH₂)₀₋₄N(R^{◯})₂, or phenyl. In some embodiments, R⁴ is C₁-C₆ aliphatic substituted with halogen, —OH, —OCH₃, —CN, or 3- to 6-membered saturated, partially unsaturated, or aryl ring having 0-4 heteroatoms independently selected from nitrogen, oxygen, or sulfur. In some embodiments, R⁴ is C₁-C₆ aliphatic optionally substituted with halogen. In some embodiments, R⁴ is C₁-C₃ alkyl optionally substituted with halogen. In some embodiments, R⁴ is methyl, ethyl, propyl, butyl, pentyl, hexyl, —CH₂F, —CHF₂, —CH₂CHF₂, —CH₂CH₂CHF₂, —CH₂CH₂CF₃, —CH₂OCH₃, —CH₂CH₂CH₂OH, —CH₂CH₂CH₂F, —CH₂CH₂CH(CH₃)₂, —CH(CH₃)₂, —CH₂CH₂Ph, —C(CH₃)₂—OH, —CH₂CH₂OCH₃, —CH₂CH₂OCH₂CH₃, —CH₂CH₂CH₂OCH₂CH₃, —CH₂C≡CH, —CH₂C≡CHCH₃, —CH₂CN, —CH₂CH₂CN, —CH₂CH₂CH₂OCF₃,

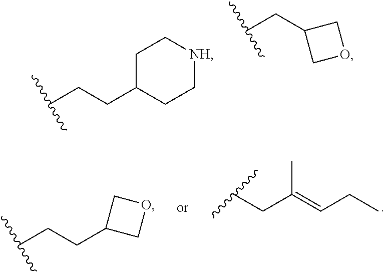

In some embodiments, R⁴ is methyl, ethyl, propyl, —CH₂F, —CHF₂, —CH₂OCH₃, —C(CH₃)₂—OH, —CH(CH₃)₂, and —CH₂CH₂CH₂F. In some embodiments, R⁴ is methyl, ethyl, propyl, —CH₂F, —CH₂CN, and —CH₂CH₂CH₂F. In some embodiments, R⁴ is methyl or —CH₂F.

In some embodiments, R⁴ is an optionally substituted 3- to 12-membered heterocyclyl comprising 1 to 3 heteroatoms selected from N, O, and S. In some embodiments, R⁴ is optionally substituted 3- to 6-membered heterocyclyl comprising 1 to 3 heteroatoms selected from N, O, and S. In some embodiments, R⁴ is optionally substituted azetidinyl.

In some embodiments, R⁴ is oxo, —OH, —OCH₃, fluoro, methyl, ethyl, propyl, isopropyl, butyl, pentyl, hexyl, —CN, —CH₂F, —CHF₂, —CH₂CHF₂, —CH₂CH₂CHF₂, —CH₂CH₂CF₃, —CH₂CH₂CH₂OH, —CH₂CH₂CH₂F, —CH₂CH₂CH(CH₃)₂, —CH₂CH₂-Ph, —C(CH₃)₂—OH, —CH₂OCH₃, —CH₂CH₂OCH₃, —CH₂CH₂OCH₂CH₃, —CH₂CH₂CH₂OCH₂CH₃, —CH₂C≡CH, —CH₂C≡CHCH₃, —CH₂CN, —CH₂CH₂CN, —CH₂CH₂CH₂OCF₃, —NHCH₂CH₃, —NHCH₂CH₂CH₃, —NHCH₂CH₂CH₃, —NHCH₃, —N(CH₃)CH₂CH₃, —N(CH₃)CH₂CH₂CH₃, —N(CH₃)₂, —NHC(O)CH₂OCH₃, —NHC(O)CH₂CH₂OCH₃, —NHC(O)CH₂OH, —C(O)CF₃,

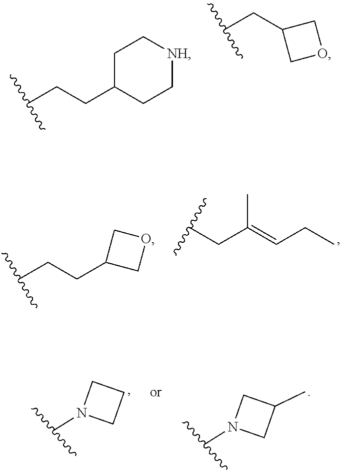

In some embodiments, each R⁴ is independently selected from fluoro, —CN, —OH, —OCH₃, methyl, ethyl, propyl, —CH₂F, —CH₂OCH₃, —C(CH₃)₂OH, —CH(CH₃)₂, and —CH₂CH₂CH₂F. In some embodiments, each R⁴ is independently selected from fluoro, —N(CH₃)₂, methyl, ethyl, propyl, —CH₂F, —CHF₂, —CH₂CN, and —CH₂CH₂CH₂F. In some embodiments, each R⁴ is independently selected from fluoro, methyl, and —CH₂F.

In some embodiments, a moiety:

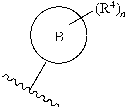

is a moiety selected from:

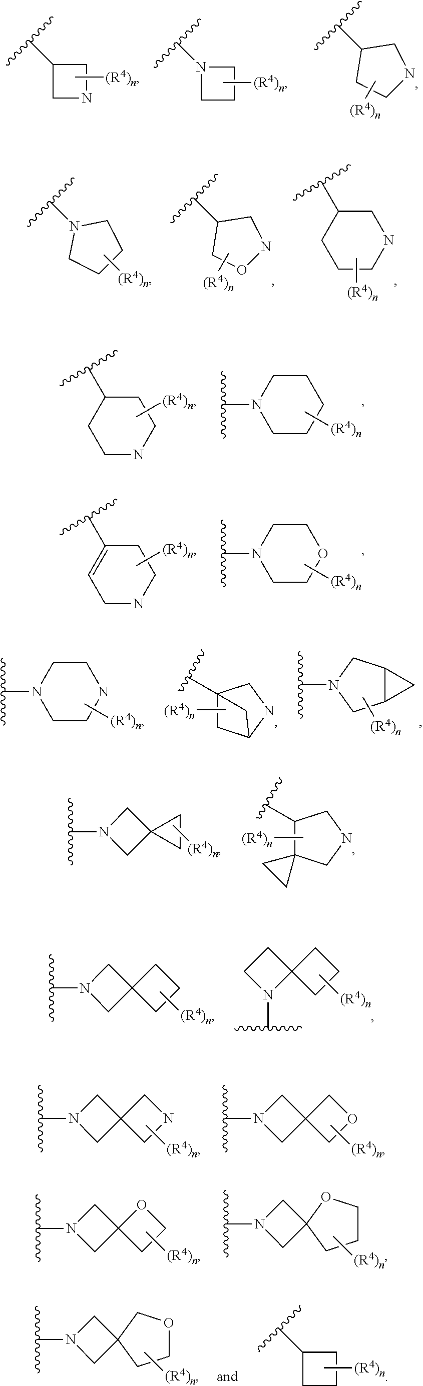

In some embodiments, a moiety:

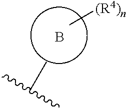

is a moiety selected from:

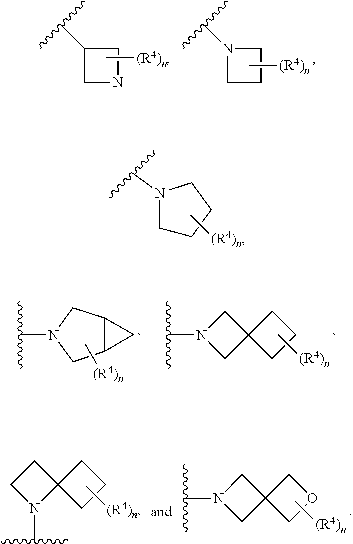

In some embodiments, a moiety:

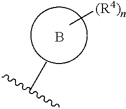

is a moiety selected from:

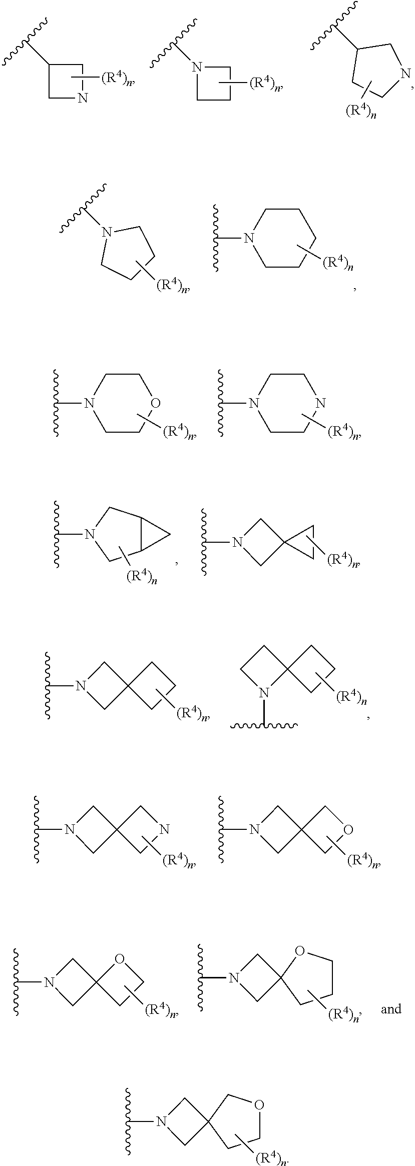

In some embodiments, a moiety:

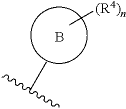

is a moiety selected from:

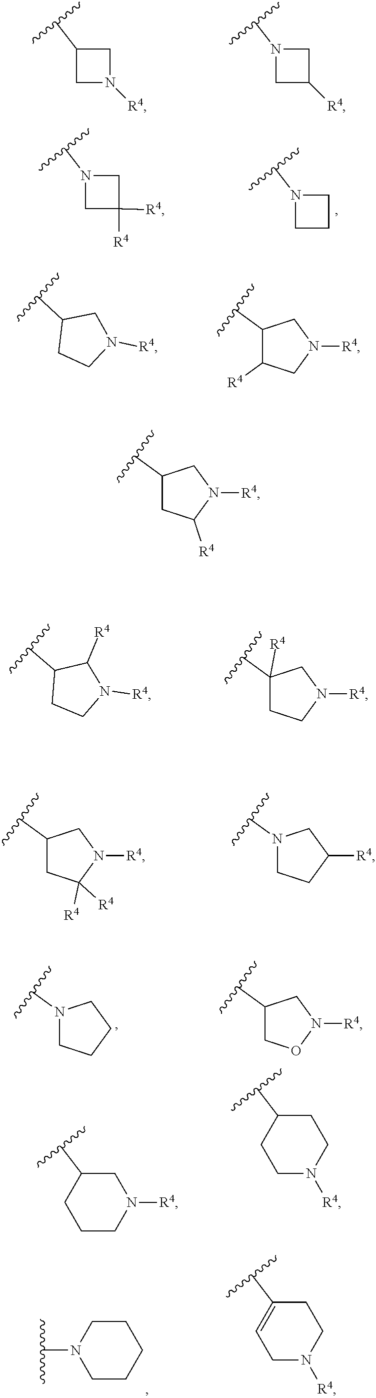

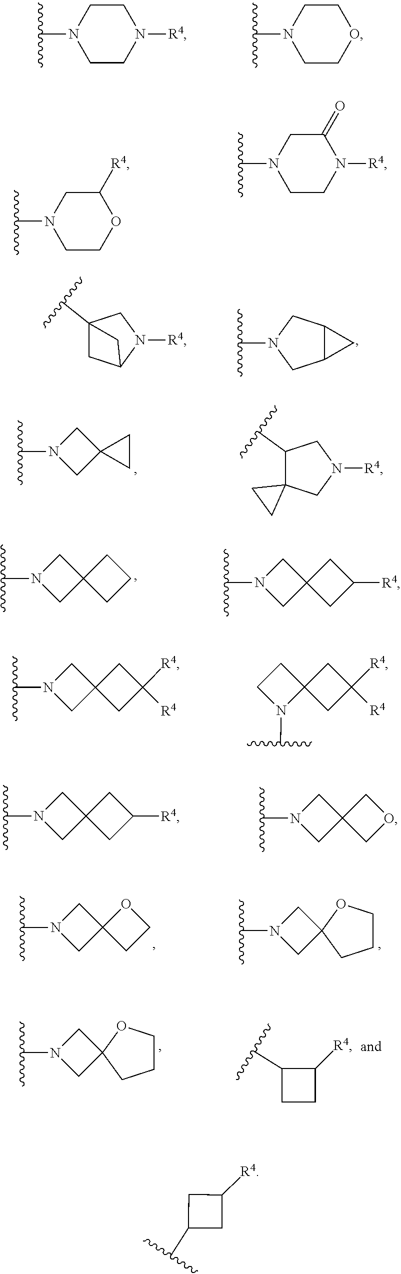

In some embodiments, a moiety:

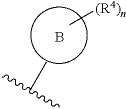

is a moiety selected from:

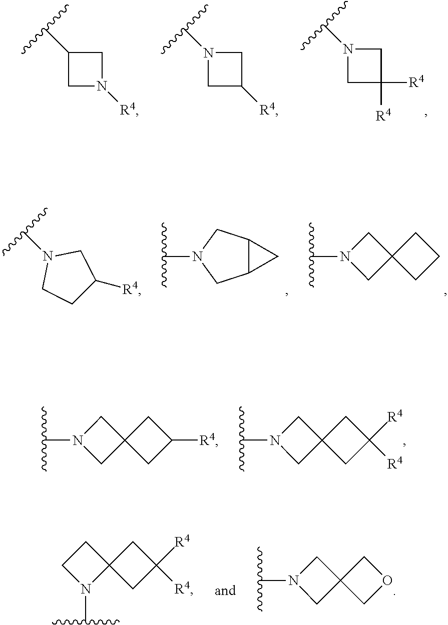

In some embodiments, a moiety:

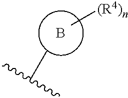

is a moiety selected from:

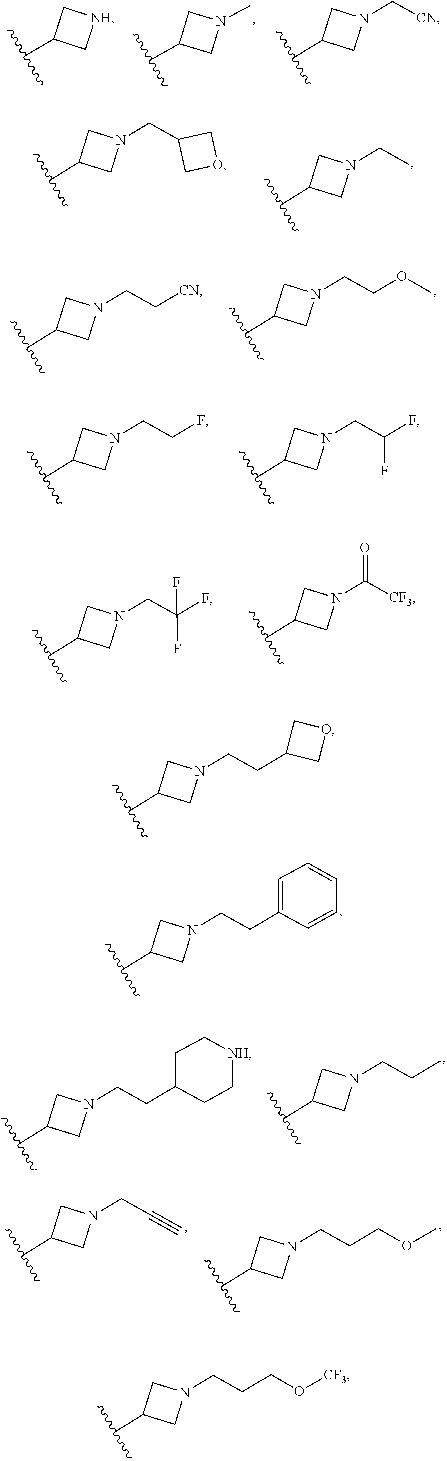

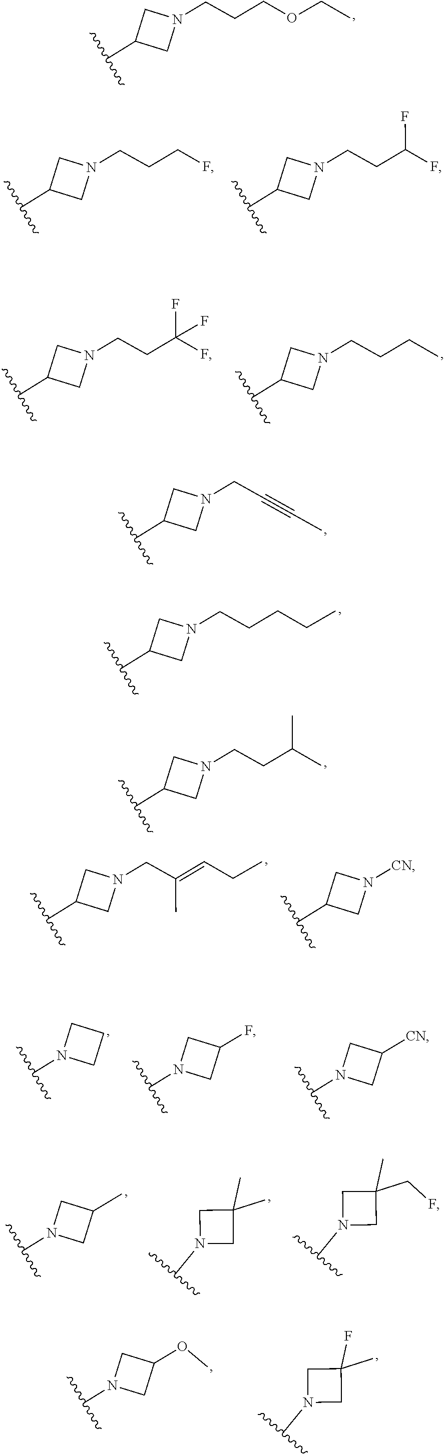

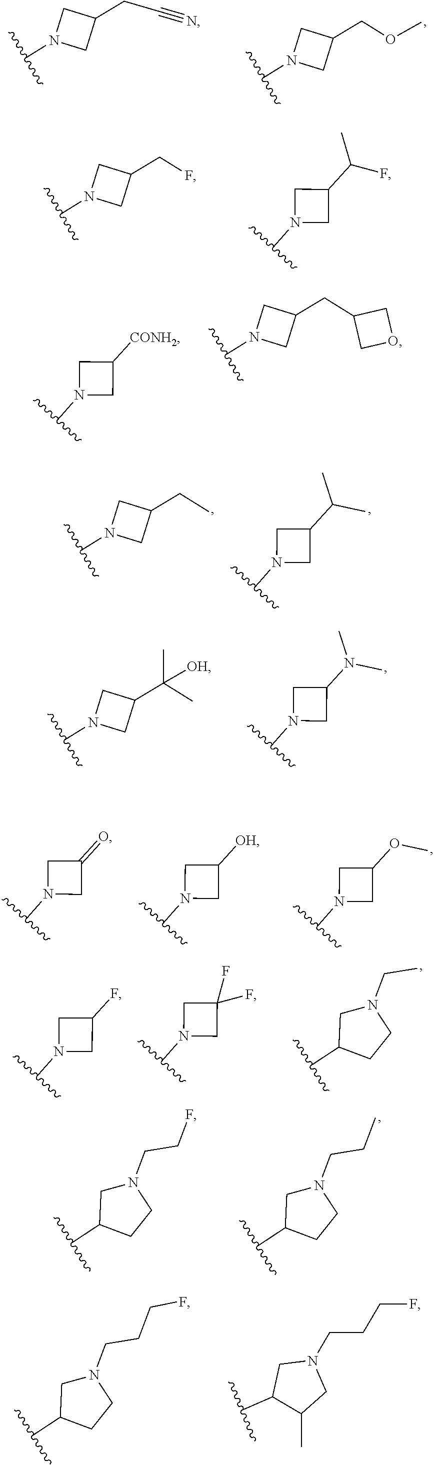

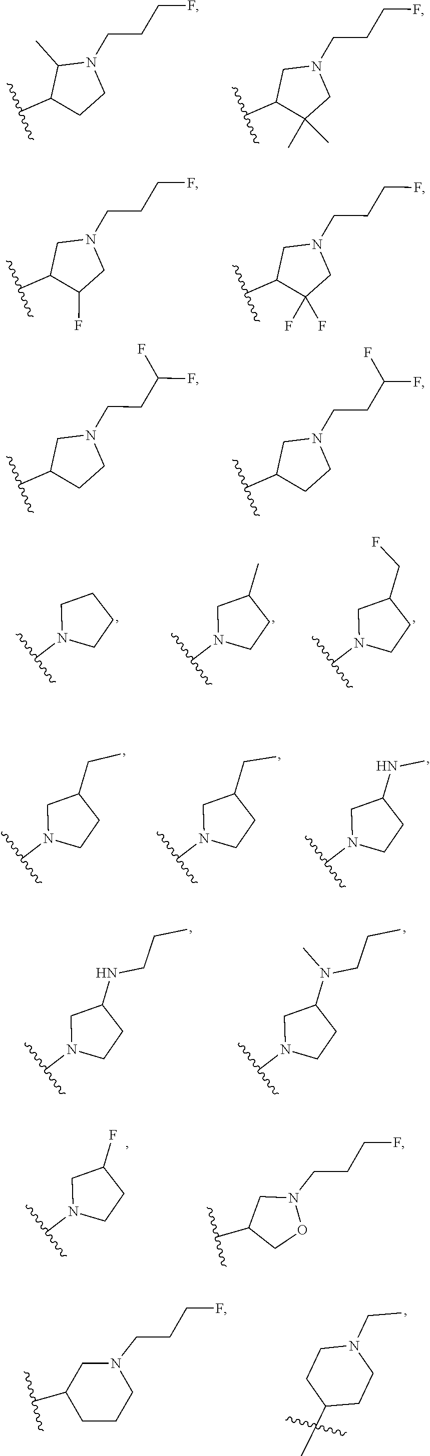

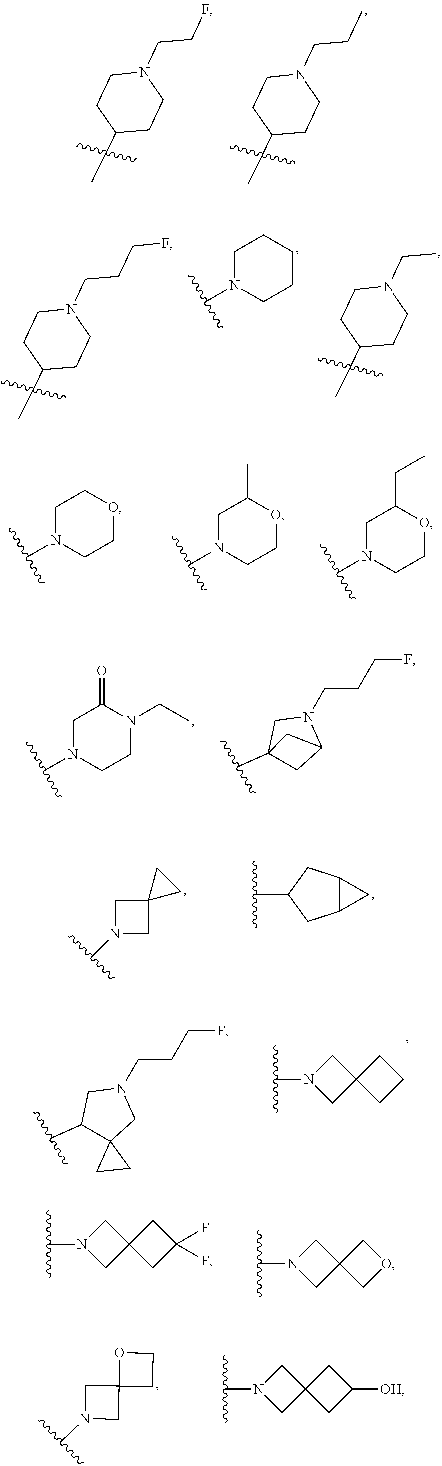

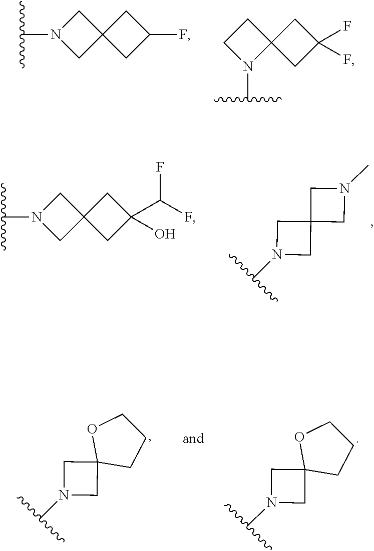

In some embodiments, a moiety:

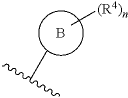

is a moiety selected from: —OH, —CO₂H, —CH₂CH₂CH₃, —CH₂CH₂CH₂F,

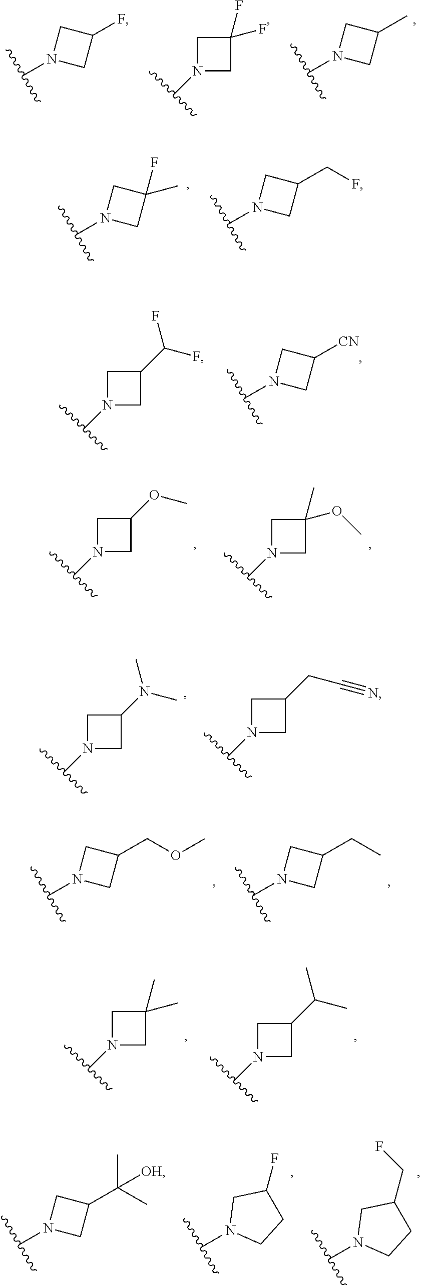

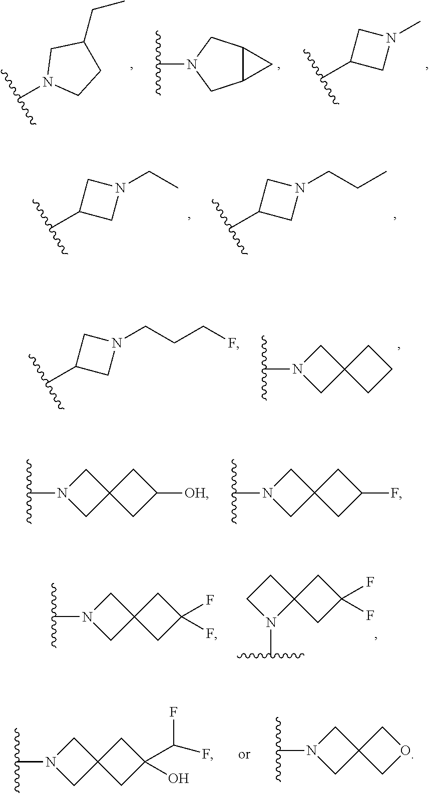

In some embodiments, a moiety:

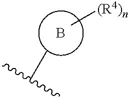

is a moiety selected from: —OH, —CO₂H, —CH₂CH₂CH₃, —CH₂CH₂CH₂F,

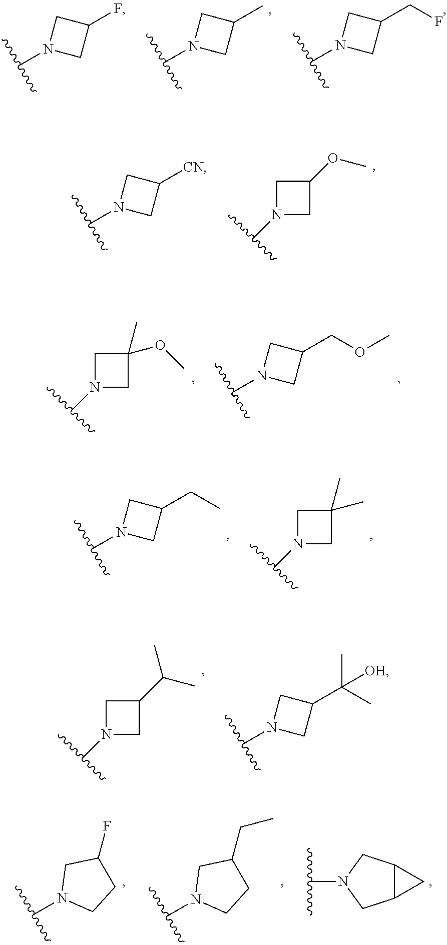

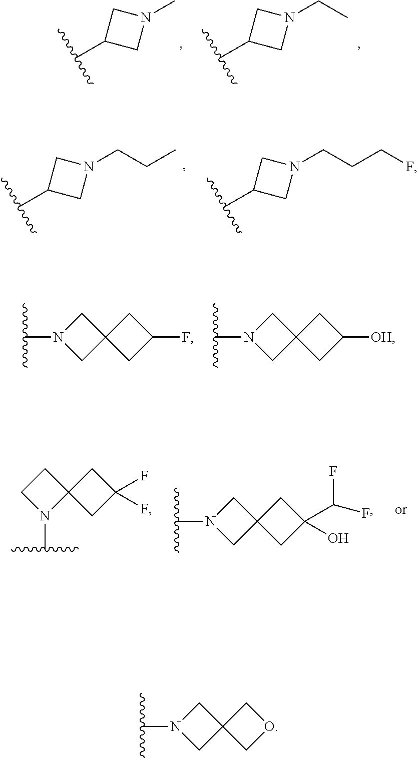

In some embodiments, a moiety:

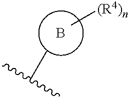

is a moiety selected from: —CO₂H,

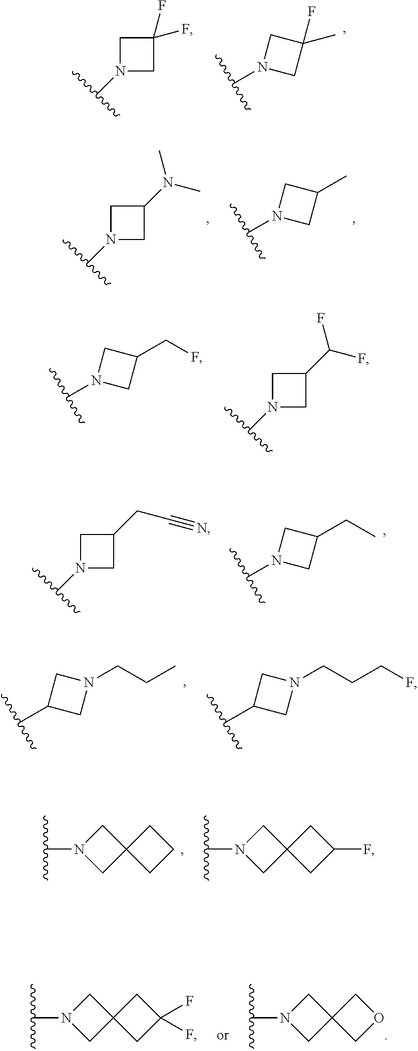

In some embodiments, a moiety:

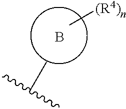

is a moiety selected from:

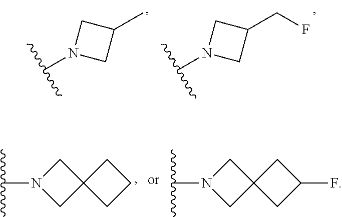

As described generally above, R¹ is selected from hydrogen and optionally substituted C₁-C₆ aliphatic. In some embodiments, R¹ is hydrogen. In some embodiments, R¹ is C₁-C₆ aliphatic optionally substituted with halogen, —(CH₂)₀₋₄R^{◯}, —(CH₂)₀₋₄OR^{◯}, or —(CH₂)₀₋₄Ph. In some embodiments, R¹ is C₁-C₆ aliphatic optionally substituted with halogen or —OH.

In some embodiments, R¹ is selected from:

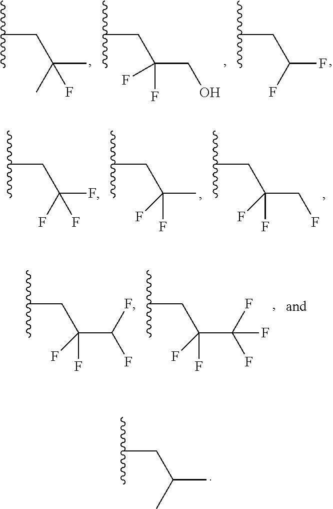

In some embodiments, R¹ is selected from:

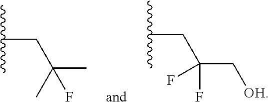

In some embodiments, R¹ is selected from:

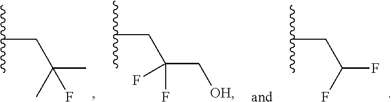

In some embodiments, R¹ is

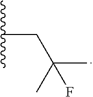 [SMILES: *CC(C)(C)F]

As described generally above, R² is selected from hydrogen and optionally substituted C₁-C₆ aliphatic. In some embodiments, R² is hydrogen. In some embodiments, R² is C₁-C₆ aliphatic. In some embodiments, R² is methyl, ethyl, propyl, butyl, pentyl, or hexyl. In some embodiments, R² is methyl.

As described generally above, R³ is selected from hydrogen, halogen, —CN, —OR^{a}, —C(O)R^{a}, —C(O)₂R^{a}, —OC(O)R^{a}, —C(O)N(R^{a})₂, —OC(O)N(R^{a})₂, —NO₂, —N(R^{a})₂, —N(R^{a})C(O)R^{a}, —N(R^{a})C(O)₂R^{a}, —N(R^{a})S(O)₂R^{a}, —SR^{a}, —S(O)₂R^{a}, —S(O)N(R^{a})₂, —S(O)₂N(R^{a})₂, and an optionally substituted C₁₋₆ aliphatic group. In some embodiments, R³ is hydrogen.

As described generally above, n is 0-5. In some embodiments, n is 0. In some embodiments, n is 1, 2, 3, 4, or 5. In some embodiments, n is 1. In some embodiments, n is 2. In some embodiments, n is 3. In some embodiments, n is 4. In some embodiments, n is 5.

In some embodiments, a compound of Formula I is a compound of Formula II:

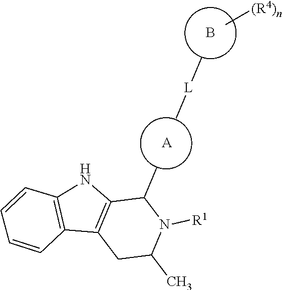

or a pharmaceutically acceptable salt thereof, wherein A, B, L, n, R¹, and R⁴ are as defined above for Formula I and described in classes and subclasses herein, both singly and in combination.

In some embodiments, a compound of Formula I is a compound of Formula II-a:

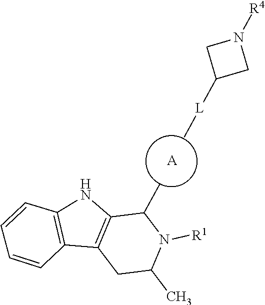

or a pharmaceutically acceptable salt thereof, wherein A, L, R¹, and R⁴ are as defined above for Formula I and described in classes and subclasses herein, both singly and in combination.

In some embodiments, a compound of Formula I is a compound of Formula II-b:

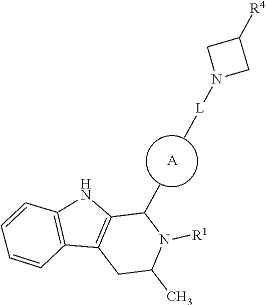

or a pharmaceutically acceptable salt thereof, wherein A, L, R¹, and R⁴ are as defined above for Formula I and described in classes and subclasses herein, both singly and in combination.

In some embodiments, a compound of Formula I is a compound of Formula II-c:

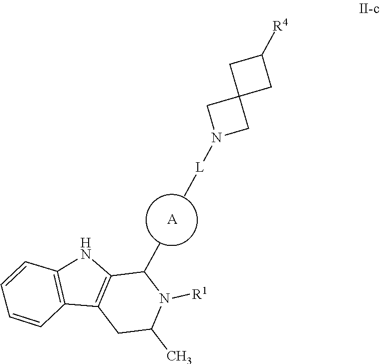

or a pharmaceutically acceptable salt thereof, wherein A, L, R¹, and R⁴ are as defined above for Formula I and described in classes and subclasses herein, both singly and in combination.

In some embodiments, a compound of Formula I is a compound of Formula II-d:

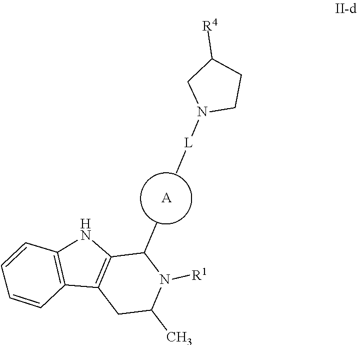

or a pharmaceutically acceptable salt thereof, wherein A, L, R¹, and R⁴ are as defined above for Formula I and described in classes and subclasses herein, both singly and in combination.

In some embodiments, a compound of Formula I is a compound of Formula II-e:

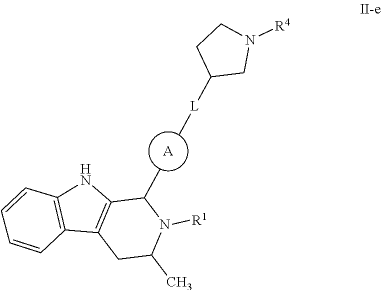

or a pharmaceutically acceptable salt thereof, wherein A, L, R¹, and R⁴ are as defined above for Formula I and described in classes and subclasses herein, both singly and in combination.

In some embodiments, a compound of Formula I is a compound of Formula II-f:

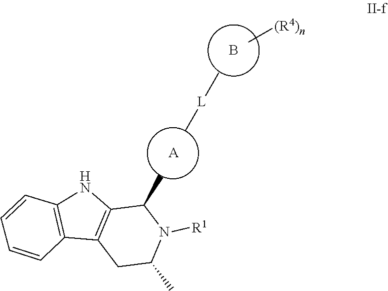

or a pharmaceutically acceptable salt thereof, wherein A, B, L, n, R¹, and R⁴ are as defined above for Formula I and described in classes and subclasses herein, both singly and in combination.

In some embodiments, a compound of Formula I is a compound of Formula III:

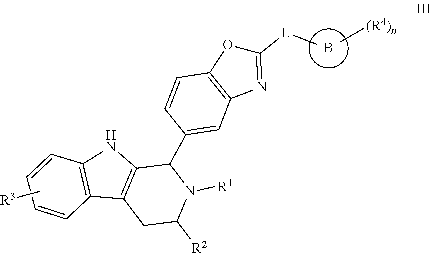

or a pharmaceutically acceptable salt thereof, wherein B, L, n, R¹, R², R³, and R⁴ are as defined above for Formula I and described in classes and subclasses herein, both singly and in combination.

In some embodiments, a compound of Formula I is a compound of Formula IV:

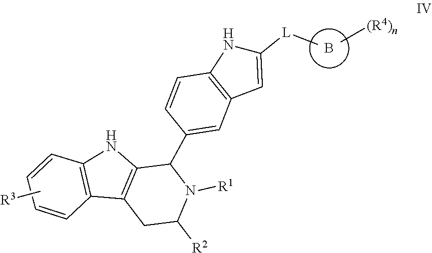

or a pharmaceutically acceptable salt thereof, wherein B, L, n, R¹, R², R³, and R⁴ are as defined above for Formula I and described in classes and subclasses herein, both singly and in combination.

In some embodiments, a compound of Formula I is a compound of Formula V:

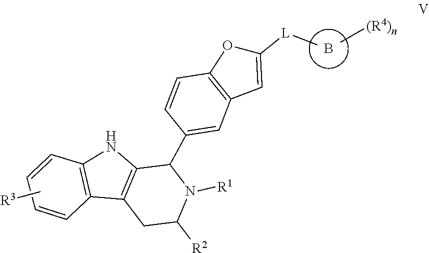

or a pharmaceutically acceptable salt thereof, wherein B, L, n, R¹, R², R³, and R⁴ are as defined above for Formula I and described in classes and subclasses herein, both singly and in combination.

In some embodiments, a compound of Formula I is a compound of Formula VI:

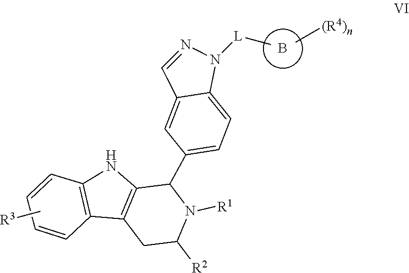

or a pharmaceutically acceptable salt thereof, wherein B, L, n, R¹, R², R³, and R⁴ are as defined above for Formula I and described in classes and subclasses herein, both singly and in combination.

In some embodiments, a compound of Formula I is a compound of Formula VII-1 or VII-2:

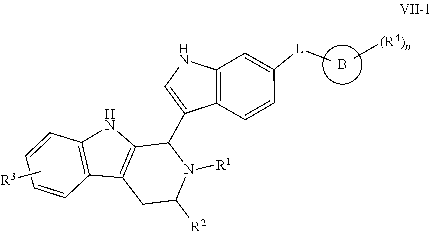

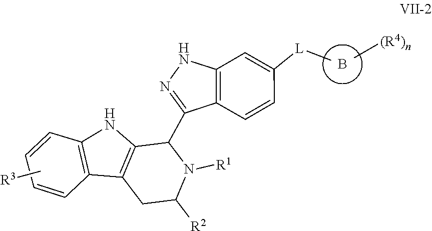

or a pharmaceutically acceptable salt thereof, wherein B, L, n, R¹, R², R³, and R⁴ are as defined above for Formula I and described in classes and subclasses herein, both singly and in combination

In some embodiments, the present disclosure provides compounds selected from Table 1, or a pharmaceutically acceptable salt thereof:

TABLE 1

| | Compound |
|---|---|
| Structure | No. |
| 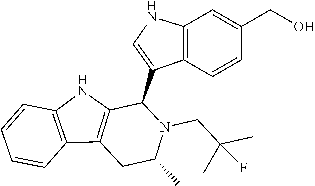 [SMILES: C[C@@H]1Cc2c([nH]c3ccccc23)[C@@H](c2c[nH]c3cc(CO)ccc23)N1CC(C)(C)F] | I-1 |
| 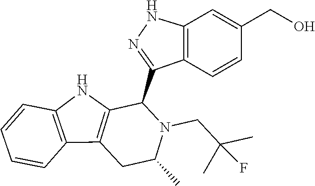 [SMILES: C[C@@H]1Cc2c([nH]c3ccccc23)[C@@H](c2n[nH]c3cc(CO)ccc23)N1CC(C)(C)F] | I-2 |
| 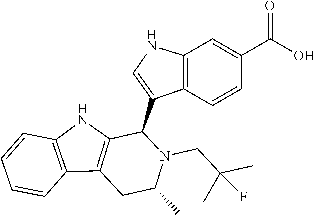 [SMILES: C[C@@H]1Cc2c([nH]c3ccccc23)[C@@H](c2c[nH]c3cc(C(=O)O)ccc23)N1CC(C)(C)F] | I-3 |
| 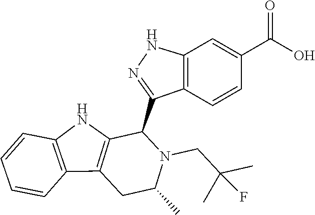 [SMILES: C[C@@H]1Cc2c([nH]c3ccccc23)[C@@H](c2n[nH]c3cc(C(=O)O)ccc23)N1CC(C)(C)F] | I-4 |
| 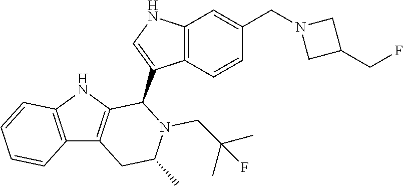 [SMILES: C[C@@H]1Cc2c([nH]c3ccccc23)[C@@H](c2c[nH]c3cc(CN4CC(CF)C4)ccc23)N1CC(C)(C)F] | I-5 |
| 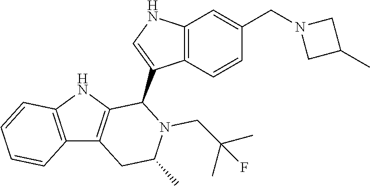 [SMILES: CC1CN(Cc2ccc3c([C@@H]4c5[nH]c6ccccc6c5C[C@@H](C)N4CC(C)(C)F)c[nH]c3c2)C1] | I-6 |
| 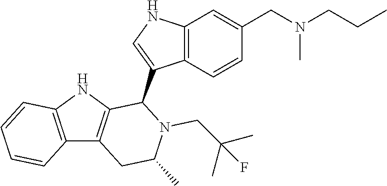 [SMILES: CCCN(C)Cc1ccc2c([C@@H]3c4[nH]c5ccccc5c4C[C@@H](C)N3CC(C)(C)F)c[nH]c2c1] | I-7 |
| 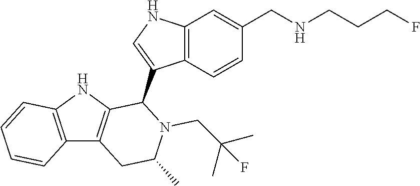 [SMILES: C[C@@H]1Cc2c([nH]c3ccccc23)[C@@H](c2c[nH]c3cc(CNCCCF)ccc23)N1CC(C)(C)F] | I-8 |
| 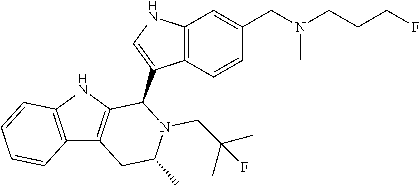 [SMILES: C[C@@H]1Cc2c([nH]c3ccccc23)[C@@H](c2c[nH]c3cc(CN(C)CCCF)ccc23)N1CC(C)(C)F] | I-9 |
| 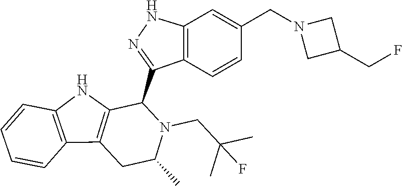 [SMILES: C[C@@H]1Cc2c([nH]c3ccccc23)[C@@H](c2n[nH]c3cc(CN4CC(CF)C4)ccc23)N1CC(C)(C)F] | I-10 |
| 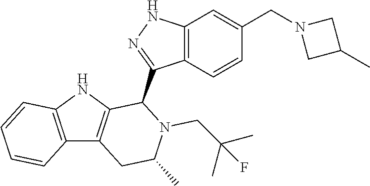 [SMILES: CC1CN(Cc2ccc3c([C@@H]4c5[nH]c6ccccc6c5C[C@@H](C)N4CC(C)(C)F)n[nH]c3c2)C1] | I-11 |
| 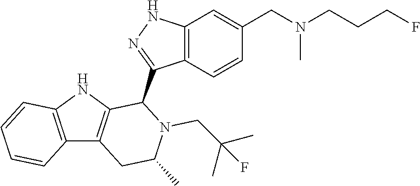 [SMILES: C[C@@H]1Cc2c([nH]c3ccccc23)[C@@H](c2n[nH]c3cc(CN(C)CCCF)ccc23)N1CC(C)(C)F] | I-12 |
| 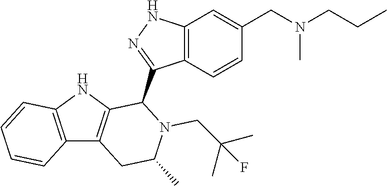 [SMILES: CCCN(C)Cc1ccc2c([C@@H]3c4[nH]c5ccccc5c4C[C@@H](C)N3CC(C)(C)F)n[nH]c2c1] | I-13 |
| 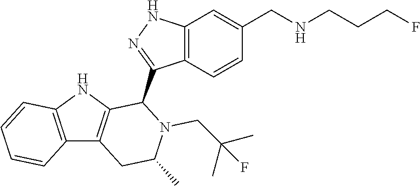 [SMILES: C[C@@H]1Cc2c([nH]c3ccccc23)[C@@H](c2n[nH]c3cc(CNCCCF)ccc23)N1CC(C)(C)F] | I-14 |
| 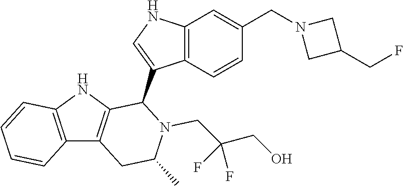 [SMILES: C[C@@H]1Cc2c([nH]c3ccccc23)[C@@H](c2c[nH]c3cc(CN4CC(CF)C4)ccc23)N1CC(F)(F)CO] | I-15 |
| 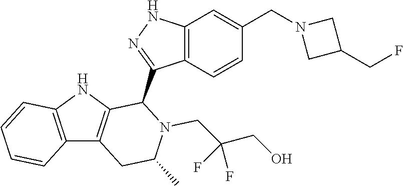 [SMILES: C[C@@H]1Cc2c([nH]c3ccccc23)[C@@H](c2n[nH]c3cc(CN4CC(CF)C4)ccc23)N1CC(F)(F)CO] | I-16 |
| 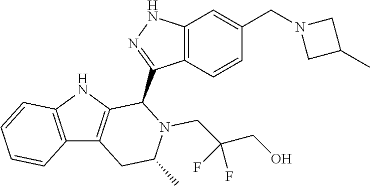 [SMILES: CC1CN(Cc2ccc3c([C@@H]4c5[nH]c6ccccc6c5C[C@@H](C)N4CC(F)(F)CO)n[nH]c3c2)C1] | I-17 |
| 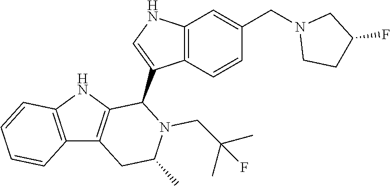 [SMILES: C[C@@H]1Cc2c([nH]c3ccccc23)[C@@H](c2c[nH]c3cc(CN4CC[C@@H](F)C4)ccc23)N1CC(C)(C)F] | I-18 |
| 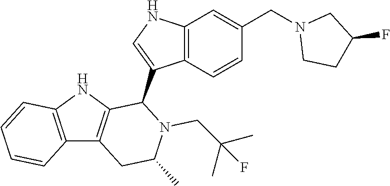 [SMILES: C[C@@H]1Cc2c([nH]c3ccccc23)[C@@H](c2c[nH]c3cc(CN4CC[C@H](F)C4)ccc23)N1CC(C)(C)F] | I-19 |
| 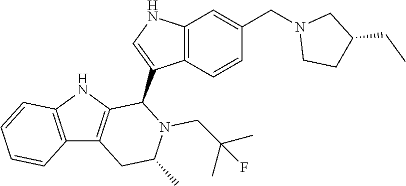 [SMILES: CC[C@@H]1CCN(Cc2ccc3c([C@@H]4c5[nH]c6ccccc6c5C[C@@H](C)N4CC(C)(C)F)c[nH]c3c2)C1] | I-20 |
| 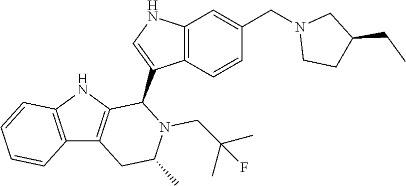 [SMILES: CC[C@H]1CCN(Cc2ccc3c([C@@H]4c5[nH]c6ccccc6c5C[C@@H](C)N4CC(C)(C)F)c[nH]c3c2)C1] | I-21 |
| 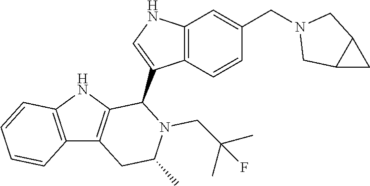 [SMILES: C[C@@H]1Cc2c([nH]c3ccccc23)[C@@H](c2c[nH]c3cc(CN4CC5CC5C4)ccc23)N1CC(C)(C)F] | I-22 |
| 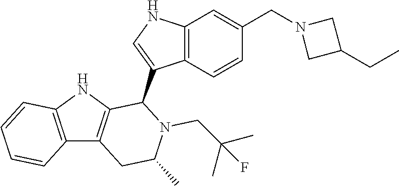 [SMILES: CCC1CN(Cc2ccc3c([C@@H]4c5[nH]c6ccccc6c5C[C@@H](C)N4CC(C)(C)F)c[nH]c3c2)C1] | I-23 |
| 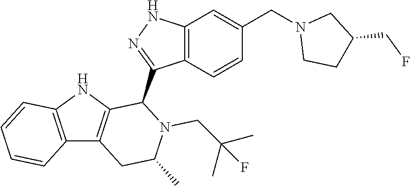 [SMILES: C[C@@H]1Cc2c([nH]c3ccccc23)[C@@H](c2n[nH]c3cc(CN4CC[C@@H](CF)C4)ccc23)N1CC(C)(C)F] | I-24 |
| 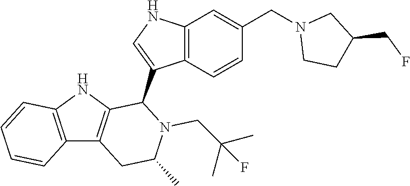 [SMILES: C[C@@H]1Cc2c([nH]c3ccccc23)[C@@H](c2c[nH]c3cc(CN4CC[C@H](CF)C4)ccc23)N1CC(C)(C)F] | I-25 |
| 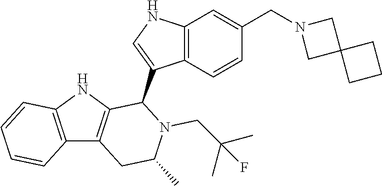 [SMILES: C[C@@H]1Cc2c([nH]c3ccccc23)[C@@H](c2c[nH]c3cc(CN4CC5(CCC5)C4)ccc23)N1CC(C)(C)F] | I-26 |
| 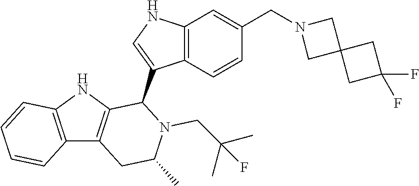 [SMILES: C[C@@H]1Cc2c([nH]c3ccccc23)[C@@H](c2c[nH]c3cc(CN4CC5(C4)CC(F)(F)C5)ccc23)N1CC(C)(C)F] | I-27 |
| 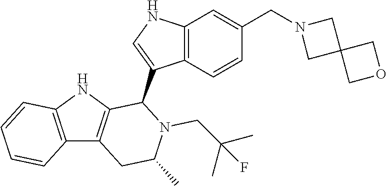 [SMILES: C[C@@H]1Cc2c([nH]c3ccccc23)[C@@H](c2c[nH]c3cc(CN4CC5(COC5)C4)ccc23)N1CC(C)(C)F] | I-28 |
| 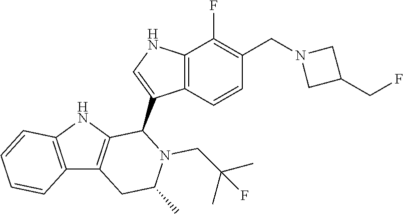 [SMILES: C[C@@H]1Cc2c([nH]c3ccccc23)[C@@H](c2c[nH]c3c(F)c(CN4CC(CF)C4)ccc23)N1CC(C)(C)F] | I-29 |
| 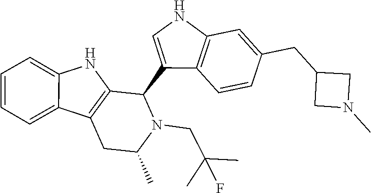 [SMILES: C[C@@H]1Cc2c([nH]c3ccccc23)[C@@H](c2c[nH]c3cc(CC4CN(C)C4)ccc23)N1CC(C)(C)F] | I-30 |
| 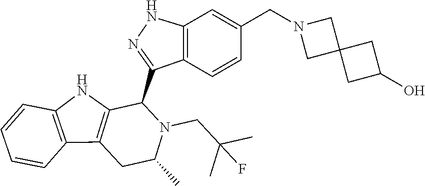 [SMILES: C[C@@H]1Cc2c([nH]c3ccccc23)[C@@H](c2n[nH]c3cc(CN4CC5(CC(O)C5)C4)ccc23)N1CC(C)(C)F] | I-31 |
| 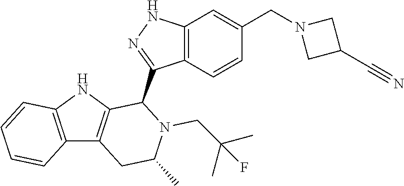 [SMILES: C[C@@H]1Cc2c([nH]c3ccccc23)[C@@H](c2n[nH]c3cc(CN4CC(C#N)C4)ccc23)N1CC(C)(C)F] | I-32 |
| 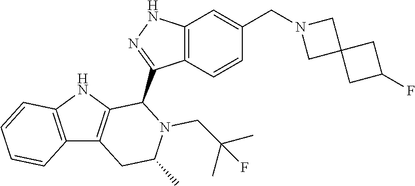 [SMILES: C[C@@H]1Cc2c([nH]c3ccccc23)[C@@H](c2n[nH]c3cc(CN4CC5(CC(F)C5)C4)ccc23)N1CC(C)(C)F] | I-33 |
| 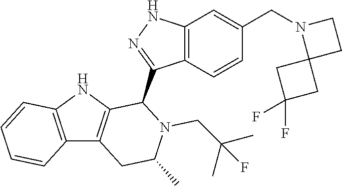 [SMILES: C[C@@H]1Cc2c([nH]c3ccccc23)[C@@H](c2n[nH]c3cc(CN4CCC45CC(F)(F)C5)ccc23)N1CC(C)(C)F] | I-34 |
| 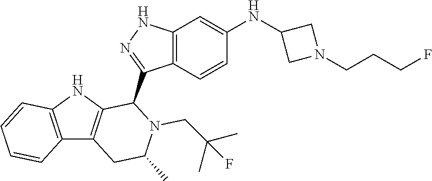 [SMILES: C[C@@H]1Cc2c([nH]c3ccccc23)[C@@H](c2n[nH]c3cc(NC4CN(CCCF)C4)ccc23)N1CC(C)(C)F] | I-35 |
| 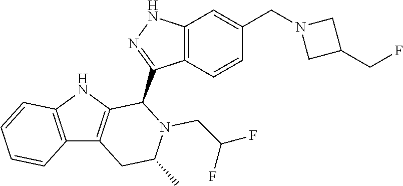 [SMILES: C[C@@H]1Cc2c([nH]c3ccccc23)[C@@H](c2n[nH]c3cc(CN4CC(CF)C4)ccc23)N1CC(F)F] | I-36 |
| 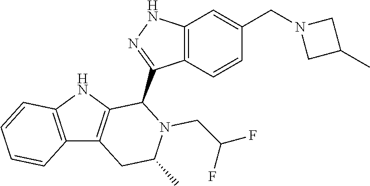 [SMILES: CC1CN(Cc2ccc3c([C@@H]4c5[nH]c6ccccc6c5C[C@@H](C)N4CC(F)F)n[nH]c3c2)C1] | I-37 |
| 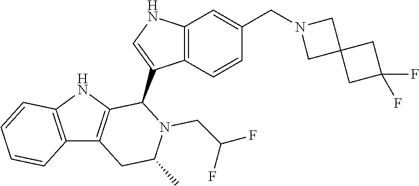 [SMILES: C[C@@H]1Cc2c([nH]c3ccccc23)[C@@H](c2c[nH]c3cc(CN4CC5(C4)CC(F)(F)C5)ccc23)N1CC(F)F] | I-38 |
| 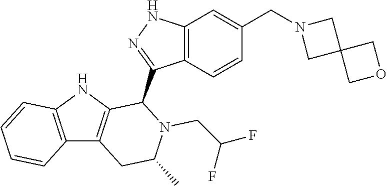 [SMILES: C[C@@H]1Cc2c([nH]c3ccccc23)[C@@H](c2n[nH]c3cc(CN4CC5(COC5)C4)ccc23)N1CC(F)F] | I-39 |
| 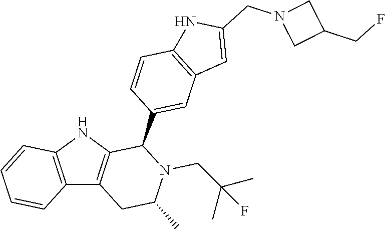 [SMILES: C[C@@H]1Cc2c([nH]c3ccccc23)[C@@H](c2ccc3[nH]c(CN4CC(CF)C4)cc3c2)N1CC(C)(C)F] | I-40 |
| 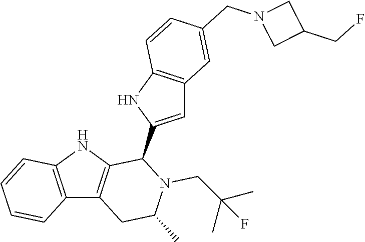 [SMILES: C[C@@H]1Cc2c([nH]c3ccccc23)[C@@H](c2cc3cc(CN4CC(CF)C4)ccc3[nH]2)N1CC(C)(C)F] | I-41 |
| 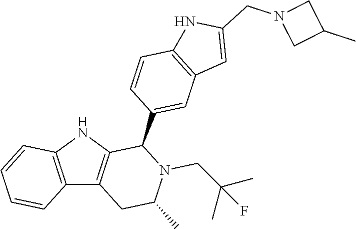 [SMILES: CC1CN(Cc2cc3cc([C@@H]4c5[nH]c6ccccc6c5C[C@@H](C)N4CC(C)(C)F)ccc3[nH]2)C1] | I-42 |
| 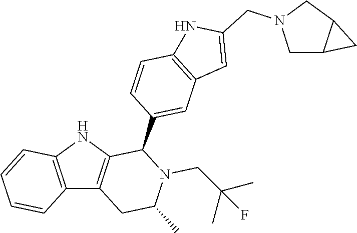 [SMILES: C[C@@H]1Cc2c([nH]c3ccccc23)[C@@H](c2ccc3[nH]c(CN4CC5CC5C4)cc3c2)N1CC(C)(C)F] | I-43 |
| 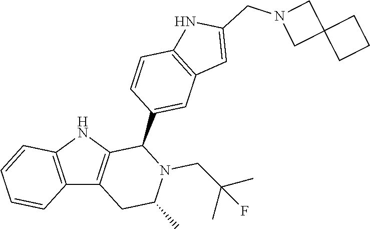 [SMILES: C[C@@H]1Cc2c([nH]c3ccccc23)[C@@H](c2ccc3[nH]c(CN4CC5(CCC5)C4)cc3c2)N1CC(C)(C)F] | I-44 |
| 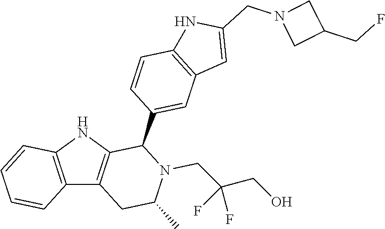 [SMILES: C[C@@H]1Cc2c([nH]c3ccccc23)[C@@H](c2ccc3[nH]c(CN4CC(CF)C4)cc3c2)N1CC(F)(F)CO] | I-45 |
| 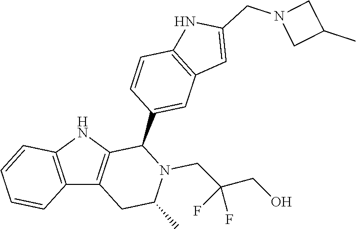 [SMILES: CC1CN(Cc2cc3cc([C@@H]4c5[nH]c6ccccc6c5C[C@@H](C)N4CC(F)(F)CO)ccc3[nH]2)C1] | I-46 |
| 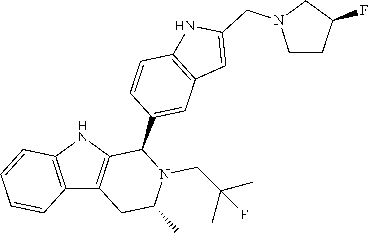 [SMILES: C[C@@H]1Cc2c([nH]c3ccccc23)[C@@H](c2ccc3[nH]c(CN4CC[C@H](F)C4)cc3c2)N1CC(C)(C)F] | I-47 |
| 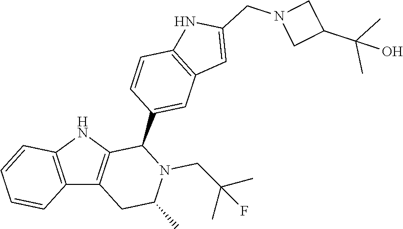 [SMILES: C[C@@H]1Cc2c([nH]c3ccccc23)[C@@H](c2ccc3[nH]c(CN4CC(C(C)(C)O)C4)cc3c2)N1CC(C)(C)F] | I-48 |
| 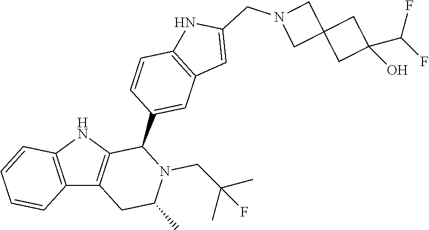 [SMILES: C[C@@H]1Cc2c([nH]c3ccccc23)[C@@H](c2ccc3[nH]c(CN4CC5(C4)CC(O)(C(F)F)C5)cc3c2)N1CC(C)(C)F] | I-49 |
| 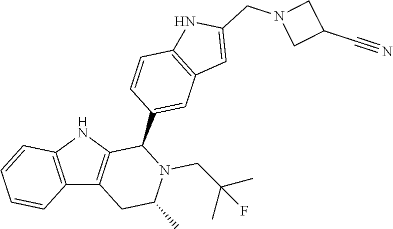 [SMILES: C[C@@H]1Cc2c([nH]c3ccccc23)[C@@H](c2ccc3[nH]c(CN4CC(C#N)C4)cc3c2)N1CC(C)(C)F] | I-50 |
| 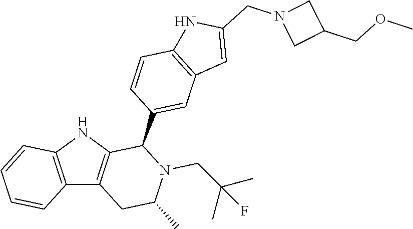 [SMILES: COCC1CN(Cc2cc3cc([C@@H]4c5[nH]c6ccccc6c5C[C@@H](C)N4CC(C)(C)F)ccc3[nH]2)C1] | I-51 |
| 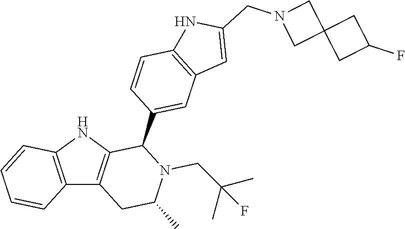 [SMILES: C[C@@H]1Cc2c([nH]c3ccccc23)[C@@H](c2ccc3[nH]c(CN4CC5(CC(F)C5)C4)cc3c2)N1CC(C)(C)F] | I-52 |
| 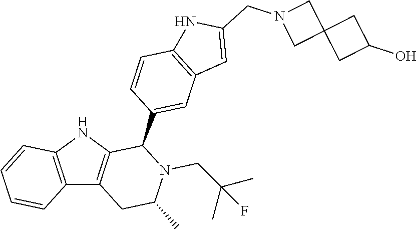 [SMILES: C[C@@H]1Cc2c([nH]c3ccccc23)[C@@H](c2ccc3[nH]c(CN4CC5(CC(O)C5)C4)cc3c2)N1CC(C)(C)F] | I-53 |
| 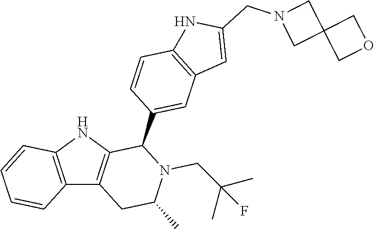 [SMILES: C[C@@H]1Cc2c([nH]c3ccccc23)[C@@H](c2ccc3[nH]c(CN4CC5(COC5)C4)cc3c2)N1CC(C)(C)F] | I-54 |
| 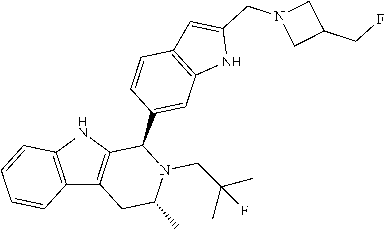 [SMILES: C[C@@H]1Cc2c([nH]c3ccccc23)[C@@H](c2ccc3cc(CN4CC(CF)C4)[nH]c3c2)N1CC(C)(C)F] | I-55 |
| 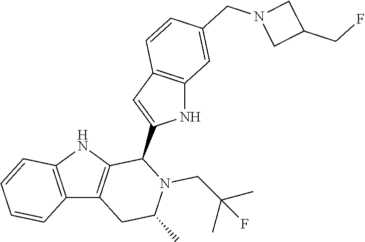 [SMILES: C[C@@H]1Cc2c([nH]c3ccccc23)[C@@H](c2cc3ccc(CN4CC(CF)C4)cc3[nH]2)N1CC(C)(C)F] | I-56 |
| 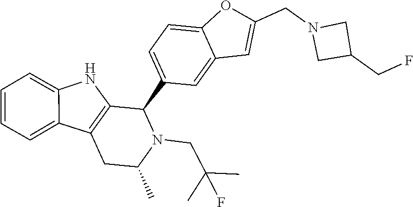 [SMILES: C[C@@H]1Cc2c([nH]c3ccccc23)[C@@H](c2ccc3oc(CN4CC(CF)C4)cc3c2)N1CC(C)(C)F] | I-57 |
| 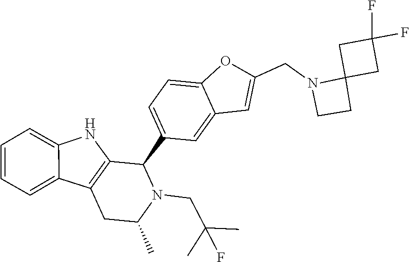 [SMILES: C[C@@H]1Cc2c([nH]c3ccccc23)[C@@H](c2ccc3oc(CN4CCC45CC(F)(F)C5)cc3c2)N1CC(C)(C)F] | I-58 |
| 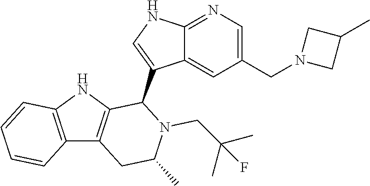 [SMILES: CC1CN(Cc2cnc3[nH]cc([C@@H]4c5[nH]c6ccccc6c5C[C@@H](C)N4CC(C)(C)F)c3c2)C1] | I-59 |
| 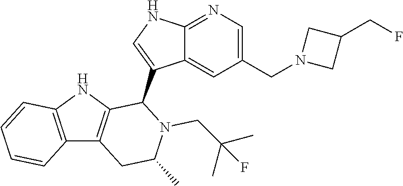 [SMILES: C[C@@H]1Cc2c([nH]c3ccccc23)[C@@H](c2c[nH]c3ncc(CN4CC(CF)C4)cc23)N1CC(C)(C)F] | I-60 |
| 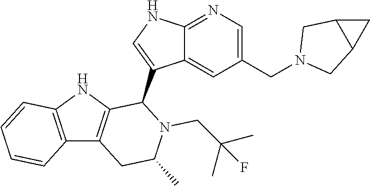 [SMILES: C[C@@H]1Cc2c([nH]c3ccccc23)[C@@H](c2c[nH]c3ncc(CN4CC5CC5C4)cc23)N1CC(C)(C)F] | I-61 |
| 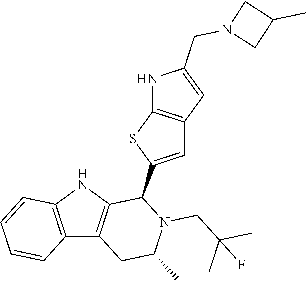 [SMILES: CC1CN(Cc2cc3cc([C@@H]4c5[nH]c6ccccc6c5C[C@@H](C)N4CC(C)(C)F)sc3[nH]2)C1] | I-62 |
| 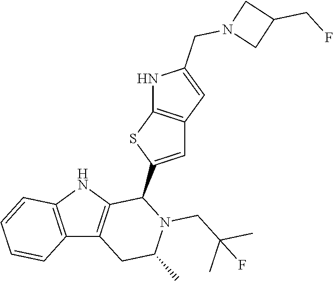 [SMILES: C[C@@H]1Cc2c([nH]c3ccccc23)[C@@H](c2cc3cc(CN4CC(CF)C4)[nH]c3s2)N1CC(C)(C)F] | I-63 |
| 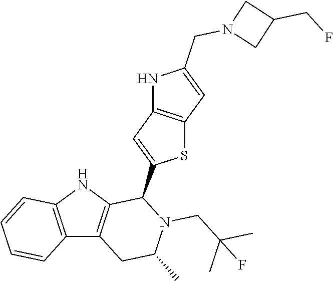 [SMILES: C[C@@H]1Cc2c([nH]c3ccccc23)[C@@H](c2cc3[nH]c(CN4CC(CF)C4)cc3s2)N1CC(C)(C)F] | I-64 |
| 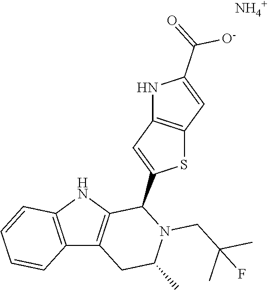 [SMILES: [NH4+] ; C[C@@H]1Cc2c([nH]c3ccccc23)[C@@H](c2cc3[nH]c(C(=O)[O-])cc3s2)N1CC(C)(C)F] | I-65 |
| 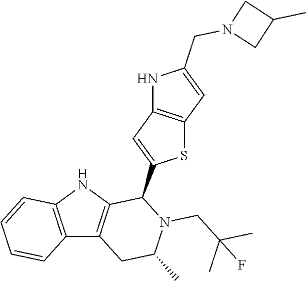 [SMILES: CC1CN(Cc2cc3sc([C@@H]4c5[nH]c6ccccc6c5C[C@@H](C)N4CC(C)(C)F)cc3[nH]2)C1] | I-66 |
| 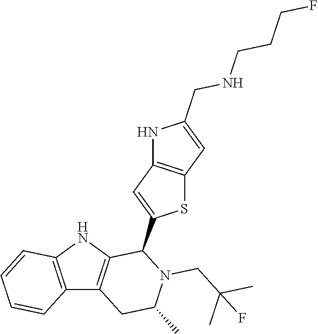 [SMILES: C[C@@H]1Cc2c([nH]c3ccccc23)[C@@H](c2cc3[nH]c(CNCCCF)cc3s2)N1CC(C)(C)F] | I-67 |
| 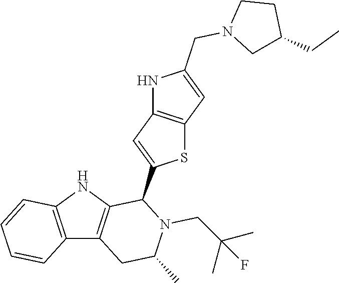 [SMILES: CC[C@H]1CCN(Cc2cc3sc([C@@H]4c5[nH]c6ccccc6c5C[C@@H](C)N4CC(C)(C)F)cc3[nH]2)C1] | I-68 |
| 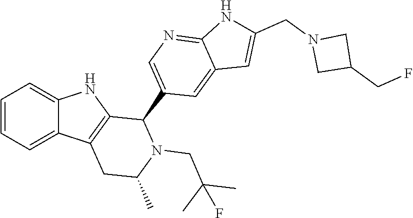 [SMILES: C[C@@H]1Cc2c([nH]c3ccccc23)[C@@H](c2cnc3[nH]c(CN4CC(CF)C4)cc3c2)N1CC(C)(C)F] | I-69 |
| 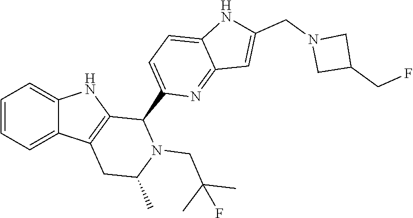 [SMILES: C[C@@H]1Cc2c([nH]c3ccccc23)[C@@H](c2ccc3[nH]c(CN4CC(CF)C4)cc3n2)N1CC(C)(C)F] | I-70 |
| 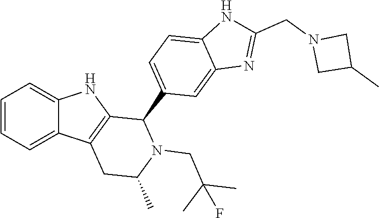 [SMILES: CC1CN(Cc2nc3cc([C@@H]4c5[nH]c6ccccc6c5C[C@@H](C)N4CC(C)(C)F)ccc3[nH]2)C1] | I-71 |
| 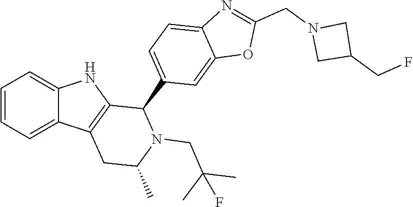 [SMILES: C[C@@H]1Cc2c([nH]c3ccccc23)[C@@H](c2ccc3nc(CN4CC(CF)C4)oc3c2)N1CC(C)(C)F] | I-72 |
| 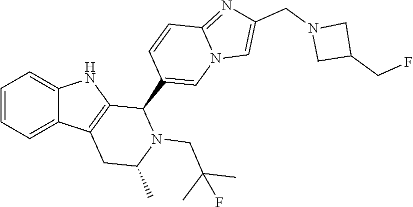 [SMILES: C[C@@H]1Cc2c([nH]c3ccccc23)[C@@H](c2ccc3nc(CN4CC(CF)C4)cn3c2)N1CC(C)(C)F] | I-73 |
| 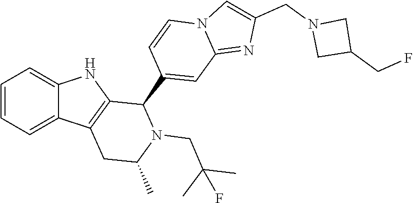 [SMILES: C[C@@H]1Cc2c([nH]c3ccccc23)[C@@H](c2ccn3cc(CN4CC(CF)C4)nc3c2)N1CC(C)(C)F] | I-74 |
| 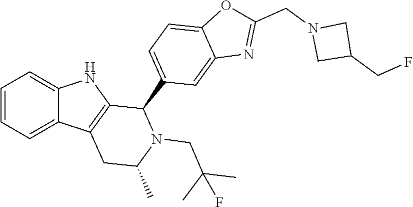 [SMILES: C[C@@H]1Cc2c([nH]c3ccccc23)[C@@H](c2ccc3oc(CN4CC(CF)C4)nc3c2)N1CC(C)(C)F] | I-75 |
| 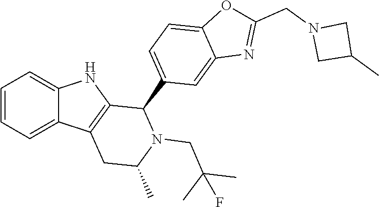 [SMILES: CC1CN(Cc2nc3cc([C@@H]4c5[nH]c6ccccc6c5C[C@@H](C)N4CC(C)(C)F)ccc3o2)C1] | I-76 |
| 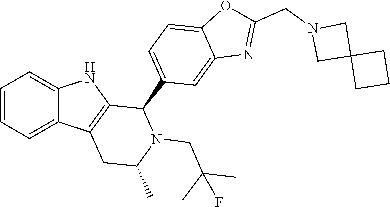 [SMILES: C[C@@H]1Cc2c([nH]c3ccccc23)[C@@H](c2ccc3oc(CN4CC5(CCC5)C4)nc3c2)N1CC(C)(C)F] | I-77 |
| 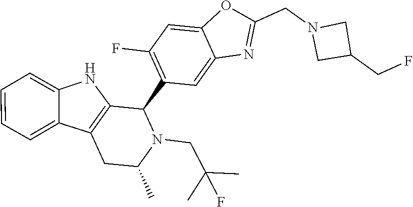 [SMILES: C[C@@H]1Cc2c([nH]c3ccccc23)[C@@H](c2cc3nc(CN4CC(CF)C4)oc3cc2F)N1CC(C)(C)F] | I-78 |
| 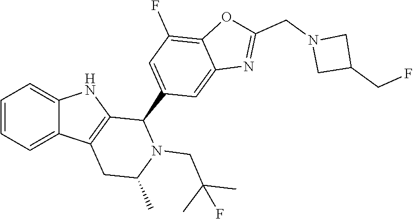 [SMILES: C[C@@H]1Cc2c([nH]c3ccccc23)[C@@H](c2cc(F)c3oc(CN4CC(CF)C4)nc3c2)N1CC(C)(C)F] | I-79 |
| 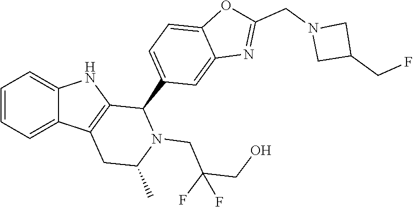 [SMILES: C[C@@H]1Cc2c([nH]c3ccccc23)[C@@H](c2ccc3oc(CN4CC(CF)C4)nc3c2)N1CC(F)(F)CO] | I-80 |
| 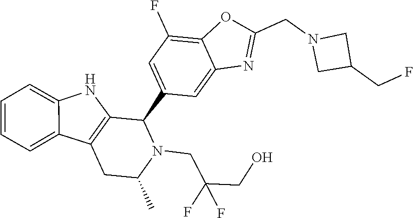 [SMILES: C[C@@H]1Cc2c([nH]c3ccccc23)[C@@H](c2cc(F)c3oc(CN4CC(CF)C4)nc3c2)N1CC(F)(F)CO] | I-81 |
| 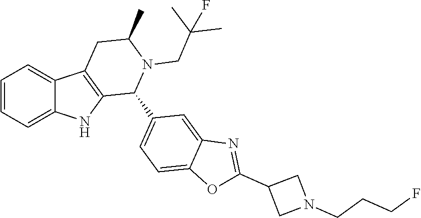 [SMILES: C[C@@H]1Cc2c([nH]c3ccccc23)[C@@H](c2ccc3oc(C4CN(CCCF)C4)nc3c2)N1CC(C)(C)F] | I-82 |
| 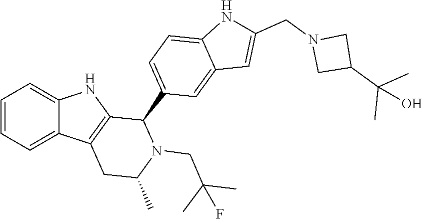 [SMILES: C[C@@H]1Cc2c([nH]c3ccccc23)[C@@H](c2ccc3[nH]c(CN4CC(C(C)(C)O)C4)cc3c2)N1CC(C)(C)F] | I-83 |
| 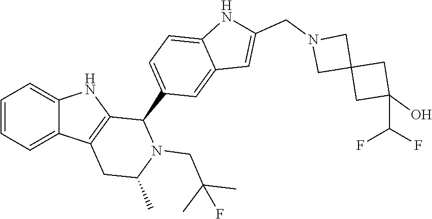 [SMILES: C[C@@H]1Cc2c([nH]c3ccccc23)[C@@H](c2ccc3[nH]c(CN4CC5(C4)CC(O)(C(F)F)C5)cc3c2)N1CC(C)(C)F] | I-84 |
| 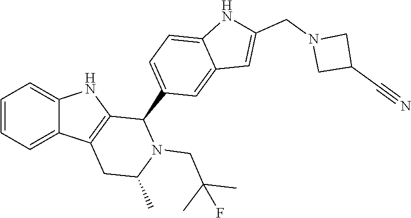 [SMILES: C[C@@H]1Cc2c([nH]c3ccccc23)[C@@H](c2ccc3[nH]c(CN4CC(C#N)C4)cc3c2)N1CC(C)(C)F] | I-85 |
| 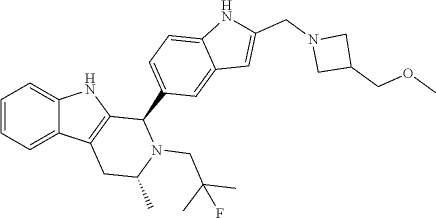 [SMILES: COCC1CN(Cc2cc3cc([C@@H]4c5[nH]c6ccccc6c5C[C@@H](C)N4CC(C)(C)F)ccc3[nH]2)C1] | I-86 |
| 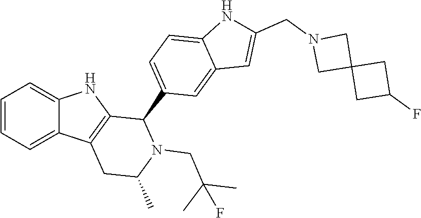 [SMILES: C[C@@H]1Cc2c([nH]c3ccccc23)[C@@H](c2ccc3[nH]c(CN4CC5(CC(F)C5)C4)cc3c2)N1CC(C)(C)F] | I-87 |
| 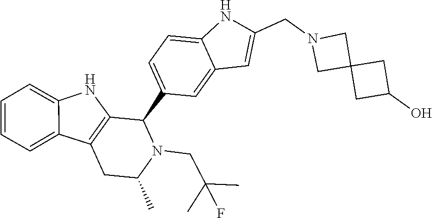 [SMILES: C[C@@H]1Cc2c([nH]c3ccccc23)[C@@H](c2ccc3[nH]c(CN4CC5(CC(O)C5)C4)cc3c2)N1CC(C)(C)F] | I-88 |
| 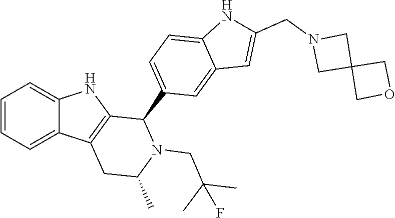 [SMILES: C[C@@H]1Cc2c([nH]c3ccccc23)[C@@H](c2ccc3[nH]c(CN4CC5(COC5)C4)cc3c2)N1CC(C)(C)F] | I-89 |
| 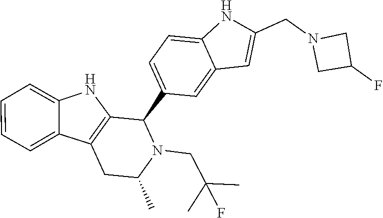 [SMILES: C[C@@H]1Cc2c([nH]c3ccccc23)[C@@H](c2ccc3[nH]c(CN4CC(F)C4)cc3c2)N1CC(C)(C)F] | I-90 |
| 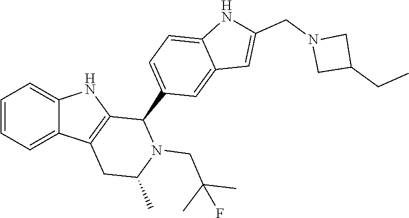 [SMILES: CCC1CN(Cc2cc3cc([C@@H]4c5[nH]c6ccccc6c5C[C@@H](C)N4CC(C)(C)F)ccc3[nH]2)C1] | I-91 |
| 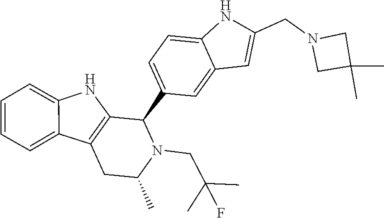 [SMILES: C[C@@H]1Cc2c([nH]c3ccccc23)[C@@H](c2ccc3[nH]c(CN4CC(C)(C)C4)cc3c2)N1CC(C)(C)F] | I-92 |
| 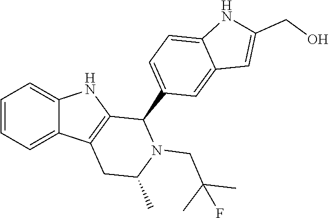 [SMILES: C[C@@H]1Cc2c([nH]c3ccccc23)[C@@H](c2ccc3[nH]c(CO)cc3c2)N1CC(C)(C)F] | I-93 |
| 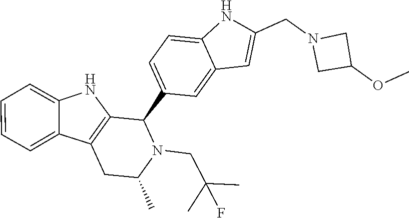 [SMILES: COC1CN(Cc2cc3cc([C@@H]4c5[nH]c6ccccc6c5C[C@@H](C)N4CC(C)(C)F)ccc3[nH]2)C1] | I-94 |
| 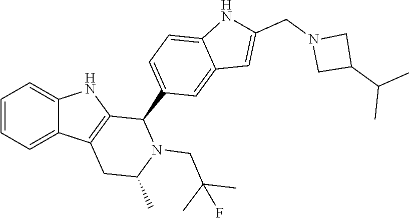 [SMILES: CC(C)C1CN(Cc2cc3cc([C@@H]4c5[nH]c6ccccc6c5C[C@@H](C)N4CC(C)(C)F)ccc3[nH]2)C1] | I-95 |
| 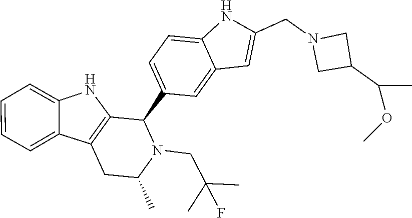 [SMILES: COC(C)C1CN(Cc2cc3cc([C@@H]4c5[nH]c6ccccc6c5C[C@@H](C)N4CC(C)(C)F)ccc3[nH]2)C1] | I-96 |
| 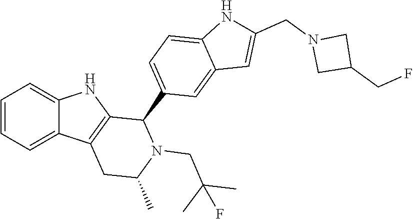 [SMILES: C[C@@H]1Cc2c([nH]c3ccccc23)[C@@H](c2ccc3[nH]c(CN4CC(CF)C4)cc3c2)N1CC(C)(C)F] | I-97 |
| 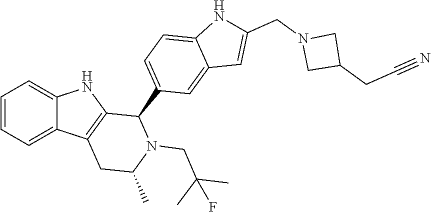 [SMILES: C[C@@H]1Cc2c([nH]c3ccccc23)[C@@H](c2ccc3[nH]c(CN4CC(CC#N)C4)cc3c2)N1CC(C)(C)F] | I-98 |
| 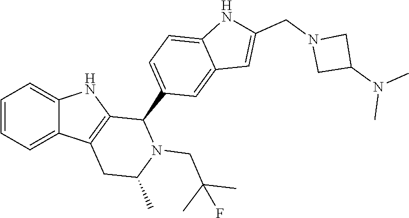 [SMILES: C[C@@H]1Cc2c([nH]c3ccccc23)[C@@H](c2ccc3[nH]c(CN4CC(N(C)C)C4)cc3c2)N1CC(C)(C)F] | I-99 |
| 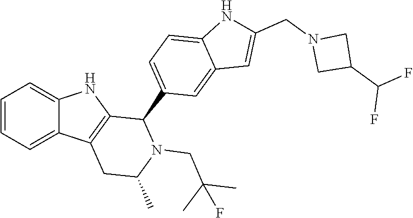 [SMILES: C[C@@H]1Cc2c([nH]c3ccccc23)[C@@H](c2ccc3[nH]c(CN4CC(C(F)F)C4)cc3c2)N1CC(C)(C)F] | I-100 |
| 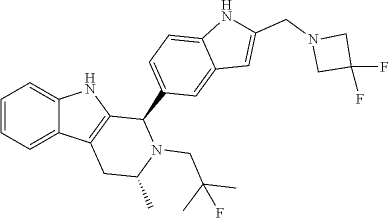 [SMILES: C[C@@H]1Cc2c([nH]c3ccccc23)[C@@H](c2ccc3[nH]c(CN4CC(F)(F)C4)cc3c2)N1CC(C)(C)F] | I-101 |
| 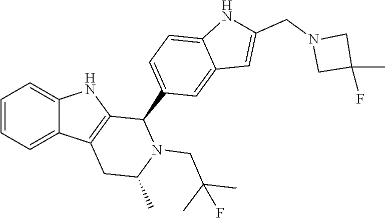 [SMILES: C[C@@H]1Cc2c([nH]c3ccccc23)[C@@H](c2ccc3[nH]c(CN4CC(C)(F)C4)cc3c2)N1CC(C)(C)F] | I-102 |
| 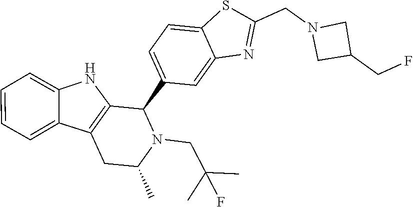 [SMILES: C[C@@H]1Cc2c([nH]c3ccccc23)[C@@H](c2ccc3sc(CN4CC(CF)C4)nc3c2)N1CC(C)(C)F] | I-103 |
| 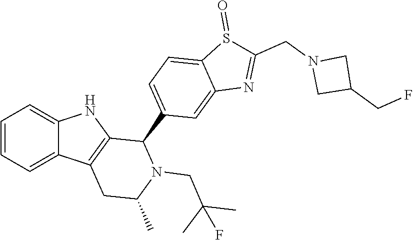 [SMILES: C[C@@H]1Cc2c([nH]c3ccccc23)[C@@H](c2ccc3c(c2)N=C(CN2CC(CF)C2)S3=O)N1CC(C)(C)F] | I-104 |
| 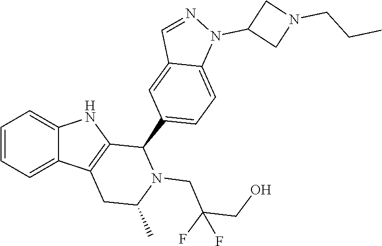 [SMILES: CCCN1CC(n2ncc3cc([C@@H]4c5[nH]c6ccccc6c5C[C@@H](C)N4CC(F)(F)CO)ccc32)C1] | I-105 |
| 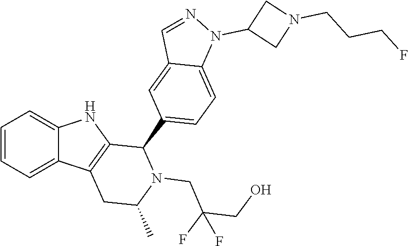 [SMILES: C[C@@H]1Cc2c([nH]c3ccccc23)[C@@H](c2ccc3c(cnn3C3CN(CCCF)C3)c2)N1CC(F)(F)CO] | I-106 |
| 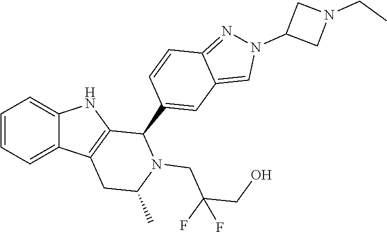 [SMILES: CCN1CC(n2cc3cc([C@@H]4c5[nH]c6ccccc6c5C[C@@H](C)N4CC(F)(F)CO)ccc3n2)C1] | I-107 |
| 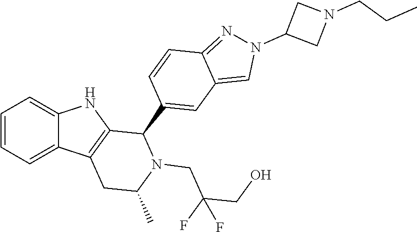 [SMILES: CCCN1CC(n2cc3cc([C@@H]4c5[nH]c6ccccc6c5C[C@@H](C)N4CC(F)(F)CO)ccc3n2)C1] | I-108 |
| 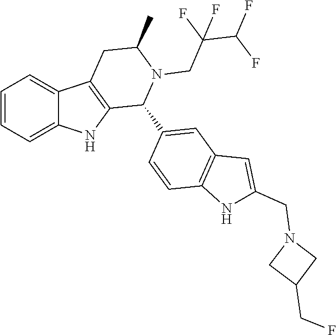 [SMILES: C[C@@H]1Cc2c([nH]c3ccccc23)[C@@H](c2ccc3[nH]c(CN4CC(CF)C4)cc3c2)N1CC(F)(F)C(F)F] | I-109 |
| 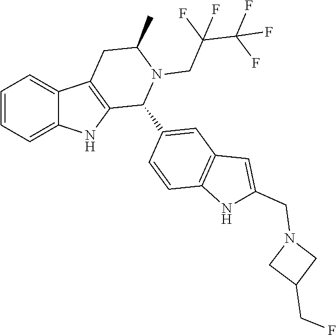 [SMILES: C[C@@H]1Cc2c([nH]c3ccccc23)[C@@H](c2ccc3[nH]c(CN4CC(CF)C4)cc3c2)N1CC(F)(F)C(F)(F)F] | I-110 |

In some embodiments, the present disclosure provides a compound of Table 2, or a pharmaceutically acceptable salt thereof:

TABLE 2

| Compound No. | Compound Name. |
|---|---|
| I-1 | [3-[(1R,3R)-2-(2-fluoro-2-methyl-propyl)-3-methyl-1,3,4,9- |
| | tetrahydropyrido[3,4-b]indol-1-yl]-1H-indol-6-yl]methanol |
| I-2 | [3-[(1S,3R)-2-(2-fluoro-2-methyl-propyl)-3-methyl-1,3,4,9- |
| | tetrahydropyrido[3,4-b]indol-1-yl]-1H-indazol-6-yl]methanol |
| I-3 | 3-[(1R,3R)-2-(2-fluoro-2-methyl-propyl)-3-methyl-1,3,4,9- |
| | tetrahydropyrido[3,4-b]indol-1-yl]-1H-indole-6-carboxylic acid |
| I-4 | 3-[(1S,3R)-2-(2-fluoro-2-methyl-propyl)-3-methyl-1,3,4,9- |
| | tetrahydropyrido[3,4-b]indol-1-yl]-1H-indazole-6-carboxylic acid |
| I-5 | (1R,3R)-1-[6-[[3-(fluoromethyl)azetidin-1-yl]methyl]-1H-indol-3-yl]-2-(2- |
| | fluoro-2-methyl-propyl)-3-methyl-1,3,4,9-tetrahydropyrido[3,4-b]indole |
| I-6 | (1R,3R)-2-(2-fluoro-2-methyl-propyl)-3-methyl-1-[6-[(3-methylazetidin-1- |
| | yl)methyl]-1H-indol-3-yl]-1,3,4,9-tetrahydropyrido[3,4-b]indole |
| I-7 | N-[[3-[(1R,3R)-2-(2-fluoro-2-methyl-propyl)-3-methyl-1,3,4,9- |
| | tetrahydropyrido[3,4-b]indol-1-yl]-1H-indol-6-yl]methyl]-N-methyl-propan- |
| | 1-amine |
| I-8 | 3-fluoro-N-[[3-[(1R,3R)-2-(2-fluoro-2-methyl-propyl)-3-methyl-1,3,4,9- |
| | tetrahydropyrido[3,4-b]indol-1-yl]-1H-indol-6-yl]methyl]propan-1-amine |
| I-9 | 3-fluoro-N-[[3-[(1R,3R)-2-(2-fluoro-2-methyl-propyl)-3-methyl-1,3,4,9- |
| | tetrahydropyrido[3,4-b]indol-1-yl]-1H-indol-6-yl]methyl]-N-methyl-propan- |
| | 1-amine |
| I-10 | (1S,3R)-1-[6-[[3-(fluoromethyl)azetidin-1-yl]methyl]-1H-indazol-3-yl]-2-(2- |
| | fluoro-2-methyl-propyl)-3-methyl-1,3,4,9-tetrahydropyrido[3,4-b]indole |
| I-11 | (1S,3R)-2-(2-fluoro-2-methyl-propyl)-3-methyl-1-[6-[(3-methylazetidin-1- |
| | yl)methyl]-1H-indazol-3-yl]-1,3,4,9-tetrahydropyrido[3,4-b]indole |
| I-12 | 3-fluoro-N-[[3-[(1S,3R)-2-(2-fluoro-2-methyl-propyl)-3-methyl-1,3,4,9- |
| | tetrahydropyrido[3,4-b]indol-1-yl]-1H-indazol-6-yl]methyl]-N-methyl- |
| | propan-1-amine |
| I-13 | N-[[3-[(1S,3R)-2-(2-fluoro-2-methyl-propyl)-3-methyl-1,3,4,9- |
| | tetrahydropyrido[3,4-b]indol-1-yl]-1H-indazol-6-yl]methyl]-N-methyl- |
| | propan-1-amine |
| I-14 | 3-fluoro-N-[[3-[(1S,3R)-2-(2-fluoro-2-methyl-propyl)-3-methyl-1,3,4,9- |
| | tetrahydropyrido[3,4-b]indol-1-yl]-1H-indazol-6-yl]methyl]propan-1-amine |
| I-15 | 2,2-difluoro-3-[(1R,3R)-1-[6-[[3-(fluoromethyl)azetidin-1-yl]methyl]-1H- |
| | indol-3-yl]-3-methyl-1,3,4,9-tetrahydropyrido[3,4-b]indol-2-yl]propan-1-ol |
| I-16 | 2,2-difluoro-3-[(1S,3R)-1-[6-[[3-(fluoromethyl)azetidin-1-yl]methyl]-1H- |
| | indazol-3-yl]-3-methyl-1,3,4,9-tetrahydropyrido[3,4-b]indol-2-yl]propan-1-ol |
| I-17 | 2,2-difluoro-3-((1S,3R)-3-methyl-1-(6-((3-methylazetidin-1-yl)methyl)-1H- |
| | indazol-3-yl)-1,3,4,9-tetrahydro-2H-pyrido[3,4-b]indol-2-yl)propan-1-ol |
| I-18 | (1S,3R)-2-(2-fluoro-2-methyl-propyl)-1-[6-[[(3R)-3-fluoropyrrolidin-1- |
| | yl]methyl]-1H-indazol-3-yl]-3-methyl-1,3,4,9-tetrahydropyrido[3,4-b]indole |
| I-19 | (1S,3R)-2-(2-fluoro-2-methyl-propyl)-1-[6-[[(3S)-3-fluoropyrrolidin-1- |
| | yl]methyl]-1H-indazol-3-yl]-3-methyl-1,3,4,9-tetrahydropyrido[3,4-b]indole |
| I-20 | (1S,3R)-1-[6-[[(3R)-3-ethylpyrrolidin-1-yl]methyl]-1H-indazol-3-yl]-2-(2- |
| | fluoro-2-methyl-propyl)-3-methyl-1,3,4,9-tetrahydropyrido[3,4-b]indole |
| I-21 | (1S,3R)-1-[6-[[(3S)-3-ethylpyrrolidin-1-yl]methyl]-1H-indazol-3-yl]-2-(2- |
| | fluoro-2-methyl-propyl)-3-methyl-1,3,4,9-tetrahydropyrido[3,4-b]indole |
| I-22 | (1S,3R)-1-[6-(3-azabicyclo[3.1.0]hexan-3-ylmethyl)-1H-indazol-3-yl]-2-(2- |
| | fluoro-2-methyl-propyl)-3-methyl-1,3,4,9-tetrahydropyrido[3,4-b]indole |
| I-23 | (1S,3R)-1-[6-[(3-ethylazetidin-1-yl)methyl]-1H-indazol-3-yl]-2-(2-fluoro-2- |
| | methyl-propyl)-3-methyl-1,3,4,9-tetrahydropyrido[3,4-b]indole |
| I-24 | (1S,3R)-2-(2-fluoro-2-methyl-propyl)-1-[6-[[(3R)-3- |
| | (fluoromethyl)pyrrolidin-1-yl]methyl]-1H-indazol-3-yl]-3-methyl-1,3,4,9- |
| | tetrahydropyrido[3,4-b]indole |
| I-25 | (1S,3R)-2-(2-fluoro-2-methyl-propyl)-1-[6-[[(3S)-3-(fluoromethyl)pyrrolidin- |
| | 1-yl]methyl]-1H-indazol-3-yl]-3-methyl-1,3,4,9-tetrahydropyrido[3,4- |
| | b]indole |
| I-26 | (1S,3R)-1-[6-(2-azaspiro[3.3]heptan-2-ylmethyl)-1H-indazol-3-yl]-2-(2- |
| | fluoro-2-methyl-propyl)-3-methyl-1,3,4,9-tetrahydropyrido[3,4-b]indole |
| I-27 | (1S,3R)-1-[6-[(6,6-difluoro-2-azaspiro[3.3]heptan-2-yl)methyl]-1H-indazol- |
| | 3-yl]-2-(2-fluoro-2-methyl-propyl)-3-methyl-1,3,4,9-tetrahydropyrido[3,4- |
| | b]indole |
| I-28 | 6-[[3-[(1S,3R)-2-(2-fluoro-2-methyl-propyl)-3-methyl-1,3,4,9- |
| | tetrahydropyrido[3,4-b]indol-1-yl]-1H-indazol-6-yl]methyl]-2-oxa-6- |
| | azaspiro[3.3]heptane |
| I-29 | (1R,3R)-1-[7-fluoro-6-[[3-(fluoromethyl)azetidin-1-yl]methyl]-1H-indol-3- |
| | yl]-2-(2-fluoro-2-methyl-propyl)-3-methyl-1,3,4,9-tetrahydropyrido[3,4- |
| | b]indole |
| I-30 | (1R,3R)-2-(2-fluoro-2-methyl-propyl)-3-methyl-1-[6-[(1-methylazetidin-3- |
| | yl)methyl]-1H-indol-3-yl]-1,3,4,9-tetrahydropyrido[3,4-b]indole |
| I-31 | 2-[[3-[(1S,3R)-2-(2-fluoro-2-methyl-propyl)-3-methyl-1,3,4,9- |
| | tetrahydropyrido[3,4-b]indol-1-yl]-1H-indazol-6-yl]methyl]-2- |
| | azaspiro[3.3]heptan-6-ol |
| I-32 | 1-[[3-[(1S,3R)-2-(2-fluoro-2-methyl-propyl)-3-methyl-1,3,4,9- |
| | tetrahydropyrido[3,4-b]indol-1-yl]-1H-indazol-6-yl]methyl]azetidine-3- |
| | carbonitrile |
| I-33 | (1S,3R)-1-[6-[(6-fluoro-2-azaspiro[3.3]heptan-2-yl)methyl]-1H-indazol-3- |
| | yl]-2-(2-fluoro-2-methyl-propyl)-3-methyl-1,3,4,9-tetrahydropyrido[3,4- |
| | b]indole |
| I-34 | (1S,3R)-1-[6-[(6,6-difluoro-1-azaspiro[3.3]heptan-1-yl)methyl]-1H-indazol- |
| | 3-yl]-2-(2-fluoro-2-methyl-propyl)-3-methyl-1,3,4,9-tetrahydropyrido[3,4- |
| | b]indole |
| I-35 | 3-((1R,3R)-2-(2-fluoro-2-methylpropyl)-3-methyl-2,3,4,9-tetrahydro-1H- |
| | pyrido[3,4-b]indol-1-yl)-N-(1-(3-fluoropropyl)azetidin-3-yl)-1H-indol-6- |
| | amine |
| I-36 | (1S,3R)-2-(2,2-difluoroethyl)-1-(6-((3-(fluoromethyl)azetidin-1-yl)methyl)- |
| | 1H-indazol-3-yl)-3-methyl-2,3,4,9-tetrahydro-1H-pyrido[3,4-b]indole |
| I-37 | (1S,3R)-2-(2,2-difluoroethyl)-3-methyl-1-(6-((3-methylazetidin-1-yl)methyl)- |
| | 1H-indazol-3-yl)-2,3,4,9-tetrahydro-1H-pyrido[3,4-b]indole |
| I-38 | (1S,3R)-1-(6-((6,6-difluoro-2-azaspiro[3.3]heptan-2-yl)methyl)-1H-indazol- |
| | 3-yl)-2-(2,2-difluoroethyl)-3-methyl-2,3,4,9-tetrahydro-1H-pyrido[3,4- |
| | b]indole |
| I-39 | 6-((3-((1S,3R)-2-(2,2-difluoroethyl)-3-methyl-2,3,4,9-tetrahydro-1H- |
| | pyrido[3,4-b]indol-1-yl)-1H-indazol-6-yl)methyl)-2-oxa-6- |
| | azaspiro[3.3]heptane |
| I-40 | (1R,3R)-1-[2-[[3-(fluoromethyl)azetidin-1-yl]methyl]-1H-indol-5-yl]-2-(2- |
| | fluoro-2-methyl-propyl)-3-methyl-1,3,4,9-tetrahydropyrido[3,4-b]indole |
| I-41 | (1R,3R)-1-[5-[[3-(fluoromethyl)azetidin-1-yl]methyl]-1H-indol-2-yl]-2-(2- |
| | fluoro-2-methyl-propyl)-3-methyl-1,3,4,9-tetrahydropyrido[3,4-b]indole |
| I-42 | (1R,3R)-2-(2-fluoro-2-methyl-propyl)-3-methyl-1-[2-[(3-methylazetidin-1- |
| | yl)methyl]-1H-indol-5-yl]-1,3,4,9-tetrahydropyrido[3,4-b]indole |
| I-43 | (1R,3R)-1-[2-(3-azabicyclo[3.1.0]hexan-3-ylmethyl)-1H-indol-5-yl]-2-(2- |
| | fluoro-2-methyl-propyl)-3-methyl-1,3,4,9-tetrahydropyrido[3,4-b]indole |
| I-44 | (1R,3R)-1-[2-(2-azaspiro[3.3]heptan-2-ylmethyl)-1H-indol-5-yl]-2-(2-fluoro- |
| | 2-methyl-propyl)-3-methyl-1,3,4,9-tetrahydropyrido[3,4-b]indole |
| I-45 | 2,2-difluoro-3-[(1R,3R)-1-[2-[[3-(fluoromethyl)azetidin-1-yl]methyl]-1H- |
| | indol-5-yl]-3-methyl-1,3,4,9-tetrahydropyrido[3,4-b]indol-2-yl]propan-1-ol |
| I-46 | 2,2-difluoro-3-[(1R,3R)-3-methyl-1-[2-[(3-methylazetidin-1-yl)methyl]-1H- |
| | indol-5-yl]-1,3,4,9-tetrahydropyrido[3,4-b]indol-2-yl]propan-1-ol |
| I-47 | (1R,3R)-2-(2-fluoro-2-methyl-propyl)-1-[2-[[(3S)-3-fluoropyrrolidin-1- |
| | yl]methyl]-1H-indol-5-yl]-3-methyl-1,3,4,9-tetrahydropyrido[3,4-b]indole |
| I-48 | 2-[1-[[5-[(1R,3R)-2-(2-fluoro-2-methyl-propyl)-3-methyl-1,3,4,9- |
| | tetrahydropyrido[3,4-b]indol-1-yl]-1H-indol-2-yl]methyl]azetidin-3- |
| | yl]propan-2-ol |
| I-49 | 6-(difluoromethyl)-2-[[5-[(1R,3R)-2-(2-fluoro-2-methyl-propyl)-3-methyl- |
| | 1,3,4,9-tetrahydropyrido[3,4-b]indol-1-yl]-1H-indol-2-yl]methyl]-2- |
| | azaspiro[3.3]heptan-6-ol |
| I-50 | 1-[[5-[(1R,3R)-2-(2-fluoro-2-methyl-propyl)-3-methyl-1,3,4,9- |
| | tetrahydropyrido[3,4-b]indol-1-yl]-1H-indol-2-yl]methyl]azetidine-3- |
| | carbonitrile |
| I-51 | (1R,3R)-2-(2-fluoro-2-methyl-propyl)-1-[2-[[3-(methoxymethyl)azetidin-1- |
| | yl]methyl]-1H-indol-5-yl]-3-methyl-1,3,4,9-tetrahydropyrido[3,4-b]indole |
| I-52 | (1R,3R)-1-[2-[(6-fluoro-2-azaspiro[3.3]heptan-2-yl)methyl]-1H-indol-5-yl]- |
| | 2-(2-fluoro-2-methyl-propyl)-3-methyl-1,3,4,9-tetrahydropyrido[3,4-b]indole |
| I-53 | 2-[[5-[(1R,3R)-2-(2-fluoro-2-methyl-propyl)-3-methyl-1,3,4,9- |
| | tetrahydropyrido[3,4-b]indol-1-yl]-1H-indol-2-yl]methyl]-2- |
| | azaspiro[3.3]heptan-6-ol |
| I-54 | 6-[[5-[(1R,3R)-2-(2-fluoro-2-methyl-propyl)-3-methyl-1,3,4,9- |
| | tetrahydropyrido[3,4-b]indol-1-yl]-1H-indol-2-yl]methyl]-2-oxa-6- |
| | azaspiro[3.3]heptane |
| I-55 | (1R,3R)-1-[2-[[3-(fluoromethyl)azetidin-1-yl]methyl]-1H-indol-6-yl]-2-(2- |
| | fluoro-2-methyl-propyl)-3-methyl-1,3,4,9-tetrahydropyrido[3,4-b]indole |
| I-56 | (1R,3R)-1-[6-[[3-(fluoromethyl)azetidin-1-yl]methyl]-1H-indol-2-yl]-2-(2- |
| | fluoro-2-methyl-propyl)-3-methyl-1,3,4,9-tetrahydropyrido[3,4-b]indole |
| I-57 | (1R,3R)-1-[2-[[3-(fluoromethyl)azetidin-1-yl]methyl]benzofuran-5-yl]-2-(2- |
| | fluoro-2-methyl-propyl)-3-methyl-1,3,4,9-tetrahydropyrido[3,4-b]indole |
| I-58 | (1R,3R)-1-[2-[(6,6-difluoro-1-azaspiro[3.3]heptan-1-yl)methyl]benzofuran-5- |
| | yl]-2-(2-fluoro-2-methyl-propyl)-3-methyl-1,3,4,9-tetrahydropyrido[3,4- |
| | b]indole |
| I-59 | (1R,3R)-2-(2-fluoro-2-methyl-propyl)-3-methyl-1-[5-[(3-methylazetidin-1- |
| | yl)methyl]-1H-pyrrolo[2,3-b]pyridin-3-yl]-1,3,4,9-tetrahydropyrido[3,4- |
| | b]indole |
| I-60 | (1R,3R)-1-[5-[[3-(fluoromethyl)azetidin-1-yl]methyl]-1H-pyrrolo[2,3- |
| | b]pyridin-3-yl]-2-(2-fluoro-2-methyl-propyl)-3-methyl-1,3,4,9- |
| | tetrahydropyrido[3,4-b]indole |
| I-61 | (1R,3R)-1-[5-(3-azabicyclo[3.1.0]hexan-3-ylmethyl)-1H-pyrrolo[2,3- |
| | b]pyridin-3-yl]-2-(2-fluoro-2-methyl-propyl)-3-methyl-1,3,4,9- |
| | tetrahydropyrido[3,4-b]indole |
| I-62 | 2-[(1S,3R)-2-(2-fluoro-2-methyl-propyl)-3-methyl-1,3,4,9- |
| | tetrahydropyrido[3,4-b]indol-1-yl]-5-[(3-methylazetidin-1-yl)methyl]-6H- |
| | thieno[2,3-b]pyrrole |
| I-63 | 5-[[3-(fluoromethyl)azetidin-1-yl]methyl]-2-[(1S,3R)-2-(2-fluoro-2-methyl- |
| | propyl)-3-methyl-1,3,4,9-tetrahydropyrido[3,4-b]indol-1-yl]-6H-thieno[2,3- |
| | b]pyrrole |
| I-64 | 5-[[3-(fluoromethyl)azetidin-1-yl]methyl]-2-[(1S,3R)-2-(2-fluoro-2-methyl- |
| | propyl)-3-methyl-1,3,4,9-tetrahydropyrido[3,4-b]indol-1-yl]-4H-thieno[3,2- |
| | b]pyrrole |
| I-65 | ammonium; 2-[(1S,3R)-2-(2-fluoro-2-methyl-propyl)-3-methyl-1,3,4,9- |
| | tetrahydropyrido[3,4-b]indol-1-yl]-4H-thieno[3,2-b]pyrrole-5-carboxylate |
| I-66 | 2-[(1S,3R)-2-(2-fluoro-2-methyl-propyl)-3-methyl-1,3,4,9- |
| | tetrahydropyrido[3,4-b]indol-1-yl]-5-[(3-methylazetidin-1-yl)methyl]-4H- |
| | thieno[3,2-b]pyrrole |
| I-67 | 3-fluoro-N-[[2-[(1S,3R)-2-(2-fluoro-2-methyl-propyl)-3-methyl-1,3,4,9- |
| | tetrahydropyrido[3,4-b]indol-1-yl]-4H-thieno[3,2-b]pyrrol-5- |
| | yl]methyl]propan-1-amine |
| I-68 | 5-[[(3S)-3-ethylpyrrolidin-1-yl]methyl]-2-[(1S,3R)-2-(2-fluoro-2-methyl- |
| | propyl)-3-methyl-1,3,4,9-tetrahydropyrido[3,4-b]indol-1-yl]-4H-thieno[3,2- |
| | b]pyrrole |
| I-69 | (1R,3R)-1-[2-[[3-(fluoromethyl)azetidin-1-yl]methyl]-1H-pyrrolo[2,3- |
| | b]pyridin-5-yl]-2-(2-fluoro-2-methyl-propyl)-3-methyl-1,3,4,9- |
| | tetrahydropyrido[3,4-b]indole |
| I-70 | (1S,3R)-1-[2-[[3-(fluoromethyl)azetidin-1-yl]methyl]-1H-pyrrolo[3,2- |
| | b]pyridin-5-yl]-2-(2-fluoro-2-methyl-propyl)-3-methyl-1,3,4,9- |
| | tetrahydropyrido[3,4-b]indole |
| I-71 | (1R,3R)-2-(2-fluoro-2-methyl-propyl)-3-methyl-1-[2-[(3-methylazetidin-1- |
| | yl)methyl]-3H-benzimidazol-5-yl]-1,3,4,9-tetrahydropyrido[3,4-b]indole |
| I-72 | 2-[[3-(fluoromethyl)azetidin-1-yl]methyl]-6-[(1R,3R)-2-(2-fluoro-2-methyl- |
| | propyl)-3-methyl-1,3,4,9-tetrahydropyrido[3,4-b]indol-1-yl]-1,3-benzoxazole |
| I-73 | (1R,3R)-1-[2-[[3-(fluoromethyl)azetidin-1-yl]methyl]imidazo[1,2-a]pyridin- |
| | 6-yl]-2-(2-fluoro-2-methyl-propyl)-3-methyl-1,3,4,9-tetrahydropyrido[3,4- |
| | b]indole |
| I-74 | (1R,3R)-1-[2-[[3-(fluoromethyl)azetidin-1-yl]methyl]imidazo[1,2-a]pyridin- |
| | 7-yl]-2-(2-fluoro-2-methyl-propyl)-3-methyl-1,3,4,9-tetrahydropyrido[3,4- |
| | b]indole |
| I-75 | 2-[[3-(fluoromethyl)azetidin-1-yl]methyl]-5-[(1R,3R)-2-(2-fluoro-2-methyl- |
| | propyl)-3-methyl-1,3,4,9-tetrahydropyrido[3,4-b]indol-1-yl]-1,3-benzoxazole |
| I-76 | 5-[(1R,3R)-2-(2-fluoro-2-methyl-propyl)-3-methyl-1,3,4,9- |
| | tetrahydropyrido[3,4-b]indol-1-yl]-2-[(3-methylazetidin-1-yl)methyl]-1,3- |
| | benzoxazole |
| I-77 | 2-(2-azaspiro[3.3]heptan-2-ylmethyl)-5-[(1R,3R)-2-(2-fluoro-2-methyl- |
| | propyl)-3-methyl-1,3,4,9-tetrahydropyrido[3,4-b]indol-1-yl]-1,3-benzoxazole |
| I-78 | 6-fluoro-2-[[3-(fluoromethyl)azetidin-1-yl]methyl]-5-[(1R,3R)-2-(2-fluoro-2- |
| | methyl-propyl)-3-methyl-1,3,4,9-tetrahydropyrido[3,4-b]indol-1-yl]-1,3- |
| | benzoxazole |
| I-79 | 7-fluoro-2-[[3-(fluoromethyl)azetidin-1-yl]methyl]-5-[(1R,3R)-2-(2-fluoro-2- |
| | methyl-propyl)-3-methyl-1,3,4,9-tetrahydropyrido[3,4-b]indol-1-yl]-1,3- |
| | benzoxazole |
| I-80 | 2,2-difluoro-3-[(1R,3R)-1-[2-[[3-(fluoromethyl)azetidin-1-yl]methyl]-1,3- |
| | benzoxazol-5-yl]-3-methyl-1,3,4,9-tetrahydropyrido[3,4-b]indol-2-yl]propan- |
| | 1-ol |
| I-81 | 2,2-difluoro-3-[(1R,3R)-1-[7-fluoro-2-[[3-(fluoromethyl)azetidin-1- |
| | yl]methyl]-1,3-benzoxazol-5-yl]-3-methyl-1,3,4,9-tetrahydropyrido[3,4- |
| | b]indol-2-yl]propan-1-ol |
| I-82 | 5-[(1R,3R)-2-(2-fluoro-2-methyl-propyl)-3-methyl-1,3,4,9- |
| | tetrahydropyrido[3,4-b]indol-1-yl]-2-[1-(3-fluoropropyl)azetidin-3-yl]-1,3- |
| | benzoxazole |
| I-83 | 2-(1-((5-((1R,3R)-2-(2-fluoro-2-methylpropyl)-3-methyl-2,3,4,9-tetrahydro- |
| | 1H-pyrido[3,4-b]indol-1-yl)-1H-indol-2-yl)methyl)azetidin-3-yl)propan-2-ol |
| I-84 | 6-(difluoromethyl)-2-((5-((1R,3R)-2-(2-fluoro-2-methylpropyl)-3-methyl- |
| | 2,3,4,9-tetrahydro-1H-pyrido[3,4-b]indol-1-yl)-1H-indol-2-yl)methyl)-2- |
| | azaspiro[3.3]heptan-6-ol |
| I-85 | 1-((5-((1R,3R)-2-(2-fluoro-2-methylpropyl)-3-methyl-2,3,4,9-tetrahydro-1H- |
| | pyrido[3,4-b]indol-1-yl)-1H-indol-2-yl)methyl)azetidine-3-carbonitrile |
| I-86 | (1R,3R)-2-(2-fluoro-2-methylpropyl)-1-(2-((3-(methoxymethyl)azetidin-1- |
| | yl)methyl)-1H-indol-5-yl)-3-methyl-2,3,4,9-tetrahydro-1H-pyrido[3,4- |
| | b]indole |
| I-87 | (1R,3R)-1-(2-((6-fluoro-2-azaspiro[3.3]heptan-2-yl)methyl)-1H-indol-5-yl)- |
| | 2-(2-fluoro-2-methylpropyl)-3-methyl-2,3,4,9-tetrahydro-1H-pyrido[3,4- |
| | b]indole |
| I-88 | 2-((5-((1R,3R)-2-(2-fluoro-2-methylpropyl)-3-methyl-2,3,4,9-tetrahydro-1H- |
| | pyrido[3,4-b]indol-1-yl)-1H-indol-2-yl)methyl)-2-azaspiro[3.3]heptan-6-ol |
| I-89 | 6-((5-((1R,3R)-2-(2-fluoro-2-methylpropyl)-3-methyl-2,3,4,9-tetrahydro-1H- |
| | pyrido[3,4-b]indol-1-yl)-1H-indol-2-yl)methyl)-2-oxa-6-azaspiro[3.3]heptane |
| I-90 | (1R,3R)-1-[2-[(3-fluoroazetidin-1-yl)methyl]-1H-indol-5-yl]-2-(2-fluoro-2- |
| | methyl-propyl)-3-methyl-1,3,4,9-tetrahydropyrido[3,4-b]indole |
| I-91 | (1R,3R)-1-[2-[(3-ethylazetidin-1-yl)methyl]-1H-indol-5-yl]-2-(2-fluoro-2- |
| | methyl-propyl)-3-methyl-1,3,4,9-tetrahydropyrido[3,4-b]indole |
| I-92 | (1R,3R)-1-[2-[(3,3-dimethylazetidin-1-yl)methyl]-1H-indol-5-yl]-2-(2-fluoro- |
| | 2-methyl-propyl)-3-methyl-1,3,4,9-tetrahydropyrido[3,4-b]indole |
| I-93 | [5-[(1R,3R)-2-(2-fluoro-2-methyl-propyl)-3-methyl-1,3,4,9- |
| | tetrahydropyrido[3,4-b]indol-1-yl]-1H-indol-2-yl]methanol |
| I-94 | (1R,3R)-2-(2-fluoro-2-methyl-propyl)-1-[2-[(3-methoxyazetidin-1- |
| | yl)methyl]-1H-indol-5-yl]-3-methyl-1,3,4,9-tetrahydropyrido[3,4-b]indole |
| I-95 | (1R,3R)-2-(2-fluoro-2-methyl-propyl)-1-[2-[(3-isopropylazetidin-1- |
| | yl)methyl]-1H-indol-5-yl]-3-methyl-1,3,4,9-tetrahydropyrido[3,4-b]indole |
| I-96 | (1R,3R)-2-(2-fluoro-2-methyl-propyl)-1-[2-[(3-methoxy-3-methyl-azetidin-1- |
| | yl)methyl]-1H-indol-5-yl]-3-methyl-1,3,4,9-tetrahydropyrido[3,4-b]indole |
| I-97 | (1R,3R)-1-[2-[[3-(fluoromethyl)azetidin-1-yl]methyl]-1H-indol-5-yl]-3- |
| | methyl-2-(2,2,2-trifluoroethyl)-1,3,4,9-tetrahydropyrido[3,4-b]indole |
| I-98 | 2-[1-[[5-[(1R,3R)-2-(2-fluoro-2-methyl-propyl)-3-methyl-1,3,4,9- |
| | tetrahydropyrido[3,4-b]indol-1-yl]-1H-indol-2-yl]methyl]azetidin-3- |
| | yl]acetonitrile |
| I-99 | 1-[[5-[(1R,3R)-2-(2-fluoro-2-methyl-propyl)-3-methyl-1,3,4,9- |
| | tetrahydropyrido[3,4-b]indol-1-yl]-1H-indol-2-yl]methyl]-N,N-dimethyl- |
| | azetidin-3-amine |
| I-100 | (1R,3R)-1-[2-[[3-(difluoromethyl)azetidin-1-yl]methyl]-1H-indol-5-yl]-2-(2- |
| | fluoro-2-methyl-propyl)-3-methyl-1,3,4,9-tetrahydropyrido[3,4-b]indole |
| I-101 | (1R,3R)-1-[2-[(3,3-difluoroazetidin-1-yl)methyl]-1H-indol-5-yl]-2-(2-fluoro- |
| | 2-methyl-propyl)-3-methyl-1,3,4,9-tetrahydropyrido[3,4-b]indole |
| I-102 | (1R,3R)-1-[2-[(3-fluoro-3-methyl-azetidin-1-yl)methyl]-1H-indol-5-yl]-2-(2- |
| | fluoro-2-methyl-propyl)-3-methyl-1,3,4,9-tetrahydropyrido[3,4-b]indole |
| I-103 | 2-[[3-(fluoromethyl)azetidin-1-yl]methyl]-5-[(1R,3R)-2-(2-fluoro-2-methyl- |
| | propyl)-3-methyl-1,3,4,9-tetrahydropyrido[3,4-b]indol-1-yl]-1,3- |
| | benzothiazole |
| I-104 | 2-[[3-(fluoromethyl)azetidin-1-yl]methyl]-5-[(1R,3R)-2-(2-fluoro-2-methyl- |
| | propyl)-3-methyl-1,3,4,9-tetrahydropyrido[3,4-b]indol-1-yl]-1,3- |
| | benzothiazole 1-oxide |
| I-105 | 2,2-difluoro-3-[rac-(1R,3R)-3-methyl-1-[1-(1-propylazetidin-3-yl)indazol-5- |
| | yl]-1,3,4,9-tetrahydropyrido[3,4-b]indol-2-yl]propan-1-ol |
| I-106 | 2,2-difluoro-3-[rac-(1R,3R)-1-[1-[1-(3-fluoropropyl)azetidin-3-yl]indazol-5- |
| | yl]-3-methyl-1,3,4,9-tetrahydropyrido[3,4-b]indol-2-yl]propan-1-ol |
| I-107 | 2,2-difluoro-3-[rac-(1R,3R)-1-[2-(1-ethylazetidin-3-yl)indazol-5-yl]-3- |
| | methyl-1,3,4,9-tetrahydropyrido[3,4-b]indol-2-yl]propan-1-ol |
| I-108 | 2,2-difluoro-3-[rac-(1R,3R)-3-methyl-1-[2-(1-propylazetidin-3-yl)indazol-5- |
| | yl]-1,3,4,9-tetrahydropyrido[3,4-b]indol-2-yl]propan-1-ol |
| I-109 | (1R,3R)-1-[2-[[3-(fluoromethyl)azetidin-1-yl]methyl]-1H-indol-5-yl]-3- |
| | methyl-2-(2,2,3,3-tetrafluoropropyl)-1,3,4,9-tetrahydropyrido[3,4-b]indole |
| I-110 | (1R,3R)-1-[2-[[3-(fluoromethyl)azetidin-1-yl]methyl]-1H-indol-5-yl]-3- |
| | methyl-2-(2,2,3,3,3-pentafluoropropyl)-1,3,4,9-tetrahydropyrido[3,4-b]indole |

In some embodiments, the present disclosure provides a compound selected from Table 3, or a pharmaceutically acceptable salt thereof:

TABLE 3
Compound No.

I-1
I-2
I-3
I-4
I-7
I-8
I-9
I-12
I-13
I-14
I-18
I-19
I-20
I-21
I-22
I-24
I-25
I-30
I-31
I-32
I-34
I-35
I-43
I-47
I-48
I-49
I-50
I-51
I-53
I-54
I-55
I-56
I-58
I-59
I-60
I-61
I-66
I-67
I-68
I-70
I-72
I-73
I-78
I-79
I-81
I-82
I-83
I-84
I-85
I-86
I-87
I-88
I-89
I-90
I-91
I-92
I-93
I-94
I-95
I-96
I-97
I-103
I-104
I-107
I-108

In some embodiments, a compound provided herein is an agonist, and is selected from Table 3.

In some embodiments, the present disclosure provides a compound selected from Table 4, or a pharmaceutically acceptable salt thereof:

TABLE 4
Compound No.

I-5
I-6
I-10
I-11
I-15
I-16
I-17
I-23
I-26
I-27
I-28
I-29
I-33
I-36
I-37
I-38
I-39
I-40
I-41
I-42
I-44
I-45
I-46
I-52
I-57
I-62
I-63
I-64
I-65
I-69
I-71
I-74
I-75
I-76
I-77
I-80
I-98
I-99
I-100
I-101
I-102
I-105
I-106
I-109
I-110

In some embodiments, a compound provided herein is an antagonist, and is selected from Table 4.

In some embodiments, the present disclosure provides a compound selected from Table 5, or a pharmaceutically acceptable salt thereof:

TABLE 5
Compound No.

I-10
I-17
I-26
I-33
I-40
I-45
I-46
I-57
I-74
I-75
I-76
I-77
I-80
I-109
I-110

In some embodiments, a compound provided herein is an antagonist, and is selected from Table 5.

In some embodiments, provided compounds are provided and/or utilized in a salt form (e.g., a pharmaceutically acceptable salt form). Reference to a compound provided herein is understood to include reference to salts thereof, unless otherwise indicated.

It will be appreciated that throughout the present disclosure, unless otherwise indicated, reference to a compound of Formula I is intended to also include Formulae I-VII, and compound species of such formulas disclosed herein.

### Preparing Provided Compounds

Provided compounds may generally be made by the processes described in the ensuing schemes and examples. In some embodiments, provided compounds are prepared according to Schemes 1-4:

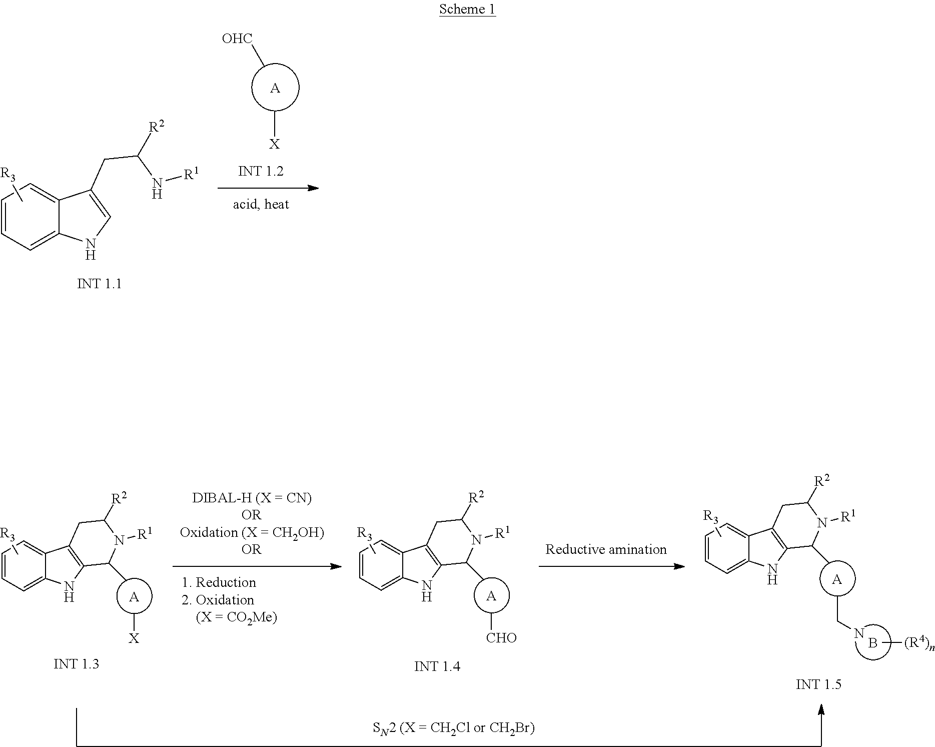

In some embodiments, compounds described herein are prepared via Pictet-Spengler reaction between an appropriately substituted tryptamine (INT 1.1) and a heterocyclic aldehyde of interest (INT 1.2), wherein R¹, R², R³, and A are as defined in classes and subclasses herein with respect to Formula I both singly and in combination, and X is —CN, —CH₂OH, —CO₂Me, —CH₂Cl, or —CH₂Br. In some embodiments, INT 1.3 then undergoes selective reduction (when X is —CN), selective oxidation (when X is —CH₂OH), or a two-step reduction-oxidation sequence (when X is —CO₂Me). In some embodiments, a B group comprising an amine and substituted with (R⁴)ₙ is coupled with INT 1.4 by reductive amination to provide INT 1.5 (wherein B, R⁴, and n are as defined in classes and subclasses herein with respect to Formula I both singly and in combination). Alternatively, INT 1.3 undergoes a substitution (when X is —CH₂Cl, or —CH₂Br) with a B group comprising an amine and substituted with (R⁴)ₙ to provide INT 1.5 directly.

As another alternative, in some embodiments, the X group of TNT 1.3 is an amine precursor such as —CN or —CH₂NHBoc. In some such embodiments, after conversion of the amine precursor to an amine by selective reduction or acidic deprotection, the intermediates are subjected to reductive amination to provide INT 1.5.

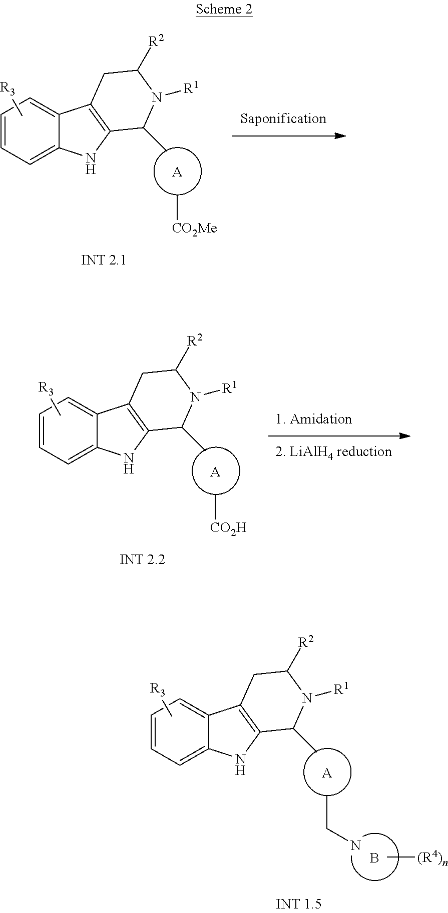

In some embodiments, target compounds are prepared via a late-stage, two-step conversion of advanced intermediate carboxylic acids to amines. For example, in some embodiments, a compound INT 2.1 is saponified to provide carboxylic acid INT 2.2, wherein R¹, R², R³, and A are as defined in classes and subclasses herein with respect to Formula 1. In some embodiments, INT 2.2 is subjected to amidation with amines of interest (e.g., a B group comprising an amine group and substituted with (R⁴)ₙ), followed by amide reduction to provide compound INT 1.5.

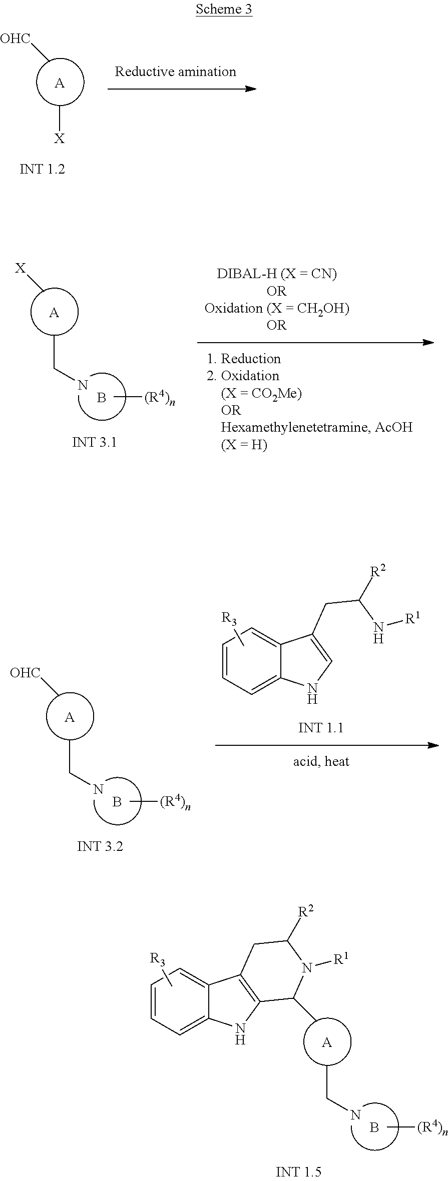

In some embodiments, preparation of targeted compounds begins with reductive amination between appropriately substituted heterocyclic aldehydes (INT 1.2) and amines of interest (e.g., a B group comprising an amine group and substituted with (R⁴)ₙ), to provide INT 3.1. In some embodiments, INT 3.1 undergoes selective reduction (when X is —CN), selective oxidation (when X is —CH₂OH), a two-step reduction-oxidation sequence (when X is —CO₂Me), or formylation (when X is H) to provide INT 3.2. In some embodiments, INT 3.2 is reacted with INT 1.1 in the presence of acid and heat to undergo a Pictet-Spengler reaction to provide INT 1.5.

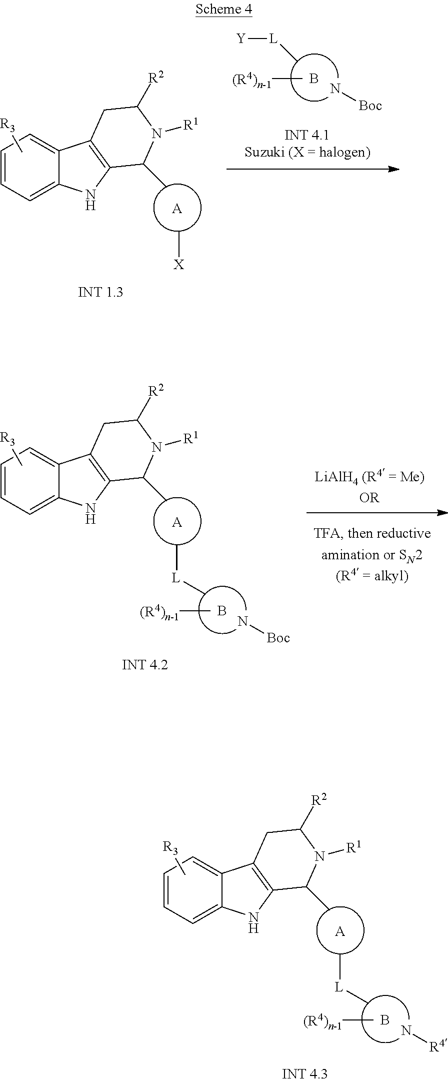

In some embodiments, compounds described herein are prepared as shown in Scheme 4. In some embodiments, INT 1.3 undergoes a Suzuki-type cross coupling reaction (when X is halogen) with INT 4.1, wherein Y is boronate ester or boronic acid and L is a covalent bond or contains at least one carbon atom (e.g., L is —CH₂—). In some embodiments, INT 4.2 undergoes reduction with LiAlH₄ to give INT 4.3, wherein R^{4′} is Me, or acidic deprotection, followed by further functionalization, for example, via reductive amination, S_{N}2 alkylation, or various acylation reactions to give INT 4.3, wherein R^{4′} is alkyl.

### Uses, Formulation, and Administration

The present disclosure provides uses for compounds and compositions described herein. In some embodiments, provided compounds and compositions are useful in medicine (e.g., as therapy). In some embodiments, provided compounds and compositions are useful in research as, for example, analytical tools and/or control compounds in biological assays.

### Pharmaceutically Acceptable Compositions

In some embodiments, the present disclosure provides a composition comprising a compound described herein or a pharmaceutically acceptable salt thereof and a pharmaceutically acceptable carrier, adjuvant, or vehicle. In some embodiments, the amount of compound in compositions described herein is such that it is effective to measurably induce degradation of a target in a biological sample or in a patient. In some embodiments, a composition described herein is formulated for administration to a patient in need of such composition. In some embodiments, a composition described herein is formulated for oral administration to a patient.

Pharmaceutical compositions typically contain an active agent (e.g., a compound described herein) in an amount effective to achieve a desired therapeutic effect while avoiding or minimizing adverse side effects. In some embodiments, provided pharmaceutical compositions comprise a compound described herein and one or more fillers, disintegrants, lubricants, glidants, anti-adherents, and/or anti-statics, etc. Provided pharmaceutical compositions can be in a variety of forms including oral dosage forms, topical creams, topical patches, iontophoresis forms, suppository, nasal spray and/or inhaler, eye drops, intraocular injections forms, deport forms, as well as injectable and infusible solutions. Methods of preparing pharmaceutical compositions are well known in the art.

In some embodiments, provided compounds are formulated in a unit dosage form for ease of administration and uniformity of dosage. A unit dosage form may be, for example, a liquid pharmaceutical composition containing a predetermined quantity of one or more active agents, a solid pharmaceutical composition (e.g., a tablet, a capsule, or the like) containing a predetermined quantity of one or more active agents, a sustained release formulation containing a predetermined quantity of one or more active agents, or a drug delivery device containing a predetermined amount of one or more active agents, etc.

Provided compositions may be administered using any amount and any route of administration effective for treating or lessening the severity of any disease or disorder described herein. For example, compositions of the present disclosure may be administered orally, parenterally, by inhalation spray, topically, rectally, nasally, buccally, vaginally, intraperitoneally, intracisternallyor via an implanted reservoir. In some embodiments, the compositions are administered orally, intraperitoneally or intravenously.

Sterile injectable forms of the compositions described herein may be aqueous or oleaginous suspension. These suspensions may be formulated according to techniques known in the art using suitable dispersing or wetting agents and suspending agents. The sterile injectable preparation may also be a sterile injectable solution or suspension in a non-toxic parenterally acceptable diluent or solvent, for example as a solution in 1,3-butanediol. Among the acceptable vehicles and solvents that may be employed are water, Ringer's solution and isotonic sodium chloride solution. In addition, sterile, fixed oils are conventionally employed as a solvent or suspending medium.

For this purpose, any bland fixed oil may be employed including synthetic mono- or di-glycerides. Fatty acids, such as oleic acid and its glyceride derivatives are useful in the preparation of injectables, as are natural pharmaceutically acceptable oils, such as olive oil or castor oil, especially in their polyoxyethylated versions. These oil solutions or suspensions may also contain a long-chain alcohol diluent or dispersant, such as carboxymethyl cellulose or similar dispersing agents that are commonly used in the formulation of pharmaceutically acceptable dosage forms including emulsions and suspensions. Other commonly used surfactants, such as Tweens, Spans and other emulsifying agents or bioavailability enhancers which are commonly used in the manufacture of pharmaceutically acceptable solid, liquid, or other dosage forms may also be used for the purposes of formulation.

Injectable formulations can be sterilized, for example, by filtration through a bacterial-retaining filter, or by incorporating sterilizing agents in the form of sterile solid compositions which can be dissolved or dispersed in sterile water or other sterile injectable medium prior to use.

In order to prolong the effect of a compound of the present disclosure, it is often desirable to slow the absorption of the compound from subcutaneous or intramuscular injection. This may be accomplished by the use of a liquid suspension of crystalline or amorphous material with poor water solubility. The rate of absorption of the compound then depends upon its rate of dissolution that, in turn, may depend upon crystal size and crystalline form. Alternatively, delayed absorption of a parenterally administered compound form is accomplished by dissolving or suspending the compound in an oil vehicle. Injectable depot forms are made by forming microencapsule matrices of the compound in biodegradable polymers such as polylactide-polyglycolide. Depending upon the ratio of compound to polymer and the nature of the particular polymer employed, the rate of compound release can be controlled. Examples of other biodegradable polymers include poly(orthoesters) and poly(anhydrides). Depot injectable formulations are also prepared by entrapping the compound in liposomes or microemulsions that are compatible with body tissues.

In some embodiments, provided pharmaceutically acceptable compositions are formulated for oral administration. Such formulations may be administered with or without food. In some embodiments, pharmaceutically acceptable compositions described herein are administered without food. In other embodiments, pharmaceutically acceptable compositions described herein are administered with food. Pharmaceutically acceptable compositions described herein may be orally administered in any orally acceptable dosage form including, but not limited to, capsules, tablets, aqueous suspensions or solutions. In the case of tablets for oral use, carriers commonly used include lactose and corn starch. Lubricating agents, such as magnesium stearate, are also typically added. For oral administration in a capsule form, useful diluents include lactose and dried cornstarch. When aqueous suspensions are required for oral use, the active ingredient is combined with emulsifying and suspending agents. If desired, certain sweetening, flavoring or coloring agents may also be added.

Solid dosage forms for oral administration include capsules, tablets, pills, powders, and granules. In such solid dosage forms, the active compound is mixed with at least one inert, pharmaceutically acceptable excipient or carrier such as sodium citrate or dicalcium phosphate and/or a) fillers or extenders such as starches, lactose, sucrose, glucose, mannitol, and silicic acid, b) binders such as, for example, carboxymethylcellulose, alginates, gelatin, polyvinylpyrrolidinone, sucrose, and acacia, c) humectants such as glycerol, d) disintegrating agents such as agar-agar, calcium carbonate, potato or tapioca starch, alginic acid, certain silicates, and sodium carbonate, e) solution retarding agents such as paraffin, f) absorption accelerators such as quaternary ammonium compounds, g) wetting agents such as, for example, cetyl alcohol and glycerol monostearate, h) absorbents such as kaolin and bentonite clay, and/or i) lubricants such as talc, calcium stearate, magnesium stearate, solid polyethylene glycols, sodium lauryl sulfate, and mixtures thereof. In the case of capsules, tablets and pills, the dosage form may also comprise buffering agents.

Solid compositions of a similar type may also be employed as fillers in soft and hard-filled gelatin capsules using such excipients as lactose or milk sugar as well as high molecular weight polyethylene glycols and the like. The solid dosage forms of tablets, dragees, capsules, pills, and granules can be prepared with coatings and shells such as enteric coatings and other coatings well known in the pharmaceutical formulating art. They may optionally contain opacifying agents and can also be of a composition that they release the active ingredient(s) only, or preferentially, in a certain part of the intestinal tract, optionally, in a delayed manner. Examples of embedding compositions that can be used include polymeric substances and waxes. Solid compositions of a similar type may also be employed as fillers in soft and hard-filled gelatin capsules using such excipients as lactose or milk sugar as well as high molecular weight polethylene glycols and the like.

The active compounds can also be in micro-encapsulated form with one or more excipients as noted above. The solid dosage forms of tablets, dragees, capsules, pills, and granules can be prepared with coatings and shells such as enteric coatings, release controlling coatings and other coatings well known in the pharmaceutical formulating art. In such solid dosage forms the active compound may be admixed with at least one inert diluent such as sucrose, lactose or starch. Such dosage forms may also comprise, as is normal practice, additional substances other than inert diluents, e.g., tableting lubricants and other tableting aids such a magnesium stearate and microcrystalline cellulose. In the case of capsules, tablets and pills, the dosage forms may also comprise buffering agents. They may optionally contain opacifying agents and can also be of a composition that they release the active ingredient(s) only, or preferentially, in a certain part of the intestinal tract, optionally, in a delayed manner. Examples of embedding compositions that can be used include polymeric substances and waxes.

Liquid dosage forms for oral administration include, but are not limited to, pharmaceutically acceptable emulsions, microemulsions, solutions, suspensions, syrups and elixirs. In addition to the active compounds, the liquid dosage forms may contain inert diluents commonly used in the art such as, for example, water or other solvents, solubilizing agents and emulsifiers such as ethyl alcohol, isopropyl alcohol, ethyl carbonate, ethyl acetate, benzyl alcohol, benzyl benzoate, propylene glycol, 1,3-butylene glycol, dimethylformamide, oils (in particular, cottonseed, groundnut, corn, germ, olive, castor, and sesame oils), glycerol, tetrahydrofurfuryl alcohol, polyethylene glycols and fatty acid esters of sorbitan, and mixtures thereof. Besides inert diluents, the oral compositions can also include adjuvants such as wetting agents, emulsifying and suspending agents, sweetening, flavoring, and perfuming agents.

Alternatively, pharmaceutically acceptable compositions described herein may be administered in the form of suppositories for rectal administration. These can be prepared by mixing the agent with a suitable non-irritating excipient that is solid at room temperature but liquid at rectal temperature and therefore will melt in the rectum to release the drug. Such materials include cocoa butter, beeswax and polyethylene glycols.

Compositions for rectal or vaginal administration are preferably suppositories which can be prepared by mixing the compounds described herein with suitable non-irritating excipients or carriers such as cocoa butter, polyethylene glycol or a suppository wax which are solid at ambient temperature but liquid at body temperature and therefore melt in the rectum or vaginal cavity and release the active compound.

Pharmaceutically acceptable compositions described herein may also be administered topically, especially when the target of treatment includes areas or organs readily accessible by topical application, including diseases of the eye, the skin, or the lower intestinal tract. Suitable topical formulations are readily prepared for each of these areas or organs.

Topical application for the lower intestinal tract can be effected in a rectal suppository formulation (see above) or in a suitable enema formulation. Topically-transdermal patches may also be used.

For topical applications, provided pharmaceutically acceptable compositions may be formulated in a suitable ointment containing the active component suspended or dissolved in one or more carriers. Carriers for topical administration of compounds described herein include, but are not limited to, mineral oil, liquid petrolatum, white petrolatum, propylene glycol, polyoxyethylene, polyoxypropylene compound, emulsifying wax and water. Alternatively, provided pharmaceutically acceptable compositions can be formulated in a suitable lotion or cream containing the active components suspended or dissolved in one or more pharmaceutically acceptable carriers. Suitable carriers include, but are not limited to, mineral oil, sorbitan monostearate, polysorbate 60, cetyl esters wax, cetearyl alcohol, 2-octyldodecanol, benzyl alcohol and water.

For ophthalmic use, provided pharmaceutically acceptable compositions may be formulated as micronized suspensions in isotonic, pH adjusted sterile saline, or, preferably, as solutions in isotonic, pH adjusted sterile saline, either with or without a preservative such as benzylalkonium chloride. Alternatively, for ophthalmic uses, the pharmaceutically acceptable compositions may be formulated in an ointment such as petrolatum.

Pharmaceutically acceptable compositions described herein may also be administered by nasal aerosol or inhalation. Such compositions are prepared according to techniques well-known in the art of pharmaceutical formulation and may be prepared as solutions in saline, employing benzyl alcohol or other suitable preservatives, absorption promoters to enhance bioavailability, fluorocarbons, and/or other conventional solubilizing or dispersing agents.

Dosage forms for topical or transdermal administration of a compound disclosed herein include ointments, pastes, creams, lotions, gels, powders, solutions, sprays, inhalants or patches. The active component is admixed under sterile conditions with a pharmaceutically acceptable carrier and any needed preservatives or buffers as may be required. Ophthalmic formulation, ear drops, and eye drops are also contemplated as being within the scope of this disclosure. Additionally, the present disclosure contemplates the use of transdermal patches, which have the added advantage of providing controlled delivery of a compound to the body. Such dosage forms can be made by dissolving or dispensing the compound in the proper medium. Absorption enhancers can also be used to increase the flux of the compound across the skin. The rate can be controlled by either providing a rate controlling membrane or by dispersing the compound in a polymer matrix or gel.

### Estrogen Receptor-Associated Diseases and Disorders

The estrogen receptor (“ER”) is involved in a variety of biological processes, relating, for example, to development of the female reproductive system, maintenance of bone mass, protection of cardiovascular and/or central nervous system components, etc. (see, for example, Pearce & Jordan Crit. Rev. Onc/Hem 50:3, 2004; Heldring Phys. Rev. 87:905, 2007). The ER has been implicated in a variety of cancers. In many tumors that express the estrogen receptor (i.e., ER⁺ tumors), active ERα signaling has been demonstrated to drive cell proliferation (although ERβ signaling has been reported to be able to achieve tumor suppressor effects; see, for example, Nilsson & Gustafson Clin. Pharmacol. Ther. 89:44, 2011). Typically, tumors (e.g., breast tumors) with as few as 1% of cells staining positive for ER are classified as “ER⁺”. Therapies targeting the ER are standard of care for many patients with ER⁺ tumors (see, for example, Cardoso et al Annals Onc. <https://doi.org/10.1093/announc/mdmx036>, 2017; Rugo et al. J. Clin. Oncol. 34:3069, 2016; Senkus et al Annal Onc. 26:v8, 2015; Sareddy & Vadlamudi Clin. J Nat. Med, 13:801, 2015). For early stage breast cancer patients, for example, recommended therapy typically involves tumor resection, followed by ER-targeted therapy (e.g., as discussed below). For advanced breast cancer, including metastatic breast cancer, ER-targeted therapy is the mainstay.

Given the importance of ER signaling in many cancers, as well as in certain cardiovascular, inflammatory, and neurodegenerative diseases, significant effort has been invested in developing therapeutic agents and modalities that target the ER. There is some fluidity/flexibility in terminology that has been used to describe ER-targeting agents, but a variety of agents, with different mechanisms, have been developed and/or studied.

For example, some ER-targeting agents are designed and/or documented to reduce levels of estrogen (i.e., 17β estradiol) production. In other embodiments, some ER-targeting agents are designed and/or documented to increase levels of estrogen production.

Some ER-targeting agents are designed and/or documented to bind directly to the ER; in some cases, such agents compete with estrogen for binding to the ER and/or interfere with the allosteric changes that estrogen binding would naturally produce. Often, the term “antiestrogen” is used to refer to agents that bind to the ER, and sometimes is specifically used to indicate those agents that compete with estrogen for ER binding.

The term “selective estrogen receptor modulator, “SERM”, has been used to refer to compounds that are designed and/or documented to alter some aspect of ER activity. Some writings refer to “SERMs” as representing a particular type of anti-estrogens; other writings, however, use the term “SERM” more generally, to refer to a compound that specifically impacts some feature of ER (particularly ERα) expression and/or activity.

The term “selective estrogen receptor degrader” (“SERD”) has been used to refer to compounds that are designed and/or documented to trigger or enhance degradation of the ER. In many instances, if presence of a compound correlates with reduced level of ER, the compound may be referred to as a SERD. Some writings classify compounds either as SERMs or as SERDs; others refer to SERDs as a particular type, or species, of compounds that are SERMs.

Regardless of mechanism of action of a particular agent, clinical experience thus far has revealed that incomplete effects (e.g., within an individual patient and/or across patient populations) and/or development of resistance remain a problem.

Among other things, presence or development of certain ER mutations has been reported to impact effectiveness of various ER-targeted therapies (see, for example, Jeselsohn et al Nature Rev. Clin. Onc. 12, 573, 2015; Gelsomino et al. Breast Cancer Res. Treat 157:253, 2016; Toy et al. 2013). Some particularly problematic mutations are those that “activate” one or more aspects of ER expression and/or function; some activating mutations have been reported that can render the ER ligand-independent (i.e., constitutively active). For example, particular mutations in the ER ligand binding domain, including D538G and Y537S, have been demonstrated to constitutively activate the ER; other mutations including deletions and/or fusions that remove the ligand binding domain, can have similar effects (see, for example, Li et al. Cell Repts 4:1116, 2013; Veeraraghavan et al Breast Cancer Research and Treatment 158, 219-232, 2016; Veeraraghavan, et al. Nature Comms 5:4577, 2014). Some reports have indicated that as many as 50% of women with metastatic breast cancer may have activating ER mutations detectible in circulating tumor DNA.

### Estrogen Receptor Antagonists

In some embodiments, compounds provided herein are estrogen receptor antagonists. As used herein, an “estrogen receptor antagonist” refers to a compound or composition that produces an antagonistic effect when contacting the estrogen receptor of a subject or biological sample. In some embodiments, an estrogen receptor antagonist is characterized by having:
1. (i) between 10% and 80% increase in the E2-normalized signal in the AP assay (agonist mode) of Example 209 and (ii) between 10% and 80% reduction in the E2-normalized signal in the AP assay (antagonist mode) of Example 209;
2. (i) a pIC₅₀ greater than 6.4 and at least a 10% reduction in the E2-normalized signal in the AP assay (antagonist mode) of Example 209; and (ii) no more than 10% increase in E2-normalized signal in the AP assay (agonist mode) of Example 209; or
3. (i) a pIC₅₀ greater than 7.5 and at least a 10% reduction in the E2-normalized signal in the AP assay (antagonist mode) of Example 209; and (ii) no more than 10% increase in E2-normalized signal in the AP assay (agonist mode) of Example 209.

In some embodiments, compounds provided herein are complete estrogen receptor antagonists. As described herein, a complete estrogen receptor antagonist (a “CERAN”) is one that (1) inhibits both AF l and AF2, and in particular inhibits AF l activity that remains present in constitutively active ER mutants; (2) promotes ER degradation; and (3) lacks the partial ER agonist activity observed with certain other agents. The present disclosure further appreciates that many previous therapies, including for example, ARN-810, AZD9496, tamoxifen, and others, are less effective than CERANs at least in part because they only partially antagonize ER, and specifically because they inhibit activation of AF2 but not AF1. In some embodiments, an estrogen receptor antagonist is characterized by having (i) a pIC₅₀ greater than 6.4 and at least a 10% reduction in the E2-normalized signal in the AP assay (antagonist mode) of Example 209; and (ii) no more than 10% increase in E2-normalized signal in the AP assay (agonist mode) of Example 209. In some embodiments, an estrogen receptor antagonist is characterized by having (i) a pIC₅₀ greater than 7.5 and at least a 10% reduction in the E2-normalized signal in the AP assay (antagonist mode) of Example 209; and (ii) no more than 10% increase in E2-normalized signal in the AP assay (agonist mode) of Example 209.

### Estrogen Receptor Agonists

In some embodiments, compounds provided herein are estrogen receptor agonists. As used herein, an “estrogen receptor agonist” refers to a compound or composition that produces an agonistic effect when contacting the estrogen receptor of a subject or biological sample. In some embodiments, an estrogen receptor agonist is characterized by having (i) at least 80% increase in the E2-normalized signal in the AP assay (agonist mode) of Example 209 and (ii) no more than 80% reduction in the E2-normalized signal in the AP assay (antagonist mode) of Example 209.

### Diseases, Disorders, and Conditions

The present disclosure encompasses the insight that provided compounds have a number of uses, including treatment of an ER-associated disorder (e.g., an ER-associated cancer such as breast cancer, osteoporosis, or menopause symptoms), detection, and/or characterization of certain tumors. In some embodiments, a disease, disorder, or condition is a cancer. In some embodiments, a disease, disorder, or condition is associated with a mutation in an estrogen receptor.

In some embodiments, provided compounds are useful for treating a disorder associated with increased ER activity (e.g., an ER-associated cancer such as breast cancer). In some embodiments, provided estrogen receptor antagonists (e.g., complete estrogen receptor antagonists) are useful for treating such disorders.

In some embodiments, provided compounds are useful for treating a disorder associated with decreased ER activity (e.g., menopause-related conditions or symptoms, or osteoporosis). In some embodiments, provided estrogen receptor agonists are useful for treating such disorders. Other uses of estrogen receptor agonists exist; see, e.g., Harrison, R. F. and Bonnar, J., Pharmac. Ther., 1980, 11, 451-67.

In some embodiments, the present disclosure provides a method of treating a disorder mediated by an estrogen receptor in a subject, comprising administering to the subject a therapeutically effective amount of a compound described herein.

In some embodiments, a disorder is selected from the group consisting of breast cancer, ovarian cancer, endometrial cancer, vaginal cancer, lung cancer, bone cancer, uterine cancer, and endometriosis. In some embodiments, a disorder is breast cancer. In some embodiments, a disorder is ovarian cancer. In some embodiments, a disorder is endometrial cancer. In some embodiments, a disorder is vaginal cancer. In some embodiments, a disorder is lung cancer. In some embodiments, a disorder is bone cancer. In some embodiments, a disorder is uterine cancer. In some embodiments, a disorder is endometriosis.

In some embodiments, the present disclosure provides a method of treating a disorder associated with a mutation of an estrogen receptor in a subject, comprising administering to the subject a therapeutically effective amount of a compound described herein. In some embodiments, such a disorder is selected from the group consisting of breast cancer, ovarian cancer, endometrial cancer, vaginal cancer, lung cancer, bone cancer, uterine cancer, and endometriosis. In some embodiments, a disorder is breast cancer. In some embodiments, a disorder is ovarian cancer. In some embodiments, a disorder is endometrial cancer. In some embodiments, a disorder is vaginal cancer. In some embodiments, a disorder is lung cancer. In some embodiments, a disorder is bone cancer. In some embodiments, a disorder is uterine cancer. In some embodiments, a disorder is endometriosis.

In some embodiments, a method of treating a disorder in a subject described herein comprises administering to the subject a compound described herein in combination with or alternation with an anti-cancer agent. In some embodiments, an anti-cancer agent is selected from an mTOR inhibitor, a CDK4/6 inhibitor, a P13 kinase inhibitor, an aromatase inhibitor, an antibody to or inhibitor of PD-1, PD-L1 or CTLA-4, or an antibody to or inhibitor of EGFR, PGFR, or IGFR.

In some embodiments, an anti-cancer agent is a HER2 inhibitor. In some embodiments, a HER2 inhibitor is selected from tucatinib, trastuzumab, pertuzumab, ado-trastuzumab, trastuzumab emtansine, ado-trastuzumab emtansine, trastuzumab deruxtecan pertuzumab, lapatinib, and neratinib.

In some embodiments, an anti-cancer agent is an mTOR inhibitor. In some embodiments, an mTOR inhibitor is selected from everolimus sirolimus, temsirolimus, and LY3023414.

In some embodiments, an anti-cancer agent is a CDK4/6 inhibitor. In some embodiments, a CDK4/6 inhibitor is selected from palbociclib, abemaciclib, ribociclib, lerociclib, trilaciclib, and SHR6390.

In some embodiments, an anti-cancer agent is a PI3 kinase inhibitor. In some embodiments, a PI3 kinase inhibitor is selected from perifosine, CAL101, BEZ235, XL147, XL765, GDC-0941, and IPI-145.

In some embodiments, a PI3 kinase inhibitor is a PIK3CA inhibitor. In some embodiments, a PIK3CA inhibitor is selected from alpelisib, taselisib, and LY3023414.

In some embodiments, an anti-cancer agent is an aromatase inhibitor. In some embodiments, an aromatase inhibitor is selected from aminoglutethimide, testolactone, anastrozole, letrozole, exemestane, vorozole, formestane, fadrozole, 4-hydroxyandrostenedione, 1,4,6-androstatrien-3,17-dione, and 4-androstene-3,6,17-trione.

In some embodiments, an anti-cancer agent is an antibody to or inhibitor of PD-1, PD-L1 or CTLA-4.

In some embodiments, an anti-cancer agent is an antibody to or inhibitor of EGFR, PGFR, or IGFR. In some embodiments, an anti-cancer agent is erlotinib or gefitinib.

In some embodiments, a method described herein comprises administering a compound reported herein in combination or in alternation with an estrogen receptor antagonist or a partial estrogen receptor antagonist.

In some embodiments, the present disclosure provides a method of preventing recurrence of a cancer in a subject comprising administering to the subject a compound described herein. In some embodiments, a cancer is selected from breast cancer, ovarian cancer, endometrial cancer, vaginal cancer, lung cancer, bone cancer, and uterine cancer. In some embodiments, a compound described herein is administered as an adjunctive therapy after or instead of chemotherapy, radiation, or surgery. In some embodiments, a compound is administered after surgery. In some embodiments, a compound is administered prior to surgery. In some embodiments, a cancer is a breast cancer that has progressed in the presence of endocrine or aromatase therapy.

### EXAMPLES

As described in the Examples below, in certain exemplary embodiments, compounds are prepared according to the following general procedures. It will be appreciated that, although the general methods depict the synthesis of certain compounds of the present disclosure, the following general methods and other methods known to one of ordinary skill in the art can be applied to all compounds and subclasses and species of each of these compounds, as described herein.

### Example 1: Methyl 3-((1R,3R)-2-(2-fluoro-2-methylpropyl)-3-methyl-2,3,4,9-tetrahydro-JH-pyrido[3,4-b]indol-1-yl)-1H-indole-6-carboxylate

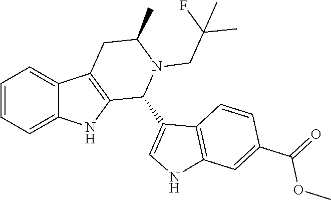 [SMILES: COC(=O)c1ccc2c([C@@H]3c4[nH]c5ccccc5c4C[C@@H](C)N3CC(C)(C)F)c[nH]c2c1]

Under nitrogen atmosphere, methyl 3-formyl-1H-indole-6-carboxylate (393.00 mg, 1.93 mmol) and acetic acid (0.23 mL, 4.03 mmol) were added sequentially to the solution of 2-fluoro-N-[(1R)-2-(1H-indol-3-yl)-1-methyl-ethyl]-2-methyl-propan-1-amine (400 mg, 1.61 mmol) in 1,2-dichloroethane (20 mL) in microwave vial, exchanged the air in the vial with nitrogen 3 times, the reaction mixture was heated at 100 °C in microwave reactor for 4 h. After cooling to room temperature, the reaction mixture was diluted with dichloromethane and washed with saturated NaHCO₃ solution, H₂O and brine, dried with anhydrous Na₂SO₄, filtered and concentrated under vacuum to afford crude product. The crude product was purified by normal phase Combiflash chromatography (0%-30% ethyl acetate in hexane) to afford the title compound (320.3 mg, 45.9% yield) as pale-yellow solid. LC-MS: m/z=434 [M+H]⁺.

### Example 2: 3-((1R,3R)-2-(2-Fluoro-2-methylpropyl)-3-methyl-2,3,4,9-tetrahydro-1H-pyrido[3,4-b]indol-1-yl)-1H-indole-6-carboxylic acid

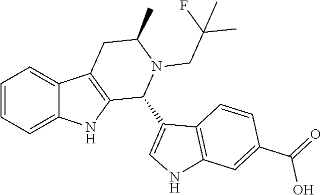 [SMILES: C[C@@H]1Cc2c([nH]c3ccccc23)[C@@H](c2c[nH]c3cc(C(=O)O)ccc23)N1CC(C)(C)F]

Under nitrogen atmosphere, a solution of lithium hydroxide monohydrate (19.36 mg, 0.36 mmol) in H₂O (0.5 mL) was added to a solution of methyl 3-((1R,3R)-2-(2-fluoro-2-methylpropyl)-3-methyl-2,3,4,9-tetrahydro-1H-pyrido[3,4-b]indol-1-yl)-1H-indole-6-carboxylate (50 mg, 0.12 mmol) in THE (1 mL) at room temperature, then heated at 65 °C and stirred overnight. The reaction mixture was concentrated under vacuum, diluted with H₂O (6 mL), adjusted pH to 2-3 with 1 N HCl solution, and extracted with ethyl acetate. The organic phase was washed with brine, dried with anhydrous Na₂SO₄, filtered, and concentrated under vacuum to afford the title compound (20.0 mg, 41.3% yield) as a pale-yellow solid. LC-MS: m/z=420 [M+H]⁺.

### Example 3: (3-((1R,3R)-2-(2-Fluoro-2-methylpropyl)-3-methyl-2,3,4,9-tetrahydro-1H-pyrido[3,4-b]indol-1-yl)-1H-indol-6-yl)methanol

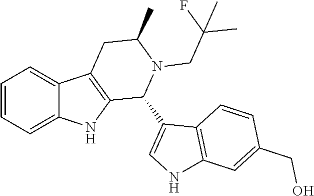 [SMILES: C[C@@H]1Cc2c([nH]c3ccccc23)[C@@H](c2c[nH]c3cc(CO)ccc23)N1CC(C)(C)F]

Under nitrogen atmosphere, a 1 M solution of lithium aluminum hydride (2.08 mL, 2.08 mmol) in THF was added dropwise to a solution of methyl 3-((1R,3R)-2-(2-fluoro-2-methylpropyl)-3-methyl-2,3,4,9-tetrahydro-1H-pyrido[3,4-b]indol-1-yl)-1H-indole-6-carboxylate (300.0 mg, 0.69 mmol) in anhydrous THF (10 mL) at 0 °C, then stirred at room temperature for 3 h. The reaction mixture was cooled to 0 °C, 0.06 mL H₂O was added dropwise to the reaction mixture, followed by 0.12 mL 10% NaOH solution, then 0.24 mL H₂O, and stirred at room temperature for 30 min. The mixture was then filtered and concentrated under vacuum to afford crude product. The crude product was purified by normal phase Combiflash chromatography (0%-60% ethyl acetate in hexane) to afford the title compound (268.3 mg, 95.6% yield) as pale-yellow solid. ¹H NMR (300 MHz, DMSO-d₆) δ 10.77 (s, 1H), 10.62 (s, 1H), 7.68-7.63 (m, 1H), 7.41 (d, J=7.3 Hz, 1H), 7.28 (s, 1H), 7.19 (d, J=7.3 Hz, 1H), 7.04-6.91 (m, 2H), 6.86 (d, J=8.5 Hz, 1H), 6.64-6.59 (m, 1H), 5.26 (s, 2H), 5.01 (t, J=5.7 Hz, IH), 4.55-4.50 (m, 2H), 2.81-2.50 (m, 5H), 1.39 (d, J=21.6 Hz, 3H), 1.25 (d, J=21.3 Hz, 3H), 0.99 (d, J=6.7 Hz, 3H); LC-MS: m/z=406 [M+H]⁺.

### Example 4: 3-((1R,3R)-2-(2-Fluoro-2-methylpropyl)-3-methyl-2,3,4,9-tetrahydro-1H-pyrido[3,4-b]indol-1-yl)-1H-indole-6-carbaldehyde

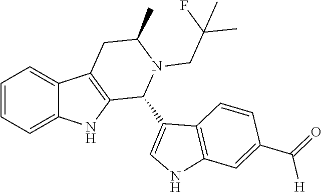 [SMILES: C[C@@H]1Cc2c([nH]c3ccccc23)[C@@H](c2c[nH]c3cc(C=O)ccc23)N1CC(C)(C)F]

Under nitrogen atmosphere, (3-((1R,3R)-2-(2-fluoro-2-methylpropyl)-3-methyl-2,3,4,9-tetrahydro-1H-pyrido[3,4-b]indol-1-yl)-1H-indol-6-yl)methanol (220.00 mg, 0.54 mmol) was dissolved in anhydrous dichloromethane (12 mL) and the reaction mixture was cooled to 0 °C. (1,1,1-Triacetoxy)-1,1-dihydro-1,2-benziodoxol-3(1H)-one (276.13 mg, 0.65 mmol) was added and the mixture was stirred at 0 °C for 30 min, then stirred at room temperature for 2 h. The mixture was diluted with dichloromethane (40 mL) and poured into saturated solutions of NaHCO₃ (20 mL) and saturated solutions of Na₂S₂O₃ (20 mL). It was stirred at room temperature for 60 min. The organic layer was washed with H₂O (60 mL) and brine (60 mL), dried over anhydrous Na₂SO₄, and filtrated, then the solvent was evaporated under reduced pressure to afford the crude product. The crude product was purified by normal phase Combiflash chromatography (0%-60% ethyl acetate in hexane) to afford the title compound (198.7 mg, 90.8% yield) as pale-yellow solid. LC-MS: m/z=404 [M+H]⁺.

### Example 5: (1R,3R)-2-(2-Fluoro-2-methylpropyl)-1-(6-((3-(fluoromethyl)azetidin-1-yl)methyl)-1H-indol-3-yl)-3-methyl-2,3,4,9-tetrahydro-1H-pyrido[3,4-b]indole

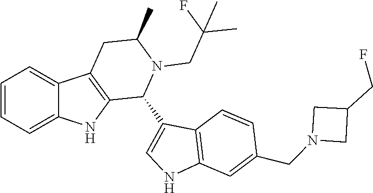 [SMILES: C[C@@H]1Cc2c([nH]c3ccccc23)[C@@H](c2c[nH]c3cc(CN4CC(CF)C4)ccc23)N1CC(C)(C)F]

Under nitrogen atmosphere, the solution of 3-((1R,3R)-2-(2-fluoro-2-methylpropyl)-3-methyl-2,3,4,9-tetrahydro-1H-pyrido[3,4-b]indol-1-yl)-1H-indole-6-carbaldehyde (30.00 mg, 0.074 mmol) in anhydrous dichloromethane (0.5 mL) was added to the reaction solution of 3-(fluoromethyl)azetidine hydrochloride (18.67 mg, 0.15 mmol) and triethylamine (0.020 mL, 0.15 mmol) in anhydrous dichloromethane (1 mL), then acetic acid (0.017 mL, 0.30 mmol) was added to the reaction mixture, then stirred at room temperature for 1 h. At 0 °C, sodium triacetoxyborohydride (47.27 mg, 0.22 mmol) was added to the reaction mixture, then stirred at room temperature for 12 h. The reaction mixture was quenched with MeOH (0.1 mL), diluted with dichloromethane (10 mL), and the organic phase was washed with saturated NaHCO₃ solution and brine. The organic layer was dried with anhydrous Na₂SO₄, filtered, and concentrated under vacuum to afford crude product. The crude product was purified by normal phase Combiflash chromatography (0%-10% MeOH in dichloromethane) to afford the title compound (13.60 mg, 38.4% yield) as pale-yellow solid. ¹H NMR (300 MHz, DMSO-d₆) δ 10.76 (s, 1H), 10.62 (s, 1H), 7.63 (d, J=7.5 Hz, 1H), 7.41 (d, J=7.3 Hz, 1H), 7.20 (d, J=8.9 Hz, 2H), 7.03-6.90 (m, 2H), 6.83 (d, J=8.1 Hz, 1H), 6.61 (s, 1H), 5.26 (s, 1H), 4.58 (d, J=6.2 Hz, 1H), 4.42 (d, J=6.2 Hz, 1H), 3.58 (s, 2H), 3.45-3.36 (m, 1H), 3.24 (t, J=7.0 Hz, 2H), 2.94 (s, 2H), 2.78-2.53 (m, 5H), 1.40 (d, J=21.9 Hz, 3H), 1.25 (d, J=20.8 Hz, 3H), 1.00 (d, J=6.7 Hz, 3H); LC-MS: m/z=477 [M+H]⁺.

### Example 6: (1R,3R)-2-(2-Fluoro-2-methylpropyl)-3-methyl-1-(6-((3-methylazetidin-1-yl)methyl)-1H-indol-3-yl)-2,3,4,9-tetrahydro-1H-pyrido[3,4-b]indole

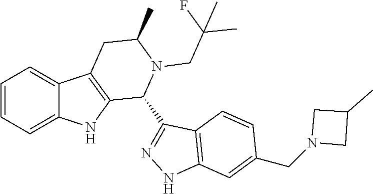 [SMILES: CC1CN(Cc2ccc3c([C@@H]4c5[nH]c6ccccc6c5C[C@@H](C)N4CC(C)(C)F)n[nH]c3c2)C1]

Under nitrogen atmosphere, a solution of 3-((1R,3R)-2-(2-fluoro-2-methylpropyl)-3-methyl-2,3,4,9-tetrahydro-1H-pyrido[3,4-b]indol-1-yl)-1H-indole-6-carbaldehyde (30.00 mg, 0.074 mmol) in anhydrous dichloromethane (0.5 mL) was added to a reaction solution of 3-methylazetidine hydrochloride (16.00 mg, 0.15 mmol) and triethylamine (0.020 mL, 0.15 mmol) in anhydrous dichloromethane (1 mL). Acetic acid (0.017 mL, 0.30 mmol) was added to the reaction mixture then stirred at room temperature for 1 h. At 0 °C, sodium triacetoxyborohydride (47.27 mg, 0.22 mmol) was added to the reaction mixture, then stirred at room temperature for 12 h. The reaction mixture was quenched with MeOH (0.1 mL), diluted with dichloromethane (10 mL), and the organic phase was washed with saturated NaHCO₃ solution and brine, then dried with anhydrous Na₂SO₄, filtered, and concentrated under vacuum to afford crude product. The crude product was purified by normal phase Combiflash chromatography (0%-10% MeOH in dichloromethane) to afford the title compound (13.20 mg, 38.7% yield) as pale-yellow solid. LC-MS: m/z=459 [M+H]⁻.

### Example 7: 3-Fluoro-N-((3-((1R,3R)-2-(2-fluoro-2-methylpropyl)-3-methyl-2,3,4,9-tetrahydro-1H-pyrido[3,4-b]indol-1-yl)-1H-indol-6-yl)methyl)-N-methylpropan-1-amine

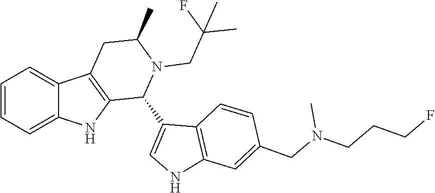 [SMILES: C[C@@H]1Cc2c([nH]c3ccccc23)[C@@H](c2c[nH]c3cc(CN(C)CCCF)ccc23)N1CC(C)(C)F]

Under nitrogen atmosphere, a solution of 3-((1R,3R)-2-(2-fluoro-2-methylpropyl)-3-methyl-2,3,4,9-tetrahydro-1H-pyrido[3,4-b]indol-1-yl)-1H-indole-6-carbaldehyde (25.00 mg, 0.062 mmol) in anhydrous dichloromethane (0.5 mL) was added to a reaction solution of 3-fluoro-N-methyl-propan-1-amine (11.29 mg, 0.12 mmol) and triethylamine (0.017 mL, 0.12 mmol) in anhydrous dichloromethane (1 mL). Acetic acid (0.014 mL, 0.25 mmol) was added to the reaction mixture, then stirred at room temperature for 1 h. At 0 °C, sodium triacetoxyborohydride (39.40 mg, 0.19 mmol) was added to the reaction mixture, then stirred at room temperature for 12 h. The reaction mixture was quenched with MeOH (0.1 mL), diluted with dichloromethane (10 mL), the organic phase was washed with saturated NaHCO₃ solution and brine, the organic layer was dried with anhydrous Na₂SO₄, filtered, and concentrated under vacuum to afford crude product. The crude product was purified by normal phase Combiflash chromatography (0%-10% MeOH in dichloromethane) to afford the title compound (10.00 mg, 33.7% yield) as pale-yellow solid. LC-MS: m/z=479 [M+H]⁺.

### Example 8: N-((3-((1R,3R)-2-(2-Fluoro-2-methylpropyl)-3-methyl-2,3,4,9-tetrahydro-1H-pyrido[3,4-b]indol-1-yl)-1H indol-6-yl)methyl)-N-methylpropan-1-amine

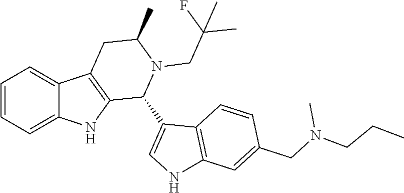 [SMILES: CCCN(C)Cc1ccc2c([C@@H]3c4[nH]c5ccccc5c4C[C@@H](C)N3CC(C)(C)F)c[nH]c2c1]

Under nitrogen atmosphere, a solution of 3-((1R,3R)-2-(2-fluoro-2-methylpropyl)-3-methyl-2,3,4,9-tetrahydro-1H-pyrido[3,4-b]indol-1-yl)-1H-indole-6-carbaldehyde (25.00 mg, 0.062 mmol) in anhydrous dichloromethane (0.5 mL) was added to a solution of N-methylpropan-1-amine (9.06 mg, 0.12 mmol) and triethylamine (0.017 mL, 0.12 mmol) in anhydrous dichloromethane (1 mL), then acetic acid (0.014 mL, 0.25 mmol) was added to the reaction mixture, then stirred at room temperature for 1 h. At 0 °C, sodium triacetoxyborohydride (39.40 mg, 0.19 mmol) was added to the reaction mixture, then stirred at room temperature for 12 h. The reaction mixture was quenched with MeOH (0.1 mL), diluted with dichloromethane (10 mL), the organic phase was washed with saturated NaHCO₃ solution and brine, the organic layer was dried with anhydrous Na₂SO₄, filtered, and concentrated under vacuum to afford crude product. The crude product was purified by normal phase Combiflash chromatography (0%-10% MeOH in dichloromethane) to afford the title compound (10.60 mg, 37.1% yield) as pale-yellow solid. LC-MS: m/z=461 [M+H]⁻.

### Example 9: 3-Fluoro-N-((3-((1R,3R)-2-(2-fluoro-2-methylpropyl)-3-methyl-2,3,4,9-tetrahydro-1H-pyrido[3,4-b]indol-1-yl)-1H-indol-6-yl)methyl)propan-1-amine

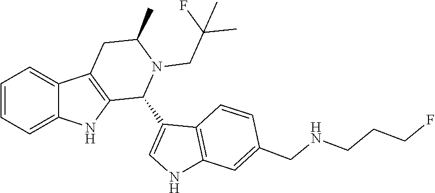 [SMILES: C[C@@H]1Cc2c([nH]c3ccccc23)[C@@H](c2c[nH]c3cc(CNCCCF)ccc23)N1CC(C)(C)F]

Under nitrogen atmosphere, a solution of 3-((1R,3R)-2-(2-fluoro-2-methylpropyl)-3-methyl-2,3,4,9-tetrahydro-1H-pyrido[3,4-b]indol-1-yl)-1H-indole-6-carbaldehyde (30.00 mg, 0.074 mmol) in anhydrous dichloromethane (0.5 mL) was added to a solution of 3-fluoro-1-propanamine hydrochloride (16.89 mg, 0.15 mmol) and triethylamine (0.021 mL, 0.15 mmol) in anhydrous dichloromethane (1 mL), then acetic acid (0.017 mL, 0.30 mmol) was added to the reaction mixture and stirred at room temperature for 1 h. At 0 °C, sodium triacetoxyborohydride (47.27 mg, 0.22 mmol) was added to the reaction mixture, then stirred at room temperature for 12 h. The reaction mixture was quenched with MeOH (0.1 mL), diluted with dichloromethane (10 mL), the organic phase was washed with saturated NaHCO₃ solution and brine, the organic layer was dried with anhydrous Na₂SO₄, filtered, and concentrated under vacuum to afford crude product. The crude product was purified by normal phase Combiflash chromatography (0%-10% MeOH in dichloromethane) to afford the title compound (10.40 mg, 30.1% yield) as pale-yellow solid. LC-MS: m/z=465 [M+H]⁺.

### Example 10: Methyl 3-((1S,3R)-2-(2-Fluoro-2-methylpropyl)-3-methyl-2,3,4,9-tetrahydro-II-pyrido[3,4-b]indol-1-yl)-1H-indazole-6-carboxylate

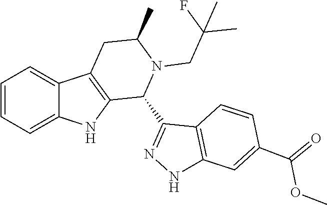 [SMILES: COC(=O)c1ccc2c([C@@H]3c4[nH]c5ccccc5c4C[C@@H](C)N3CC(C)(C)F)n[nH]c2c1]

Under nitrogen atmosphere, methyl 3-formyl-1H-indazole-6-carboxylate (400.00 mg, 1.96 mmol) and acetic acid (0.28 mL, 4.90 mmol) were added sequentially to the solution of 2-fluoro-N-[(1R)-2-(1H-indol-3-yl)-1-methyl-ethyl]-2-methyl-propan-1-amine (632.47 mg, 2.55 mmol) in 1,2-dichloroethane (20 mL) in a microwave vial, exchanged the air in the vial with nitrogen 3 times, the reaction mixture was heated at 100 °C in microwave reactor for 2 h. After cooling to room temperature, the reaction mixture was diluted with dichloromethane and washed with saturated NaHCO₃ solution, H₂O and brine, dried with anhydrous Na₂SO₄, filtered and concentrated under vacuum to afford crude product. The crude product was purified by normal phase Combiflash chromatography (0%-30% ethyl acetate in hexane) to afford the title compound (628.00 mg, 73.8% yield) as pale-yellow solid. LC-MS: m/z=435 [M+H]⁺.

### Example 11: 3-((1S,3R)-2-(2-Fluoro-2-methylpropyl)-3-methyl-2,3,4,9-tetrahydro-1H-pyrido[3,4-b]indol-1-yl)-1H-indazole-6-carboxylic acid

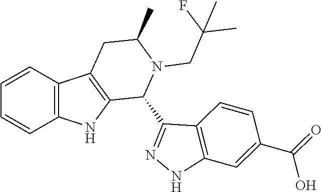 [SMILES: C[C@@H]1Cc2c([nH]c3ccccc23)[C@@H](c2n[nH]c3cc(C(=O)O)ccc23)N1CC(C)(C)F]

Under nitrogen atmosphere, a solution of lithium hydroxide monohydrate (6.18 mg, 0.15 mmol) in H₂O (0.5 mL) was added to the solution of methyl 3-((1S,3R)-2-(2-fluoro-2-methylpropyl)-3-methyl-2,3,4,9-tetrahydro-1H-pyrido[3,4-b]indol-1-yl)-1H-indazole-6-carboxylate (16.00 mg, 0.037 mmol) in THF (1 mL) at room temperature, then heated at 65 °C and stirred overnight. The reaction mixture was concentrated under vacuum, diluted with H₂O (6 mL), adjusted to pH 2-3 with 1 N HCl solution, extracted with ethyl acetate, the organic phase was washed with brine, dried with anhydrous Na₂SO₄, filtered, and concentrated under vacuum to afford the title compound (6.00 mg, 38.8% yield) as pale-yellow solid. LC-MS: m/z=421 [M+H]⁺.

### Example 12: (3-((1S,3R)-2-(2-Fluoro-2-methylpropyl)-3-methyl-2,3,4,9-tetrahydro-1H-pyrido[3,4-b]indol-1-yl)-1H-indazol-6-yl)methanol

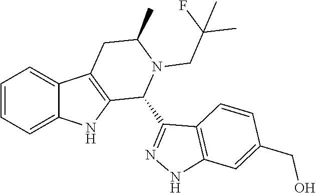 [SMILES: C[C@@H]1Cc2c([nH]c3ccccc23)[C@@H](c2n[nH]c3cc(CO)ccc23)N1CC(C)(C)F]

Under nitrogen atmosphere, a solution of 1 M lithium aluminum hydride (3.80 mL, 3.80 mmol) in THE was added dropwise to a solution of methyl 3-((1S,3R)-2-(2-fluoro-2-methylpropyl)-3-methyl-2,3,4,9-tetrahydro-1H-pyrido[3,4-b]indol-1-yl)-1H-indazole-6-carboxylate (550.00 mg, 1.27 mmol) in anhydrous THE (20 mL) at 0 °C, then stirred at room temperature for 3 h. The reaction mixture was cooled to 0 °C, 0.12 mL H₂O was added dropwise to the reaction mixture, then added 0.24 mL of 10% NaOH solution, followed by 0.48 mL of H₂O, stirred at room temperature for 30 min, then filtered and concentrated under vacuum to afford crude product. The crude product was purified by normal phase Combiflash chromatography (0%-60% ethyl acetate in hexane) to afford the title compound (350.00 mg, 68.0% yield) as pale-yellow solid. ¹H NMR (300 MHz, DMSO-d₆) δ 12.67 (s, 1H), 10.60 (s, 1H), 7.57-7.51 (m, 1H), 7.45-7.37 (m, 2H), 7.18 (d, J=8.3 Hz, 1H), 6.95 (dt, J=14.5, 6.4 Hz, 3H), 5.36 (s, 1H), 5.21 (s, 1H), 4.56 (d, J=4.5 Hz, 2H), 2.73 (m, 5H), 1.33 (d, J=20.8 Hz, 3H), 1.21 (d, J=22.4 Hz, 3H), 1.03 (d, J=6.7 Hz, 3H); LC-MS: m/z=407 [M+H]⁻.

### Example 13: 3-((1S,3R)-2-(2-fluoro-2-methylpropyl)-3-methyl-2,3,4,9-tetrahydro-1H-pyrido[3,4-b]indol-1-yl)-1H-indazole-6-carbaldehyde

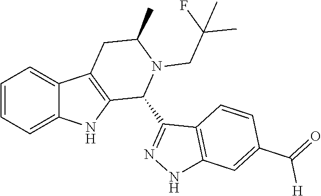 [SMILES: C[C@@H]1Cc2c([nH]c3ccccc23)[C@@H](c2n[nH]c3cc(C=O)ccc23)N1CC(C)(C)F]

Under nitrogen atmosphere, (3-((1S,3R)-2-(2-fluoro-2-methylpropyl)-3-methyl-2,3,4,9-tetrahydro-1H-pyrido[3,4-b]indol-1-yl)-1H-indazol-6-yl)methanol (350.00 mg, 0.86 mmol) was dissolved in anhydrous dichloromethane (20 mL) and the reaction mixture was cooled to 0 °C. (1,1,1-Triacetoxy)-1,1-dihydro-1,2-benziodoxol-3(1H)-one (438.23 mg, 1.03 mmol) was added and the mixture was stirred at 0 °C for 30 min, then stirred at room temperature for 2 h. The mixture was diluted with dichloromethane (40 mL) and poured into saturated solutions of NaHCO₃ (20 mL) and saturated solutions of Na₂S₂O₃ (20 mL). It was stirred at room temperature for 60 min. The organic layer was washed with H₂O (60 mL) and brine (60 mL), dried over anhydrous Na₂SO₄, and filtrated, then the solvent was evaporated under reduced pressure to afford the crude product. The crude product was purified by normal phase Combiflash chromatography (0%-60% ethyl acetate in hexane) to afford the title compound (118.68 mg, 34.1% yield) as pale-yellow solid. LC-MS: m/z=405 [M+H]⁺.

### Example 14: (1S,3R)-2-(2-Fluoro-2-methylpropyl)-1-(6-((3-(fluoromethyl)azetidin-1-yl)methyl)-1H-indazol-3-yl)-3-methyl-2,3,4,9-tetrahydro-1H-pyrido[3,4-b]indole

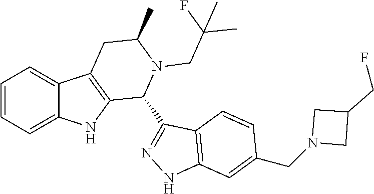 [SMILES: C[C@@H]1Cc2c([nH]c3ccccc23)[C@@H](c2n[nH]c3cc(CN4CC(CF)C4)ccc23)N1CC(C)(C)F]

Under nitrogen atmosphere, the solution of 3-((1S,3R)-2-(2-fluoro-2-methylpropyl)-3-methyl-2,3,4,9-tetrahydro-1H-pyrido[3,4-b]indol-1-yl)-1H-indazole-6-carbaldehyde (30.00 mg, 0.074 mmol) in anhydrous dichloromethane (0.5 mL) was added to the reaction solution of 3-(fluoromethyl)azetidine hydrochloride (18.63 mg, 0.15 mmol) and triethylamine (0.021 mL, 0.15 mmol) in anhydrous dichloromethane (1 mL), then acetic acid (0.017 mL, 0.30 mmol) was added to the reaction mixture and stirred at room temperature for 1 h. At 0 °C, sodium triacetoxyborohydride (47.27 mg, 0.22 mmol) was added to the reaction mixture, then stirred at room temperature for 12 h. The reaction mixture was quenched with MeOH (0.1 mL), diluted with dichloromethane (10 mL), the organic phase was washed with saturated NaHCO₃ solution and brine, the organic layer was dried with anhydrous Na₂SO₄, filtered, and concentrated under vacuum to afford crude product. The crude product was purified by normal phase Combiflash chromatography (0%-10% MeOH in dichloromethane) to afford the title compound (14.40 mg, 40.7% yield) as pale-yellow solid. ¹H NMR (300 MHz, DMSO-d₆) δ 12.66 (s, 1H), 10.60 (s, 1H), 7.52 (d, J=7.9 Hz, 1H), 7.42 (d, J=7.0 Hz, 1H), 7.32 (s, 1H), 7.18 (d, J=7.2 Hz, 1H), 7.03-6.91 (m, 2H), 6.88 (d, J=8.3 Hz, 1H), 5.37 (s, 1H), 4.59 (d, J=6.1 Hz, 1H), 4.43 (d, J=6.2 Hz, 1H), 3.62 (s, 2H), 3.50-3.36 (m, 1H), 3.25 (d, J=4.7 Hz, 2H), 2.96 (s, 2H), 2.88-2.52 (m, 5H), 1.34 (d, J=21.6 Hz, 3H), 1.21 (d, J=21.5 Hz, 3H), 1.03 (d, J=6.7 Hz, 3H); LC-MS: m/z=479 [M+H]⁺.

### Example 15: (1S,3R)-2-(2-Fluoro-2-methylpropyl)-3-methyl-1-(6-((3-methylazetidin-1-yl)methyl)-1H-indazol-3-yl)-2,3,4,9-tetrahydro-1H-pyrido[3,4-b]indole

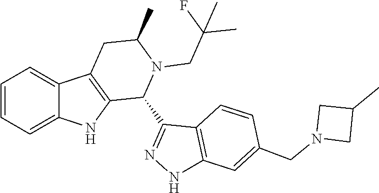 [SMILES: CC1CN(Cc2ccc3c([C@@H]4c5[nH]c6ccccc6c5C[C@@H](C)N4CC(C)(C)F)n[nH]c3c2)C1]

Under nitrogen atmosphere, the solution of 3-((1S,3R)-2-(2-fluoro-2-methylpropyl)-3-methyl-2,3,4,9-tetrahydro-1H-pyrido[3,4-b]indol-1-yl)-1H-indazole-6-carbaldehyde (30.00 mg, 0.074 mmol) in anhydrous dichloromethane (0.5 mL) was added to the reaction solution of 3-methylazetidine hydrochloride (15.96 mg, 0.15 mmol) and triethylamine (0.021 mL, 0.15 mmol) in anhydrous dichloromethane (1 mL), then acetic acid (0.017 mL, 0.30 mmol) was added to the reaction mixture then stirred at room temperature for 1 h. At 0 °C, sodium triacetoxyborohydride (47.27 mg, 0.22 mmol) was added to the reaction mixture, then stirred at room temperature for 12 h. The reaction mixture was quenched with MeOH (0.1 mL), diluted with dichloromethane (10 mL), the organic phase was washed with saturated NaHCO₃ solution and brine, the organic layer was dried with anhydrous Na₂SO₄, filtered and concentrated under vacuum to afford crude product. The crude product was purified by normal phase Combiflash chromatography (0%-10% MeOH in dichloromethane) to afford the title compound (13.10 mg, 38.4% yield) as pale-yellow solid. LC-MS: m/z=460 [M+H]⁻.

### Example 16: (1S,3R)-1-(6-((3-ethylazetidin-1-yl)methyl)-1H-indazol-3-yl)-2-(2-fluoro-2-methyl-propyl)-3-methyl-2,3,4,9-tetrahydro-1H-pyrido[3,4-b]indole

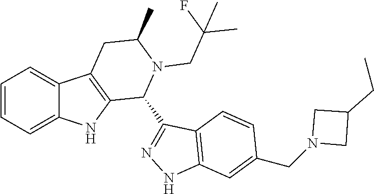 [SMILES: CCC1CN(Cc2ccc3c([C@@H]4c5[nH]c6ccccc6c5C[C@@H](C)N4CC(C)(C)F)n[nH]c3c2)C1]

Under nitrogen atmosphere, a solution of 3-((1S,3R)-2-(2-fluoro-2-methylpropyl)-3-methyl-2,3,4,9-tetrahydro-1H-pyrido[3,4-b]indol-1-yl)-1H-indazole-6-carbaldehyde (40.00 mg, 0.099 mmol) in anhydrous dichloromethane (1 mL) was added to the reaction solution of 3-ethylazetidine, trifluoroacetic acid (39 mg, 0.20 mmol) and triethylamine (0.028 mL, 0.20 mmol) in anhydrous dichloromethane (2 mL), then acetic acid (0.023 mL, 0.40 mmol) was added to the reaction mixture and stirred at room temperature for 1 h. At 0 °C, sodium triacetoxyborohydride (41.92 mg, 0.20 mmol) was added to the reaction mixture, then stirred at room temperature for 12 h. The reaction mixture was quenched with MeOH (0.1 mL), diluted with dichloromethane (10 mL), the organic phase was washed with saturated NaHCO₃ solution and brine, the organic layer was dried with anhydrous Na₂SO₄, filtered, and concentrated under vacuum to afford crude product. The crude product was purified by normal phase Combiflash chromatography (0%-10% MeOH in dichloromethane) to afford the title compound (16.28 mg, 34.8% yield) as pale-yellow solid. LC-MS: m/z=474 [M+1]⁺.

### Example 17: 3-Fluoro-N-((3-((1S,3R)-2-(2-fluoro-2-methylpropyl)-3-methyl-2,3,4,9-tetrahydro-1H-pyrido[3,4-b]indol-1-yl)-1H-indazol-6-yl)methyl)-N-methylpropan-1-amine

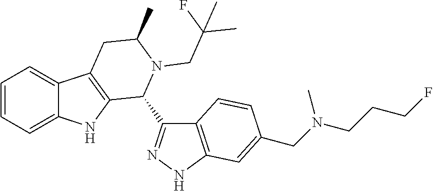 [SMILES: C[C@@H]1Cc2c([nH]c3ccccc23)[C@@H](c2n[nH]c3cc(CN(C)CCCF)ccc23)N1CC(C)(C)F]

Under nitrogen atmosphere, the solution of 3-((1S,3R)-2-(2-fluoro-2-methylpropyl)-3-methyl-2,3,4,9-tetrahydro-1H-pyrido[3,4-b]indol-1-yl)-1H-indazole-6-carbaldehyde (30.00 mg, 0.072 mmol) in anhydrous dichloromethane (0.5 mL) was added to the reaction solution of 3-fluoro-N-methyl-propan-1-amine (13.52 mg, 0.15 mmol) and triethylamine (0.021 mL, 0.15 mmol) in anhydrous dichloromethane (1 mL), then acetic acid (0.017 mL, 0.30 mmol) was added to the reaction mixture and stirred at room temperature for 1 h. At 0 °C, sodium triacetoxyborohydride (47.16 mg, 0.22 mmol) was added to the reaction mixture, then stirred at room temperature for 12 h. The reaction mixture was quenched with MeOH (0.1 mL), diluted with dichloromethane (10 mL), the organic phase was washed with saturated NaHCO₃ solution and brine, the organic layer was dried with anhydrous Na₂SO₄, filtered, and concentrated under vacuum to afford crude product. The crude product was purified by normal phase Combiflash chromatography (0%-10% MeOH in dichloromethane) to afford the title compound (14.18 mg, 39.9% yield) as pale-yellow solid. LC-MS: m/z=480 [M+H]⁻.

### Example 18: N-((3-((1S,3R)-2-(2-Fluoro-2-methylpropyl)-3-methyl-2,3,4,9-tetrahydro-1H-pyrido[3,4-b]indol-1-yl)-1H indazol-6-yl)methyl)-N-methylpropan-1-amine

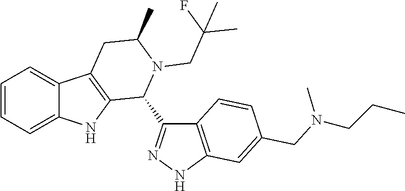 [SMILES: CCCN(C)Cc1ccc2c([C@@H]3c4[nH]c5ccccc5c4C[C@@H](C)N3CC(C)(C)F)n[nH]c2c1]

Under nitrogen atmosphere, a solution of 3-((1S,3R)-2-(2-fluoro-2-methylpropyl)-3-methyl-2,3,4,9-tetrahydro-1H-pyrido[3,4-b]indol-1-yl)-1H-indazole-6-carbaldehyde (30.00 mg, 0.072 mmol) in anhydrous dichloromethane (0.5 mL) was added to the reaction solution of N-methylpropan-1-amine (10.88 mg, 0.15 mmol) and triethylamine (0.021 mL, 0.15 mmol) in anhydrous dichloromethane (1 mL), then acetic acid (0.017 mL, 0.30 mmol) was added to the reaction mixture and stirred at room temperature for 1 h. At 0 °C, sodium triacetoxyborohydride (47.16 mg, 0.22 mmol) was added to the reaction mixture, then stirred at room temperature for 12 h. The reaction mixture was quenched with MeOH (0.1 mL), diluted with dichloromethane (10 mL), the organic phase was washed with saturated NaHCO₃ solution and brine, the organic layer was dried with anhydrous Na₂SO₄, filtered, and concentrated under vacuum to afford crude product. The crude product was purified by normal phase Combiflash chromatography (0%-10% MeOH in dichloromethane) to afford the title compound (16.00 mg, 46.6% yield) as pale-yellow solid. LC-MS: m/z=462 [M+H]⁻.

### Example 19: 3-Fluoro-N-((3-((1S,3R)-2-(2-fluoro-2-methylpropyl)-3-methyl-2,3,4,9-tetrahydro-1H-pyrido[3,4-b]indol-1-yl)-1H-indazol-6-yl)methyl)propan-1-amine

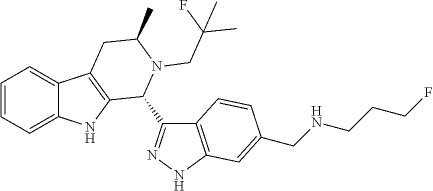 [SMILES: C[C@@H]1Cc2c([nH]c3ccccc23)[C@@H](c2n[nH]c3cc(CNCCCF)ccc23)N1CC(C)(C)F]

Under nitrogen atmosphere, the solution of 3-((1S,3R)-2-(2-fluoro-2-methylpropyl)-3-methyl-2,3,4,9-tetrahydro-1H-pyrido[3,4-b]indol-1-yl)-1H-indazole-6-carbaldehyde (30.00 mg, 0.072 mmol) in anhydrous dichloromethane (0.5 mL) was added to a solution of 3-fluoro-1-propanamine hydrochloride (16.85 mg, 0.15 mmol) and triethylamine (0.021 mL, 0.15 mmol) in anhydrous dichloromethane (1 mL), then acetic acid (0.017 mL, 0.30 mmol) was added to the reaction mixture and stirred at room temperature for 1 h. At 0 °C, sodium triacetoxyborohydride (47.16 mg, 0.22 mmol) was added to the reaction mixture, then stirred at room temperature for 12 h. The reaction mixture was quenched with MeOH (0.1 mL), diluted with dichloromethane (10 mL), the organic phase was washed with saturated NaHCO₃ solution and brine, the organic layer was dried with anhydrous Na₂SO₄, filtered, and concentrated under vacuum to afford crude product. The crude product was purified by normal phase Combiflash chromatography (0%-10% MeOH in dichloromethane) to afford the title compound (15.60 mg, 45.2% yield) as pale-yellow solid. LC-MS: m/z=466 [M+H]⁺.

### Example 20: (1S,3R)-2-(2-fluoro-2-methylpropyl)-1-(6-(((R)-3-fluoropyrrolidin-1-yl)methyl)-1H-indazol-3-yl)-3-methyl-2,3,4,9-tetrahydro-1H-pyrido[3,4-b]indole

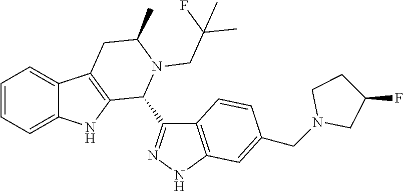 [SMILES: C[C@@H]1Cc2c([nH]c3ccccc23)[C@@H](c2n[nH]c3cc(CN4CC[C@@H](F)C4)ccc23)N1CC(C)(C)F]

Under nitrogen atmosphere, a solution of 3-((1S,3R)-2-(2-fluoro-2-methylpropyl)-3-methyl-2,3,4,9-tetrahydro-1H-pyrido[3,4-b]indol-1-yl)-1H-indazole-6-carbaldehyde (50.00 mg, 0.12 mmol) in anhydrous dichloromethane (1 mL) was added to a solution of (R)-(−)-3-fluoropyrrolidine hydrochloride (31.05 mg, 0.25 mmol) and triethylamine (0.035 mL, 0.25 mmol) in anhydrous dichloromethane (2 mL), then acetic acid (0.028 mL, 0.49 mmol) was added to the reaction mixture then stirred at room temperature for 1 h. At 0 °C, sodium triacetoxyborohydride (52.40 mg, 0.25 mmol) was added to the reaction mixture, then stirred at room temperature for 12 h. The reaction mixture was quenched with MeOH (0.1 mL), diluted with dichloromethane (10 mL), the organic phase was washed with saturated NaHCO₃ solution and brine, the organic layer was dried with anhydrous Na₂SO₄, filtered, and concentrated under vacuum to afford crude product. The crude product was purified by normal phase Combiflash chromatography (0%-10% MeOH in dichloromethane) to afford the title compound (22.55 mg, 38.2% yield) as pale-yellow solid. LC-MS: m/z=478 [M+H]⁻.

### Example 21: (1S,3R)-2-(2-fluoro-2-methylpropyl)-1-(6-(((S)-3-fluoropyrrolidin-1-yl)methyl)-1H-indazol-3-yl)-3-methyl-2,3,4,9-tetrahydro-1H-pyrido[3,4-b]indole

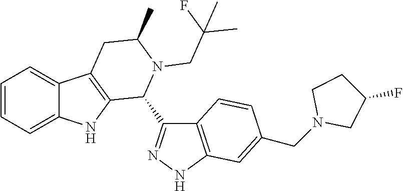 [SMILES: C[C@@H]1Cc2c([nH]c3ccccc23)[C@@H](c2n[nH]c3cc(CN4CC[C@H](F)C4)ccc23)N1CC(C)(C)F]

Under nitrogen atmosphere, a solution of 3-((1S,3R)-2-(2-fluoro-2-methylpropyl)-3-methyl-2,3,4,9-tetrahydro-1H-pyrido[3,4-b]indol-1-yl)-1H-indazole-6-carbaldehyde (50.00 mg, 0.12 mmol) in anhydrous dichloromethane (1 mL) was added to a solution of (3S)-(+)-3-fluoropyrrolidine hydrochloride (31.05 mg, 0.25 mmol) and triethylamine (0.035 mL, 0.25 mmol) in anhydrous dichloromethane (2 mL), then acetic acid (0.028 mL, 0.49 mmol) was added to the reaction mixture, then stirred at room temperature for 1 h. At 0 °C, sodium triacetoxyborohydride (52.40 mg, 0.25 mmol) was added to the reaction mixture, then stirred at room temperature for 12 h. The reaction mixture was quenched with MeOH (0.1 mL), diluted with dichloromethane (10 mL), the organic phase was washed with saturated NaHCO₃ solution and brine, the organic layer was dried with anhydrous Na₂SO₄, filtered, and concentrated under vacuum to afford crude product. The crude product was purified by normal phase Combiflash chromatography (0%-10% MeOH in dichloromethane) to afford the title compound (24.86 mg, 42.1% yield) as pale-yellow solid. LC-MS: m/z=478 [M+H]⁻.

### Example 22: (1S,3R)-1-(6-(((R)-3-ethylpyrrolidin-1-yl)methyl)-1H-indazol-3-yl)-2-(2-fluoro-2-methylpropyl)-3-methyl-2,3,4,9-tetrahydro-1H-pyrido[3,4-b]indole

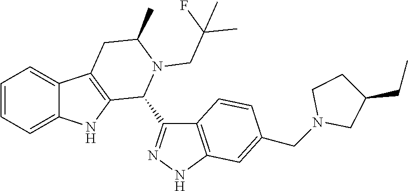 [SMILES: CC[C@@H]1CCN(Cc2ccc3c([C@@H]4c5[nH]c6ccccc6c5C[C@@H](C)N4CC(C)(C)F)n[nH]c3c2)C1]

Under nitrogen atmosphere, a solution of 3-((1S,3R)-2-(2-fluoro-2-methylpropyl)-3-methyl-2,3,4,9-tetrahydro-1H-pyrido[3,4-b]indol-1-yl)-1H-indazole-6-carbaldehyde (40.00 mg, 0.099 mmol) in anhydrous dichloromethane (1 mL) was added to a solution of (R)-3-ethylpyrrolidine hydrochloride (26.83 mg, 0.20 mmol) and triethylamine (0.028 mL, 0.20 mmol) in anhydrous dichloromethane (2 mL), then acetic acid (0.023 mL, 0.40 mmol) was added to the reaction mixture, then stirred at room temperature for 1 h. At 0 °C, sodium triacetoxyborohydride (41.92 mg, 0.20 mmol) was added to the reaction mixture, then stirred at room temperature for 12 h. The reaction mixture was quenched with MeOH (0.1 mL), diluted with dichloromethane (10 mL), the organic phase was washed with saturated NaHCO₃ solution and brine, the organic layer was dried with anhydrous Na₂SO₄, filtered, and concentrated under vacuum to afford crude product. The crude product was purified by normal phase Combiflash chromatography (0%-10% MeOH in dichloromethane) to afford the title compound (14.00 mg, 29.0% yield) as pale-yellow solid. LC-MS. m/z=488 [M+H]⁺.

### Example 23: (1S,3R)-1-(6-(((S)-3-ethylpyrrolidin-1-yl)methyl)-1H-indazol-3-yl)-2-(2-fluoro-2-methylpropyl)-3-methyl-2,3,4,9-tetrahydro-1H-pyrido[3,4-b]indole

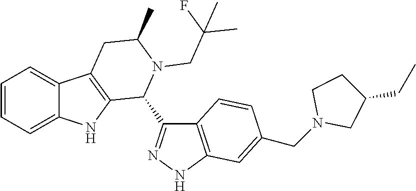 [SMILES: CC[C@H]1CCN(Cc2ccc3c([C@@H]4c5[nH]c6ccccc6c5C[C@@H](C)N4CC(C)(C)F)n[nH]c3c2)C1]

Under nitrogen atmosphere, the solution of 3-((1S,3R)-2-(2-fluoro-2-methylpropyl)-3-methyl-2,3,4,9-tetrahydro-1H-pyrido[3,4-b]indol-1-yl)-1H-indazole-6-carbaldehyde (40.00 mg, 0.099 mmol) in anhydrous dichloromethane (1 mL) was added to the reaction solution of(S)-3-ethylpyrrolidine hydrochloride (26.83 mg, 0.20 mmol) and triethylamine (0.028 mL, 0.20 mmol) in anhydrous dichloromethane (2 mL), then acetic acid (0.023 mL, 0.40 mmol) was added to the reaction mixture and stirred at room temperature for 1 h. At 0 °C, sodium triacetoxyborohydride (41.92 mg, 0.20 mmol) was added to the reaction mixture, then stirred at room temperature for 12 h. The reaction mixture was quenched with MeOH (0.1 mL), diluted with dichloromethane (10 mL), the organic phase was washed with saturated NaHCO₃ solution and brine, the organic layer was dried with anhydrous Na₂SO₄, filtered, and concentrated under vacuum to afford crude product. The crude product was purified by normal phase Combiflash chromatography (0%-10% MeOH in dichloromethane) to afford the title compound (20.00 mg, 41.5% yield) as pale-yellow solid. LC-MS: m/z=488 [M+H]⁻.

### Example 24: (1S,3R)-1-(6-((3-azabicyclo[3.1.0]hexan-3-yl)methyl)-1H-indazol-3-yl)-2-(2-fluoro-2-methylpropyl)-3-methyl-2,3,4,9-tetrahydro-1H-pyrido[3,4-b]indole

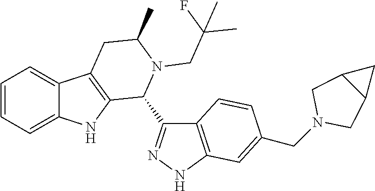 [SMILES: C[C@@H]1Cc2c([nH]c3ccccc23)[C@@H](c2n[nH]c3cc(CN4CC5CC5C4)ccc23)N1CC(C)(C)F]

Under nitrogen atmosphere, a solution of 3-((1S,3R)-2-(2-fluoro-2-methylpropyl)-3-methyl-2,3,4,9-tetrahydro-1H-pyrido[3,4-b]indol-1-yl)-1H-indazole-6-carbaldehyde (40.00 mg, 0.099 mmol) in anhydrous dichloromethane (1 mL) was added to a solution of 3-azabicyclo[3.1.0]hexane hydrochloride (23.65 mg, 0.20 mmol) and triethylamine (0.028 mL, 0.20 mmol) in anhydrous dichloromethane (2 mL), then acetic acid (0.023 mL, 0.40 mmol) was added to the reaction mixture then stirred at room temperature for 1 h. At 0 °C, sodium triacetoxyborohydride (41.92 mg, 0.20 mmol) was added to the reaction mixture, then stirred at room temperature for 12 h. The reaction mixture was quenched with MeOH (0.1 mL), diluted with dichloromethane (10 mL), the organic phase was washed with saturated NaHCO₃ solution and brine, the organic layer was dried with anhydrous Na₂SO₄, filtered, and concentrated under vacuum to afford crude product. The crude product was purified by normal phase Combiflash chromatography (0%-10% MeOH in dichloromethane) to afford the title compound (15.00 mg, 32.2% yield) as pale-yellow solid. LC-MS: m/z=472 [M+H]⁻.

### Example 25: (1S,3R)-2-(2-fluoro-2-methylpropyl)-1-(6-(((R)-3-(fluoromethyl)pyrrolidin-1-yl)methyl)-1H-indazol-3-yl)-3-methyl-2,3,4,9-tetrahydro-1H-pyrido[3,4-b]indole

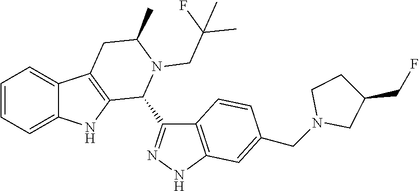 [SMILES: C[C@@H]1Cc2c([nH]c3ccccc23)[C@@H](c2n[nH]c3cc(CN4CC[C@@H](CF)C4)ccc23)N1CC(C)(C)F]

Under nitrogen atmosphere, a solution of 3-((1S,3R)-2-(2-fluoro-2-methylpropyl)-3-methyl-2,3,4,9-tetrahydro-1H-pyrido[3,4-b]indol-1-yl)-1H-indazole-6-carbaldehyde (40.00 mg, 0.099 mmol) in anhydrous dichloromethane (1 mL) was added to a solution of (R)-3-(fluoromethyl)pyrrolidine hydrochloride (27.61 mg, 0.20 mmol) and triethylamine (0.028 mL, 0.20 mmol) in anhydrous dichloromethane (2 mL), then acetic acid (0.023 mL, 0.40 mmol) was added to the reaction mixture then stirred at room temperature for 1 h. At 0 °C, sodium triacetoxyborohydride (41.92 mg, 0.20 mmol) was added to the reaction mixture, then stirred at room temperature for 12 h. The reaction mixture was quenched with MeOH (0.1 mL), diluted with dichloromethane (10 mL), the organic phase was washed with saturated NaHCO₃ solution and brine, the organic layer was dried with anhydrous Na₂SO₄, filtered, and concentrated under vacuum to afford crude product. The crude product was purified by normal phase Combiflash chromatography (0%-10% MeOH in dichloromethane) to afford the title compound (23.00 mg, 47.3% yield) as pale-yellow solid. LC-MS: m/z=492 [M+H]⁻.

### Example 26: (1S,3R)-2-(2-fluoro-2-methylpropyl)-1-(6-(((S)-3-(fluoromethyl)pyrrolidin-1-yl)methyl)-1H-indazol-3-yl)-3-methyl-2,3,4,9-tetrahydro-1H-pyrido[3,4-b]indole

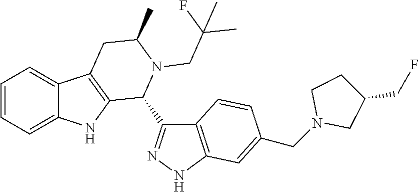 [SMILES: C[C@@H]1Cc2c([nH]c3ccccc23)[C@@H](c2n[nH]c3cc(CN4CC[C@H](CF)C4)ccc23)N1CC(C)(C)F]

Under nitrogen atmosphere, a solution of 3-((1S,3R)-2-(2-fluoro-2-methylpropyl)-3-methyl-2,3,4,9-tetrahydro-1H-pyrido[3,4-b]indol-1-yl)-1H-indazole-6-carbaldehyde (40.00 mg, 0.099 mmol) in anhydrous dichloromethane (1 mL) was added to a solution of(S)-3-(fluoromethyl)pyrrolidine hydrochloride (27.61 mg, 0.20 mmol) and triethylamine (0.028 mL, 0.20 mmol) in anhydrous dichloromethane (2 mL), then acetic acid (0.023 mL, 0.40 mmol) was added to the reaction mixture, then stirred at room temperature for 1 h. At 0 °C, sodium triacetoxyborohydride (41.92 mg, 0.20 mmol) was added to the reaction mixture, then stirred at room temperature for 12 h. The reaction mixture was quenched with MeOH (0.1 mL), diluted with dichloromethane (10 mL), the organic phase was washed with saturated NaHCO₃ solution and brine, the organic layer was dried with anhydrous Na₂SO₄, filtered, and concentrated under vacuum to afford crude product. The crude product was purified by normal phase Combiflash chromatography (0%-10% MeOH in dichloromethane) to afford the title compound (15.16 mg, 31.2% yield) as pale-yellow solid. LC-MS: m/z=492 [M+H]⁻.

### Example 27: (1S,3R)-1-(6-((2-azaspiro[3.3]heptan-2-yl)methyl)-1H-indazol-3-yl)-2-(2-fluoro-2-methylpropyl)-3-methyl-2,3,4,9-tetrahydro-1H-pyrido[3,4-b]indole

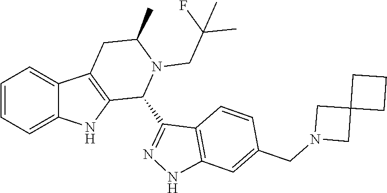 [SMILES: C[C@@H]1Cc2c([nH]c3ccccc23)[C@@H](c2n[nH]c3cc(CN4CC5(CCC5)C4)ccc23)N1CC(C)(C)F]

Under nitrogen atmosphere, a solution of 3-((1 S,3R)-2-(2-fluoro-2-methylpropyl)-3-methyl-2,3,4,9-tetrahydro-1H-pyrido[3,4-b]indol-1-yl)-1H-indazole-6-carbaldehyde (40.00 mg, 0.099 mmol) in anhydrous dichloromethane (1 mL) was added to a solution of 2-azaspiro[3.3]heptane hydrochloride (26.43 mg, 0.20 mmol) and triethylamine (0.028 mL, 0.20 mmol) in anhydrous dichloromethane (2 mL), then acetic acid (0.023 mL, 0.40 mmol) was added to the reaction mixture and stirred at room temperature for 1 h. At 0 °C, sodium triacetoxyborohydride (41.92 mg, 0.20 mmol) was added to the reaction mixture, then stirred at room temperature for 12 h. The reaction mixture was quenched with MeOH (0.1 mL), diluted with dichloromethane (10 mL), the organic phase was washed with saturated NaHCO₃ solution and brine, the organic layer was dried with anhydrous Na₂SO₄, filtered, and concentrated under vacuum to afford crude product. The crude product was purified by normal phase Combiflash chromatography (0%-10% MeOH in dichloromethane) to afford the title compound (16.18 mg, 33.7% yield) as pale yellow solid. LC-MS: m/z=486 [M+1]⁺.

### Example 28: (1S,3R)-1-(6-((6,6-difluoro-2-azaspiro[3.3]heptan-2-yl)methyl)-1H-indazol-3-yl)-2-(2-fluoro-2-methylpropyl)-3-methyl-2,3,4,9-tetrahydro-1H-pyrido[3,4-b]indole

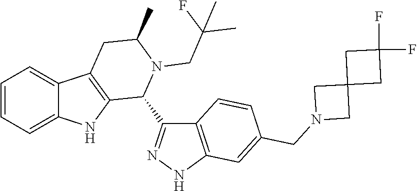 [SMILES: C[C@@H]1Cc2c([nH]c3ccccc23)[C@@H](c2n[nH]c3cc(CN4CC5(C4)CC(F)(F)C5)ccc23)N1CC(C)(C)F]

Under nitrogen atmosphere, a solution of 3-((1S,3R)-2-(2-fluoro-2-methylpropyl)-3-methyl-2,3,4,9-tetrahydro-1H-pyrido[3,4-b]indol-1-yl)-1H-indazole-6-carbaldehyde (40.00 mg, 0.099 mmol) in anhydrous dichloromethane (1 mL) was added to a solution of 6,6-difluoro-2-azaspiro[3.3]heptane 2,2,2-trifluoroacetate (48.88 mg, 0.20 mmol) and triethylamine (0.028 mL, 0.20 mmol) in anhydrous dichloromethane (2 mL), then acetic acid (0.023 mL, 0.40 mmol) was added to the reaction mixture and stirred at room temperature for 1 h. At 0 °C, sodium triacetoxyborohydride (41.92 mg, 0.20 mmol) was added to the reaction mixture, then stirred at room temperature for 12 h. The reaction mixture was quenched with MeOH (0.1 mL), diluted with dichloromethane (10 mL), the organic phase was washed with saturated NaHCO₃ solution and brine, the organic layer was dried with anhydrous Na₂SO₄, filtered, and concentrated under vacuum to afford crude product. The crude product was purified by normal phase Combiflash chromatography (0%-10% MeOH in dichloromethane) to afford the title compound (22.16 mg, 43.0% yield) as pale yellow solid. LC-MS: m/z=522 [M+1]⁺.

### Example 29: 6-((3-((1S,3R)-2-(2-fluoro-2-methylpropyl)-3-methyl-2,3,4,9-tetrahydro-1H pyrido-[3,4-b]indol-1-yl)-1H-indazol-6-yl)methyl)-2-oxa-6-azaspiro[3.3]heptane

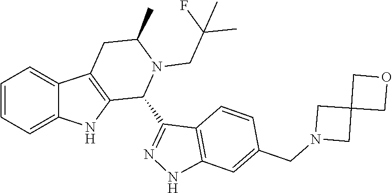 [SMILES: C[C@@H]1Cc2c([nH]c3ccccc23)[C@@H](c2n[nH]c3cc(CN4CC5(COC5)C4)ccc23)N1CC(C)(C)F]

Under nitrogen atmosphere, a solution of 3-((1S,3R)-2-(2-fluoro-2-methylpropyl)-3-methyl-2,3,4,9-tetrahydro-1H-pyrido[3,4-b]indol-1-yl)-1H-indazole-6-carbaldehyde (40.00 mg, 0.099 mmol) in anhydrous dichloromethane (1 mL) was added to the reaction solution of 2-oxa-6-azaspiro[3.3]heptane oxalate (37.42 mg, 0.20 mmol) and triethylamine (0.028 mL, 0.20 mmol) in anhydrous dichloromethane (2 mL), then acetic acid (0.023 mL, 0.40 mmol) was added to the reaction mixture and stirred at room temperature for 1 h. At 0 °C, sodium triacetoxyborohydride (41.92 mg, 0.20 mmol) was added to the reaction mixture, then stirred at room temperature for 12 h. The reaction mixture was quenched with MeOH (0.1 mL), diluted with dichloromethane (10 mL), the organic phase was washed with saturated NaHCO₃ solution and brine, the organic layer was dried with anhydrous Na₂SO₄, filtered, and concentrated under vacuum to afford crude product. The crude product was purified by normal phase Combiflash chromatography (0%-10% MeOH in dichloromethane) to afford the title compound (19.56 mg, 40.6% yield) as pale yellow solid. LC-MS: m/z=488 [M+1]⁺.

### Example 30: 2-((3-((1S,3R)-2-(2-fluoro-2-methylpropyl)-3-methyl-2,3,4,9-tetrahydro-1H-pyrido[3,4-b]-indol-1-yl)-1H-indazol-6-yl)methyl)-2-azaspiro[3.3]heptan-6-ol

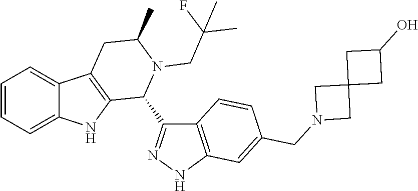 [SMILES: C[C@@H]1Cc2c([nH]c3ccccc23)[C@@H](c2n[nH]c3cc(CN4CC5(CC(O)C5)C4)ccc23)N1CC(C)(C)F]

Under nitrogen atmosphere, a solution of 3-((1S,3R)-2-(2-fluoro-2-methylpropyl)-3-methyl-2,3,4,9-tetrahydro-1H-pyrido[3,4-b]indol-1-yl)-1H-indazole-6-carbaldehyde (30.00 mg, 0.072 mmol) in anhydrous dichloromethane (1 mL) was added to a solution of 2-azaspiro[3.3]heptan-6-ol hydrochloride (22.19 mg, 0.15 mmol) and triethylamine (0.020 mL, 0.15 mmol) in anhydrous dichloromethane (2 mL), then acetic acid (0.017 mL, 0.30 mmol) was added to the reaction mixture and stirred at room temperature for 1 h. At 0 °C, sodium triacetoxyborohydride (31.44 mg, 0.15 mmol) was added to the reaction mixture, then stirred at room temperature for 12 h. The reaction mixture was quenched with MeOH (0.1 mL), diluted with dichloromethane (10 mL), the organic phase was washed with saturated NaHCO₃ solution and brine, the organic layer was dried with anhydrous Na₂SO₄, filtered, and concentrated under vacuum to afford crude product. The crude product was purified by normal phase Combiflash chromatography (0%-10% MeOH in dichloromethane) to afford the title compound (5.00 mg, 13.4% yield) as pale yellow solid. LC-MS: m/z=502[M+1]⁺.

### Example 31: 1-((3-((1S,3R)-2-(2-fluoro-2-methylpropyl)-3-methyl-2,3,4,9-tetrahydro-1H-pyrido-[3,4-b]indol-1-yl)-1H-indazol-6-yl)methyl)azetidine-3-carbonitrile

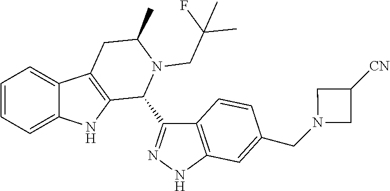 [SMILES: C[C@@H]1Cc2c([nH]c3ccccc23)[C@@H](c2n[nH]c3cc(CN4CC(C#N)C4)ccc23)N1CC(C)(C)F]

Under nitrogen atmosphere, a solution of 3-((1S,3R)-2-(2-fluoro-2-methylpropyl)-3-methyl-2,3,4,9-tetrahydro-1H-pyrido[3,4-b]indol-1-yl)-1H-indazole-6-carbaldehyde (30.00 mg, 0.072 mmol) in anhydrous dichloromethane (1 mL) was added to a solution of azetidine-3-carbonitrile hydrochloride (17.59 mg, 0.15 mmol) and triethylamine (0.020 mL, 0.15 mmol) in anhydrous dichloromethane (2 mL), then acetic acid (0.017 mL, 0.30 mmol) was added to the reaction mixture and stirred at room temperature for 1 h. At 0 °C, sodium triacetoxyborohydride (31.44 mg, 0.15 mmol) was added to the reaction mixture, then stirred at room temperature for 12 h. The reaction mixture was quenched with MeOH (0.1 mL), diluted with dichloromethane (10 mL), the organic phase was washed with saturated NaHCO₃ solution and brine, the organic layer was dried with anhydrous Na₂SO₄, filtered, and concentrated under vacuum to afford crude product. The crude product was purified by normal phase Combiflash chromatography (0%-10% MeOH in dichloromethane) to afford the title compound (12.36 mg, 35.4% yield) as pale yellow solid. LC-MS: m/z=471[M+1]⁺.

### Example 32: (1S,3R)-1-(6-((6-fluoro-2-azaspiro[3.3]heptan-2-yl)methyl)-1H-indazol-3-yl)-2-(2-fluoro-2-methylpropyl)-3-methyl-2,3,4,9-tetrahydro-1H-pyrido[3,4-b]indole

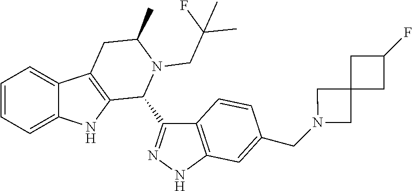 [SMILES: C[C@@H]1Cc2c([nH]c3ccccc23)[C@@H](c2n[nH]c3cc(CN4CC5(CC(F)C5)C4)ccc23)N1CC(C)(C)F]

Under nitrogen atmosphere, a solution of 3-((1S,3R)-2-(2-fluoro-2-methylpropyl)-3-methyl-2,3,4,9-tetrahydro-1H-pyrido[3,4-b]indol-1-yl)-1H-indazole-6-carbaldehyde (20.00 mg, 0.049 mmol) in anhydrous dichloromethane (1 mL) was added to a solution of 6-fluoro-2-azaspiro[3.3]heptane 2,2,2-trifluoroacetate (22.66 mg, 0.099 mmol) and triethylamine (0.014 mL, 0.099 mmol) in anhydrous dichloromethane (2 mL), then acetic acid (0.011 mL, 0.20 mmol) was added to the reaction mixture then stirred at room temperature for 1 h. At 0 °C, sodium triacetoxyborohydride (20.96 mg, 0.099 mmol) was added to the reaction mixture, then stirred at room temperature for 12 h. The reaction mixture was quenched with MeOH (0.1 mL), diluted with dichloromethane (10 mL), the organic phase was washed with saturated NaHCO₃ solution and brine, the organic layer was dried with anhydrous Na₂SO₄, filtered, and concentrated under vacuum to afford crude product. The crude product was purified by normal phase Combiflash chromatography (0%-10% MeOH in dichloromethane) to afford the title compound (13.56 mg, 54.5% yield) as pale yellow solid. LC-MS: m/z=504[M+1]⁺.

### Example 33: (1S,3R)-1-(6-((6,6-difluoro-1-azaspiro[3.3]heptan-1-yl)methyl)-1H-indazol-3-yl)-2-(2-fluoro-2-methylpropyl)-3-methyl-2,3,4,9-tetrahydro-1H-pyrido[3,4-b]indole

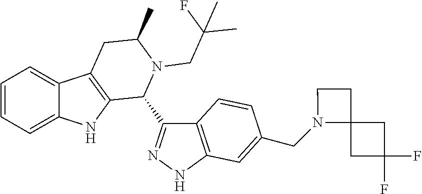 [SMILES: C[C@@H]1Cc2c([nH]c3ccccc23)[C@@H](c2n[nH]c3cc(CN4CCC45CC(F)(F)C5)ccc23)N1CC(C)(C)F]

Under nitrogen atmosphere, a solution of 3-((1S,3R)-2-(2-fluoro-2-methylpropyl)-3-methyl-2,3,4,9-tetrahydro-1H-pyrido[3,4-b]indol-1-yl)-1H-indazole-6-carbaldehyde (20.00 mg, 0.049 mmol) in anhydrous dichloromethane (1 mL) was added to a solution of 6,6-difluoro-1-azaspiro[3.3]heptane oxalate (17.62 mg, 0.049 mmol) and triethylamine (0.014 mL, 0.099 mmol) in anhydrous dichloromethane (2 mL), then acetic acid (0.011 mL, 0.20 mmol) was added to the reaction mixture and stirred at room temperature for 1 h. At 0 °C, sodium triacetoxyborohydride (20.96 mg, 0.099 mmol) was added to the reaction mixture, then stirred at room temperature for 12 h. The reaction mixture was quenched with MeOH (0.1 mL), diluted with dichloromethane (10 mL), the organic phase was washed with saturated NaHCO₃ solution and brine, the organic layer was dried with anhydrous Na₂SO₄, filtered, and concentrated under vacuum to afford crude product. The crude product was purified by normal phase Combiflash chromatography (0%-10% MeOH in dichloromethane) to afford the title compound (6.32 mg, 24.5% yield) as pale yellow solid. LC-MS: m/z=522[M+1]⁺.

### Example 34: Methyl 3-((1R,3R)-2-(3-((tert-butyldiphenylsilyl)oxy)-2,2-difluoropropyl)-3-methyl-2,3,4,9-tetrahydro-1H-pyrido[3,4-b]indol-1-yl)-1H-indole-6-carboxylate

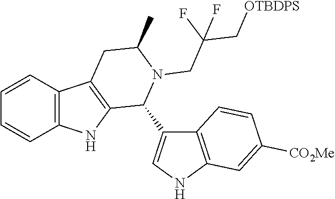

Under nitrogen atmosphere, methyl 3-formyl-1H-indole-6-carboxylate (325.10 mg, 1.60 mmol) and acetic acid (0.16 mL, 2.86 mmol) were added sequentially to a solution of (R)—N-(1-(1H-indol-3-yl)propan-2-yl)-3-((tert-butyldiphenylsilyl)oxy)-2,2-difluoropropan-1-amine (506.70 mg, 1.00 mmol) in anhydrous toluene (12 mL) in microwave vial, purged with nitrogen 3 times, the reaction mixture was heated at 130 °C in microwave reactor for 2 h. After cooling to room temperature, the reaction mixture was concentrated under vacuum and extracted with ethyl acetate and H₂O, washed with saturated NaHCO₃ solution, H₂O, and brine, dried with anhydrous Na₂SO₄, filtered, and concentrated under vacuum to afford crude product. The crude product was purified by normal phase Combiflash chromatography (0%-30% ethyl acetate in hexane) to afford the title compound (0.21 g, 30.4% yield) as pale-yellow solid. LC-MS: m/z=692 [M+H]⁺.

### Example 35: (3-((1R,3R)-2-(3-((tert-butyldiphenylsilyl)oxy)-2,2-difluoropropyl)-3-methyl-2,3,4,9-tetrahydro-1H-pyrido[3,4-b]indol-1-yl)-1H-indol-6-yl)methanol

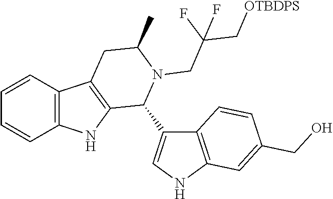

Under nitrogen atmosphere, a 1 M solution of lithium aluminum hydride (0.87 mL, 0.87 mmol) in THF was added dropwise to a solution of methyl 3-((1R,3R)-2-(3-((tert-butyldiphenylsilyl)oxy)-2,2-difluoropropyl)-3-methyl-2,3,4,9-tetrahydro-1H-pyrido[3,4-b]indol-1-yl)-1H-indole-6-carboxylate (200.00 mg, 0.29 mmol) in anhydrous THF (6 mL) at 0 °C, then stirred at room temperature for 3 h. The reaction mixture was cooled to 0 °C, 0.12 mL H₂O was dropwise to the reaction mixture, then added 0.24 mL 10% NaOH solution, followed by 0.48 mL H₂O, stirred at room temperature for 30 min, then filtered and concentrated under vacuum to afford crude product. The crude product was purified by normal phase Combiflash chromatography (0%-60% ethyl acetate in hexane) to afford the title compound (0.12 g, 62.5% yield) as pale-yellow solid. LC-MS: m/z=664[M+H]⁺.

### Example 36: 3-((1R,3R)-2-(3-((tert-butyldiphenylsilyl)oxy)-2,2-difluoropropyl)-3-methyl-2,3,4,9-tetrahydro-1H-pyrido[3,4-b]indol-1-yl)-1H-indole-6-carbaldehyde

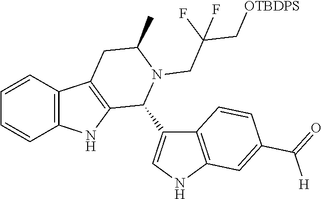

Under nitrogen atmosphere, (3-((1R,3R)-2-(3-((tert-butyldiphenylsilyl)oxy)-2,2-difluoropropyl)-3-methyl-2,3,4,9-tetrahydro-1H-pyrido[3,4-b]indol-1-yl)-1H-indol-6-yl)methanol (120.00 mg, 0.18 mmol) was dissolved in anhydrous dichloromethane (4 mL) and the reaction mixture was cooled to 0 °C. (1,1,1-Triacetoxy)-1,1-dihydro-1,2-benziodoxol-3(1H)-one (92.00 mg, 0.22 mmol) was added and the mixture was stirred at 0 °C for 30 min, then stirred at room temperature for 2 h. The mixture was diluted with dichloromethane (40 mL) and poured into saturated solutions of NaHCO₃ (20 mL) and saturated solutions of Na₂S₂O₃ (20 mL). It was stirred at room temperature for 60 min. The organic layer was washed with H₂O (60 mL) and brine (60 mL), dried over anhydrous Na₂SO₄, and filtrated, then the solvent was evaporated under reduced pressure to afford the crude product. The crude product was purified by normal phase Combiflash chromatography (0%-60% ethyl acetate in hexane) to afford the title compound (80.00 mg, 66.9% yield) as pale-yellow solid. LC-MS: m/z=662 [M+H]⁺.

### Example 37: 2,2-difluoro-3-((1R,3R)-1-(6-((3-(fluoromethyl)azetidin-1-yl)methyl)-1H-indol-3-yl)-3-methyl-1,3,4,9-tetrahydro-2H-pyrido[3,4-b]indol-2-yl)propan-1-ol

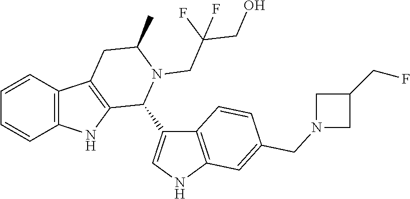 [SMILES: C[C@@H]1Cc2c([nH]c3ccccc23)[C@@H](c2c[nH]c3cc(CN4CC(CF)C4)ccc23)N1CC(F)(F)CO]

Under nitrogen atmosphere, a solution of 3-((1R,3R)-2-(3-((tert-butyldiphenylsilyl)oxy)-2,2-difluoropropyl)-3-methyl-2,3,4,9-tetrahydro-1H-pyrido[3,4-b]indol-1-yl)-1H-indole-6-carbaldehyde (60.00 mg, 0.091 mmol) in anhydrous dichloromethane (1 mL) was added to the reaction solution of 3-(fluoromethyl)azetidine hydrochloride (22.77 mg, 0.18 mmol) and triethylamine (0.025 mL, 0.18 mmol) in anhydrous dichloromethane (1 mL), then acetic acid (0.021 mL, 0.36 mmol) was added to the reaction mixture then stirred at room temperature for 1 h. At 0 °C, sodium triacetoxyborohydride (38.43 mg, 0.18 mmol) was added to the reaction mixture, then stirred at room temperature for 12 h. The reaction mixture was quenched with MeOH (0.1 mL), diluted with dichloromethane (10 mL), the organic phase was washed with saturated NaHCO₃ solution and brine, the organic layer was dried with anhydrous Na₂SO₄, filtered, and concentrated under vacuum to afford crude product, which was used for the subsequent reaction without further purification.

Under nitrogen atmosphere, a 1 M solution of tetrabutylammonium fluoride (0.18 mL, 0.18 mmol) in THF was added to a solution of crude product described above in anhydrous THE (4 mL) at room temperature, then stirred overnight. The reaction mixture was quenched with H₂O, extracted with ethyl acetate, the organic phase was washed with saturated H₂O and brine, the organic layer was dried with anhydrous Na₂SO₄, filtered, and concentrated under vacuum to afford crude product. The crude product was purified by normal phase Combiflash chromatography (0%-20% MeOH in dichloromethane) to afford the title compound (6.50 mg, 14.4% yield) as pale-yellow solid. LC-MS: m/z=497 [M+H]⁺.

### Example 38: Methyl 3-((1S,3R)-2-(3-((tert-butyldiphenylsilyl)oxy)-2,2-difluoropropyl)-3-methyl-2,3,4,9-tetrahydro-1H-pyrido[3,4-b]indol-1-yl)-1H-indazole-6-carboxylate

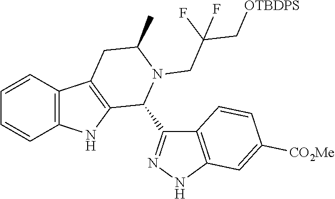

Under nitrogen atmosphere, methyl 3-formyl-1H-indazole-6-carboxylate (193.42 mg, 0.95 mmol) and acetic acid (0.13 mL, 2.26 mmol) were added sequentially to a solution of (R)—N-(1-(1H-indol-3-yl)propan-2-yl)-3-((tert-butyldiphenylsilyl)oxy)-2,2-difluoropropan-1-amine (400.00 mg, 0.79 mmol) in anhydrous toluene (10 mL) in microwave vial, exchanged the air in the vial with nitrogen 3 times, the reaction mixture was heated at 90 °C overnight. After cooling to room temperature, the reaction mixture was concentrated under vacuum and extracted with ethyl acetate and H₂O, washed with saturated NaHCO₃ solution, H₂O and brine, dried with anhydrous Na₂SO₄, filtered and concentrated under vacuum to afford crude product. The crude product was purified by normal phase Combiflash chromatography (0%-30% ethyl acetate in hexane) to afford the title compound (0.50 g, 91.4% yield) as pale-yellow solid. LC-MS: m/z=693 [M+H]⁺.

### Example 39: (3-((1S,3R)-2-(3-((tert-butyldiphenylsilyl)oxy)-2,2-difluoropropyl)-3-methyl-2,3,4,9-tetrahydro-1H-pyrido[3,4-b]indol-1-yl)-1H-indazol-6-yl)methanol

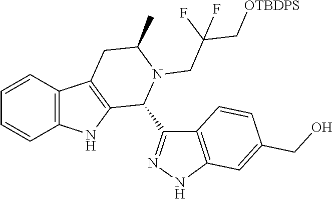

Under nitrogen atmosphere, a 1 M solution of lithium aluminum hydride (2.16 mL, 2.16 mmol) in THF was added dropwise to the solution of methyl 3-((1S,3R)-2-(3-((tert-butyldiphenylsilyl)oxy)-2,2-difluoropropyl)-3-methyl-2,3,4,9-tetrahydro-1H-pyrido[3,4-b]indol-1-yl)-1H-indazole-6-carboxylate (500.00 mg, 0.72 mmol) in anhydrous THF (12 mL) at 0 °C, then stirred at room temperature for 3 h. The reaction mixture was cooled to 0 °C, 0.40 mL H₂O was dropwise to the reaction mixture, then added 0.80 mL 10% NaOH solution, then added 1.20 mL H₂O, stirred at room temperature for 30 min, then filtered and concentrated under vacuum to afford crude product. The crude product was purified by normal phase Combiflash chromatography (0%-60% ethyl acetate in hexane) to afford the title compound (0.45 g, 93.8% yield) as pale-yellow solid. LC-MS: m/z=665[M+H]⁺.

### Example 40: 3-((1S,3R)-2-(3-((tert-butyldiphenylsilyl)oxy)-2,2-difluoropropyl)-3-methyl-2,3,4,9-tetrahydro-1H-pyrido[3,4-b]indol-1-yl)-1H-indazole-6-carbaldehyde

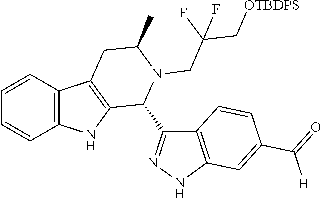

Under nitrogen atmosphere, (3-((1S,3R)-2-(3-((tert-butyldiphenylsilyl)oxy)-2,2-difluoropropyl)-3-methyl-2,3,4,9-tetrahydro-1H-pyrido[3,4-b]indol-1-yl)-1H-indazol-6-yl)methanol (0.45 g, 0.68 mmol) was dissolved in anhydrous dichloromethane (16 mL) and manganese dioxide (2.35 g, 27.07 mmol) was added, and the mixture was stirred at room temperature for 24 h. The mixture was filtered, then the solvent was evaporated under reduced pressure to afford the crude product. The crude product was purified by normal phase Combiflash chromatography (0%-50% ethyl acetate in hexane) to afford the title compound (0.21 g, 46.8% yield) as pale-yellow solid. LC-MS: m/z=663 [M+H]⁺.

### Example 41: 2,2-difluoro-3-((1S,3R)-1-(6-((3-(fluoromethyl)azetidin-1-yl)methyl)-1H-indazol-3-yl)-3-methyl-1,3,4,9-tetrahydro-2H-pyrido[3,4-b]indol-2-yl)propan-1-ol

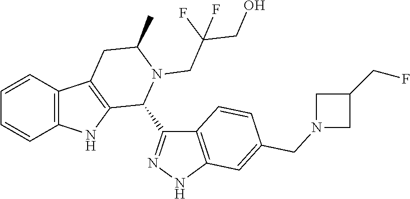 [SMILES: C[C@@H]1Cc2c([nH]c3ccccc23)[C@@H](c2n[nH]c3cc(CN4CC(CF)C4)ccc23)N1CC(F)(F)CO]

Under nitrogen atmosphere, a solution of 3-((1S,3R)-2-(3-((tert-butyldiphenylsilyl)oxy)-2,2-difluoropropyl)-3-methyl-2,3,4,9-tetrahydro-1H-pyrido[3,4-b]indol-1-yl)-1H-indazole-6-carbaldehyde (60.09 mg, 0.097 mmol) in anhydrous dichloromethane (1 mL) was added to a solution of 3-(fluoromethyl)azetidine hydrochloride (22.77 mg, 0.18 mmol) and triethylamine (0.025 mL, 0.18 mmol) in anhydrous dichloromethane (1 mL), then acetic acid (0.021 mL, 0.36 mmol) was added to the reaction mixture and stirred at room temperature for 1 h. At 0 °C, sodium triacetoxyborohydride (38.43 mg, 0.18 mmol) was added to the reaction mixture, then stirred at room temperature for 12 h. The reaction mixture was quenched with MeOH (0.1 mL), diluted with dichloromethane (10 mL), the organic phase was washed with saturated NaHCO₃ solution and brine, the organic layer was dried with anhydrous Na₂SO₄, filtered, and concentrated under vacuum to afford crude product, which was used for the following reaction without further purification.

Under nitrogen atmosphere, a 1 M solution of tetrabutylammonium fluoride (0.18 mL, 0.18 mmol) in THE was added to the solution of crude product described above in anhydrous THE (4 mL) at room temperature, then stirred overnight. The reaction mixture was quenched with H₂O, extracted with ethyl acetate, the organic phase was washed with saturated H₂O and brine, the organic layer was dried with anhydrous Na₂SO₄, filtered, and concentrated under vacuum to afford crude product. The crude product was purified by normal phase Combiflash chromatography (0%-20% MeOH in dichloromethane) to afford the title compound (14.00 mg, 31.0% yield) as pale-yellow solid. LC-MS: m/z=498 [M+H]⁻.

### Example 42: 7-Fluoro-6-[[3-(fluoromethyl)azetidin-1-yl]methyl]-1H-indole

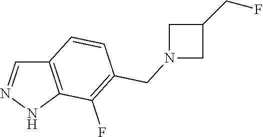 [SMILES: FCC1CN(Cc2ccc3cn[nH]c3c2F)C1]

In DMF (0.5 mL) was dissolved 7-fluoro-1H-indole-6-carbaldehyde (100 mg, 0.61 mmol) and acetic acid (70 uL, 1.22 mmol). To this was added 3-(fluoromethyl)azetidine hydrochloride (92 mg, 1.23 mmol) dissolved in DMF (0.5 mL) and TEA (170 μL, 1.23 mmol) and stirred for 30 min. Sodium triacetoxyborohydride (390 mg, 0.1.84 mmol) was added and stirred for 2 hours. The reaction was quenched with 1 N NaOH solution and extracted with EtOAc. The combined organic layers were washed with brine solution five times, dried over sodium sulfate, filtered, and dried in vacuo to give the title product (140 mg, 97% yield).

### Example 43: 7-Fluoro-6-[[3-(fluoromethyl)azetidin-1-yl]methyl]-1H-indole-3-carbaldehyde

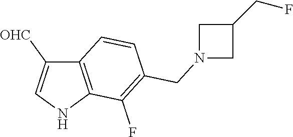 [SMILES: O=Cc1c[nH]c2c(F)c(CN3CC(CF)C3)ccc12]

7-Fluoro-6-[[3-(fluoromethyl)azetidin-1-yl]methyl]-1H-indole (115 mg, 0.49 mmol) and hexamethylenetetramine (218 mg, 1.56 mmol) were dissolved in 3 mL ethanol, acetic acid (128 uL, 2.24 mmol) was added and stirred at 100 °C for 6 hours. The reaction was quenched with saturated sodium bicarbonate solution and extracted with EtOAc. The combined organics were dried over sodium sulfate, filtered, and dried in vacuo. The crude was purified via flash chromatography using a 24 g silica column in a 0-15% MeOH in DCM gradient to give the title compound (30 mg, 23% yield). LCMS: m/z=265.3 [M+H]⁺.

### Example 44: (1R,3R)-1-[7-Fluoro-6-[[3-(fluoromethyl)azetidin-1-yl]methyl]-1H-indol-3-yl]-2-(2-fluoro-2-methyl-propyl)-3-methyl-1,3,4,9-tetrahydropyrido[3,4-b]indole

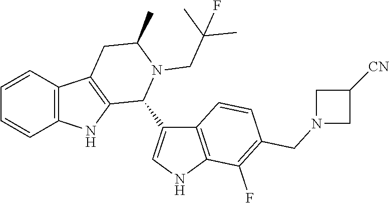 [SMILES: C[C@@H]1Cc2c([nH]c3ccccc23)[C@@H](c2c[nH]c3c(F)c(CN4CC(C#N)C4)ccc23)N1CC(C)(C)F]

7-Fluoro-6-[[3-(fluoromethyl)azetidin-1-yl]methyl]-1H-indole-3-carbaldehyde (30 mg, 0.11 mmol) and 2-fluoro-N-[(1R)-2-(1H-indol-3-yl)-1-methyl-ethyl]-2-methyl-propan-1-amine (56 mg, 0.22 mmol) were dissolved in 10 mL of dioxane, acetic acid (26 uL, 0.45 mmol) was added and stirred at 120 °C for 4 hours. The reaction crude was neutralized with saturated sodium bicarbonate solution and the organic layer was separated. The aqueous layer was extracted with EtOAc and the combined organic layers were washed with brine solution. It was then dried over sodium sulfate, filtered, and dried in vacuo. The crude was first purified via flash chromatography on an 11 g Biotage™ Sfar KP-Amino column in 0-15% IPA in DCM with 1% TEA as modifier. The resulting material was dried, then dissolved in 1.5 mL DMA and purified by HPLC on Kintetex 5 μm C18 100 Å column (size: 100×30.0 mm; gradient: 5-50% 0.1% formic acid in ACN in 0.1% formic acid in water) then lyophilized to give the title compound as a white, fluffy, solid (1.0 mg, 1.8% yield). LCMS: m/z=495.5 [M+H]⁺.

### Example 45: (1R,3R)-1-(6-bromo-1H-indol-3-yl)-2-(2-fluoro-2-methylpropyl)-3-methyl-2,3,4,9-tetrahydro-1H-pyrido[3,4-b]indole

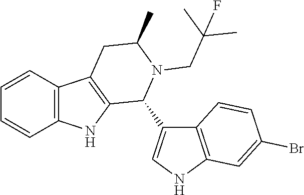 [SMILES: C[C@@H]1Cc2c([nH]c3ccccc23)[C@@H](c2c[nH]c3cc(Br)ccc23)N1CC(C)(C)F]

A solution of 6-bromo-1H-indazole-3-carbaldehyde (1.00 equiv, 338 mg, 1.50 mmol), 2-fluoro-N-[(1R)-2-(1H-indol-3-yl)-1-methyl-ethyl]-2-methyl-propan-1-amine (1.20 equiv, 447 mg, 1.80 mmol) and acetic acid (4.00 equiv, 0.34 mL, 6.00 mmol) in DCE (4 mL) was heated at 100 °C for 90 min. Na₂CO₃ (aq, 10 mL) was added and the reaction extracted with DCM (5 mL). The solvent was removed and the reaction was purified via silica gel (40 g, Gold) eluting at 25% hexanes:EtOAc to afford (3R)-1-(6-bromo-1H-indazol-3-yl)-2-(2-fluoro-2-methyl-propyl)-3-methyl-1,3,4,9-tetrahydropyrido[3,4-b]indole (368 mg, 0.808 mmol, 53.88% yield) as a yellow solid. LCMS: m/z=455.1, 456.9 [M+H]⁺.

### Example 46: tert-butyl 3-((3-((1R,3R)-2-(2-fluoro-2-methylpropyl)-3-methyl-2,3,4,9-tetrahydro-1H-pyrido[3,4-b]indol-1-yl)-1H-indol-6-yl)methyl)azetidine-1-carboxylate

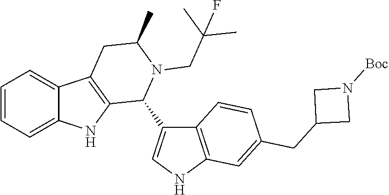 [SMILES: C[C@@H]1Cc2c([nH]c3ccccc23)[C@@H](c2c[nH]c3cc(CC4CN(C(=O)OC(C)(C)C)C4)ccc23)N1CC(C)(C)F]

A solution of (3R)-1-(6-bromo-1H-indazol-3-yl)-2-(2-fluoro-2-methyl-propyl)-3-methyl-1,3,4,9-tetrahydropyrido[3,4-b]indole (1.00 equiv, 140 mg, 0.307 mmol), potassium; (1-tert-butoxycarbonylazetidin-3-yl)methyl-trifluoro-boranuide (1.95 equiv, 166 mg, 0.599 mmol), cataCXium Pd G4 (0.200 equiv, 46 mg, 0.0615 mmol), cesium carbonate (3.00 equiv, 301 mg, 0.922 mmol) in 6:1 toluene:water (3 mL:500 μL). The reaction was microwaved at 120 °C, 140 °C and 160 °C for 5 minutes each. Water (2 mL) was added and the reaction extracted with EtOAc (3 mL×2). The combined organic layers were dried over sodium sulfate and purified twice via silica gel (25 g, Sfar and 28 g, KP-Amino) eluting at 70% hexanes:EtOAc to afford tert-butyl 3-[[3-[(3R)-2-(2-fluoro-2-methyl-propyl)-3-methyl-1,3,4,9-tetrahydropyrido[3,4-b]indol-1-yl]-1H-indazol-6-yl]methyl]azetidine-1-carboxylate (10 mg, 0.0183 mmol, 5.96% yield) as a beige solid. LCMS: m/z=546.0 [M+H]⁺.

### Example 47: (1R,3R)-2-(2-fluoro-2-methylpropyl)-3-methyl-1-(6-((1-methylazetidin-3-yl)methyl)-1H-indol-3-yl)-2,3,4,9-tetrahydro-1H-pyrido[3,4-b]indole

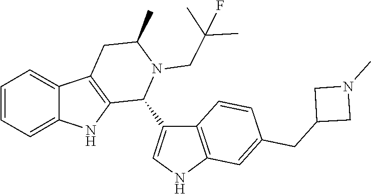 [SMILES: C[C@@H]1Cc2c([nH]c3ccccc23)[C@@H](c2c[nH]c3cc(CC4CN(C)C4)ccc23)N1CC(C)(C)F]

To a solution of lithium aluminum hydride (4.81 equiv, 0.15 mL, 0.150 mmol) in THF (1 mL) at 0 °C was added tert-butyl 3-[[3-[(3R)-2-(2-fluoro-2-methyl-propyl)-3-methyl-1,3,4,9-tetrahydropyrido[3,4-b]indol-1-yl]-1H-indol-6-yl]methyl]azetidine-1-carboxylate (1.00 equiv, 17 mg, 0.0312 mmol). After stirring for 4 d, sodium sulfate decahydrate was added along with THE (1 mL). After 1.5 h, water (2 mL) was added and the reaction was extracted with EtOAc (2 mL×2). The combined organic layers were dried over sodium sulfate, concentrated and purified via HPLC eluting with water (0.1% formic acid):ACN (0.1% formic acid) to afford (3R)-2-(2-fluoro-2-methyl-propyl)-3-methyl-1-[6-[(1-methylazetidin-3-yl)methyl]-1H-indol-3-yl]-1,3,4,9-tetrahydropyrido[3,4-b]indole (4.7 mg, 0.0102 mmol, 32.84% yield) as an orange solid. LCMS: m/z=459.3 [M+H]⁺.

### Example 48: Methyl 2-[(3R)-2-(2-fluoro-2-methyl-propyl)-3-methyl-1,3,4,9-tetrahydropyrido[3,4-b]indol-1-yl]-1H-indole-5-carboxylate

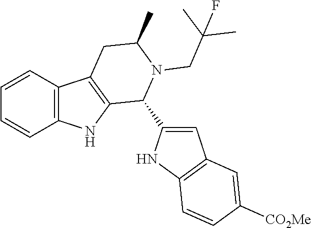 [SMILES: COC(=O)c1ccc2[nH]c([C@@H]3c4[nH]c5ccccc5c4C[C@@H](C)N3CC(C)(C)F)cc2c1]

To 2-fluoro-N-[(1R)-2-(1H-indol-3-yl)-1-methyl-ethyl]-2-methyl-propan-1-amine (1.00 equiv, 122 mg, 0.492 mmol) and methyl 2-formyl-1H-indole-5-carboxylate (1.00 equiv, 100 mg, 0.492 mmol) in DCE (4 mL) under nitrogen was added acetic acid (2.00 equiv, 0.056 mL, 0.984 mmol) and heated at 110 °C for 3 h in the microwave. The reaction mixture was extracted with DCM, washed with saturated sodium bicarbonate, dried over sodium sulfate, filtered and concentrated. The crude was purified by silica gel chromatography using 0-40% EtOAc in hexane to give title compound (160 mg, 0.369 mmol, 74.99% yield). LCMS: m/z=434.5 [M+H]⁺.

### Example 49: [2-[(1R,3R)-2-(2-fluoro-2-methyl-propyl)-3-methyl-1,3,4,9-tetrahydropyrido[3,4-b]indol-1-yl]-1H-indol-5-yl]methanol

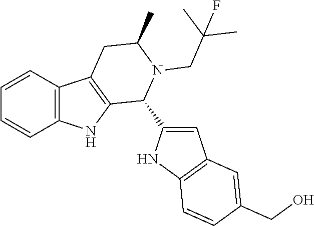 [SMILES: C[C@@H]1Cc2c([nH]c3ccccc23)[C@@H](c2cc3cc(CO)ccc3[nH]2)N1CC(C)(C)F]

To methyl 2-[(3R)-2-(2-fluoro-2-methyl-propyl)-3-methyl-1,3,4,9-tetrahydropyrido[3,4-b]indol-1-yl]-1H-indole-5-carboxylate (1.00 equiv, 160 mg, 0.369 mmol) in THF (10 mL) at 0 °C under nitrogen was added lithium aluminum hydride (2.00 equiv, 0.74 mL, 0.738 mmol). The reaction was stirred at 0 °C for 2 h, diluted with 15 mL of MTBE and treated with 30 μL of water, followed by 30 μL of 15% aqueous NaOH and then 90 μL of water. The reaction was warmed to room temperature and stirred for 15 mins and to this was added anhydrous sodium sulfate stirred and the reaction mixture filtered through Celite®. The filtrated was concentrated and the crude was purified by silica gel chromatography using 0-50% EtOAc in hexane to give title compound (120 mg, 0.296 mmol, 80.18% yield). LCMS: m/z=406.3 [M+H]⁺.

### Example 50: 2-[(1R,3R)-2-(2-fluoro-2-methyl-propyl)-3-methyl-1,3,4,9-tetrahydropyrido[3,4-b]indol-1-yl]-1H-indole-5-carbaldehyde

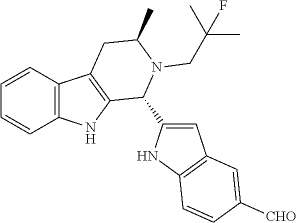 [SMILES: C[C@@H]1Cc2c([nH]c3ccccc23)[C@@H](c2cc3cc(C=O)ccc3[nH]2)N1CC(C)(C)F]

To [2-[(1R,3R)-2-(2-fluoro-2-methyl-propyl)-3-methyl-1,3,4,9-tetrahydropyrido[3,4-b]indol-1-yl]-1H-indol-5-yl]methanol (1.00 equiv, 70 mg, 0.173 mmol) in anhydrous DMSO (1 mL) was added 1-hydroxy-1,2-benziodoxol-3(1H)-one 1-oxide (1.05 equiv, 113 mg, 0.181 mmol) and stirred at room temperature for 2 h. The reaction was diluted with water, extracted with ethyl acetate and the layers separated. The EtOAc layer was washed with water, brine, dried over sodium sulfate, filtered, and concentrated. The crude was purified by silica gel chromatography using 0-50% EtOAc in hexane to give title compound (40 mg, 0.0991 mmol, 57.43% yield). LCMS: m/z=404.2 [M+H]⁺.

### Example 51: (1R,3R)-1-[5-[[3-(fluoromethyl)azetidin-1-yl]methyl]-1H-indol-2-yl]-2-(2-fluoro-2-methyl-propyl)-3-methyl-1,3,4,9-tetrahydropyrido[3, 4-b]indole

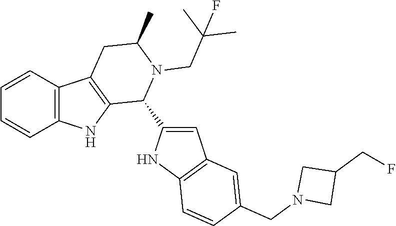 [SMILES: C[C@@H]1Cc2c([nH]c3ccccc23)[C@@H](c2cc3cc(CN4CC(CF)C4)ccc3[nH]2)N1CC(C)(C)F]

To a solution of 2-[(1R,3R)-2-(2-fluoro-2-methyl-propyl)-3-methyl-1,3,4,9-tetrahydropyrido[3,4-b]indol-1-yl]-1H-indole-5-carbaldehyde (1.00 equiv, 20 mg, 0.0496 mmol) in DMF (0.5 mL) was added the 3-(fluoromethyl)azetidin-1-ium; chloride (1.00 equiv, 6.2 mg, 0.0496 mmol) dissolved in DMF (0.5 mL) and triethylamine (2.00 equiv, 0.014 mL, 0.0991 mmol). To the reaction mixture was added acetic acid (2.00 equiv, 0.0057 mL, 0.0991 mmol) and stirred at room temperature for 1 h and then treated with sodium triacetoxyborohydride (3.00 equiv, 32 mg, 0.149 mmol) and stirred for 1 h. The reaction was quenched with 1 mL of methanol and the crude directly purified by C-18 reverse phase chromatography using prep HPLC, eluting with 10-50% ACN in water with 0.1% formic acid as modifier, to give title compound (6.0 mg, 0.0126 mmol, 25.40% yield). LCMS: m/z=477.9 [M+H]⁺.

### Example 52: 2-(hydroxymethyl)-1H-indole-6-carbonitrile

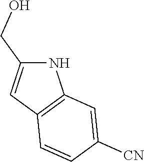 [SMILES: N#Cc1ccc2cc(CO)[nH]c2c1]

Methyl 6-cyano-1H-indole-2-carboxylate (1.00 equiv, 0.13 g, 0.624 mmol) was added to solution of lithium borohydride (2.00 equiv, 0.62 mL, 1.25 mmol) in THE (5 mL) and water (0.5 mL). The reaction was stirred for 16 h at 25 °C, then quenched with water (1 mL) and made acidic with 1 N HCl. The reaction mixture was extracted with ethyl acetate, dried over Na₂SO₄, filtered, and concentrated under vacuum to give title compound (90 mg, 0.523 mmol, 83.71% yield) which was used without further purification. LCMS: m/z=173.2 [M+H]⁺.

### Example 53: 2-formyl-1H-indole-6-carbonitrile

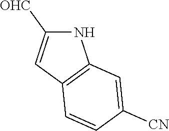 [SMILES: N#Cc1ccc2cc(C=O)[nH]c2c1]

To 2-(hydroxymethyl)-1H-indole-6-carbonitrile (1.00 equiv, 90 mg, 0.523 mmol) in anhydrous DMSO (1 mL) was added 1-hydroxy-1,2-benxiodoxol-3(1H)-one 1-oxide (1.00 equiv, 325 mg, 0.523 mmol) and stirred at 25 °C for 2 h. The reaction mixture was diluted with water, extracted with ethyl acetate, and the EtOAc layer was washed with water, brine, dried over Na₂SO₄, filtered and concentrated. The crude was purified by silica gel chromatography, eluting with 10-40% EtOAc in hexane, to give title compound (65 mg, 0.382 mmol, 73.08% yield). LCMS: m/z=171.2 [M+H]⁺.

### Example 54: 2-formyl-1H-indole-6-carbonitrile

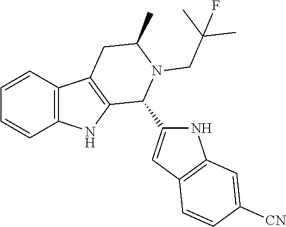 [SMILES: C[C@@H]1Cc2c([nH]c3ccccc23)[C@@H](c2cc3ccc(C#N)cc3[nH]2)N1CC(C)(C)F]

To 2-(hydroxymethyl)-1H-indole-6-carbonitrile (1.00 equiv, 90 mg, 0.523 mmol) in anhydrous DMSO (1 mL) was added 1-hydroxy-1,2-benxiodoxol-3(1H)-one 1-oxide (1.00 equiv, 325 mg, 0.523 mmol) and stirred at 25 °C for 2 h. The reaction mixture was diluted with water, extracted with ethyl acetate, and the EtOAc layer was washed with water, brine, dried over Na₂SO₄, filtered and concentrated. The crude was purified by silica gel chromatography, eluting with 10-40% EtOAc in hexane, to give title compound (65 mg, 0.382 mmol, 73.08% yield). LCMS: m/z=171.2 [M+H]⁺.

### Example 55: 2-[(1R,3R)-2-(2-fluoro-2-methyl-propyl)-3-methyl-1,3,4,9-tetrahydropyrido[3,4-b]indol-1-yl]-1H-indole-6-carbaldehyde

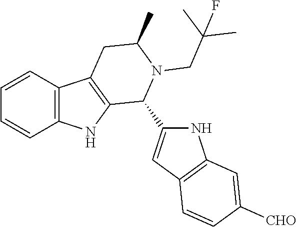 [SMILES: C[C@@H]1Cc2c([nH]c3ccccc23)[C@@H](c2cc3ccc(C=O)cc3[nH]2)N1CC(C)(C)F]

To 2-[(1R,3R)-2-(2-fluoro-2-methyl-propyl)-3-methyl-1,3,4,9-tetrahydropyrido[3,4-b]indol-1-yl]-1H-indole-6-carbonitrile (1.00 equiv, 105 mg, 0.262 mmol) in DCM (10 mL) under an atmosphere of nitrogen at −76 °C was added the diisobutylaluminum hydride (3.00 equiv, 0.79 mL, 0.787 mmol) and stirred for 2 h. The reaction was the quenched with ethyl acetate, warmed to 0 °C and then treated with saturated solution of Rochelle's salt. The reaction mixture was stirred for 30 mins, and the layers separated. The aqueous phase was extracted with EtOAc, washed with brine, dried over sodium sulfate, filtered, and concentrated. The crude was purified by silica gel chromatography using 0-30% EtOAc in hexane to give title compound (60 mg, 0.149 mmol, 56.72% yield). 404.2 [M+H]⁺.

### Example 56: (1R,3R)-1-[6-[[3-(fluoromethyl)azetidin-1-yl]methyl]-1H-indol-2-yl]-2-(2-fluoro-2-methyl-propyl)-3-methyl-1,3,4,9-tetrahydropyrido[3,4-b]indole

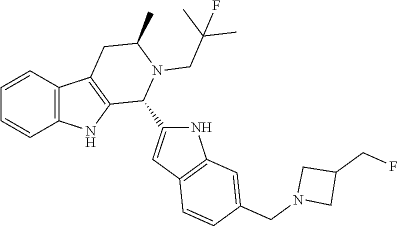 [SMILES: C[C@@H]1Cc2c([nH]c3ccccc23)[C@@H](c2cc3ccc(CN4CC(CF)C4)cc3[nH]2)N1CC(C)(C)F]

To solution of 3-(fluoromethyl)azetidine hydrochloride (2.00 equiv, 12 mg, 0.0991 mmol) in DMF (0.5 mL) was added triethylamine (2.00 equiv, 0.014 mL, 0.0991 mmol), followed by a solution of 2-[(1R,3R)-2-(2-fluoro-2-methyl-propyl)-3-methyl-1,3,4,9-tetrahydropyrido[3,4-b]indol-1-yl]-1H-indole-6-carbaldehyde (1.00 equiv, 20 mg, 0.0496 mmol) in DMF (0.5 mL). To the reaction was added acetic acid (2.00 equiv, 0.0057 mL, 0.0991 mmol) and stirred at room temperature for 1 h. The reaction mixture was treated with sodium triacetoxyborohydride (3.00 equiv, 32 mg, 0.149 mmol) and stirred for 1 h. The reaction was quenched with 1 mL of methanol and purified by C-18 reverse phase chromatography using a Phenomenex Luna column, eluting with 10-50% ACN in water with 0.1% formic acid as modifier, to give title compound (4.0 mg, 0.00839 mmol, 16.93% yield). 477.2 [M+H]⁺.

### Example 57: 2-(Hydroxymethyl)-1H-indole-5-carbaldehyde

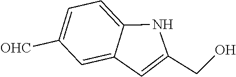 [SMILES: O=Cc1ccc2[nH]c(CO)cc2c1]

To methyl 5-cyano-1H-indole-2-carboxylate (1.00 equiv, 1.00 g, 5.00 mmol) in DCM (30 mL) at 0 °C under nitrogen was added the diisobutylaluminum hydride (4.00 equiv, 20 mL, 20.0 mmol) and stirred at 0 °C for 2 h. The reaction was quenched with ethyl acetate, followed by saturated solution of Rochelle's salt, and stirred for 30 mins. The organic layer was separated and aqueous layer, extracted with additional DCM twice. The organic layer was dried over sodium sulfate, filtered, and concentrated. The crude was purified by silica gel chromatography using 0-10% MeOH in DCM to give title compound (600 mg, 3.43 mmol, 68.6% yield). LCMS: m/z=176.1 [M+H]⁺.

### Example 58: [5-[(1R,3R)-2-(2-fluoro-2-methyl-propyl)-3-methyl-1,3,4,9-tetrahydropyrido[3,4-b]indol-1-yl]-1H-indol-2-yl]methanol

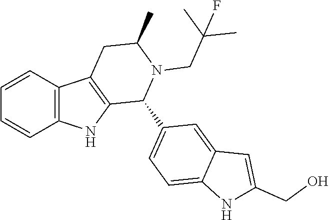 [SMILES: C[C@@H]1Cc2c([nH]c3ccccc23)[C@@H](c2ccc3[nH]c(CO)cc3c2)N1CC(C)(C)F]

To 2-(hydroxymethyl)-1H-indole-5-carbaldehyde (1.00 equiv, 200 mg, 1.14 mmol) in DCE (3 mL)/1,4-dioxane (3 mL) was added the 2-fluoro-N-[(1R)-2-(1H-indol-3-yl)-1-methyl-ethyl]-2-methyl-propan-1-amine (1.00 equiv, 284 mg, 1.14 mmol) and acetic acid (2.00 equiv, 0.13 mL, 2.28 mmol) and heated at 110 °C in microwave for 6 h. The reaction mixture was extracted with DCM, washed with saturated sodium bicarbonate solution, dried over sodium sulfate, filtered, and concentrated. The crude was purified by silica gel chromatography using a 24 g RediSep gold column eluting with 0-10% methanol in DCM to give title compound (150 mg, 0.370 mmol, 32.40% yield). LCMS: m/z=406.9 [M+H]⁺.

### Example 59: 5-[(1R,3R)-2-(2-fluoro-2-methyl-propyl)-3-methyl-1,3,4,9-tetrahydropyrido[3,4-b]indol-1-yl]-1H-indole-2-carbaldehyde

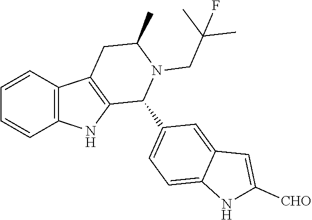 [SMILES: C[C@@H]1Cc2c([nH]c3ccccc23)[C@@H](c2ccc3[nH]c(C=O)cc3c2)N1CC(C)(C)F]

To [5-[(1R,3R)-2-(2-fluoro-2-methyl-propyl)-3-methyl-1,3,4,9-tetrahydropyrido[3,4-b]indol-1-yl]-1H-indol-2-yl]methanol (1.00 equiv, 150 mg, 0.370 mmol) in DMSO (2.5 mL) was added 1-hydroxy-1,2-benziodoxol-3(1H)-one 1-oxide (1.00 equiv, 230 mg, 0.370 mmol) and stirred at room temperature for 1 h. The reaction was diluted with water, extracted with ethyl acetate, and the layers separated. The EtOAc layer was washed with water, brine, dried over sodium sulfate, filtered, and concentrated. The crude was purified by silica gel chromatography using 0-40% EtOAc in hexane to give title compound (130 mg, 0.322 mmol, 87.1% yield). LCMS: m/z=404.1 [M+H]⁺.

### Example 60: (1R,3R)-1-[2-[[3-(fluoromethyl)azetidin-1-yl]methyl]-1H-indol-5-yl]-2-(2-fluoro-2-methyl-propyl)-3-methyl-1,3,4,9-tetrahydropyrido[3,4-b]indole

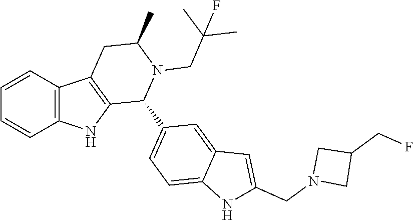 [SMILES: C[C@@H]1Cc2c([nH]c3ccccc23)[C@@H](c2ccc3[nH]c(CN4CC(CF)C4)cc3c2)N1CC(C)(C)F]

To solution of 5-[(1R,3R)-2-(2-fluoro-2-methyl-propyl)-3-methyl-1,3,4,9-tetrahydropyrido[3,4-b]indol-1-yl]-1H-indole-2-carbaldehyde (1.00 equiv, 60 mg, 0.149 mmol) in DMF (2 mL) was added 3-(fluoromethyl)azetidin-1-ium; chloride (2.00 equiv, 37 mg, 0.297 mmol) dissolved in DMF (2 mL) and triethylamine (2.00 equiv, 0.041 mL, 0.297 mmol). To the reaction mixture was added acetic acid (2.00 equiv, 0.017 mL, 0.297 mmol) and stirred at room temperature for 1 h and then treated with sodium triacetoxyborohydride (3.00 equiv, 95 mg, 0.446 mmol) and stirred for additional 2 h. The reaction was quenched with 1 mL of methanol and the crude directly purified by C-18 reverse phase chromatography on a Phenomenex Luna column, eluting with 10-50% ACN in water with 0.1% formic acid as modifier, to give title compound (30 mg, 0.0629 mmol, 42.3% yield). LCMS: m/z=477.3 [M+H]⁺.

### Example 61: (1R,3R)-2-(2-fluoro-2-methyl-propyl)-3-methyl-1-[2-[(3-methylazetidin-1-yl)methyl]-1H-indol-5-yl]-1,3,4,9-tetrahydropyrido[3,4-b]indole

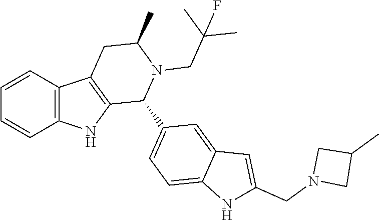 [SMILES: CC1CN(Cc2cc3cc([C@@H]4c5[nH]c6ccccc6c5C[C@@H](C)N4CC(C)(C)F)ccc3[nH]2)C1]

3-Methylazetidin-1-ium chloride was used to synthesize the title compound using general procedure from Example 60 to give title compound (5.0 mg, 0.0109 mmol, 29.33% yield). LCMS: m/z=459.2 [M+H]⁺.

### Example 62: (1R,3R)-1-[2-(3-azabicyclo[3.1.0]hexan-3-ylmethyl)-11H-indol-5-yl]-2-(2-fluoro-2-methyl-propyl)-3-methyl-1,3,4,9-tetrahydropyrido[3,4-b]indole

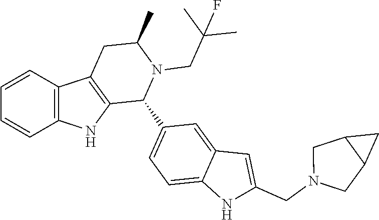 [SMILES: C[C@@H]1Cc2c([nH]c3ccccc23)[C@@H](c2ccc3[nH]c(CN4CC5CC5C4)cc3c2)N1CC(C)(C)F]

3-Azabicyclo[3.1.0]hexane hydrochloride (2.00 equiv, 8.9 mg, 0.0744 mmol) was used to synthesize the title compound using general procedure from Example 60 to give title compound (9.0 mg, 0.0191 mmol, 51.44% yield). LCMS: m/z=471.3 [M+H]⁺.

### Example 63: (1R,3R)-1-[2-(2-azaspiro[3.3]heptan-2-ylmethyl)-1H-indol-5-yl]-2-(2-fluoro-2-methyl-propyl)-3-methyl-1,3,4,9-tetrahydropyrido[3,4-b]indole

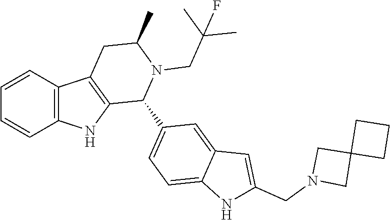 [SMILES: C[C@@H]1Cc2c([nH]c3ccccc23)[C@@H](c2ccc3[nH]c(CN4CC5(CCC5)C4)cc3c2)N1CC(C)(C)F]

2-Azoniaspiro[3.3]heptane; chloride (2.00 equiv, 9.9 mg, 0.0744 mmol) was used to synthesize the title compound using the general procedure from Example 60 to give title compound (5.0 mg, 0.0103 mmol, 27.75% yield). LCMS: m/z=485.1 [M+H]⁺.

### Example 64: (1R,3R)-2-(2-fluoro-2-methyl-propyl)-1-[2-[[(3S)-3-fluoropyrrolidin-1-yl]methyl]-1H-indol-5-yl]-3-methyl-1,3,4,9-tetrahydropyrido[3,4-b]indole

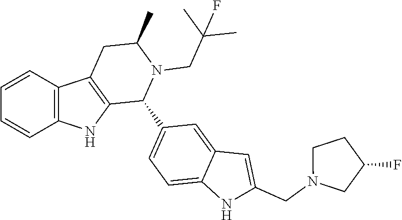 [SMILES: C[C@@H]1Cc2c([nH]c3ccccc23)[C@@H](c2ccc3[nH]c(CN4CC[C@H](F)C4)cc3c2)N1CC(C)(C)F]

(3S)-3-Fluoropyrrolidin-1-ium; chloride (2.00 equiv, 9.3 mg, 0.0744 mmol)) was used to synthesize the title compound using general procedure from Example 60 to give title compound (5.0 mg, 0.0105 mmol, 28.22% yield). LCMS: m/z=477.3 [M+H]⁺.

### Example 65: [5-[(1R,3R)-2-[3-[tert-butyl(diphenyl)silyl]oxy-2,2-difluoro-propyl]-3-methyl-1,3,4,9-tetrahydropyrido[3,4-b]indol-1-yl]-1H-indol-2-yl]methanol

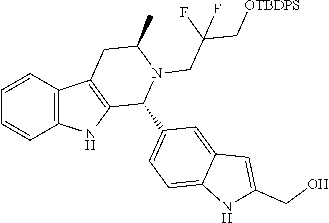

To 2-(hydroxymethyl)-1H-indole-5-carbaldehyde (1.20 equiv, 166 mg, 0.947 mmol) in DCE (4 mL)/1,4-dioxane (4 mL) was added 3-[tert-butyl(diphenyl)silyl]oxy-2,2-difluoro-N-[(1R)-2-(1H-indol-3-yl)-1-methyl-ethyl]propan-1-amine (1.00 equiv, 400 mg, 0.789 mmol) and acetic acid (2.00 equiv, 0.090 mL, 1.58 mmol) and stirred at 110 °C in microwave for 6 h. The reaction mixture was extracted with DCM, washed with saturated sodium bicarbonate solution, dried over sodium sulfate, filtered, and concentrated. The crude was purified by silica gel chromatography using a 24 g column eluting with 0-30% EtOAc in hexane to give title compound (200 mg, 0.301 mmol, 38.16% yield). LCMS: m/z=664.1 [M+H]⁺.

### Example 66: 5-[(1R,3R)-2-[3-[tert-butyl(diphenyl)silyl]oxy-2,2-difluoro-propyl]-3-methyl-1,3,4,9-tetrahydropyrido[3,4-b]indol-1-yl]-1H-indole-2-carbaldehyde

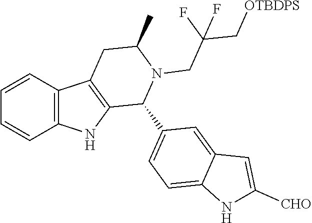

To [5-[(1R,3-R)-2-[3-[tert-butyl(diphenyl)siyl]oxy-2,2-difluoro-propyl]-3-methyl-1,3,4,9-tetrahydropyrido[3,4-b]indol-1-yl]-1H-indol-2-yl]methanol (1.00 equiv, 200 mg, 0.301 mmol) in DMSO (3 mL) was added the 1-hydroxy-1,2-benziodoxol-3(1H)-one 1-oxide (1.00 equiv, 187 mg, 0.301 mmol) and stirred at room temperature for 2 h. The reaction was quenched with water, extracted with ethyl acetate and layers separated. The EtOAc layer was washed with water, brine, dried over sodium sulfate, filtered, and concentrated. The crude was purified by silica gel chromatography using 0-10% EtOAc in hexane to give title compound (130 mg, 0.196 mmol, 65.2% yield). LCMS: m/z=662.1 [M+H]⁺.

### Example 67: 2,2-difluoro-3-[(1R,3R)-1-[2-[[3-(fluoromethyl)azetidin-1-yl]methyl]-1H-indol-5-yl]-3-methyl-1,3,4,9-tetrahydropyrido[3,4-b]indol-2-yl]propan-1-ol

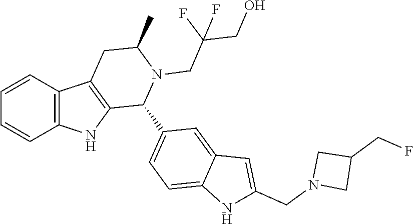 [SMILES: C[C@@H]1Cc2c([nH]c3ccccc23)[C@@H](c2ccc3[nH]c(CN4CC(CF)C4)cc3c2)N1CC(F)(F)CO]

To a solution of 5-[(1R,3R)-2-[3-[tert-butyl(diphenyl)silyl]oxy-2,2-difluoro-propyl]-3-methyl-1,3,4,9-tetrahydropyrido[3,4-b]indol-1-yl]-1H-indole-2-carbaldehyde (1.00 equiv, 50 mg, 0.0755 mmol) in DMF (0.5 mL) was added 3-(fluoromethyl)azetidine hydrochloride (2.00 equiv, 19 mg, 0.151 mmol) dissolved in DMF (0.5 mL) and triethylamine (2.00 equiv, 0.021 mL, 0.151 mmol). To the reaction mixture was added acetic acid (2.00 equiv, 0.0086 mL, 0.151 mmol) and stirred at room temperature for 1 h and then treated with sodium triacetoxyborohydride (3.00 equiv, 48 mg, 0.227 mmol) and stirred for 1 h. The reaction was quenched with 1 mL of methanol and extracted with ethyl acetate, washed with water, brine, dried over sodium sulfate, filtered, and concentrated. The crude was dissolved in THF (2 mL) and treated with tetra-N-butylammonium fluoride (2.00 equiv, 0.15 mL, 0.151 mmol) and stirred at room temp for 1 h. The reaction was extracted with 1:1 EtOAc/MTBE, washed with water and concentrated. The crude was purified by C-18 reverse phase chromatography on a Luna column, using 10-50% ACN/water with 0.1% TFA as additive to give title compound (9.0 mg, 0.0181 mmol, 23.99% yield). LCMS: m/z=497.3 [M+H]⁺.

### Example 68: 2,2-difluoro-3-[(1R,3R)-3-methyl-1-[2-[(3-methylazetidin-1-yl)methyl]-1H-indol-5-yl]-1,3,4,9-tetrahydropyrido[3,4-b]indol-2-yl]propan-1-ol

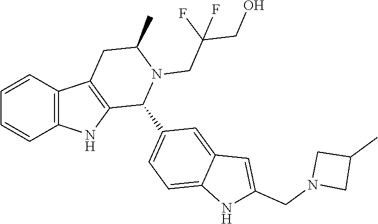 [SMILES: CC1CN(Cc2cc3cc([C@@H]4c5[nH]c6ccccc6c5C[C@@H](C)N4CC(F)(F)CO)ccc3[nH]2)C1]

3-Methylazetidin-1-ium; chloride was used to synthesize the title compound using the procedure described for Example 67 (7.0 mg, 0.0146 mmol, 19.36% yield) LCMS: m/z=479.2 [M+H]⁺.

### Example 69: 2-(hydroxymethyl)-1H-indole-6-carbaldehyde

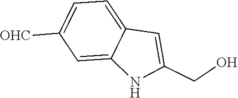 [SMILES: O=Cc1ccc2cc(CO)[nH]c2c1]

To methyl 6-cyano-1H-indole-2-carboxylate (1.00 equiv, 0.13 g, 0.624 mmol) in DCM (10 mL) at 0 °C under nitrogen was added diisobutylaluminum hydride (4.00 equiv, 2.5 mL, 2.50 mmol) and stirred at 0 °C for 2 h. The reaction was quenched with ethyl acetate, followed by solution of Rochelle's salt, and stirred for 30 mins. The organic layer was separated and aqueous layer was extracted with additional DCM twice. The organic layer was dried over sodium sulfate, filtered, and concentrated. The crude was purified by silica gel chromatography using 0-10% MeOH in DCM to give title compound (30 mg, 0.171 mmol, 27.43% yield). 176.1 [M+H]⁺.

### Example 70: [6-[(1R,3R)-2-(2-fluoro-2-methyl-propyl)-3-methyl-1,3,4,9-tetrahydropyrido[3,4-b]indol-1-yl]-1H-indol-2-yl]methanol

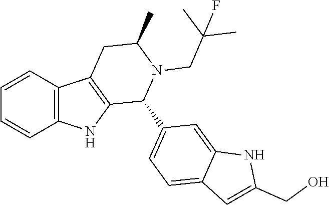 [SMILES: C[C@@H]1Cc2c([nH]c3ccccc23)[C@@H](c2ccc3cc(CO)[nH]c3c2)N1CC(C)(C)F]

To 2-fluoro-N-[(1R)-2-(1H-indol-3-yl)-1-methyl-ethyl]-2-methyl-propan-1-amine (1.00 equiv, 43 mg, 0.171 mmol) in DCE (3 mL) was added 2-(hydroxymethyl)-1H-indole-6-carbaldehyde (1.00 equiv, 30 mg, 0.171 mmol) and acetic acid (2.00 equiv, 0.020 mL, 0.343 mmol) and stirred at 110 °C in microwave for 3 h. The reaction mixture was extracted with DCM, washed with saturated sodium bicarbonate solution, dried over sodium sulfate, filtered, and concentrated. The crude was purified by silica gel chromatography using a 12 g column, eluting with 10-80% EtOAc in hexane, to give title compound (30 mg, 0.0740 mmol, 43.20% yield). 406.2 [M+H]⁺.

### Example 71: 6-[(1R,3R)-2-(2-fluoro-2-methyl-propyl)-3-methyl-1,3,4,9-tetrahydropyrido[3,4-b]indol-1-yl]-1H-indole-2-carbaldehyde

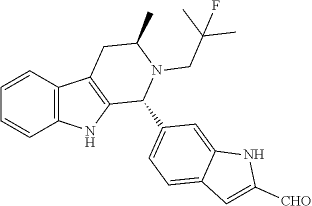 [SMILES: C[C@@H]1Cc2c([nH]c3ccccc23)[C@@H](c2ccc3cc(C=O)[nH]c3c2)N1CC(C)(C)F]

To [6-[(1R,3R)-2-(2-fluoro-2-methyl-propyl)-3-methyl-1,3,4,9-tetrahydropyrido[3,4-b]indol-1-yl]-1H-indol-2-yl]methanol (1.00 equiv, 30 mg, 0.0740 mmol) in DMSO (0.5 mL) was added the 1-hydroxy-1,2-benxiodoxol-3(1H)-one 1-oxide (1.20 equiv, 55 mg, 0.089 mmol) and stirred at room temperature for 2 h. The reaction was quenched with water, extracted with ethyl acetate and layers separated. The EtOAc layer was washed with water, brine, dried over sodium sulfate, filtered, and concentrated. The crude was purified by silica gel chromatography using 0-40% EtOAc in hexane to give title compound (25 mg, 0.0620 mmol, 83.75% yield). 404.2 [M+H]⁺.

### Example 72: (1R,3R)-1-[2-[[3-(fluoromethyl)azetidin-1-yl]methyl]-1H-indol-6-yl]-2-(2-fluoro-2-methyl-propyl)-3-methyl-1,3,4,9-tetrahydropyrido[3,4-b]indole

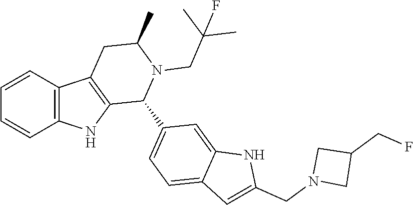 [SMILES: C[C@@H]1Cc2c([nH]c3ccccc23)[C@@H](c2ccc3cc(CN4CC(CF)C4)[nH]c3c2)N1CC(C)(C)F]

To a solution of 3-(fluoromethyl)azetidine hydrochloride (2.00 equiv, 16 mg, 0.124 mmol) in DMF (0.5 mL) was added triethylamine (2.00 equiv, 0.017 mL, 0.124 mmol) followed by 6-[(1R,3R)-2-(2-fluoro-2-methyl-propyl)-3-methyl-1,3,4,9-tetrahydropyrido[3,4-b]indol-1-yl]-1H-indole-2-carbaldehyde (1.00 equiv, 25 mg, 0.0620 mmol) in DMF (0.5 mL) and acetic acid (2.00 equiv, 0.0071 mL, 0.124 mmol). The reaction mixture was stirred for 1 h and treated with sodium triacetoxyborohydride (3.00 equiv, 39 mg, 0.186 mmol), and then stirred for additional 2 h. The reaction was quenched with 1 mL of methanol and purified by C-18 reverse phase chromatography using a Phenomenex Luna column, eluting with 10-50% ACN in water with 0.1% formic acid as modifier, to give title compound (4.5 mg, 0.00944 mmol, 15.24% yield). 477.2 [M+H]⁺.

### Example 73: Methyl 5-[(1R,3R)-2-(2-fluoro-2-methyl-propyl)-3-methyl-1,3,4,9-tetrahydropyrido[3,4-b]indol-1-yl]benzofuran-2-carboxylate

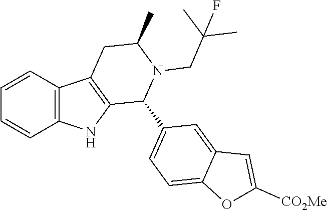 [SMILES: COC(=O)c1cc2cc([C@@H]3c4[nH]c5ccccc5c4C[C@@H](C)N3CC(C)(C)F)ccc2o1]

Methyl 5-formylbenzofuran-2-carboxylate (250 mg, 1.22 mmol) and 2-fluoro-N-[(1R)-2-(1H-indol-3-yl)-1-methyl-ethyl]-2-methyl-propan-1-amine (320 mg, 1.29 mmol) were dissolved in DCE (10 mL) and acetic acid (280 μL, 4.9 mmol) was added. The solution was refluxed at 120 °C for 4 hours then cooled to ambient temperature. The solution was neutralized with saturated sodium bicarbonate solution and the organic layer was separated then washed with saturated brine solution. It was then dried over sodium sulfate, filtered, and dried in vacuo to yield the title compound (523 mg, 98% yield). LCMS: m/z=435.2 [M+H]⁺.

### Example 74: [5-[(1R,3R)-2-(2-Fluoro-2-methyl-propyl)-3-methyl-1,3,4,9-tetrahydropyrido[3,4-b]indol-1-yl]benzofuran-2-yl]methanol

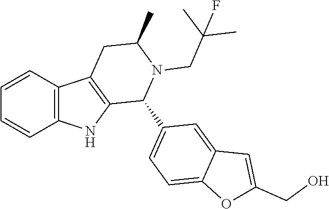 [SMILES: C[C@@H]1Cc2c([nH]c3ccccc23)[C@@H](c2ccc3oc(CO)cc3c2)N1CC(C)(C)F]

Methyl 5-[(1R,3R)-2-(2-fluoro-2-methyl-propyl)-3-methyl-1,3,4,9-tetrahydropyrido[3,4-b]indol-1-yl]benzofuran-2-carboxylate (110 mg, 0.25 mmol) was dissolved in dry THE under a nitrogen atmosphere and cooled to −78 °C. DIBAL-H (1.0 mmol in toluene) was added and stirred for 2 hours. The reaction was quenched with saturated ammonium chloride solution and diluted with EtOAc. The organic phase was separated and washed with saturated brine solution, dried over sodium sulfate and the volatiles were evaporated in vacuo. The crude was purified via flash chromatography in 0-40% EtOAc in hexanes to give the title product (58 mg, 56% yield). LCMS: m/z=407.5 [M+H]⁺.

### Example 75: 5-[(1R,3R)-2-(2-fluoro-2-methyl-propyl)-3-methyl-1,3,4,9-tetrahydropyrido[3,4-b]indol-1-yl]benzofuran-2-carbaldehyde

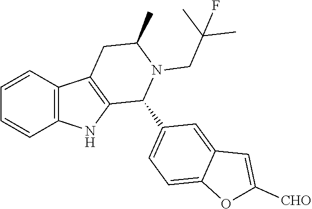 [SMILES: C[C@@H]1Cc2c([nH]c3ccccc23)[C@@H](c2ccc3oc(C=O)cc3c2)N1CC(C)(C)F]

[5-[(1R,3R)-2-(2-Fluoro-2-methyl-propyl)-3-methyl-1,3,4,9-tetrahydropyrido[3,4-b]indol-1-yl]benzofuran-2-yl]methanol (58 mg, 0.14 mmol) and IBX (160 mg, 0.57 mmol) were dissolved in DMSO (1 mL) and stirred for 18 h. The reaction was quenched with saturated sodium bicarbonate solution and diluted with EtOAc. The organic layer was separated and washed with saturated brine solution, then dried over sodium sulfate. The volatiles were removed under reduced pressure. The crude was purified via flash chromatography in 0-40% EtOAc in hexanes to give the title compound (37 mg, 64% yield). LCMS: m/z=405.3 [M+H]⁺.

### Example 76: (1R,3R)-1-[2-[[3-(fluoromethyl)azetidin-1-yl]methyl]benzofuran-5-yl]-2-(2-fluoro-2-methyl-propyl)-3-methyl-1,3,4,9-tetrahydropyrido[3,4-b]indole

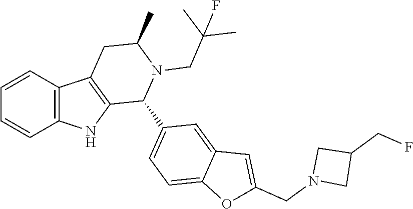 [SMILES: C[C@@H]1Cc2c([nH]c3ccccc23)[C@@H](c2ccc3oc(CN4CC(CF)C4)cc3c2)N1CC(C)(C)F]

5-[(1R,3R)-2-(2-Fluoro-2-methyl-propyl)-3-methyl-1,3,4,9-tetrahydropyrido[3,4-b]indol-1-yl]benzofuran-2-carbaldehyde (0.05 mmol) was dissolved in DMF (0.5 mL) and acetic acid (6 μL, 0.10 mmol) was added. Separately, 3-(fluoromethyl)azetidine hydrochloride (0.05 mmol) was dissolved in DMF (0.5 mL) and TEA (14 μL, 0.10 mmol) was added. These solutions were combined and stirred for 30 minutes. Sodium triacetoxyborohydride (32 mg, 0.15 mmol) was added and stirred for 2 hours. The reaction was quenched with 1 mL MeOH and directly purified by HPLC on a Kinetix 5 μm C18 100 Å column (size: 100×30.0 mm; gradient: 5-40% 0.1% formic acid in ACN in 0.1% formic acid in water) then lyophilized to give the title compound as a white, fluffy, solid (7.3 mg, 31% yield). LCMS: m/z=478.3 [M+H]⁺.

### Example 77: (1R,3R)-1-[2-[(6,6-difluoro-1-azaspiro[3.3]heptan-1-yl)methyl]benzofuran-5-yl]-2-(2-fluoro-2-methyl-propyl)-3-methyl-1,3,4,9-tetrahydropyrido[3,4-b]indole

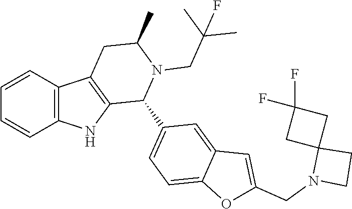 [SMILES: C[C@@H]1Cc2c([nH]c3ccccc23)[C@@H](c2ccc3oc(CN4CCC45CC(F)(F)C5)cc3c2)N1CC(C)(C)F]

5-[(1R,3R)-2-(2-Fluoro-2-methyl-propyl)-3-methyl-1,3,4,9-tetrahydropyrido[3,4-b]indol-1-yl]benzofuran-2-carbaldehyde (17 mg, 0.042 mmol) was dissolved in DMF (0.5 mL) and acetic acid (5 μL, 0.084 mmol) was added. Separately, bis(6,6-difluoro-1-azaspiro[3.3]heptane); oxalic acid (7.5 mg, 0.025 mmol) was dissolved in DMF (0.5 mL) and TEA (12 μL, 0.084 mmol) was added. These solutions were combined and stirred for 30 minutes. Sodium triacetoxyborohydride (27 mg, 0.12 mmol) was added and stirred for 2 hours. The reaction was quenched with 1 mL MeOH and directly purified by HPLC on a Kinetix 5 μm C18 100 Å column (size: 100×30.0 mm; gradient: 5-40% 0.1% formic acid in ACN in 0.1% formic acid in water) then lyophilized to give the title compound as a white, fluffy, solid (8.5 mg, 39% yield). LCMS: m/z=522.3 [M+H]⁺.

### Example 78: Methyl 3-[(1R,3R)-2-(2-fluoro-2-methyl-propyl)-3-methyl-1,3,4,9-tetrahydropyrido[3,4-b]indol-1-yl]-1H-pyrrolo[2,3-b]pyridine-5-carboxylate

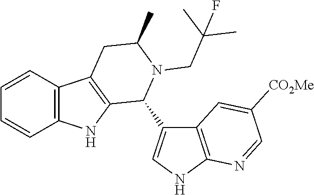 [SMILES: COC(=O)c1cnc2[nH]cc([C@@H]3c4[nH]c5ccccc5c4C[C@@H](C)N3CC(C)(C)F)c2c1]

To a microwave vial equipped with a magnetic stir bar was added a solution of 2-fluoro-N-[(1R)-2-(1H-indol-3-yl)-1-methyl-ethyl]-2-methyl-propan-1-amine (200 mg, 0.805 mmol) in anhydrous DCE (4 mL). To the solution was added methyl 3-formyl-1H-pyrrolo[2,3-b]pyridine-5-carboxylate (160 mg, 0.784 mmol) and acetic acid (0.20 mL, 3.49 mmol). The reaction vessel was closed and purged with nitrogen. The reaction was heated in microwave reactor at 110 °C for 1 h. Saturated solution of sodium bicarbonate (20 mL) was added to the reaction mixture and the aqueous layer was extracted with EtOAc (15 mL×3). The combined organic layer was washed with brine (10 mL), dried over anhydrous Na₂SO₄, filtered and then concentrated under reduced pressure to give crude product. The crude was purified by Combiflash column chromatography (silica gel 12 g, EtOAc:Hex 0-100%) to give 256 g (73%) of titled compound. LCMS: m/z=435.4 [M+H]⁺.

### Example 79: [3-[(1S,3R)-2-(2-fluoro-2-methyl-propyl)-3-methyl-1,3,4,9-tetrahydropyrido[3,4-b]indol-1-yl]-1H-indazol-5-yl]methanol

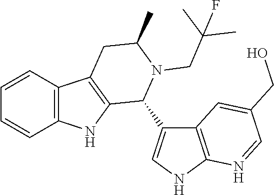 [SMILES: C[C@@H]1Cc2c([nH]c3ccccc23)[C@@H](c2c[nH]c3ncc(CO)cc23)N1CC(C)(C)F]

To a reaction vial equipped with a magnetic stir bar and a nitrogen inlet was added a solution of methyl 3-[(1S,3R)-2-(2-fluoro-2-methyl-propyl)-3-methyl-1,3,4,9-tetrahydropyrido[3,4-b]indol-1-yl]-1H-indazole-5-carboxylate (236 mg, 0.543 mmol) in anhydrous THE (5 mL). To the solution was added 1 N lithium aluminum hydride in THF (1.5 mL, 1.50 mmol) dropwise at 0 °C. The reaction mixture was then warmed to room temperature and stirred for 30 minutes. Upon completion, the reaction was cooled to 0 °C. To the solution was added, 0.10 mL H₂O was dropwise, then 0.20 mL 10% NaOH solution and lastly followed by addition of 0.40 mL H₂O. The reaction mixture was stirred at rt for 30 min, filtered and then concentrated under vacuum to afford crude product as pale-yellow solid. The crude was purified by Combiflash column chromatography (DCM:MeOH 0-20% and 2% TEA as modifier) to give the titled product (171 mg, 0.421 mmol, 77% yield) as pale-yellow oil. LCMS: m/z=407.2 [M+H]⁺.

### Example 80: 3-[(1S,3R)-2-(2-fluoro-2-methyl-propyl)-3-methyl-1,3,4,9-tetrahydropyrido[3,4-b]indol-1-yl]-1H-indazole-5-carbaldehyde

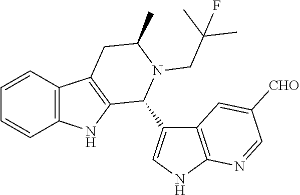 [SMILES: C[C@@H]1Cc2c([nH]c3ccccc23)[C@@H](c2c[nH]c3ncc(C=O)cc23)N1CC(C)(C)F]

To a solution of [3-[(1S,3R)-2-(2-fluoro-2-methyl-propyl)-3-methyl-1,3,4,9-tetrahydropyrido[3,4-b]indol-1-yl]-1H-indazol-5-yl]methanol (164 mg, 0.403 mmol) in anhydrous DMSO (2 mL) was added 2-iodoxybenzoic acid (250 mg, 0.402 mmol). The reaction mixture was stirred for 1 hour. Upon completion, water (20 mL) was added to the reaction mixture and the aqueous layer was extracted with DCM (15 mL×3). The combined organic layer was dried over anhydrous Na₂SO₄, filtered and then concentrated. The crude was purified by Teledyne Isco System (silica gel 12 g, MeOH:DCM 0-20%) to give title compound (110 mg, 0.272 mmol, 67.41% yield) as pure and desired product. LCMS: m/z=405.2 [M+H]⁺.

### Example 81: (1R,3R)-2-(2-fluoro-2-methyl-propyl)-3-methyl-1-[5-[(3-methylazetidin-1-yl)methyl]-1H-pyrrolo[2,3-b]pyridin-3-yl]-1,3,4,9-tetrahydropyrido[3,4-b]indole

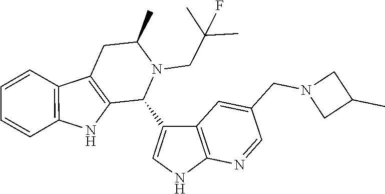 [SMILES: CC1CN(Cc2cnc3[nH]cc([C@@H]4c5[nH]c6ccccc6c5C[C@@H](C)N4CC(C)(C)F)c3c2)C1]

To a solution of 3-[(1R,3R)-2-(2-fluoro-2-methyl-propyl)-3-methyl-1,3,4,9-tetrahydropyrido[3,4-b]indol-1-yl]-1H-pyrrolo[2,3-b]pyridine-5-carbaldehyde (34 mg, 0.0841 mmol) in DMF (0.50 mL) was added a solution of 3-methylazetidine hydrochloride (18 mg, 0.167 mmol) in DMF (0.50 mL) and triethylamine (0.023 mL, 0.165 mmol). To the reaction mixture was added acetic acid (0.019 mL, 0.332 mmol) and stirred at room temperature for 1 h and then treated with sodium triacetoxyborohydride (53 mg, 0.250 mmol) and triethylamine (0.023 mL, 0.165 mmol) and stirred for 1 h. The reaction was quenched with 1 mL of methanol and the crude directly purified by C-18 reverse phase chromatography using prep HPLC, eluting with 10-50% ACN in water with 0.1% formic acid as modifier to give title compound (20 mg, 0.0418 mmol, 49.70% yield) as yellow solid. LCMS: m/z=460.3 [M+H]⁺.

### Example 82: (1R,3R)-1-[5-[[3-(fluoromethyl)azetidin-1-yl]methyl]-1H-pyrrolo[2,3-b]pyridin-3-yl]-2-(2-fluoro-2-methyl-propyl)-3-methyl-1,3,4,9-tetrahydropyrido[3,4-b]indole

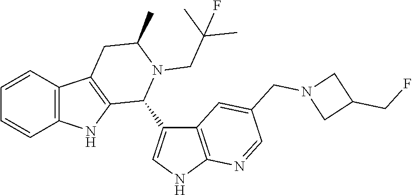 [SMILES: C[C@@H]1Cc2c([nH]c3ccccc23)[C@@H](c2c[nH]c3ncc(CN4CC(CF)C4)cc23)N1CC(C)(C)F]

To a solution of 3-[(1R,3R)-2-(2-fluoro-2-methyl-propyl-3-methyl-1,3,4,9-tetrahydropyrido[3,4-b]indol-1-yl]-1H-pyrrolo[2,3-b]pyridine-5-carbaldehyde (34 mg, 0.0841 mmol) in DMF (0.5 mL) was added a solution of 3-(fluoromethyl)azetidine hydrochloride (21 mg, 0 167 mmol) in DMF (0.50 mL) and triethylamine (0.023 mL, 0.165 mmol). To the reaction mixture was added acetic acid (0.019 mL, 0.332 mmol) and stirred at room temperature for 1 h. The reaction mixture was then treated with sodium triacetoxyborohydride (53 mg, 0.250 mmol) and triethylamine (0.023 mL, 0.165 mmol) and stirred for 1 h. The reaction was quenched with 1 mL of methanol and the crude directly purified by C-18 reverse phase chromatography using prep HPLC, eluting with 10-50% ACN in water with 0.1% formic acid as modifier to give title compound (16 mg, 0.0322 mmol, 38.26% yield) as yellow solid. LCMS: m/z=478.4 [M+H]⁺.

### Example 83: (1R,3R)-1-[5-(3-azabicyclo[3.1.0]hexan-3-ylmethyl)-1H-pyrrolo[2,3-b]pyridin-3-yl]-2-(2-fluoro-2-methyl-propyl)-3-methyl-1,3,4,9-tetrahydropyrido[3,4-b]indole

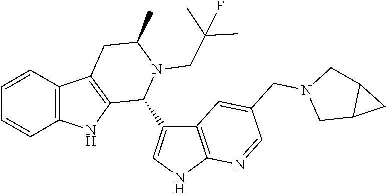 [SMILES: C[C@@H]1Cc2c([nH]c3ccccc23)[C@@H](c2c[nH]c3ncc(CN4CC5CC5C4)cc23)N1CC(C)(C)F]

To a solution of 3-[(1R,3R)-2-(2-fluoro-2-methyl-propyl)-3-methyl-1,3,4,9-tetrahydropyrido[3,4-b]indol-1-yl]-1H-pyrrolo[2,3-b]pyridine-5-carbaldehyde (33 mg, 0.0816 mmol) in DMF (0.5 mL) was added a solution of 3-azabicyclo[3.1.0]hexane hydrochloride (20 mg, 0.167 mmol) in DMF (0.5 mL) and triethylamine (0.023 mL, 0.165 mmol). To the reaction mixture was added acetic acid (0.019 mL, 0.332 mmol) and stirred at room temperature for 1 h. The reaction mixture was then treated with sodium triacetoxyborohydride (52 mg, 0.245 mmol) and triethylamine (0.023 mL, 0.165 mmol) and stirred for 1 h. The reaction was quenched with 1 mL of methanol and the crude directly purified by C-18 reverse phase chromatography using prep HPLC, eluting with 10-50% ACN in water with 0.1% formic acid as modifier, to give title compound (20 mg, 0.0394 mmol, 48.34% yield) as yellow solid. LCMS: m/z=473.0 [M+H]⁺.

### Example 84: Methyl 2-[(1S,3R)-2-(2-fluoro-2-methyl-propyl)-3-methyl-1,3,4,9-tetrahydropyrido[3,4-b]indol-1-yl]-6H-thieno[2,3-b]pyrrole-5-carboxylate

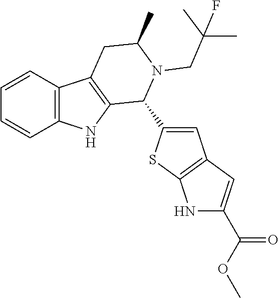 [SMILES: COC(=O)c1cc2cc([C@@H]3c4[nH]c5ccccc5c4C[C@@H](C)N3CC(C)(C)F)sc2[nH]1]

2-Fluoro-N-[(1R)-2-(1H-indol-3-yl)-1-methyl-ethyl]-2-methyl-propan-1-amine (550 mg, 2.2 mmol) was dissolved in DCE (13 mL). Methyl 2-formyl-6H-thieno[2,3-b]pyrrole-5-carboxylate (500 mg, 2.4 mmol) and acetic acid (1.27 mL, 22.1 mmol) were added to the solution and microwaved at 150 °C for 30 min. The volatiles were removed under reduced pressure and the crude was purified by flash chromatography on silica with 0-50% EtOAc in hexanes to yield the title compound (174 mg, 18% yield). LCMS: m/z=440.9 [M+H]⁺.

### Example 85: [2-[(1S,3R)-2-(2-Fluoro-2-methyl-propyl)-3-methyl-1,3,4,9-tetrahydropyrido[3,4-b]indol-1-yl]-6H-thieno[2,3-b]pyrrol-5-yl]methanol

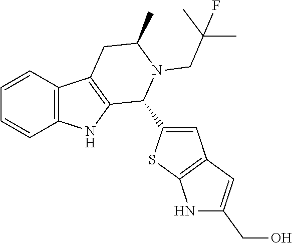 [SMILES: C[C@@H]1Cc2c([nH]c3ccccc23)[C@@H](c2cc3cc(CO)[nH]c3s2)N1CC(C)(C)F]

Methyl 2-[(1S,3R)-2-(2-fluoro-2-methyl-propyl)-3-methyl-1,3,4,9-tetrahydropyrido[3,4-b]indol-1-yl]-6H-thieno[2,3-b]pyrrole-5-carboxylate (160 mg, 0.36 mmol) was dissolved under an atmosphere of nitrogen in anhydrous THF and cooled to 0 °C. 1 M LAH (1.2 mmol) in THE was added and stirred for 3 hours. The reaction was quenched with EtOAc, then washed with saturated Rochelle's salt solution, then water. The combined organic layers were dried over sodium sulfate, filtered, then evaporated under reduced pressure. The crude was purified by flash chromatography on 12 g silica column with 0-50% EtOAc in hexanes to give the title compound (35 mg, 23% yield). LCMS: m/z=412.2 [M+H]⁺.

### Example 86: 2-[(1S,3R)-2-(2-Fluoro-2-methyl-propyl)-3-methyl-1,3,4,9-tetrahydropyrido[3,4-b]indol-1-yl]-6H-thieno[2,3-b]pyrrole-5-carbaldehyde

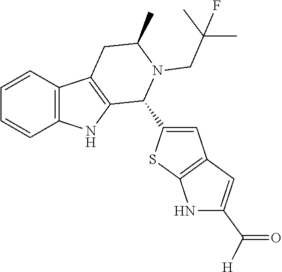 [SMILES: C[C@@H]1Cc2c([nH]c3ccccc23)[C@@H](c2cc3cc(C=O)[nH]c3s2)N1CC(C)(C)F]

[2-[(1S,3R)-2-(2-Fluoro-2-methyl-propyl)-3-methyl-1,3,4,9-tetrahydropyrido[3,4-b]indol-1-yl]-6H-thieno[2,3-b]pyrrol-5-yl]methanol (35 mg, 0.085 mmol) and IBX (23.8 mg, 0.085 mmol) were dissolved in DMSO and stirred for 1.5 hours. The reaction was quenched with water, then extracted with EtOAc. The combined organic layers were washed with saturated brine solution and water, dried over sodium sulfate, filtered, and evaporated under reduced pressure. The crude was purified by flash chromatography on a TEA-rinsed 12 g silica column with 0-30% EtOAc in hexanes to yield the title compound (22 mg, 63% yield). LCMS: m/z=410.2 [M+H]⁺.

### Example 87: 2-[(1S,3R)-2-(2-Fluoro-2-methyl-propyl)-3-methyl-1,3,4,9-tetrahydropyrido[3,4-b]indol-1-yl]-5-[(3-methylazetidin-1-yl)methyl]-6H-thieno[2,3-b]pyrrole

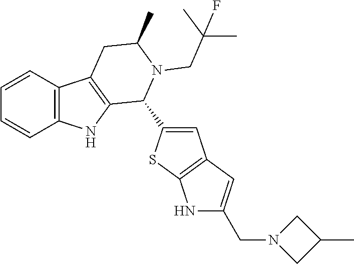 [SMILES: CC1CN(Cc2cc3cc([C@@H]4c5[nH]c6ccccc6c5C[C@@H](C)N4CC(C)(C)F)sc3[nH]2)C1]

To solution of 2-[(1S,3R)-2-(2-fluoro-2-methyl-propyl)-3-methyl-1,3,4,9-tetrahydropyrido[3,4-b]indol-1-yl]-6H-thieno[2,3-b]pyrrole-5-carbaldehyde (9.0 mg, 0.022 mmol) in DMF (0.5 mL) was added acetic acid (2.6 mg, 0.044 mmol). Separately, 3-methylazetidin-1-ium chloride (2.4 mg, 0.022 mmol) was dissolved in DMF (0.5 mL) and TEA (4.4 mg, 0.044 mmol) was added. These solutions were combined and stirred for 30 min. Sodium triacetoxyborohydride (14.0 mg, 0.066 mmol) was added and stirred for 2 hours. The reaction was quenched with 1 mL of MeOH and directly purified by HPLC on Kinetix 5 μm C18 100 Å column (size 100×30.0 mm; gradient 5-50% 0.1% formic acid in ACN in 0.1% formic acid in water) then lyophilized to obtain the title compound as an off-white powder (2.2 mg, 21% yield). LCMS: m/z=465.2 [M+H]⁺.

### Example 88: 5-[[3-(Fluoromethyl)azetidin-1-yl]methyl]-2-[(1S,3R)-2-(2-fluoro-2-methyl-propyl)-3-methyl-1,3,4,9-tetrahydropyrido[3,4-b]indol-1-yl]-6H-thieno[2,3-b]pyrrole

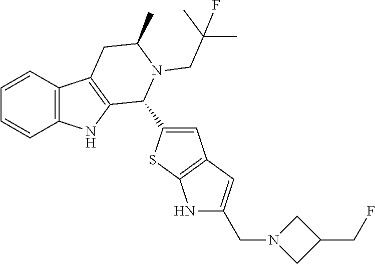 [SMILES: C[C@@H]1Cc2c([nH]c3ccccc23)[C@@H](c2cc3cc(CN4CC(CF)C4)[nH]c3s2)N1CC(C)(C)F]

To solution of 2-[(1 S,3R)-2-(2-fluoro-2-methyl-propyl)-3-methyl-1,3,4,9-tetrahydropyrido[3,4-b]indol-1-yl]-6H-thieno[2,3-b]pyrrole-5-carbaldehyde (12.6 mg, 0.030 mmol) in DMF (0.5 mL) was added acetic acid (3.7 mg, 0.062 mmol). Separately, 3-(fluoromethyl)azetidin-1-ium chloride (3.9 mg, 0.031 mmol) was dissolved in DMF (0.5 mL) and TEA (6.2 mg, 0.062 mmol) was added. These solutions were combined and stirred for 30 min. Sodium triacetoxyborohydride (20.0 mg, 0.092 mmol) was added and stirred for 2 hours. The reaction was quenched with 1 mL of MeOH and directly purified by HPLC on Kinetix 5 μm C18 100 Å column (size 100×30.0 mm; gradient 5-50% 0.1% formic acid in ACN in 0.1% formic acid in water) then lyophilized to obtain the title compound as a pink powder (2.7 mg, 18% yield). LCMS: m/z=483.2 [M+H]⁺.

### Example 89: Methyl 2-[(1S,3R)-2-(2-fluoro-2-methyl-propyl)-3-methyl-1,3,4,9-tetrahydropyrido[3,4-b]indol-1-yl]-4H-thieno[3,2-b]pyrrole-5-carboxylate

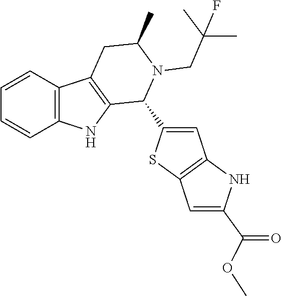 [SMILES: COC(=O)c1cc2sc([C@@H]3c4[nH]c5ccccc5c4C[C@@H](C)N3CC(C)(C)F)cc2[nH]1]

To 2-fluoro-N-[(1R)-2-(1H-indol-3-yl)-1-methyl-ethyl]-2-methyl-propan-1-amine (1.00 equiv, 300 mg, 1.21 mmol) in DCE (20 mL) was added the methyl 2-formyl-4H-thieno[3,2-b]pyrrole-5-carboxylate (1.20 equiv, 303 mg, 1.45 mmol), followed by acetic acid (2.50 equiv, 0.17 mL, 3.02 mmol) in a microwave tube, flushed with nitrogen. The reaction tube was capped and heated at 100 °C in the microwave for 3 h. The reaction was cooled to room temperature and extracted with DCM. The organic layer was washed with saturated sodium bicarbonate, dried over sodium sulfate, filtered and concentrated. The crude was purified by silica gel chromatography using 0-10% EtOAc in DCM to give the title compound (380 mg, 0.865 mmol, 71% yield). LCMS: m/z=440.2 [M+H]⁺.

### Example 90: 2-[(1S,3R)-2-(2-fluoro-2-methyl-propyl)-3-methyl-1,3,4,9-tetrahydropyrido[3,4-b]indol-1-yl]-4H-thieno[3,2-b]pyrrole-5-carboxylic acid

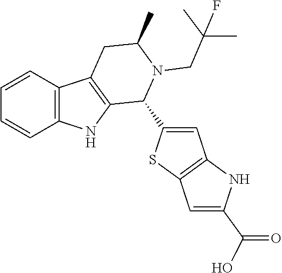 [SMILES: C[C@@H]1Cc2c([nH]c3ccccc23)[C@@H](c2cc3[nH]c(C(=O)O)cc3s2)N1CC(C)(C)F]

To methyl 2-[(1S,3R)-2-(2-fluoro-2-methyl-propyl)-3-methyl-1,3,4,9-tetrahydropyrido[3,4-b]indol-1-yl]-4H-thieno[3,2-b]pyrrole-5-carboxylate (1.00 equiv, 23 mg, 0.0523 mmol) in THF (3 mL)/methanol (1 mL)/water (0.5 mL) was added the lithium hydroxide (16.0 equiv, 20 mg, 0.835 mmol) and stirred at 60 °C for 16 h. The reaction mixture was treated with formic acid (16.0 equiv, 0.032 mL, 0.837 mmol), and concentrated under reduced pressure. The crude was taken up in methanol and purified by reverse phase chromatography using Luna C-18 column eluting with water/ACN with 0.1% formic acid as additive to give the title compound (8.0 mg, 0.0181 mmol, 34.5% yield). LCMS: m/z=426.0 [M+H]⁺.

### Example 91: [2-[(1S,3R)-2-(2-fluoro-2-methyl-propyl)-3-methyl-1,3,4,9-tetrahydropyrido[3,4-b]indol-1-yl]-4H-thieno[3,2-b]pyrrol-5-yl]methanol

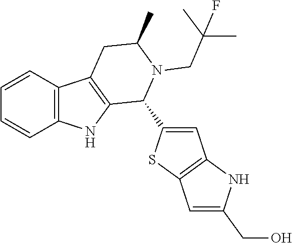 [SMILES: C[C@@H]1Cc2c([nH]c3ccccc23)[C@@H](c2cc3[nH]c(CO)cc3s2)N1CC(C)(C)F]

To methyl 2-[(1S,3R)-2-(2-fluoro-2-methyl-propyl)-3-methyl-1,3,4,9-tetrahydropyrido[3,4-b]indol-1-yl]-4H-thieno[3,2-b]pyrrole-5-carboxylate (1.00 equiv, 200 mg, 0.455 mmol) in THF (5 mL) under an atmosphere of nitrogen at 0 °C was added the lithium aluminum hydride (3.00 equiv, 1.4 mL, 1.37 mmol) dropwise. The reaction mixture was allowed to warm up to room temperature and stirred for 2 h. The reaction mixture was cooled in an ice water bath and diluted with MTBE and few drops of water, then a solution of sodium hydroxide (0.659 equiv, 0.080 mL, 0.300 mmol) was added. The reaction mixture was stirred vigorously for 15 mins and then filtered through Celite®. The filtrate was concentrated, the residue taken up in ethyl acetate, the EtOAc layer was washed with brine, dried over sodium sulfate, filtered and concentrated. The crude was purified by silica gel chromatography using 10-80% EtOAc in DCM to give the title compound (140 mg, 0.340 mmol, 74% yield). LCMS: m/z=413.3 [M+H]⁺.

### Example 92: 2-[(1S,3R)-2-(2-fluoro-2-methyl-propyl)-3-methyl-1,3,4,9-tetrahydropyrido[3,4-b]indol-1-yl]-4H-thieno[3,2-b]pyrrole-5-carbaldehyde

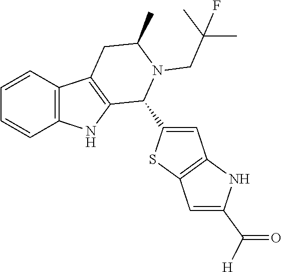 [SMILES: C[C@@H]1Cc2c([nH]c3ccccc23)[C@@H](c2cc3[nH]c(C=O)cc3s2)N1CC(C)(C)F]

To [2-[(1S,3R)-2-(2-fluoro-2-methyl-propyl)-3-methyl-1,3,4,9-tetrahydropyrido[3,4-b]indol-1-yl]-4H-thieno[3,2-b]pyrrol-5-yl]methanol (1.00 equiv, 50 mg, 0.121 mmol) in DCM was added manganese dioxide (20.0 equiv, 211 mg, 2.43 mmol) and stirred at room temperature for 48 h. The reaction mixture was filtered through Celite® and concentrated. The crude was purified by silica gel chromatography using 0-50% EtOAc in DCM to give the title compound (20 mg, 0.0488 mmol, 40.2% yield). LCMS: m/z=410.2 [M+H]⁺.

### Example 93: 5-[[3-(fluoromethyl)azetidin-1-yl]methyl]-2-[(1S,3R)-2-(2-fluoro-2-methyl-propyl)-3-methyl-1,3,4,9-tetrahydropyrido[3,4-b]indol-1-yl]-4H-thieno[3,2-b]pyrrole

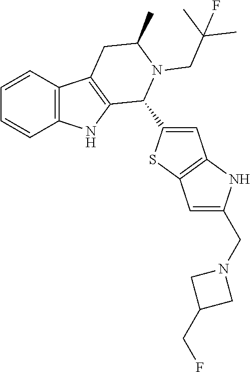 [SMILES: C[C@@H]1Cc2c([nH]c3ccccc23)[C@@H](c2cc3[nH]c(CN4CC(CF)C4)cc3s2)N1CC(C)(C)F]

To 2-[(1S,3R)-2-(2-fluoro-2-methyl-propyl)-3-methyl-1,3,4,9-tetrahydropyrido[3,4-b]indol-1-yl]-4H-thieno[3,2-b]pyrrole-5-carbaldehyde (1.00 equiv, 20 mg, 0.0488 mmol) in DMF (1 mL) and acetic acid (2.00 equiv, 0.0056 mL, 0.0977 mmol) was added 3-(fluoromethyl)azetidin-1-ium; chloride (2.0 equiv, 12 mg, 0.0977 mmol) dissolved in DMF (0.5 mL) and triethylamine (2.00 equiv, 0.014 mL, 0.0977 mmol) under nitrogen. The reaction mixture was stirred at room temperature for 1 h and to this was added the sodium triacetoxyborohydride (3.00 equiv, 31 mg, 0.147 mmol). The reaction mixture was stirred at room temp for 2 h, then quenched with methanol. The crude was purified by C-18 reverse phase chromatography eluting with ACN/Water with 0.1% formic acid as modifier to give the title compound (4.3 mg, 0.00891 mmol, 18.2% yield). LCMS: m/z=483.0 [M+H].

### Example 94: 2-[(1S,3R)-2-(2-fluoro-2-methyl-propyl)-3-methyl-1,3,4,9-tetrahydropyrido[3,4-b]indol-1-yl]-5-[(3-methylazetidin-1-yl)methyl]-4H-thieno[3,2-b]pyrrole

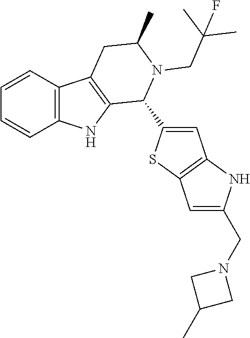 [SMILES: CC1CN(Cc2cc3sc([C@@H]4c5[nH]c6ccccc6c5C[C@@H](C)N4CC(C)(C)F)cc3[nH]2)C1]

Prepared by the method of Example 93 using 3-methylazetidin-1-ium; chloride LCMS: m/z=465.0 [M+H]⁺.

### Example 95: 3-fluoro-N-[[2-[(1S,3R)-2-(2-fluoro-2-methyl-propyl)-3-methyl-1,3,4,9-tetrahydropyrido[3,4-b]indol-1-yl]-4H-thieno[3,2-b]pyrrol-5-yl]methyl]propan-1-amine

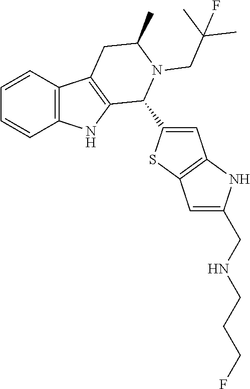 [SMILES: C[C@@H]1Cc2c([nH]c3ccccc23)[C@@H](c2cc3[nH]c(CNCCCF)cc3s2)N1CC(C)(C)F]

Prepared by the method of Example 93 using 3-fluoropropylammonium; chloride. LCMS: m/z=471.0 [M+H]⁺.

### Example 96: 5-[[(3S)-3-ethylpyrrolidin-1-yl]methyl]-2-[(1S,3R)-2-(2-fluoro-2-methyl-propyl)-3-methyl-1,3,4,9-tetrahydropyrido[3,4-b]indol-1-yl]-4H-thieno[3,2-b]pyrrole

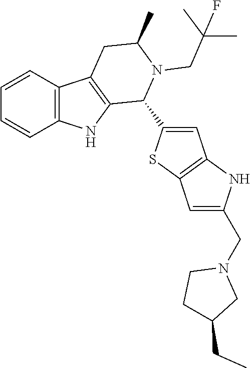 [SMILES: CC[C@H]1CCN(Cc2cc3sc([C@@H]4c5[nH]c6ccccc6c5C[C@@H](C)N4CC(C)(C)F)cc3[nH]2)C1]

Prepared by the method of Example 93 using (3S)-3-ethylpyrrolidin-1-ium; chloride. LCMS: m/z=493.0 [M+H]⁺.

### Example 97: Methyl 5-bromo-1-(2-trimethylsilylethoxymethyl)pyrrolo[2,3-b]pyridine-2-carboxylate

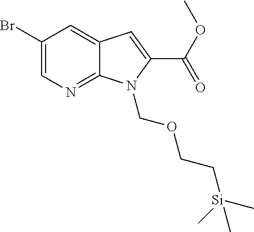 [SMILES: COC(=O)c1cc2cc(Br)cnc2n1COCC[Si](C)(C)C]

Methyl 5-bromo-1H-pyrrolo[2,3-b]pyridine-2-carboxylate (944 mg, 3.7 mmol) was dissolved in DMF (10 mL) and brought to 0 °C. Sodium hydride (98 mg, 4.1 mmol) was added and stirred for 30 min. 2-(Trimethylsilyl)ethoxymethyl chloride (679 mg, 4.1 mmol) was added and stirred for 2 h. The reaction was quenched with saturated sodium bicarbonate solution and diluted with EtOAc. The organic layer was separated and washed with saturated brine solution 5 times. It was then dried over sodium sulfate, filtered, and concentrated in vacuo to give the title product (1.277 g, 90% yield). LCMS: m/z=386.2 [M+H]⁺.

### Example 98: Methyl 5-cyano-1-(2-trimethylsilylethoxymethyl)pyrrolo[2,3-b]pyridine-2-carboxylate

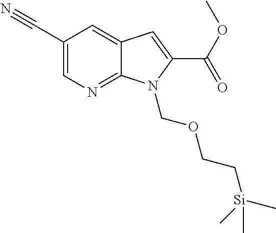 [SMILES: COC(=O)c1cc2cc(C#N)cnc2n1COCC[Si](C)(C)C]

Methyl 5-bromo-1-(2-trimethylsilylethoxymethyl)pyrrolo[2,3-b]pyridine-2-carboxylate (1.27 g, 3.3 mmol), zinc cyanide (464 mg, 4.0 mmol), [1,1′-bis(diphenylphosphino)ferrocene]dichloropalladium(II) (241 mg, 0.33 mmol), tris(dibenzylideneacetone)dipalladium(0) (151 mg, 0.16 mmol), and zinc (54 mg, 0.82 mmol) were dissolved in DMF (15 mL) under an atmosphere of nitrogen. The reaction was heated to 120 °C and stirred for 16 h. The reaction was brought to room temperature then filtered. It was diluted with EtOAc then partitioned with saturated sodium bicarbonate solution. The organic layer was washed with saturated brine solution 5 times, dried over sodium sulfate, filtered, then dried in vacuo. The crude material was purified on silica gel in 0-50% EtOAc in hexanes gradient to yield the title product (711 mg, 65% yield). LCMS: m/z=332.5 [M+H]⁺.

### Example 99: 2-(Hydroxymethyl)-1-(2-trimethylsilylethoxymethyl)pyrrolo[2,3-b]pyridine-5-carbaldehyde

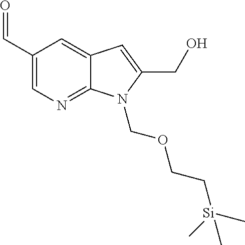 [SMILES: C[Si](C)(C)CCOCn1c(CO)cc2cc(C=O)cnc21]

Methyl 5-cyano-1-(2-trimethylsilylethoxymethyl)pyrrolo[2,3-b]pyridine-2-carboxylate (711 mg, 2.1 mmol) was dissolved in THE (7 mL) in an atmosphere of nitrogen and brought to 0 °C. DIBAL (6.4 mmol) was added dropwise and stirred for 90 min. The reaction was brought to room temperature and quenched with EtOAc. Saturated Rochelle's salt solution was added and stirred for 45 min. The organic layer was then separated and washed with saturated brine solution, dried over sodium sulfate, filtered, and dried in vacuo. The crude material was purified on silica gel in 0-10% methanol in DCM gradient to give the title product (323 mg, 49% yield). LCMS: m/z=307.4 [M+H]⁺.

### Example 100: [5-[(1R,3R)-2-(2-Fluoro-2-methyl-propyl)-3-methyl-1,3,4,9-tetrahydropyrido[3,4-b]indol-1-yl]-1-(2-trimethylsilylethoxymethyl)pyrrolo[2,3-b]pyridin-2-yl]methanol

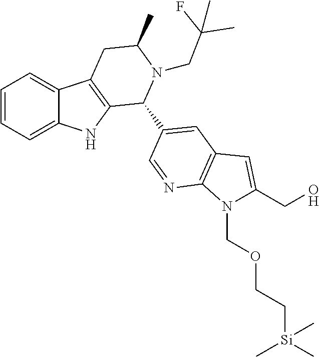 [SMILES: C[C@@H]1Cc2c([nH]c3ccccc23)[C@@H](c2cnc3c(c2)cc(CO)n3COCC[Si](C)(C)C)N1CC(C)(C)F]

2-(Hydroxymethyl)-1-(2-trimethylsilylethoxymethyl)pyrrolo[2,3-b]pyridine-5-carbaldehyde (160 mg, 0.52 mmol), (R)—N-(1-(1H-indol-3-yl)propan-2-yl)-2-fluoro-2-methylpropan-1-amine (130 mg, 0.52 mmol) were dissolved in DCE (2 mL) and acetic acid (1.5 mL, 2.6 mmol) was added. The solution was stirred at 120 °C with microwave irradiation for 2 h. The solution was poured into saturated sodium bicarbonate solution and the organic layer was separated and dried over sodium sulfate. It was filtered then dried in vacuo to give the title compound (280 mg, 100%). LCMS: m/z=537.8 [M+H]⁺.

### Example 101: 5-[(1R,3R)-2-(2-fluoro-2-methyl-propyl)-3-methyl-1,3,4,9-tetrahydropyrido[3,4-b]indol-1-yl]-1-(2-trimethylsilylethoxymethyl)pyrrolo[2,3-b]pyridine-2-carbaldehyde

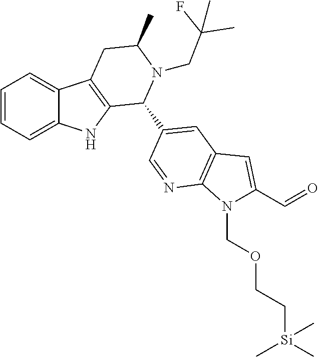 [SMILES: C[C@@H]1Cc2c([nH]c3ccccc23)[C@@H](c2cnc3c(c2)cc(C=O)n3COCC[Si](C)(C)C)N1CC(C)(C)F]

[5-[(1R,3R)-2-(2-fluoro-2-methyl-propyl)-3-methyl-1,3,4,9-tetrahydropyrido[3,4-b]indol-1-yl]-1-(2-trimethylsilylethoxymethyl)pyrrolo[2,3-b]pyridin-2-yl]methanol (280 mg, 0.52 mmol) and IBX (219 mg, 0.78 mmol) were dissolved and stirred in DMSO (2 mL) for 1.5 h. The reaction was quenched with saturated sodium bicarbonate solution and diluted with EtOAc. The organic phase was separated and washed with saturated brine solution. It was then dried over sodium sulfate, filtered, and dried in vacuo. The crude was purified on silica gel in 0-40% EtOAc in hexanes gradient to yield the title compound (101 mg, 36% yield). LCMS: m/z=535.7 [M+H]⁺.

### Example 102: 5-[(1R,3R)-2-(2-fluoro-2-methyl-propyl)-3-methyl-1,3,4,9-tetrahydropyrido[3,4-b]indol-1-yl]-1H-pyrrolo[2,3-b]pyridine-2-carbaldehyde

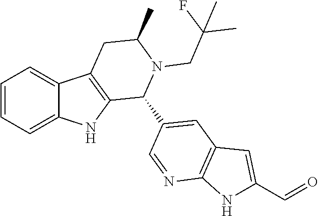 [SMILES: C[C@@H]1Cc2c([nH]c3ccccc23)[C@@H](c2cnc3[nH]c(C=O)cc3c2)N1CC(C)(C)F]

5-[(1R,3R)-2-(2-fluoro-2-methyl-propyl)-3-methyl-1,3,4,9-tetrahydropyrido[3,4-b]indol-1-yl]-1-(2-trimethylsilylethoxymethyl)pyrrolo[2,3-b]pyridine-2-carbaldehyde (101 mg, 0.19 mmol) was dissolved in THF (2 mL) and TBAF (1.9 mmol) was added. The reaction was heated to 80 °C and stirred for 16 h. The reaction was quenched with saturated sodium bicarbonate solution and diluted with EtOAc. The organic phase was separated and washed with saturated brine solution, dried over sodium sulfate, filtered, and dried in vacuo. The crude was purified on silica gel in 0-10% MeOH in DCM gradient to yield the title compound (70 mg, 92% yield). LCMS: m/z=405.4 [M+H]⁺.

### Example 103: (1R,3R)-1-[2-[[3-(fluoromethyl)azetidin-1-yl]methyl]-1H-pyrrolo[2,3-b]pyridin-5-yl]-2-(2-fluoro-2-methyl-propyl)-3-methyl-1,3,4,9-tetrahydropyrido[3,4-b]indole

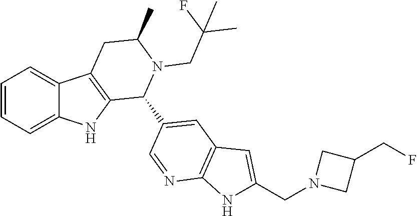 [SMILES: C[C@@H]1Cc2c([nH]c3ccccc23)[C@@H](c2cnc3[nH]c(CN4CC(CF)C4)cc3c2)N1CC(C)(C)F]

5-[(1R,3R)-2-(2-fluoro-2-methyl-propyl)-3-methyl-1,3,4,9-tetrahydropyrido[3,4-b]indol-1-yl]-1H-pyrrolo[2,3-b]pyridine-2-carbaldehyde (26 mg, 0.06 mmol) was dissolved in DMF (1 mL) and acetic acid (7.5 μL, 0.13 mmol) was added. Separately, 3-(fluoromethyl)azetidine hydrochloride (10 mg, 0.08 mmol) was dissolved in DMF (1 mL) and TEA (18 μL, 0.13 mmol) was added. These solutions were combined and heated at 60 °C for 30 min. STAB (41 mg, 0.19 mmol) was added and stirred for 2 h. The reaction was quenched with MeOH (1 mL) and directly purified by HPLC on a Kintetex 5 μm C18 100 Å column (size: 100×30.0 mm; gradient: 5-30% 0.1% formic acid in ACN in 0.1% formic acid in water) then lyophilized to give the title compound as a white solid (13 mg, 42% yield). LCMS: m/z=478.3 [M+H]⁺.

### Example 104: Ethyl 5-chloro-1-(2-trimethylsilylethoxymethyl)pyrrolo[3,2-b]pyridine-2-carboxylate

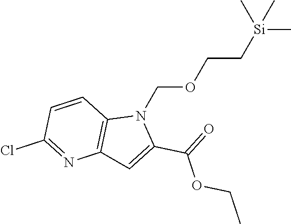 [SMILES: CCOC(=O)c1cc2nc(Cl)ccc2n1COCC[Si](C)(C)C]

To ethyl 5-chloro-1H-pyrrolo[3,2-b]pyridine-2-carboxylate (1.00 equiv, 500 mg, 2.23 mmol) in anhydrous DMF (5 mL) under nitrogen at 0 °C was added the sodium hydride (1.20 equiv, 107 mg, 2.67 mmol) and stirred for 30 min. To this was added 2-(trimethylsilyl)ethoxymethyl chloride (1.20 equiv, 0.47 mL, 2.67 mmol) and stirred for 1 h. The reaction was quenched with water, extracted with EtOAc and the layers separated. The EtOAc layer was washed with water, brine, dried over sodium sulfate, filtered and concentrated. The crude was purified by silica gel chromatography using a 24 g column, eluting with 0-15% EtOAc in hexane to give title compound (730 mg, 2.06 mmol, 92.4% yield). LCMS: m/z=355.1 [M+H]⁺.

### Example 105: Ethyl 5-cyano-1-(2-trimethylsilylethoxymethyl)pyrrolo[3,2-b]pyridine-2-carboxylate

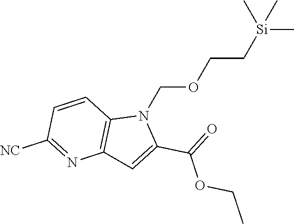 [SMILES: CCOC(=O)c1cc2nc(C#N)ccc2n1COCC[Si](C)(C)C]

To ethyl 5-chloro-1-(2-trimethylsilylethoxymethyl)pyrrolo[3,2-b]pyridine-2-carboxylate (1.00 equiv, 710 mg, 2.00 mmol) in NMP (10 mL) was added the zinc cyanide (6.00 equiv, 1409 mg, 12.0 mmol), and the reaction mixture sparged with nitrogen. To this was added the tetrakis(triphenylphosphine)palladium(0) (0.0500 equiv, 116 mg, 0.100 mmol), and the mixture sparged with nitrogen for an additional 5 min. The reaction was capped and heated at 120 °C for 18 h. The reaction was filtered through Celite®, and the residue was washed with EtOAc (50 mL). The filtrate was washed with water, brine, dried over sodium sulfate, filtered and concentrated. The crude was purified by silica gel chromatography using 24 g column, eluting with 0-20% EtOAc in hexane to give title compound (375 mg, 1.09 mmol, 54.3% yield). LCMS: m/z=346.2 [M+H]⁺.

### Example 106: Ethyl 5-formyl-1-(2-trimethylsilylethoxymethyl)pyrrolo[3,2-b]pyridine-2-carboxylate

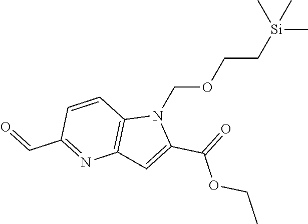 [SMILES: CCOC(=O)c1cc2nc(C=O)ccc2n1COCC[Si](C)(C)C]

To ethyl 5-cyano-1-(2-trimethylsilylethoxymethyl)pyrrolo[3,2-b]pyridine-2-carboxylate (1.00 equiv, 410 mg, 1.19 mmol) in THE (10 mL) at 0 °C under nitrogen was added the diisobutylaluminum hydride (1.50 equiv, 1.8 mL, 1.78 mmol) dropwise and stirred for 1 h after slowly warming to room temp. The reaction was cooled back to 0 °C, quenched with EtOAc, followed by saturated solution of Rochelle's salt, and stirred for 30 min. The reaction mixture was extracted with EtOAc, layers were separated, and the organic layer dried over sodium sulfate. The EtOAc layer was filtered, concentrated under vacuum, and purified by silica gel chromatography using 0-30% EtOAc in hexane to give ethyl 5-formyl-1-(2-trimethylsilylethoxymethyl)pyrrolo[3,2-b]pyridine-2-carboxylate (210 mg, 0.603 mmol, 50.8% yield). LCMS: m/z=349.1 [M+H]⁺.

### Example 107: Ethyl 5-((1S,3R)-2-(2-fluoro-2-methylpropyl)-3-methyl-2,3,4,9-tetrahydro-1H-pyrido[3,4-b]indol-1-yl)-1-((2-(trimethylsilyl)ethoxy)methyl)-1H-pyrrolo[3,2-b]pyridine-2-carboxylate

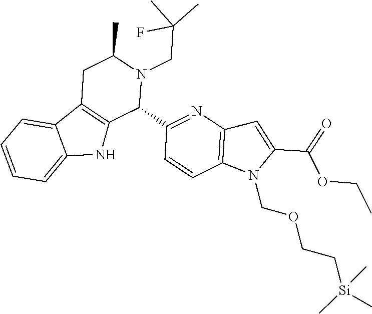 [SMILES: CCOC(=O)c1cc2nc([C@@H]3c4[nH]c5ccccc5c4C[C@@H](C)N3CC(C)(C)F)ccc2n1COCC[Si](C)(C)C]

To (R)—N-(1-(1H-indol-3-yl)propan-2-yl)-2-fluoro-2-methylpropan-1-amine (1.20 equiv, 191 mg, 0.768 mmol) in DCE (3 mL) was added ethyl 5-formyl-1-(2-trimethylsilylethoxymethyl)pyrrolo[2,3-c]pyridine-2-carboxylate (1.00 equiv, 223 mg, 0.640 mmol) and acetic acid (2.00 equiv, 0.073 mL, 1.28 mmol), and the mixture was stirred at 110 °C in microwave for 3 h. The reaction mixture was extracted with DCM, washed with saturated sodium bicarbonate solution, dried over sodium sulfate, filtered, and concentrated. The crude was purified by silica gel chromatography using a 24 g column elution with 0-30% EtOAc in hexane to give title compound (256 mg, 0.442 mmol, 69.1% yield). LCMS: m/z=579.8 [M+H]⁺.

### Example 108: (5-((1S,3R)-2-(2-fluoro-2-methylpropyl)-3-methyl-2,3,4,9-tetrahydro-II-pyrido[3,4-b]indol-1-yl)-1-((2-(trimethylsilyl)ethoxy)methyl)-1H-pyrrolo[3,2-b]pyridin-2-yl)methanol

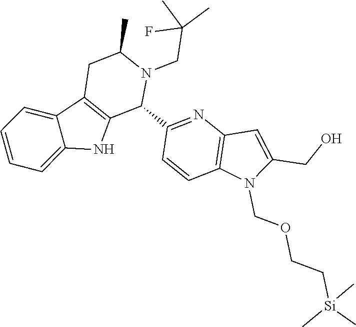 [SMILES: C[C@@H]1Cc2c([nH]c3ccccc23)[C@@H](c2ccc3c(cc(CO)n3COCC[Si](C)(C)C)n2)N1CC(C)(C)F]

To ethyl 5-((1S,3R)-2-(2-fluoro-2-methylpropyl)-3-methyl-2,3,4,9-tetrahydro-1H-pyrido[3,4-b]indol-1-yl)-1-((2-(trimethylsilyl)ethoxy)methyl)-1H-pyrrolo[3,2-b]pyridine-2-carboxylate (1.00 equiv, 256 mg, 0.442 mmol) in THF (5 mL) at 0 °C under atmosphere of nitrogen was added lithium aluminum hydride (2.00 equiv, 0.88 mL, 0.885 mmol) and stirred for 30 min. The reaction was quenched with EtOAc and saturated solution of Rochelle's salt, and stirred for 30 min. The reaction mixture was extracted with EtOAc, layers separated, and the organic layer dried over sodium sulfate. The EtOAc layer was filtered, concentrated under vacuum, and purified by silica gel chromatography using 0-30% EtOAc in DCM to give title compound (210 mg, 0.391 mmol, 88.5% yield). LCMS: m/z=537.8 [M+H]⁺.

### Example 109: (5-((1S,3R)-2-(2-fluoro-2-methylpropyl)-3-methyl-2,3,4,9-tetrahydro-1H-pyrido[3,4-b]indol-1-yl)-1H-pyrrolo[3,2-b]pyridin-2-yl)methanol

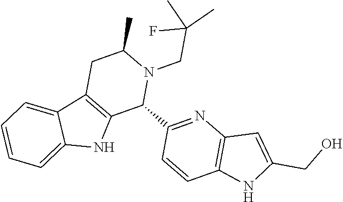 [SMILES: C[C@@H]1Cc2c([nH]c3ccccc23)[C@@H](c2ccc3[nH]c(CO)cc3n2)N1CC(C)(C)F]

A mixture of (5-((1S,3R)-2-(2-fluoro-2-methylpropyl)-3-methyl-2,3,4,9-tetrahydro-1H-pyrido[3,4-b]indol-1-yl)-1-((2-(trimethylsilyl)ethoxy)methyl)-1H-pyrrolo[3,2-b]pyridin-2-yl)methanol (1.00 equiv, 120 mg, 0.224 mmol) in DCM (2 mL) was cooled to 0 °C and treated with trifluoroacetic acid (58.1 equiv, 1.0 mL, 13.0 mmol). The reaction mixture was stirred for 1 h. The reaction mixture was concentrated under vacuum, extracted with EtOAc, neutralized with sat. NaHCO₃, dried over sodium sulfate, filtered, and concentrated. The crude was dissolved in methanol (1 mL) and treated with triethylamine (4.49 equiv, 0.14 mL, 1.00 mmol) and heated at 90 °C for 1 h, concentrated under vacuum, extracted with EtOAc, washed with water, brine, dried over sodium sulfate, filtered, and concentrated to give title compound (60 mg, 0.148 mmol, 66.0% yield). LCMS: m/z=407.2 [M+H]⁺.

### Example 110: 5-[(1S,3R)-2-(2-fluoro-2-methyl-propyl)-3-methyl-1,3,4,9-tetrahydropyrido[3,4-b]indol-1-yl]-1H-pyrrolo[3,2-b]pyridine-2-carbaldehyde

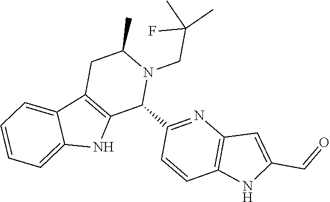 [SMILES: C[C@@H]1Cc2c([nH]c3ccccc23)[C@@H](c2ccc3[nH]c(C=O)cc3n2)N1CC(C)(C)F]

To [5-[(1 S,3R)-2-(2-fluoro-2-methyl-propyl)-3-methyl-1,3,4,9-tetrahydropyrido[3,4-b]indol-1-yl]-1H-pyrrolo[3,2-b]pyridin-2-yl]methanol (1.00 equiv, 60 mg, 0.148 mmol) in anhydrous DMSO (1 mL) was added 1,1,1-tris(acetyloxy)-1,1-dihydro-1,2-benzodioxol-3-(1 h)-one (1.50 equiv, 209 mg, 0.221 mmol) and stirred at RT for 1 h. The reaction was quenched with water and extracted with EtOAc. The EtOAc layer was washed with iN NaOH and brine, dried over sodium sulfate, filtered, and concentrated. The crude was purified by silica gel chromatography using 0-30% EtOAc in DCM to give title compound (45 mg, 0.111 mmol, 75.4% yield). LCMS: m/z=405.3 [M+H]⁺.

### Example 111: (1S,3R)-1-[2-[[3-(fluoromethyl)azetidin-1-yl]methyl]-1H-pyrrolo[3,2-b]pyridin-5-yl]-2-(2-fluoro-2-methyl-propyl)-3-methyl-1,3,4,9-tetrahydropyrido[3,4-b]indole

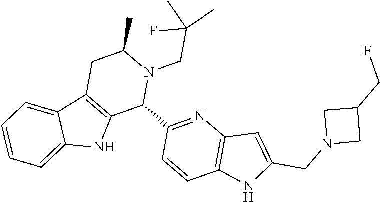 [SMILES: C[C@@H]1Cc2c([nH]c3ccccc23)[C@@H](c2ccc3[nH]c(CN4CC(CF)C4)cc3n2)N1CC(C)(C)F]

To 3-(fluoromethyl)azetidine hydrochloride (2.00 equiv, 28 mg, 0.223 mmol) in NMP (1 mL) was added the triethylamine (2.00 equiv, 0.031 mL, 0.223 mmol), stirred for 5 min, followed by addition of 5-[(1S,3R)-2-(2-fluoro-2-methyl-propyl)-3-methyl-1,3,4,9-tetrahydropyrido[3,4-b]indol-1-yl]-1H-pyrrolo[3,2-b]pyridine-2-carbaldehyde (1.00 equiv, 45 mg, 0.111 mmol) and acetic acid (2.00 equiv, 0.013 mL, 0.223 mmol). The reaction mixture was stirred at room temp for 30 min, followed by addition of sodium triacetoxyborohydride (3.00 equiv, 71 mg, 0.334 mmol) and stirred for 1 h. The reaction was quenched with methanol (0.5 mL) and purified by C-18 reverse phase chromatography using Luna column, eluting with 5-30% ACN in water with 0.1% formic acid as modifier to give title compound (29 mg, 0.0607 mmol, 54.6% yield). LCMS: m/z=478.3 [M+H]⁺.

¹H NMR (400 MHz, d₆-DMSO) δ 11.15 (d, J=2.0 Hz, 1H), 10.53 (s, 1H), 8.15 (s, 1H), 7.63 (dd, J=8.4, 0.9 Hz, 1H), 7.41 (d, J=7.7 Hz, 1H), 7.28-7.20 (m, 2H), 6.98 (dtd, J=23.6, 7.0, 1.2 Hz, 2H), 6.32 (d, J=1.8 Hz, 1H), 5.10 (s, 1H), 4.58 (d, J=6.1 Hz, 1H), 4.46 (d, J=6.1 Hz, 1H), 3.69 (s, 2H), 3.01 (t, J=6.6 Hz, 2H), 2.91-2.63 (m, 3H), 1.38 (d, J=21.7 Hz, 3H), 1.28 (d, J=21.6 Hz, 3H), 1.09 (d, J=6.7 Hz, 3H).

### Example 112: 2-[(3-methylazetidin-1-yl)methyl]-3H-benzimidazole-5-carbonitrile

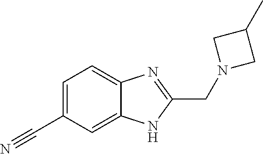 [SMILES: CC1CN(Cc2nc3ccc(C#N)cc3[nH]2)C1]

2-(Chloromethyl)-3H-benzimidazole-5-carbonitrile (161 mg, 0.84 mmol) and 3-methylazetidine hydrochloride (135 mg, 1.2 mmol) were dissolved in DCM (3 mL) and TEA (3.4 mmol) was added. The solution was stirred for 4 h at 40 °C. The solution was cooled then washed with brine solution. The organic layer dried over sodium sulfate, filtered, and dried in vacuo. The crude was purified on silica gel in 0-15% MeOH in DCM to give the titled product (60 mg, 32% yield). LCMS: m/z=227.2 [M+H]⁺.

### Example 113: 2-[(3-methylazetidin-1-yl)methyl]-3H-benzimidazole-5-carbaldehyde

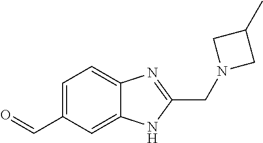 [SMILES: CC1CN(Cc2nc3ccc(C=O)cc3[nH]2)C1]

2-[(3-Methylazetidin-1-yl)methyl]-3H-benzimidazole-5-carbonitrile (60 mg, 0.27 mmol) was dissolved in THF (2 mL) under nitrogen at 0 °C. DIBAL (0.53 mmol) was added and stirred for 1 h. The reaction was quenched with EtOAc. Rochelle's Salt solution was added and stirred for 1 h. The organic layer was separated and washed with brine solution, dried over sodium sulfate, filtered, and evaporated in vacuo to yield the title compound (40 mg, 66% yield). LCMS: m/z=230.3 [M+H]⁺.

### Example 114: (1R,3R)-2-(2-fluoro-2-methyl-propyl)-3-methyl-1-[2-[(3-methylazetidin-1-yl)methyl]-3H-benzimidazol-5-yl]-1,3,4,9-tetrahydropyrido[3,4-b]indole

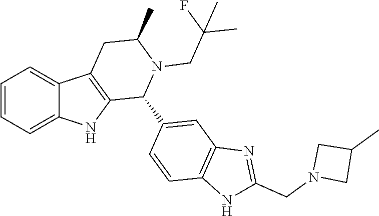 [SMILES: CC1CN(Cc2nc3cc([C@@H]4c5[nH]c6ccccc6c5C[C@@H](C)N4CC(C)(C)F)ccc3[nH]2)C1]

2-[(3-methylazetidin-1-yl)methyl]-3H-benzimidazole-5-carbaldehyde (40 mg, 0.17 mmol) and (R)—N-(1-(1H-indol-3-yl)propan-2-yl)-2-fluoro-2-methylpropan-1-amine (43 mg, 0.17 mmol) were dissolved in DCE (2 mL) and acetic acid (50 μL, 0.87 mmol) was added. The solution was stirred at 120 °C under microwave irradiation for 2 h. The reaction was quenched with saturated sodium bicarbonate solution and the organic layer was separated, dried over sodium sulfate, filtered, and dried in vacuo. The crude was dissolved in DMF (2 mL) and purified by HPLC on a Kintetex 5 μm C18 100 Å column (size: 100×30.0 Mm; gradient: 5-30% 0.1% formic acid in ACN in 0.1% formic acid in water) then lyophilized to give the title compound as a white solid (13 mg, 42% yield). LCMS: m/z=460.3 [M+H]⁺.

### Example 115: Methyl 2-[[3-(fluoromethyl)azetidin-1-yl]methyl]-1,3-benzoxazole-6-carboxylate

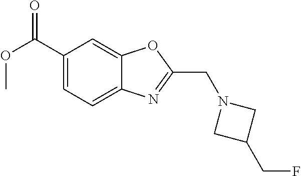 [SMILES: COC(=O)c1ccc2nc(CN3CC(CF)C3)oc2c1]

Methyl 2-(chloromethyl)-1,3-benzoxazole-6-carboxylate (250 mg, 1.1 mmol) and 3-(fluoromethyl)azetidine hydrochloride (167 mg, 1.3 mmol) were dissolved in DCM (4 mL) and triethylamine (620 μL, 4.4 mmol) was added. The solution was heated to 40 °C and stirred for 3 h. It was cooled to room temperature then poured into saturated sodium bicarbonate solution. The organic layer was separated, dried over sodium sulfate, filtered, and dried in vacuo to give the title compound (320 mg, 104% yield). LCMS: m/z=279.2 [M+H]⁺. This crude was used in the next step without further purification.

### Example 116: [2-[[3-(Fluoromethyl)azetidin-1-yl]methyl]-1,3-benzoxazol-6-yl]methanol

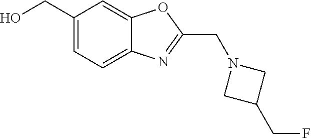 [SMILES: OCc1ccc2nc(CN3CC(CF)C3)oc2c1]

Methyl 2-[[3-(fluoromethyl)azetidin-1-yl]methyl]-1,3-benzoxazole-6-carboxylate (320 mg, 1.1 mmol) was dissolved in THE (5 mL) under nitrogen and cooled to −78 °C. LAH (1.5 mmol) was added and stirred for 1 h. The solution was warmed to 0 °C and diluted with MTBE (10 mL). While stirring, 240 μL of water was added, then 240 μL of 1N NaOH. The mixture was warmed to room temperature and stirred for 15 min. Sodium sulfate was added and stirred for another 15 min. This was then filtered and dried in vacuo to give the title compound (313 mg, 109% yield). LCMS: m/z=251.4 [M+H]⁺. The crude was used in the next step without further purification.

### Example 117: 2-[[3-(fluoromethyl)azetidin-1-yl]methyl]-1,3-benzoxazole-6-carbaldehyde

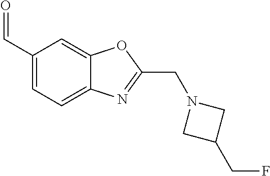 [SMILES: O=Cc1ccc2nc(CN3CC(CF)C3)oc2c1]

[2-[[3-(fluoromethyl)azetidin-1-yl]methyl]-1,3-benzoxazol-6-yl]methanol (280 mg, 1.1 mmol) and IBX (313 mg, 1.1 mmol) were dissolved in DMSO (4 mL) and stirred for 2 h. The solution was diluted with EtOAc, then poured into saturated sodium bicarbonate solution, then the organic layer was separated and extracted with EtOAc 3 times. The combined organic layers were washed with saturated brine solution 3 times, dried over sodium sulfate, filtered, and dried in vacuo. The crude was purified on silica gel in 0-10% MeOH in DCM to give the title compound (33 mg, 12% yield). LCMS: m/z=249.1 [M+H]⁺.

### Example 118: 2-[[3-(fluoromethyl)azetidin-1-yl]methyl]-6-[(1R,3R)-2-(2-fluoro-2-methyl-propyl)-3-methyl-1,3,4,9-tetrahydropyrido[3,4-b]indol-1-yl]-1,3-benzoxazole

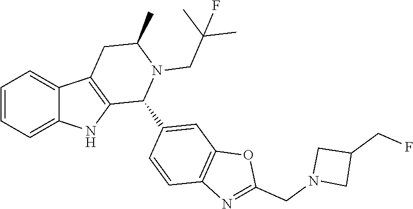 [SMILES: C[C@@H]1Cc2c([nH]c3ccccc23)[C@@H](c2ccc3nc(CN4CC(CF)C4)oc3c2)N1CC(C)(C)F]

(R)—N-(1-(1H-indol-3-yl)propan-2-yl)-2-fluoro-2-methylpropan-1-amine (33 mg, 0.13 mmol) and 2-[[3-(fluoromethyl)azetidin-1-yl]methyl]-1,3-benzoxazole-6-carbaldehyde (33 mg, 0.13 mmol) were dissolved in DCE (1.5 mL) and acetic acid (38 μL, 0.66 mmol) was added. The solution was stirred at 130 °C under microwave irradiation for 2.5 h. The solution was then cooled to room temperature, diluted with DCM, and poured into saturated sodium bicarbonate solution. The organic layers were separated, dried over sodium sulfate, filtered, and dried in vacuo. The crude material was dissolved in DMF (2 mL) and directly purified by HPLC on a Kintetex 5 μm C18 100 Å column (size: 100×30.0 mm; gradient: 5-30% 0.1% formic acid in ACN in 0.1% formic acid in water) then lyophilized to give the title compound as an off-white solid (3.1 mg, 5% yield). LCMS: m/z=479.6 [M+H]⁺. ¹H NMR (400 MHz, DMSO) δ 10.80 (s, 1H), 8.34 (s, 2H), 7.42 (d, J=7.7 Hz, 1H), 7.27 (d, J=8.0 Hz, 1H), 7.04 (ddd, J=8.1, 7.0, 1.3 Hz, 1H), 6.97 (td, J=7.4, 1.1 Hz, 1H), 6.85 (d, J=8.0 Hz, 1H), 6.74 (dd, J=8.0, 1.9 Hz, 1H), 6.67 (d, J=1.8 Hz, 1H), 4.99-4.93 (m, 1H), 4.66 (d, J=5.7 Hz, 1H), 4.54 (d, J=5.7 Hz, 1H), 4.46 (d, J=2.2 Hz, 2H), 4.14 (s, 2H), 3.86 (s, 2H), 3.19-2.98 (m, 2H), 2.72 (dd, J=31.2, 15.0 Hz, 1H), 1.45 (d, J=21.8 Hz, 3H), 1.29 (d, J=21.5 Hz, 3H), 1.06 (d, J=6.7 Hz, 3H).

### Example 119: Ethyl 6-bromoimidazo[1,2-a]pyridine-2-carboxylate

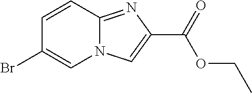

2-Amino-5-bromopyridine (3.0 g, 17.3 mmol) and sodium bicarbonate (2.9 g, 34.7 mmol) were stirred in dioxane (30 mL) at 100 °C and ethyl bromopyruvate (3.3 mL, 26.0 mmol) was added. The reaction was stirred for 16 h and brought to ambient temperature. It was diluted with EtOAc and washed with water, then saturated brine solution. The organic layer was separated and dried over sodium sulfate, then filtered, and concentrated in vacuo to afford the title compound (4.6 g, 99% yield). LCMS: m/z=270.1 [M+H]⁺.

### Example 120: (6-Bromoimidazo[1,2-a]pyridin-2-yl)methanol

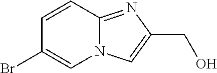 [SMILES: OCc1cn2cc(Br)ccc2n1]

Ethyl 6-bromoimidazo[1,2-a]pyridine-2-carboxylate (4.6 g, 17.1 mmol) was dissolved in THE (20 mL) under nitrogen and brought to 0 °C. DIBAL (34 mmol) was added and stirred for 1.5 h. The reaction was brought to ambient temperature and saturated Rochelle's Salt solution was added and stirred for 30 minutes. The aqueous layer was separated and extracted with EtOAc twice, and the organic layers were combined then washed with saturated brine solution. It was then dried over sodium sulfate, filtered, and dried in vacuo. The crude material was purified on silica gel in 0-10% MeOH in DCM to give the title product (1.75 g, 45% yield). LCMS: m/z=228.0 [M+H]⁺.

### Example 121: (6-Bromoimidazo[1,2-a]pyridin-2-yl)methoxy-tert-butyl-dimethyl-silane

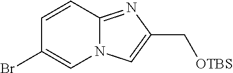 [SMILES: CC(C)(C)[Si](C)(C)OCc1cn2cc(Br)ccc2n1]

(6-Bromoimidazo[1,2-a]pyridin-2-yl)methanol (1.75 g, 7.7 mmol), imidazole (682 mg, 10.0 mmol), and TBS-Cl (10.0 mmol) were stirred in DMF (20 mL) for 2 hours at room temperature. The solution was then diluted with EtOAc and washed with saturated brine solution 5 times. The organic layer was dried over sodium sulfate, filtered, and dried in vacuo. The crude material was purified on silica gel in 0-30% MTBE in hexanes to give the title product (2.65 g, 100%). LCMS: m/z=342.9 [M+H]⁺.

### Example 122: 2-[[Tert-butyl(dimethyl)silyl]oxymethyl]imidazo[1,2-a]pyridine-6-carbaldehyde

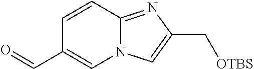 [SMILES: CC(C)(C)[Si](C)(C)OCc1cn2cc(C=O)ccc2n1]

(6-bromoimidazo[1,2-a]pyridin-2-yl)methoxy-tert-butyl-dimethyl-silane (195 mg, 0.57 mmol) was dissolved in dry ether (8 mL) and brought to −78 °C under nitrogen. N-butyllithium (0.57 mmol) was added and stirred for 1 h. DMF (3.5 mmol) was added and stirred for 30 min. Water was added then diluted with EtOAc. The organic layer was washed with brine solution, dried over sodium sulfate, filtered, and dried in vacuo to give the title compound (165 mg, 99%). LCMS: m/z=291.3 [M+H]⁺.

### Example 123: Tert-butyl-[[6-[(1R,3R)-2-(2-fluoro-2-methyl-propyl)-3-methyl-1,3,4,9-tetrahydropyrido[3,4-b]indol-1-yl]imidazo[1,2-a]pyridin-2-yl]methoxy]-dimethyl-silane

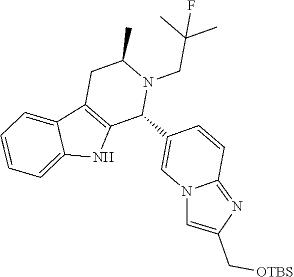 [SMILES: C[C@@H]1Cc2c([nH]c3ccccc23)[C@@H](c2ccc3nc(CO[Si](C)(C)C(C)(C)C)cn3c2)N1CC(C)(C)F]

(R)—N-(1-(1H-indol-3-yl)propan-2-yl)-2-fluoro-2-methylpropan-1-amine (141 mg, 0.57 mmol), 2-[[tert-butyl(dimethyl)silyl]oxymethyl]imidazo[1,2-a]pyridine-6-carbaldehyde (165 mg, 0.57 mmol), and acetic acid (162 μL, 2.8 mmol) were dissolved in DCE (2 mL) and stirred for 2 h at 120 °C under microwave irradiation. The solution was diluted with DCM then poured into saturated sodium bicarbonate solution and the organic layer was separated. It was dried over sodium sulfate, filtered, then dried in vacuo. The crude material was purified on silica gel in 0-35% EtOAc in hexanes to give the title product (122 mg, 41% yield). LCMS: m/z=521.6 [M+H]⁺.

### Example 124: [6-[(1R,3R)-2-(2-Fluoro-2-methyl-propyl)-3-methyl-1,3,4,9-tetrahydropyrido[3,4-b]indol-1-yl]imidazo[1,2-a]pyridin-2-yl]methanol

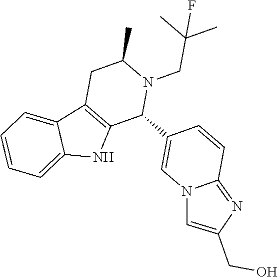 [SMILES: C[C@@H]1Cc2c([nH]c3ccccc23)[C@@H](c2ccc3nc(CO)cn3c2)N1CC(C)(C)F]

Tert-butyl-[[6-[(1R,3R)-2-(2-fluoro-2-methyl-propyl)-3-methyl-1,3,4,9-tetrahydropyrido[3,4-b]indol-1-yl]imidazo[1,2-a]pyridin-2-yl]methoxy]-dimethyl-silane (122 mg, 0.23 mmol) was dissolved in THE (3 mL) and TBAF (2.3 mmol) was added. The solution was heated to 80 °C and stirred for 2 h. The solution was cooled to room temperature then poured into a saturated solution of sodium bicarbonate. The organic layer was separated and washed with saturated brine solution 3 times. It was then dried over sodium sulfate, filtered, and dried in vacuo to yield the title product (95 mg, 100% yield). LCMS: m/z=407.4 [M+H]⁺.

### Example 125: 6-[(1R,3R)-2-(2-Fluoro-2-methyl-propyl)-3-methyl-1,3,4,9-tetrahydropyrido[3,4-b]indol-1-yl]imidazo[1,2-a]pyridine-2-carbaldehyde

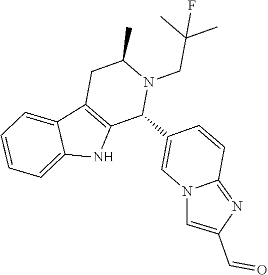 [SMILES: C[C@@H]1Cc2c([nH]c3ccccc23)[C@@H](c2ccc3nc(C=O)cn3c2)N1CC(C)(C)F]

[6-[(1R,3R)-2-(2-Fluoro-2-methyl-propyl)-3-methyl-1,3,4,9-tetrahydropyrido[3,4-b]indol-1-yl]imidazo[1,2-a]pyridin-2-yl]methanol (95 mg, 0.23 mmol) and IBX (111 mg, 1.7 mmol) were dissolved in DMSO (2 mL) and stirred for 1.5 h. The solution was diluted with EtOAc then poured into a saturated sodium bicarbonate solution and the organic layer was separated. It was washed with saturated brine solution, dried over sodium sulfate, filtered, then dried in vacuo. The crude material was purified on silica gel in 0-30% EtOAc in hexanes to give the title product (33 mg, 35% yield). LCMS: m/z=405.6 [M+H]⁺.

### Example 126: (1R,3R)-1-[2-[[3-(Fluoromethyl)azetidin-1-yl]methyl]imidazo[1,2-a]pyridin-6-yl]-2-(2-fluoro-2-methyl-propy)-3-methyl-1,3,4,9-tetrahydropyrido[3,4-b]indole

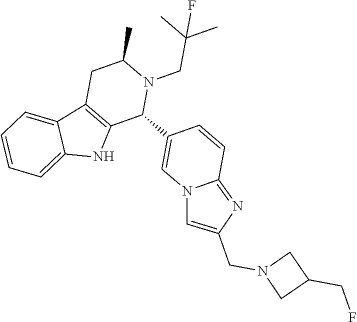 [SMILES: C[C@@H]1Cc2c([nH]c3ccccc23)[C@@H](c2ccc3nc(CN4CC(CF)C4)cn3c2)N1CC(C)(C)F]

6-[(1R,3R)-2-(2-fluoro-2-methyl-propyl)-3-methyl-1,3,4,9-tetrahydropyrido[3,4-b]indol-1-yl]imidazo[1,2-a]pyridine-2-carbaldehyde (33 mg, 0.08 mmol) was dissolved in DMF (1 mL) and acetic acid (9.3 μL, 0.16 mmol) was added. Separately, 3-(fluoromethyl)azetidine hydrochloride (12.3 mg, 0.10 mmol) was dissolved in DMF (1 mL) and TEA (23 μL, 0.16 mmol) was added. These solutions were combined and stirred for 30 min. STAB (52 mg, 0.25 mmol) was added and stirred for 2 h. The reaction was quenched with MeOH (1 mL) and directly purified by HPLC on a Kintetex 5 μm C18 100 Å column (size: 100×30.0 mm; gradient: 5-30% 0.1% formic acid in ACN in 0.1% formic acid in water) then lyophilized to give the title compound as an off-white solid (30.2 mg, 78% yield). LCMS: m/z=478.6 [M+H]⁺. ¹H NMR (400 MHz, DMSO) δ 10.81 (s, 1H), 8.31 (s, 1H), 7.88 (s, 1H), 7.73 (s, 2H), 7.49-7.43 (m, 3H), 7.34 (d, J=9.4 Hz, 1H), 7.29 (d, J=8.0 Hz, 2H), 7.08 (td, J=7.6, 1.3 Hz, 2H), 7.03-6.97 (m, 2H), 5.03 (s, 2H), 4.56 (d, J=6.3 Hz, 2H), 4.44 (d, J=6.3 Hz, 2H), 2.99 (dd, J=7.2, 5.7 Hz, 3H), 2.86-2.58 (m, 5H), 1.46 (d, J=21.8 Hz, 5H), 1.33 (s, 3H).

### Example 127: 2-(hydroxymethyl)imidazo[1,2-a]pyridine-7-carbonitrile

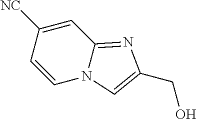 [SMILES: N#Cc1ccn2cc(CO)nc2c1]

To 7-cyano-imidazo[1,2-a]pyridine-2-carboxylic acid ethyl ester (1.00 equiv, 250 mg, 1.16 mmol) in THE (10 mL) at 0 °C under nitrogen was added diisobutylaluminum hydride (2.00 equiv, 2.3 mL, 2.32 mmol) dropwise and stirred for 1 h. After slowly warming to room temp, the reaction was cooled back to 0 °C, and quenched with EtOAc, followed by saturated solution of Rochelle's salt. The mixture was stirred for 30 min. The reaction mixture was extracted with EtOAc, layers were separated, and the organic layer dried over sodium sulfate. The organic layer was filtered, concentrated under vacuum, and purified by silica gel chromatography using 0-30% EtOAc in hexane to give title compound (70 mg, 0.404 mmol, 34.8% yield). LCMS: m/z=174.1 [M+H]⁺.

### Example 128: 2-[[tert-butyl(dimethyl)silyl]oxymethyl]imidazo[1,2-a]pyridine-7-carbonitrile

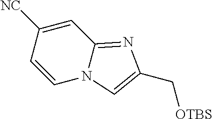 [SMILES: CC(C)(C)[Si](C)(C)OCc1cn2ccc(C#N)cc2n1]

To 2-(hydroxymethyl)imidazo[1,2-a]pyridine-7-carbonitrile (1.00 equiv, 80 mg, 0.462 mmol) in DMF (1 mL) at 0 °C was added imidazole (2.00 equiv, 63 mg, 0.924 mmol), followed by tert-butyldimethylchlorosilane (1.00 equiv, 70 mg, 0.462 mmol). The reaction mixture was stirred for 30 min. The reaction was quenched with water followed by brine, dried over sodium sulfate, filtered and concentrated. The crude was purified by silica gel chromatography using 0-30% EtOAc in hexane to give title compound (117 mg, 0.407 mmol, 88.1% yield). LCMS: m/z=288.3 [M+H]⁺.

### Example 129: 2-(hydroxymethyl)imidazo[1,2-a]pyridine-7-carbaldehyde

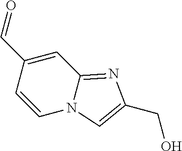 [SMILES: O=Cc1ccn2cc(CO)nc2c1]

To 2-[[tert-butyl(dimethyl)silyl]oxymethyl]imidazo[1,2-a]pyridine-7-carbonitrile (1.00 equiv, 117 mg, 0.407 mmol) in THE (5 mL) at 0 °C under nitrogen was added diisobutylaluminum hydride (1.00 equiv, 0.41 mL, 0.407 mmol) dropwise, and the reaction mixture was stirred for 1 h. After slowly warming to room temp, the reaction was cooled back to 0 °C, and then quenched with EtOAc, followed by saturated solution of Rochelle's salt. The mixture was stirred for 30 min then extracted with EtOAc. The layers were separated, and the organic layer was dried over sodium sulfate. The organic layer was filtered, concentrated under vacuum, and purified by silica gel chromatography using 0-30% EtOAc in DCM to give title compound (20 mg, 0.114 mmol, 27.9% yield). LCMS: m/z=291.3 [M+H]⁺.

### Example 130: [7-[(1R,3R)-2-(2-fluoro-2-methyl-propyl)-3-methyl-1,3,4,9-tetrahydropyrido[3,4-b]indol-1-yl]imidazo[1,2-a]165yridine-2-yl]methanol

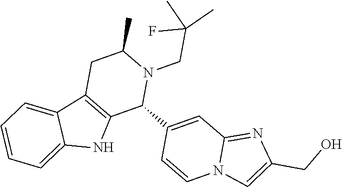 [SMILES: C[C@@H]1Cc2c([nH]c3ccccc23)[C@@H](c2ccn3cc(CO)nc3c2)N1CC(C)(C)F]

To (R)—N-(1-(1H-indol-3-yl)propan-2-yl)-2-fluoro-2-methylpropan-1-amine (1.30 equiv, 37 mg, 0.148 mmol) in DCE (2 mL) was added 2-(hydroxymethyl)imidazo[1,2-a]pyridine-7-carbaldehyde (1.00 equiv, 20 mg, 0.114 mmol) and acetic acid (2.00 equiv, 0.013 mL, 0.227 mmol), and stirred at 110 °C in microwave for 3 h. The reaction mixture was extracted with DCM, washed with saturated sodium bicarbonate solution, dried over sodium sulfate, filtered, and concentrated. The crude was purified by silica gel chromatography using a 24 g column elution with 0-30% EtOAc in DCM to give title compound (20 mg, 0.0492 mmol, 43.3% yield). LCMS: m/z=407.2 [M+H]⁺.

### Example 131: 7-[(1R,3R)-2-(2-fluoro-2-methyl-propyl)-3-methyl-1,3,4,9-tetrahydropyrido[3,4-b]indol-1-yl]imidazo[1,2-a]pyridine-2-carbaldehyde

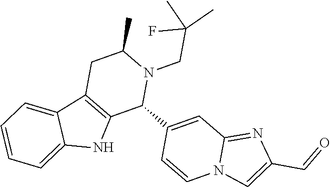 [SMILES: C[C@@H]1Cc2c([nH]c3ccccc23)[C@@H](c2ccn3cc(C=O)nc3c2)N1CC(C)(C)F]

To [7-[(1R,3R)-2-(2-fluoro-2-methyl-propyl)-3-methyl-1,3,4,9-tetrahydropyrido[3,4-b]indol-1-yl]imidazo[1,2-a]pyridin-2-yl]methanol (1.00 equiv, 20 mg, 0.0492 mmol) in anhydrous DMSO (0.5 mL) was added 1,1,1-tris(acetyloxy)-1,1-dihydro-1,2-benzodioxol-3-(1h)-one (1.20 equiv, 51 mg, 0.0590 mmol) and stirred at 25 °C for 1 h. The reaction was quenched with water and extracted with EtOAc. The EtOAc layer was washed with 1N NaOH, brine, dried over sodium sulfate, filtered, and concentrated. The crude was purified by silica gel chromatography using 0-30% EtOAc in DCM to give title compound (12 mg, 0.0297 mmol, 60.3% yield). LCMS: m/z=405.2 [M+H]⁺.

### Example 132: [7-[(1R,3R)-2-(2-fluoro-2-methyl-propyl)-3-methyl-1,3,4,9-tetrahydropyrido[3,4-b]indol-1-yl]imidazo[1,2-a]pyridin-2-yl]methanol

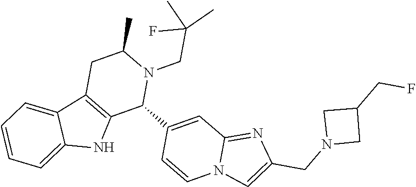 [SMILES: C[C@@H]1Cc2c([nH]c3ccccc23)[C@@H](c2ccn3cc(CN4CC(CF)C4)nc3c2)N1CC(C)(C)F]

To 3-(fluoromethyl)azetidine hydrochloride (2.00 equiv, 7.5 mg, 0.0593 mmol) in NMP (0.5 mL) was added triethylamine (2.00 equiv, 0.0083 mL, 0.0593 mmol), stirred for 5 min, followed by addition of 7-[(1R,3R)-2-(2-fluoro-2-methyl-propyl)-3-methyl-1,3,4,9-tetrahydropyrido[3,4-b]indol-1-yl]imidazo[1,2-a]pyridine-2-carbaldehyde (1.00 equiv, 12 mg, 0.0297 mmol) and acetic acid (2.00 equiv, 0.0034 mL, 0.0593 mmol). The reaction mixture was stirred at room temp for 30 min, followed by addition of sodium triacetoxyborohydride (3.00 equiv, 19 mg, 0.0890 mmol) and stirred for 1 h. The reaction was quenched with methanol (0.5 mL), and purified by C-18 reverse phase chromatography using Luna column, eluting with 5-30% ACN in water with 0.1% formic acid as modifier to give the title compound (2.0 mg, 0.00419 mmol, 14.1% yield). LCMS: m/z=478.4 [M+H]⁺.

### Example 133: [2-(chloromethyl)-1,3-benzoxazol-5-yl]methanol

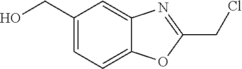 [SMILES: OCc1ccc2oc(CCl)nc2c1]

Methyl 2-(chloromethyl)-1,3-benzoxazole-5-carboxylate (300 mg, 1.3 mmol) was dissolved in anhydrous THE (4 mL) under nitrogen and brought to −78 °C. DIBAL (2.0 mmol) was added and stirred for 1 h. The solution was warmed to room temperature and diluted with EtOAc. Saturated Rochelle's Salt was added and stirred for an additional hour. The layers were separated and extracted with EtOAc. The combined organic layers were washed with saturated brine solution, dried over sodium sulfate, filtered, and dried in vacuo to yield the title product (262 mg, 99% yield). LCMS: m/z=198.5 [M+H]⁺. The crude was used directly in the next step.

### Example 134: 2-(chloromethyl)-1,3-benzoxazole-5-carbaldehyde

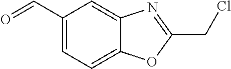 [SMILES: O=Cc1ccc2oc(CCl)nc2c1]

[2-(Chloromethyl)-1,3-benzoxazol-5-yl]methanol (262 mg, 1.3 mmol) and IBX (742 mg, 2.6 mmol) were dissolved in DMSO (2 mL) and stirred for 2 h. The solution was diluted with EtOAc then poured into saturated sodium bicarbonate solution. The organic layer was separated and washed with saturated brine solution. It was then dried over sodium sulfate, filtered, and dried in vacuo. The crude material was purified on silica gel with 0-30% EtOAc in hexanes to give the title product (180 mg, 69% yield). LCMS: m/z=196.6 [M+H]⁺.

### Example 135: 2-(chloromethyl)-5-[(1R,3R)-2-(2-fluoro-2-methyl-propyl)-3-methyl-1,3,4,9-tetrahydropyrido[3,4-b]indol-1-yl]-1,3-benzoxazole

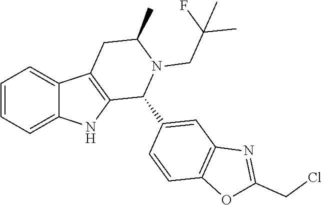 [SMILES: C[C@@H]1Cc2c([nH]c3ccccc23)[C@@H](c2ccc3oc(CCl)nc3c2)N1CC(C)(C)F]

2-(Chloromethyl)-1,3-benzoxazole-5-carbaldehyde (180 mg, 0.92 mmol) and (R)—N-(1-(1H-indol-3-yl)propan-2-yl)-2-fluoro-2-methylpropan-1-amine (228 mg, 0.92 mmol) were dissolved in DCE (4 mL) and acetic acid (263 μL, 4.6 mmol) was added. The solution was stirred at 120 °C under microwave irradiation for 2 h. The solution was poured into saturated sodium bicarbonate solution and the organic layer was separated. It was then washed with saturated brine solution, dried over sodium sulfate, filtered, and dried in vacuo. The crude material was purified in 0-30% EtOAc in hexanes to give the title product (140 mg, 36%). LCMS: m/z=426.8 [M+H]⁺.

### Example 136: 2-[[3-(fluoromethyl)azetidin-1-yl]methyl]-5-[(1R,3R)-2-(2-fluoro-2-methyl-propyl)-3-methyl-1,3,4,9-tetrahydropyrido[3,4-b]indol-1-yl]-1,3-benzoxazole

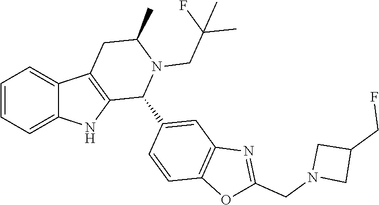 [SMILES: C[C@@H]1Cc2c([nH]c3ccccc23)[C@@H](c2ccc3oc(CN4CC(CF)C4)nc3c2)N1CC(C)(C)F]

2-(Chloromethyl)-5-[(1R,3R)-2-(2-fluoro-2-methyl-propyl)-3-methyl-1,3,4,9-tetrahydropyrido[3,4-b]indol-1-yl]-1,3-benzoxazole (36 mg, 0.08 mmol) and 3-(fluoromethyl)azetidine hydrochloride (14 mg, 0.11 mmol) were dissolved in DMF (2 mL). TEA (47 μL, 0.34 mmol) was added and stirred for 2 h at 40 °C. The reaction was quenched with formic acid (47 μL) and directly purified by HPLC on a Kintetex 5 μm C18 100 Å column (size: 100×30.0 mm; gradient: 5-30% 0.1% formic acid in ACN in 0.1% formic acid in water) then lyophilized to give the title compound as a white solid (14.3 mg, 35% yield). LCMS: m/z=479.6 [M+H]⁺. ¹H NMR (400 MHz, DMSO) δ 10.82 (s, 1H), 7.64 (d, J=8.5 Hz, 1H), 7.46 (dd, J=4.6, 3.1 Hz, 2H), 7.36 (dd, J=8.5, 1.7 Hz, 1H), 7.29 (d, J=8.0 Hz, 1H), 7.07 (ddd, J=8.1, 7.0, 1.3 Hz, 1H), 6.99 (td, J=7.4, 1.1 Hz, 1H), 5.17 (s, 1H), 4.57 (d, J=6.2 Hz, 1H), 4.46 (d, J=6.2 Hz, 1H), 3.83 (s, 2H), 3.43 (td, J=7.7, 1.5 Hz, 1H), 3.12 (d, J=6.6 Hz, 3H), 2.85-2.69 (m, 2H), 2.66-2.54 (m, 2H), 1.47 (d, J=21.8 Hz, 3H), 1.31 (d, J=21.5 Hz, 3H), 1.06 (d, J=6.7 Hz, 3H).

### Example 137: 2-[[3-methylazetidin-1-yl]methyl]-5-[(1R,3R)-2-(2-fluoro-2-methyl-propyl)-3-methyl-1,3,4,9-tetrahydropyrido[3,4-b]indol-1-yl]-1,3-benzoxazole

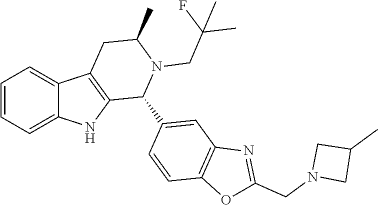 [SMILES: CC1CN(Cc2nc3cc([C@@H]4c5[nH]c6ccccc6c5C[C@@H](C)N4CC(C)(C)F)ccc3o2)C1]

2-(Chloromethyl)-5-[(1R,3R)-2-(2-fluoro-2-methyl-propyl)-3-methyl-1,3,4,9-tetrahydropyrido[3,4-b]indol-1-yl]-1,3-benzoxazole (19 mg, 0.04 mmol) and 3-methylazetidine hydrochloride (6.2 mg, 0.06 mmol) were dissolved in DMF (2 mL). TEA (25 μL, 0.18 mmol) was added and stirred for 2 h at 40 °C. The reaction was quenched with formic acid (25 μL) and directly purified by HPLC on a Kintetex 5 μm C18 100 Å column (size: 100×30.0 mm; gradient: 5-3% 0.1% formic acid in ACN in 0.1% formic acid in water) then lyophilized to give the title compound as a white solid (9.8 ng, 47% yield). LCMS: m/z=461.4 [M+H]⁺. ¹H NMR (400 MHz, DMSO) δ 10.82 (s, 1H), 7.63 (d, J=8.5 Hz, 1H), 7.46 (d, J=7.3 Hz, 2H), 7.36 (dd, J=8.5, 1.7 Hz, 1H), 7.29 (d, J=8.0 Hz, 1H), 7.07 (ddd, J=8.1, 7.0, 1.3 Hz, 1H), 6.99 (td, J=7.5, 1.1 Hz, 1H), 5.17 (s, 1H), 3.79 (s, 2H), 3.49-3.44 (m, 5H), 2.87 (t, J=6.5 Hz, 3H), 2.81-2.56 (m, 3H), 1.47 (d, J=21.8 Hz, 4H), 1.31 (d, J=21.5 Hz, 4H), 1.08 (dd, J=19.2, 6.7 Hz, 2H).

### Example 138: 2-(2-azaspiro[3.3]heptan-2-ylmethyl)-5-[(1R,3R)-2-(2-fluoro-2-methyl-propyl)-3-methyl-1,3,4,9-tetrahydropyrido[3,4-b]indol-1-yl]-1,3-benzoxazole

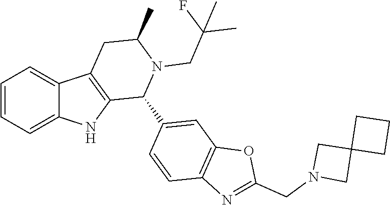 [SMILES: C[C@@H]1Cc2c([nH]c3ccccc23)[C@@H](c2ccc3nc(CN4CC5(CCC5)C4)oc3c2)N1CC(C)(C)F]

2-(Chloromethyl)-5-[(1R,3R)-2-(2-fluoro-2-methyl-propyl)-3-methyl-1,3,4,9-tetrahydropyrido[3,4-b]indol-1-yl]-1,3-benzoxazole (30 mg, 0.07 mmol) and 2-azoniaspiro[3.3]heptane hydrochloride (612 mg, 0.09 mmol) were dissolved in DMF (2 mL). TEA (39 μL, 0.28 mmol) was added and stirred for 2 h at 40 °C. The reaction was quenched with formic acid (39 μL) and directly purified by HPLC on a Kintetex 5 μm C18 100 Å column (size: 100×30.0 mm; gradient: 5-30% 0.1% formic acid in ACN in 0.1% formic acid in water) then lyophilized to give the title compound as a white solid (7.1 mg, 21% yield). LCMS: m/z=487.5 [M+H]⁺. ¹H NMR (400 MHz, DMSO) δ 10.82 (s, 1H), 7.63 (d, J=8.5 Hz, 1H), 7.46 (d, J=7.1 Hz, 2H), 7.35 (dd, J=8.5, 1.7 Hz, 1H), 7.29 (d, J=8.0 Hz, 1H), 7.07 (ddd, J=8.1, 7.0, 1.3 Hz, 1H), 6.99 (td, J=7.5, 1.1 Hz, 1H), 5.17 (s, 1H), 3.76 (s, 2H), 3.25 (s, 5H), 2.85-2.57 (m, 3H), 2.03 (t, J=7.6 Hz, 5H), 1.74 (pd, J=7.3, 1.0 Hz, 2H), 1.47 (d, J=21.8 Hz, 3H), 1.31 (d, J=21.5 Hz, 4H), 1.06 (d, J=6.7 Hz, 3H).

### Example 139: 5-Bromo-2-(Chloromethyl)-6-Fluoro-1,3-Benzoxazole

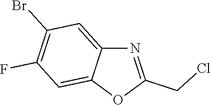 [SMILES: Fc1cc2oc(CCl)nc2cc1Br]

2-Amino-4-bromo-5-fluorophenol (1.00 equiv, 640 mg, 3.11 mmol) and ethyl 2-chloroethanimidoate hydrochloride (1.30 equiv, 638 mg, 4.04 mmol) were heated at 90 °C in ethanol (10 mL) overnight. The volatiles were removed under reduced pressure, and the crude was dissolved in DCM. The insoluble material was filtered, and the solution was concentrated under reduced pressure to give the title product (783 mg, 95% yield). LCMS: m/z=265.5 [M+H]⁺.

### Example 140: 5-Bromo-6-fluoro-2-[[3-(fluoromethyl)azetidin-1-yl]methyl]-1,3-benzoxazole

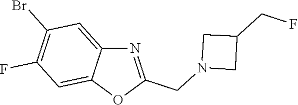 [SMILES: FCC1CN(Cc2nc3cc(Br)c(F)cc3o2)C1]

3-(Fluoromethyl)azetidine hydrochloride (1.20 equiv, 446 mg, 3.55 mmol) and 5-bromo-2-(chloromethyl)-6-fluoro-1,3-benzoxazole (1.00 equiv, 783 mg, 2.96 mmol) were dissolved in DCM (15 mL), warmed to 50 °C, and stirred for 1.5 h. The solution was then concentrated under reduced pressure. The crude was purified on silica gel in 0-10% MeOH (2% TEA) in DCM to give the title product (725 mg, 77% yield). LCMS: m/z=318.1 [M+H]⁺.

### Example 141: 6-Fluoro-2-[[3-(fluoromethyl)azetidin-1-yl]methyl]-1,3-benzoxazole-5-carbonitrile

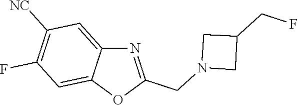 [SMILES: N#Cc1cc2nc(CN3CC(CF)C3)oc2cc1F]

5-Bromo-6-fluoro-2-[[3-(fluoromethyl)azetidin-1-yl]methyl]-1,3-benzoxazole (1.00 equiv, 460 mg, 1.45 mmol), Xantphos Pd G4 (0.0800 equiv, 112 mg, 0.116 mmol), zinc (0.200 equiv, 19 mg, 0.290 mmol) and zinc cyanide (1.00 equiv, 170 mg, 1.45 mmol) were dissolved in dry NMP (4 mL) and stirred under microwave irradiation for 10 min at 100 °C. The mixture was diluted with EtOAc and washed with saturated brine solution 3 times. The organic layers were dried over sodium sulfate, filtered, and dried in vacuo. The crude was purified on silica gel in 0-10% MeOH (2% TEA) in DCM to give the title product (346 mg, 91% yield). LCMS: m/z=264.3 [M+H]⁺.

### Example 142: 6-Fluoro-2-[[3-(fluoromethyl)azetidin-1-yl]methyl]-1,3-benzoxazole-5-carbaldehyde

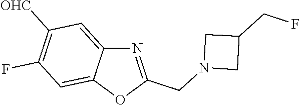 [SMILES: O=Cc1cc2nc(CN3CC(CF)C3)oc2cc1F]

6-Fluoro-2-[[3-(fluoromethyl)azetidin-1-yl]methyl]-1,3-benzoxazole-5-carbonitrile (1.00 equiv, 346 mg, 1.31 mmol) was dissolved in dry THF (10 mL) under nitrogen and cooled to 0 °C. DIBAL (4 mL, 1 M in toluene) was added dropwise and stirred for 1 h. The reaction mixture was then warmed to room temperature and quenched with EtOAc. Rochelle's Salt solution was then added and stirred for 30 min. The organic layers were then separated, dried over sodium sulfate, filtered, and dried in vacuo. The crude was purified on silica gel in 0-10% MeOH (2% TEA) in DCM to give the title product (55 mg, 16% yield). LCMS: m/z=267.3 [M+H]⁺.

### Example 143: 6-Fluoro-2-[[3-(fluoromethyl)azetidin-1-yl]methyl]-5-[(1R,3R)-2-(2-fluoro-2-methyl-propyl)-3-methyl-1,3,4,9-tetrahydropyrido[3,4-b]indol-1-yl]-1,3-benzoxazole

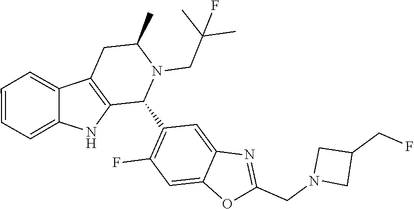 [SMILES: C[C@@H]1Cc2c([nH]c3ccccc23)[C@@H](c2cc3nc(CN4CC(CF)C4)oc3cc2F)N1CC(C)(C)F]

(R)—N-(1-(1H-indol-3-yl)propan-2-yl)-2-fluoro-2-methylpropan-1-amine (1.07 equiv, 55 mg, 0.221 mmol) and 6-fluoro-2-[[3-(fluoromethyl)azetidin-1-yl]methyl]-1,3-benzoxazole-5-carbaldehyde (1.00 equiv, 55 mg, 0.207 mmol) were dissolved in DCE (3 mL), and acetic acid (5.00 equiv, 0.059 mL, 1.03 mmol) was added. The solution was stirred under microwave irradiation for 2 h at 120 °C. The solution was diluted with DCM, poured into saturated sodium bicarbonate solution, and the organic layers were separated. The organic layers were dried over sodium sulfate, filtered, and dried in vacuo. The crude was dissolved in DMF (3 mL) and was purified by HPLC on a Kintetex 5 μm C18 100 Å column (size: 100×30.0 mm; gradient: 5-30% 0.1% formic acid in ACN in 0.1% formic acid in water) then lyophilized to give the title compound as a white solid (7.7 mg, 7% yield). LCMS: m/z=497.6 [M+H]⁺; ¹H NMR (400 MHz, DMSO) δ 10.66 (s, 1H), 7.46 (d, J=7.7 Hz, 1H), 7.29-7.18 (m, 1H), 7.02 (dtd, J=23.5, 7.1, 1.2 Hz, 2H), 6.72 (d, J=10.5 Hz, 1H), 6.10 (d, J=7.9 Hz, 1H), 5.28 (s, 1H), 4.59 (d, J=5.7 Hz, 1H), 4.48 (d, J=7.8 Hz, 2H), 4.04 (s, 2H), 3.76 (s, 4H), 3.08-2.61 (m, 4H), 1.40 (d, J=21.6 Hz, 3H), 1.26 (d, J=21.2 Hz, 3H), 1.05 (d, J=6.6 Hz, 3H).

### Example 144: 5-Bromo-2-(chloromethyl)-7-fluoro-1,3-benzoxazole

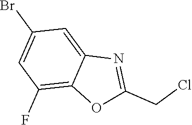 [SMILES: Fc1cc(Br)cc2nc(CCl)oc12]

2-Amino-4-bromo-6-fluoro-phenol (1.00 equiv, 640 mg, 3.11 mmol) and ethyl 2-chloroethanimidoate hydrochloride (1.30 equiv, 638 mg, 4.04 mmol) were heated at 90 °C in ethanol (10 mL) overnight. The volatiles were removed under reduced pressure, and the crude was dissolved in DCM. The insoluble material was filtered, and the solution was concentrated under reduced pressure to give the title product (821 mg, 100% yield). LCMS: m/z=265.5 [M+H]⁺.

### Example 145: 5-Bromo-7-fluoro-2-[[3-(fluoromethyl)azetidin-1-yl]methyl]-1,3-benzoxazole

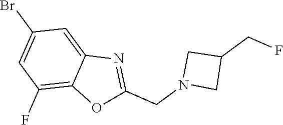 [SMILES: FCC1CN(Cc2nc3cc(Br)cc(F)c3o2)C1]

3-(Fluoromethyl)azetidine hydrochloride (1.20 equiv, 468 mg, 3.73 mmol) and 5-bromo-2-(chloromethyl)-7-fluoro-1,3-benzoxazole (1.00 equiv, 821 mg, 3.10 mmol) were dissolved in DCM (15 mL) and warmed to 50 °C and stirred for 1.5 h. The solution was then concentrated under reduced pressure. The crude material was purified on silica gel in 0-10% MeOH (2% TEA) in DCM to give the title product (845 mg, 86% yield). LCMS: m/z=318.1 [M+H]⁺.

### Example 146: 7-Fluoro-2-[[3-(fluoromethyl)azetidin-1-yl]methyl]-1,3-benzoxazole-5-carbonitrile

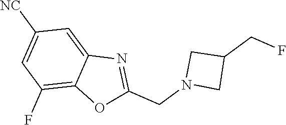 [SMILES: N#Cc1cc(F)c2oc(CN3CC(CF)C3)nc2c1]

1,1′-Bis(diphenylphosphino)ferrocene-palladium(II) dichloride dichloromethane complex (0.100 equiv, 188 mg, 0.257 mmol), 5-bromo-7-fluoro-2-[[3-(fluoromethyl)azetidin-1-yl]methyl]-1,3-benzoxazole (1.00 equiv, 815 mg, 2.57 mmol), zinc (0.250 equiv, 42 mg, 0.642 mmol), zinc cyanide (2.00 equiv, 603 mg, 5.14 mmol), and tris(dibenzylideneacetone)dipalladium(0) (0.0500 equiv, 118 mg, 0.128 mmol) were dissolved in dry DMF (10 mL) and stirred at 120 °C for 3 h. The mixture was then cooled to room temperature and diluted with EtOAc. It was then filtered and poured into saturated brine solution. The organic layers were then separated and washed with saturated brine solution 5 times. The organic layers were dried over sodium sulfate, filtered, and dried in vacuo. The crude was purified on silica gel in 0-10% MeOH (2% TEA) in DCM to give the title product (508 mg, 75% yield). LCMS: m/z=264.2 [M+H]⁺.

### Example 147: 7-Fluoro-2-[[3-(fluoromethyl)azetidin-1-yl]methyl]-1,3-benzoxazole-5-carbaldehyde

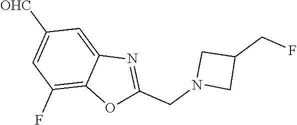 [SMILES: O=Cc1cc(F)c2oc(CN3CC(CF)C3)nc2c1]

7-Fluoro-2-[[3-(fluoromethyl)azetidin-1-yl]methyl]-1,3-benzoxazole-5-carbonitrile (1.00 equiv, 508 mg, 1.93 mmol) was dissolved in dry THE (8 mL) under nitrogen and cooled to 0 °C. DIBAL (5.7 mL, 1 M in toluene) was added dropwise and stirred for 1 h. The reaction was then warmed to room temperature and quenched with EtOAc. Rochelle's Salt solution was then added and stirred for 30 min. The organic layers were then separated, dried over sodium sulfate, filtered, and dried in vacuo. The crude was purified on silica gel in 0-10% MeOH (2% TEA) in DCM to give the title product. LCMS: m/z=267.3 [M+H]⁺.

### Example 148: 7-Fluoro-2-[[3-(fluoromethyl)azetidin-1-yl]methyl]-5-[(1R,3R)-2-(2-fluoro-2-methyl-propyl)-3-methyl-1,3,4,9-tetrahydropyrido[3,4-b]indol-1-yl]-1,3-benzoxazole

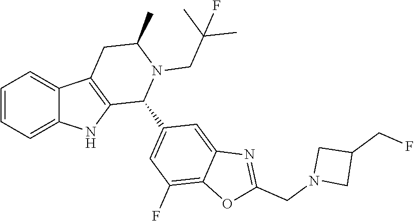 [SMILES: C[C@@H]1Cc2c([nH]c3ccccc23)[C@@H](c2cc(F)c3oc(CN4CC(CF)C4)nc3c2)N1CC(C)(C)F]

(R)—N-(1-(1H-indol-3-yl)propan-2-yl)-2-fluoro-2-methylpropan-1-amine (1.20 equiv, 62 mg, 0.248 mmol) and 7-fluoro-2-[[3-(fluoromethyl)azetidin-1-yl]methyl]-1,3-benzoxazole-5-carbaldehyde (1.00 equiv, 55 mg, 0.207 mmol) were dissolved in DCE (3 mL), and acetic acid (5.00 equiv, 62.0 mg, 1.03 mmol) was added. The solution was stirred under microwave irradiation for 2 h at 120 °C. The solution was diluted with DCM, poured into saturated sodium bicarbonate solution, and the organic layers were separated. The organic layers were dried over sodium sulfate, filtered, and dried in vacuo. The crude was dissolved in DMF (2 mL) and was directly purified by HPLC on a Kintetex 5 μm C18 100 Å column (size: 100×30.0 mm; gradient: 5-30% 0.1% formic acid in ACN in 0.1% formic acid in water) then lyophilized to give the title compound as a white solid (115.9 mg, 55% yield). LCMS: m/z=497.6 [M+H]⁺; ¹H NMR (400 MHz, DMSO) δ 10.82 (s, 1H), 7.44 (dd, J=7.7, 3.6 Hz, 1H), 7.36-7.25 (m, 1H), 7.12-7.01 (m, 1H), 6.98 (ddd, J=8.0, 7.0, 1.2 Hz, 1H), 6.80-6.66 (m, 1H), 6.51 (d, J=1.9 Hz, 1H), 4.94 (s, 1H), 4.69-4.60 (m, 1H), 4.53 (d, J=10.5 Hz, 3H), 3.99-3.64 (m, 1H), 3.20-2.89 (m, 1H), 2.82-2.54 (m, 2H), 2.49-2.38 (m, 1H), 1.47 (dd, J=21.8, 4.9 Hz, 3H), 1.30 (d, J=21.5 Hz, 3H), 1.08 (dd, J=6.7, 4.5 Hz, 3H).

### Example 149: Tert-butyl-[3-[(1R,3R)-1-[2-(chloromethyl)-1,3-benzoxazol-5-yl]-3-methyl-1,3,4,9-tetrahydropyrido[3,4-b]indol-2-yl]-2,2-difluoro-propoxy]-diphenyl-silane

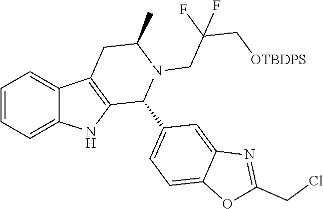

2-(Chloromethyl)-1,3-benzoxazole-5-carbaldehyde (85 mg, 0.43 mmol) and (R)—N-(1-(1H-indol-3-yl)propan-2-yl)-3-((tert-butyldiphenylsilyl)oxy)-2,2-difluoropropan-1-amine (264 mg, 0.52 mmol) were dissolved in DCE (4 mL), and acetic acid (124 μL, 2.2 mmol) was added. The solution was stirred at 120 °C under microwave irradiation for 2 h. The solution was poured into saturated sodium bicarbonate solution and the organic layer was separated. It was then washed with saturated brine solution, dried over sodium sulfate, filtered, and dried in vacuo. The crude material was purified via flash chromatography on silica gel in 0-30% EtOAc in hexanes to give the title product (218 mg, 73%). LCMS: m/z=685.2 [M+H]⁺.

### Example 150: 3-[(1R,3R)-1-[2-(Chloromethyl)-1,3-benzoxazol-5-yl]-3-methyl-1,3,4,9-tetrahydropyrido[3,4-b]indol-2-yl]-2,2-difluoro-propan-1-ol

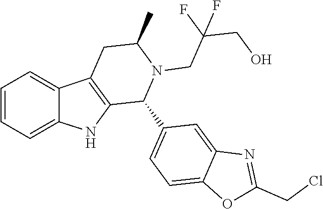 [SMILES: C[C@@H]1Cc2c([nH]c3ccccc23)[C@@H](c2ccc3oc(CCl)nc3c2)N1CC(F)(F)CO]

Tert-butyl-[3-[(1R,3R)-1-[2-(chloromethyl)-1,3-benzoxazol-5-yl]-3-methyl-1,3,4,9-tetrahydropyrido[3,4-b]indol-2-yl]-2,2-difluoro-propoxy]-diphenyl-silane (218 mg, 0.32 mmol) was dissolved in THF (3 mL), and TBAF (1.0 M in THF, 1 mL) was added and stirred for 30 min. The solution was diluted with EtOAc and poured into saturated sodium bicarbonate solution. The organic layers were separated and washed with saturated brine solution. The organic layers were then dried over sodium sulfate, filtered, and dried in vacuo. The crude was purified via flash chromatography on silica gel in 0-30% EtOAc in hexanes to give the title product (86 mg, 61% yield). LCMS: m/z=447.0 [M+H]⁺.

### Example 151: 2,2-Difluoro-3-[(1R,3R)-1-[2-[[3-(fluoromethyl)azetidin-1-yl]methyl]-1,3-benzoxazol-5-yl]-3-methyl-1,3,4,9-tetrahydropyrido[3,4-b]indol-2-yl]propan-1-ol

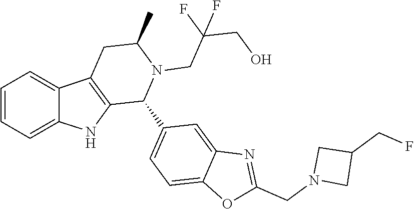 [SMILES: C[C@@H]1Cc2c([nH]c3ccccc23)[C@@H](c2ccc3oc(CN4CC(CF)C4)nc3c2)N1CC(F)(F)CO]

3-[(1R,3R)-1-[2-(Chloromethyl)-1,3-benzoxazol-5-yl]-3-methyl-1,3,4,9-tetrahydropyrido[3,4-b]indol-2-yl]-2,2-difluoro-propan-1-ol (86 mg, 0.19 mmol) and 3-(fluoromethyl)azetidine hydrochloride (36 mg, 0.29 mmol) were dissolved in DMF (2 mL). TEA (108 μL, 0.77 mmol) was added and stirred for 2 h at 40 °C. The reaction was quenched with formic acid (80 μL) and directly purified by HPLC on a Kintetex 5 μm C18 100 Å column (size: 100×30.0 mm; gradient: 5-30% 0.1% formic acid in ACN in 0.1% formic acid in water), then lyophilized to give the title compound as a white solid (28.7 mg, 29% yield). LCMS: m/z=499.6 [M+H]⁺; ¹H NMR (400 MHz, DMSO) δ 10.87 (s, 1H), 7.65 (d, J=8.5 Hz, 1H), 7.52-7.41 (m, 2H), 7.36-7.26 (m, 2H), 7.13-7.07 (m, 1H), 7.07-6.97 (m, 1H), 5.16 (s, 1H), 4.58 (d, J=6.2 Hz, 1H), 4.46 (d, J=6.2 Hz, 1H), 3.83 (s, 2H), 3.76 (td, J=14.1, 4.0 Hz, 2H), 3.43 (td, J=7.7, 1.5 Hz, 3H), 3.27-3.05 (m, 5H), 2.81-2.61 (m, 3H), 2.57 (d, J=10.0 Hz, 1H), 1.08 (d, J=6.7 Hz, 3H).

### Example 152: Tert-butyl-[2,2-difluoro-3-[(1R,3R)-1-[7-fluoro-2-[[3-(fluoromethyl)azetidin-1-yl]methyl]-1,3-benzoxazol-5-yl]-3-methyl-1,3,4,9-tetrahydropyrido[3,4-b]indol-2-yl]propoxy]-diphenyl-silane

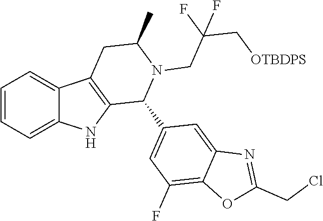

(R)—N-(1-(1H-indol-3-yl)propan-2-yl)-3-((tert-butyldiphenylsilyl)oxy)-2,2-difluoropropan-1-amine (1.20 equiv, 126 mg, 0.248 mmol) and 7-fluoro-2-[[3-(fluoromethyl)azetidin-1-yl]methyl]-1,3-benzoxazole-5-carbaldehyde (1.00 equiv, 55 mg, 0.207 mmol) were dissolved in DCE (3 mL), and acetic acid (5.00 equiv, 0.059 mL, 1.03 mmol) was added. The solution was stirred under microwave irradiation for 2 h at 120 °C. The solution was diluted with DCM, poured into saturated sodium bicarbonate solution, and the organic layers were separated. The organic layers were dried over sodium sulfate, filtered, and dried in vacuo. The crude (210 mg, 135% yield) was used directly in the next step.

### Example 153: 2,2-Difluoro-3-[(1R,3R)-1-[7-fluoro-2-[[3-(fluoromethyl)azetidin-1-yl]methyl]-1,3-benzoxazol-5-yl]-3-methyl-1,3,4,9-tetrahydropyrido[3,4-b]indol-2-yl]propan-1-ol

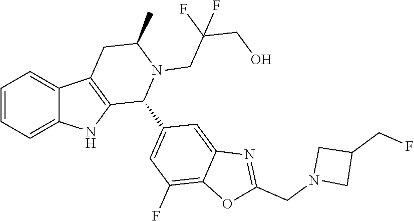 [SMILES: C[C@@H]1Cc2c([nH]c3ccccc23)[C@@H](c2cc(F)c3oc(CN4CC(CF)C4)nc3c2)N1CC(F)(F)CO]

Tert-butyl-[2,2-difluoro-3-[(1R,3R)-1-[7-fluoro-2-[[3-(fluoromethyl)azetidin-1-yl]methyl]-1,3-benzoxazol-5-yl]-3-methyl-1,3,4,9-tetrahydropyrido[3,4-b]indol-2-yl]propoxy]-diphenyl-silane (210 mg, 0.28 mmol, 45% pure) was dissolved in THE (5 mL) and TBAF (2.00 equiv, 0.556 mmol) in THF was added and stirred for 30 min. The solution was diluted in EtOAc and poured into saturated sodium bicarbonate solution. The organic layers were washed with brine solution, dried over sodium sulfate, filtered, and dried in vacuo. The crude was dissolved in DMF (2 mL) and was purified by HPLC on a Kintetex 5 μm C18 100 Å column (size: 100×30.0 mm; gradient: 5-30% 0.1% formic acid in ACN in 0.1% formic acid in water) then lyophilized to give the title compound as a white solid (7.2 mg, 11% yield). LCMS: m/z=517.4 [M+H]⁺. ¹H NMR (400 MHz, DMSO) δ 10.87 (s, 1H), 7.45 (d, J=7.7 Hz, 1H), 7.30 (d, J=7.9 Hz, 1H), 7.08 (ddd, J=8.1, 7.0, 1.3 Hz, 1H), 6.99 (td, J=7.5, 1.1 Hz, 1H), 6.76-6.58 (m, 1H), 6.54-6.43 (m, 1H), 4.93 (s, 1H), 4.69-4.58 (m, 1H), 4.53 (d, J=12.4 Hz, 3H), 3.74 (t, J=13.7 Hz, 2H), 3.24-2.93 (m, 4H), 2.73-2.54 (m, 2H), 1.09 (d, J=6.7 Hz, 3H).

### Example 154: Tert-butyl 3-[(5-bromo-2-hydroxy-phenyl)carbamoyl]azetidine-1-carboxylate

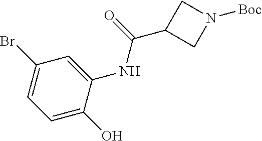 [SMILES: CC(C)(C)OC(=O)N1CC(C(=O)Nc2cc(Br)ccc2O)C1]

2-Amino-4-bromophenol (1.00 equiv, 2.00 g, 10.6 mmol), 1-Boc-azetidine-3-carboxylic acid (1.00 equiv, 2.14 g, 10.6 mmol), and HATU (1.00 equiv, 4.04 g, 10.6 mmol) were dissolved in DMF (16 mL). N,N-Diisopropylethylamine (3.00 equiv, 5.6 mL, 31.9 mmol) was added, and the reaction mixture was stirred for 1.5 h then diluted with EtOAc. The solution was then poured into saturated brine solution, and the organic layers were separated and washed with brine 5 times. The organic layers were then dried over sodium sulfate, filtered, and dried in vacuo. The crude was purified on silica gel in 0-30% EtOAc in DCM to give the desired product (2.96 g, 75% yield). LCMS: m/z=372.2 [M+H]⁺.

### Example 155: Tert-butyl 3-(5-bromo-1,3-benzoxazol-2-yl)azetidine-1-carboxylate

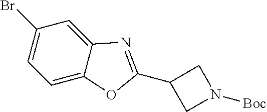 [SMILES: CC(C)(C)OC(=O)N1CC(c2nc3cc(Br)ccc3o2)C1]

Tert-butyl 3-[(5-bromo-2-hydroxy-phenyl)carbamoyl]azetidine-1-carboxylate (1.00 equiv, 2.96 g, 7.97 mmol) and triphenylphosphine (1.10 equiv, 2301 mg, 8.77 mmol) were dissolved in dry THE (15 mL) under nitrogen and stirred at room temperature. DIAD (1.10 equiv, 1.7 mL, 8.77 mmol) was added dropwise and stirred for 1.5 h at room temperature. The solution was then dried in vacuo and purified on silica gel in 0-30% EtOAc in hexanes to give the desired product (2.86 g, 102% yield). LCMS: m/z=354.2 [M+H]⁺.

### Example 156: Tert-butyl 3-(5-cyano-1,3-benzoxazol-2-yl)azetidine-1-carboxylate

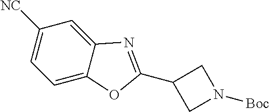 [SMILES: CC(C)(C)OC(=O)N1CC(c2nc3cc(C#N)ccc3o2)C1]

Tert-butyl 3-(5-bromo-1,3-benzoxazol-2-yl)azetidine-1-carboxylate (1.00 equiv, 536 mg, 1.52 mmol), tetrakis(triphenylphosphine)palladium(0) (0.0500 equiv, 88 mg, 0.0759 mmol), and zinc cyanide (1.00 equiv, 178 mg, 1.52 mmol) were dissolved in dry DMF and under an atmosphere of nitrogen and stirred at 80 °C overnight. The solution was cooled, diluted with EtOAc, and poured into water. The organic layer was washed with brine 5 times, dried over sodium sulfate, filtered, and dried in vacuo. The crude was purified on a 24 g silica column in 0-40% EtOAc in hexanes to give the desired product (238 mg, 52% yield). LCMS: m/z=300.3 [M+H]⁺.

### Example 157: Tert-butyl 3-(5-formyl-1,3-benzoxazol-2-yl)azetidine-1-carboxylate

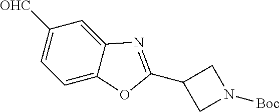 [SMILES: CC(C)(C)OC(=O)N1CC(c2nc3cc(C=O)ccc3o2)C1]

Tert-butyl 3-(5-cyano-1,3-benzoxazol-2-yl)azetidine-1-carboxylate (1.00 equiv, 238 mg, 0.795 mmol) was dissolved in dry THE (8 mL) under nitrogen and cooled to 0 °C. DIBAL (1 M in toluene, 1 mL) was added dropwise and stirred for 1 h. The reaction was then warmed to room temperature and quenched with EtOAc. Rochelle's Salt solution was then added and stirred for 30 min. The organic layers were then separated, dried over sodium sulfate, filtered, and dried in vacuo. The crude was purified on silica gel in 0-40% EtOAc in hexanes to give the title product (38 mg, 16%). LCMS: m/z=303.4 [M+H]⁺.

### Example 158: Tert-butyl3-[5-[(1R,3R)-2-(2-fluoro-2-methyl-propyl)-3-methyl-1,3,4,9-tetrahydropyrido[3,4-b]indol-1-yl]-1,3-benzoxazol-2-yl]azetidine-1-carboxylate

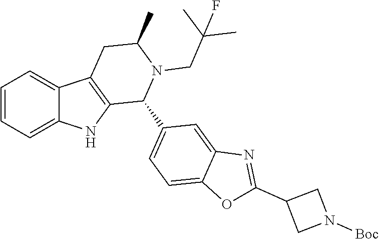 [SMILES: C[C@@H]1Cc2c([nH]c3ccccc23)[C@@H](c2ccc3oc(C4CN(C(=O)OC(C)(C)C)C4)nc3c2)N1CC(C)(C)F]

(R)—N-(1-(1H-indol-3-yl)propan-2-yl)-2-fluoro-2-methylpropan-1-amine (1.00 equiv, 31 mg, 0.126 mmol) and tert-butyl 3-(5-formyl-1,3-benzoxazol-2-yl)azetidine-1-carboxylate (1.00 equiv, 38 mg, 0.126 mmol) were dissolved in DCE (1.5 mL), and acetic acid (5.00 equiv, 0.036 mL, 0.628 mmol) was added. The solution was stirred under microwave irradiation for 2 h at 120 °C. The solution was diluted with DCM, poured into saturated sodium bicarbonate solution, and the organic layers were separated. The organic layers were dried over sodium sulfate, filtered, and dried in vacuo. The crude was purified on silica gel in 0-30% EtOAc in hexanes to give the title product (39 mg, 58% yield). LCMS: m/z=533.6 [M+H]⁺.

### Example 159: 2-(Azetidin-3-yl)-5-[(1R,3R)-2-(2-fluoro-2-methyl-propyl)-3-methyl-1,3,4,9-tetrahydropyrido[3,4-b]indol-1-yl]-1,3-benzoxazole

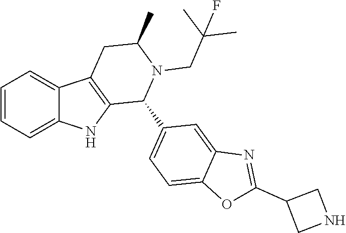 [SMILES: C[C@@H]1Cc2c([nH]c3ccccc23)[C@@H](c2ccc3oc(C4CNC4)nc3c2)N1CC(C)(C)F]

Tert-butyl 3-[5-[(1R,3R)-2-(2-fluoro-2-methyl-propyl)-3-methyl-1,3,4,9-tetrahydropyrido[3,4-b]indol-1-yl]-1,3-benzoxazol-2-yl]azetidine-1-carboxylate (1.00 equiv, 39 mg, 0.0732 mmol) was dissolved in DCM and cooled to 0 °C. TFA (40.0 equiv, 0.23 mL, 2.93 mmol) was added dropwise and stirred for 2 h. The volatiles were removed under reduced pressure then azeotroped with toluene. The crude (29 mg, 92% yield) was used directly in the next step. LCMS: m/z=433.6 [M+H]⁺.

### Example 160: 5-[(1R,3R)-2-(2-fluoro-2-methyl-propyl)-3-methyl-1,3,4,9-tetrahydropyrido[3,4-b]indol-1-yl]-2-[1-(3-fluoropropyl)azetidin-3-yl]-1,3-benzoxazole

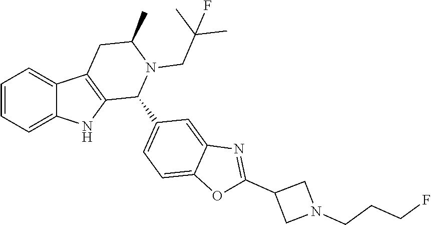 [SMILES: C[C@@H]1Cc2c([nH]c3ccccc23)[C@@H](c2ccc3oc(C4CN(CCCF)C4)nc3c2)N1CC(C)(C)F]

2-(Azetidin-3-yl)-5-[(1R,3R)-2-(2-fluoro-2-methyl-propyl)-3-methyl-1,3,4,9-tetrahydropyrido[3,4-b]indol-1-yl]-1,3-benzoxazole (1.00 equiv, 16 mg, 0.0370 mmol) was dissolved in DMF. 1-Bromo-3-fluoropropane (1.00 equiv, 5.2 mg, 0.0370 mmol) and triethylamine (3.00 equiv, 0.015 mL, 0.111 mmol) were added. The solution was stirred for 1.5 h. The reaction was directly purified by HPLC on a Kintetex 5 μm C18 100 Å column (size: 100×30.0 mm; gradient: 5-30% 0.1% formic acid in ACN in 0.1% formic acid in water), then lyophilized to give the title compound as a white solid (6.1 mg, 28% yield). LCMS: m/z=493.6 [M+H]⁺; ¹H NMR (400 MHz, CDCl₃) δ 7.63 (s, 2H), 7.59-7.50 (m, 1H), 7.46-7.35 (m, 2H), 7.31 (s, 1H), 7.17 (dtd, J=17.5, 7.1, 1.3 Hz, 2H), 5.21 (s, 1H), 4.58 (t, J=5.8 Hz, 1H), 4.46 (t, J=5.8 Hz, 1H), 4.14-3.95 (m, 3H), 3.64 (t, J=7.4 Hz, 2H), 2.85-2.55 (m, 5H), 1.86 (dq, J=26.3, 6.5 Hz, 2H), 1.45 (d, J=4.6 Hz, 3H), 1.36 (s, 2H), 1.29 (d, J=8.6 Hz, 3H), 1.10 (d, J=6.7 Hz, 3H).

### Example 161: 2-[1-[[S-[(1R)-2-(2-Fluoro-2-methyl-propyl)-3,3-dimethyl-4,9-dihydro-1H-pyrido[3,4-b]indol-1-yl]-1H-indol-2-yl]methyl]azetidin-3-yl]propan-2-ol

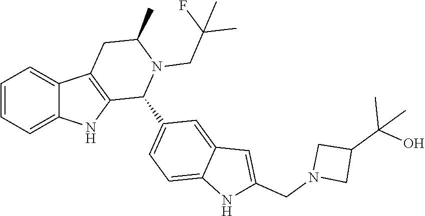 [SMILES: C[C@@H]1Cc2c([nH]c3ccccc23)[C@@H](c2ccc3[nH]c(CN4CC(C(C)(C)O)C4)cc3c2)N1CC(C)(C)F]

5-[(1R)-2-(2-Fluoro-2-methyl-propyl)-3,3-dimethyl-4,9-dihydro-1H-pyrido[3,4-b]indol-1-yl]-1H-indole-2-carbaldehyde (20 mg, 0.05 mmol) was dissolved in DMF (0.5 mL) and acetic acid (6 μL, 0.10 mmol) was added. Separately, 2-(azetidin-3-yl)propan-2-ol (6 mg, 0.05 mmol) was dissolved in DMF (0.5 mL) and TEA (14 μL, 0.10 mmol) was added. These solutions were combined and stirred for 30 minutes. STAB (32 mg, 0.15 mmol) was added and stirred for 2 hours. The reaction was quenched with 1 mL MeOH and directly purified by HPLC on a Kintetex 5 μm C18 100 Å column (size: 100×30.0 mm; gradient: 5-40% 0.1% formic acid in ACN in 0.1% formic acid in water) then lyophilized to give the title compound as an off-white solid (4.2 mg, 17% yield). LCMS: m/z=503.7 [M+H]⁻.

### Example 162: 6-(Difluoromethyl)-2-[[5-[(1R)-2-(2-fluoro-2-methyl-propyl)-3,3-dimethyl-4,9-dihydro-1H-pyrido[3,4-b]indol-1-yl]-1H-indol-2-yl]methyl]-2-azaspiro[3.3]heptan-6-ol

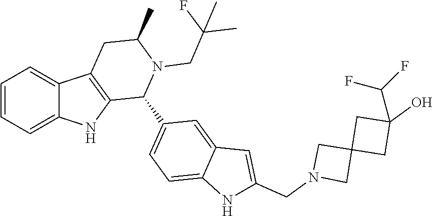 [SMILES: C[C@@H]1Cc2c([nH]c3ccccc23)[C@@H](c2ccc3[nH]c(CN4CC5(C4)CC(O)(C(F)F)C5)cc3c2)N1CC(C)(C)F]

5-[(1R)-2-(2-Fluoro-2-methyl-propyl)-3,3-dimethyl-4,9-dihydro-1H-pyrido[3,4-b]indol-1-yl]-1H-indole-2-carbaldehyde (20 mg, 0.05 mmol) was dissolved in DMF (0.5 mL) and acetic acid (6 μL, 0.10 mmol) was added. Separately, 6-(difluoromethyl)-2-azaspiro[3.3]heptan-6-ol hydrochloride (10 mg, 0.05 mmol) was dissolved in DMF (0.5 mL) and TEA (14 μL, 0.10 mmol) was added. These solutions were combined and stirred for 30 minutes. STAB (32 mg, 0.15 mmol) was added and stirred for 2 hours. The reaction was quenched with 1 mL MeOH and directly purified by HPLC on a Kintetex 5 μm C18 100 Å column (size: 100×30.0 mm; gradient: 5-40% 0.1% formic acid in ACN in 0.1% formic acid in water) then lyophilized to give the title compound as an off-white solid (12.6 mg, 47% yield). LCMS: m/z=551.3 [M+H]⁺.

### Example 163: 1-[[5-[(1R)-2-(2-Fluoro-2-methyl-propyl)-3,3-dimethyl-4,9-dihydro-1H-pyrido[3,4-b]indol-1-yl]-1H-indol-2-yl]methyl]azetidine-3-carbonitrile

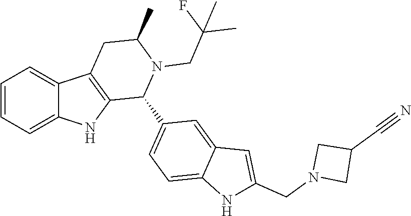 [SMILES: C[C@@H]1Cc2c([nH]c3ccccc23)[C@@H](c2ccc3[nH]c(CN4CC(C#N)C4)cc3c2)N1CC(C)(C)F]

5-[(1R)-2-(2-Fluoro-2-methyl-propyl)-3,3-dimethyl-4,9-dihydro-1H-pyrido[3,4-b]indol-1-yl]-1H-indole-2-carbaldehyde (20 mg, 0.05 mmol) was dissolved in DMF (0.5 mL) and acetic acid (6 μL, 0.10 mmol) was added. Separately, 3-cyanoazetidine hydrochloride (6 mg, 0.05 mmol) was dissolved in DMF (0.5 mL) and TEA (14 μL, 0.10 mmol) was added. These solutions were combined and stirred for 30 minutes. STAB (32 mg, 0.15 mmol) was added and stirred for 2 hours. The reaction was quenched with 1 mL MeOH and directly purified by HPLC on a Kintetex 5 μm C18 100 Å column (size: 100×30.0 mm; gradient: 5-40% 0.1% formic acid in ACN in 0.1% formic acid in water) then lyophilized to give the title compound as an off-white solid (4.9 mg, 21% yield). LCMS: m/z=470.2 [M+H]⁻.

### Example 164: (IR)-2-(2-Fluoro-2-methyl-propyl)-1-[2-[[3-(methoxymethyl)azetidin-1-yl]methyl]-1H-indol-5-yl]-3,3-dimethyl-4,9-dihydro-1H-pyrido[3,4-b]indole

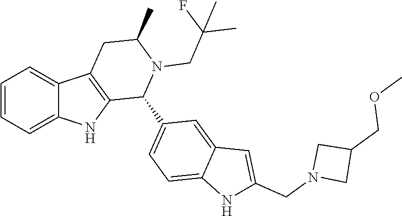 [SMILES: COCC1CN(Cc2cc3cc([C@@H]4c5[nH]c6ccccc6c5C[C@@H](C)N4CC(C)(C)F)ccc3[nH]2)C1]

5-[(1R)-2-(2-Fluoro-2-methyl-propyl)-3,3-dimethyl-4,9-dihydro-1H-pyrido[3,4-b]indol-1-yl]-1H-indole-2-carbaldehyde (20 mg, 0.05 mmol) was dissolved in DMF (0.5 mL) and acetic acid (6 μL, 0.10 mmol) was added. Separately, 3-(methoxymethyl)-azetidine hydrochloride (7 mg, 0.05 mmol) was dissolved in DMF (0.5 mL) and TEA (14 μL, 0.10 mmol) was added. These solutions were combined and stirred for 30 minutes. STAB (32 mg, 0.15 mmol) was added and stirred for 2 hours. The reaction was quenched with 1 mL MeOH and directly purified by HPLC on a Kintetex 5 μm C18 100 Å column (size: 100×30.0 mm; gradient: 5-40% 0.1% formic acid in ACN in 0.1% formic acid in water) then lyophilized to give the title compound as an off-white solid (4.8 mg, 20% yield). LCMS: m/z=489.7 [M+H]⁺.

### Example 165: (1R)-1-[2-[(6-Fluoro-2-azaspiro[3.3]heptan-2-yl)methyl]-1H-indol-5-yl]-2-(2-fluoro-2-methyl-propyl)-3,3-dimethyl-4,9-dihydro-1H-pyrido[3,4-b]indole

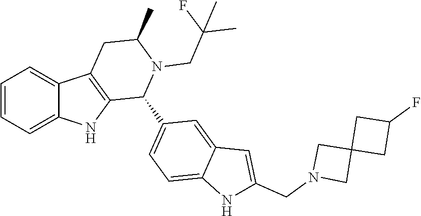 [SMILES: C[C@@H]1Cc2c([nH]c3ccccc23)[C@@H](c2ccc3[nH]c(CN4CC5(CC(F)C5)C4)cc3c2)N1CC(C)(C)F]

5-[(1R)-2-(2-Fluoro-2-methyl-propyl)-3,3-dimethyl-4,9-dihydro-1H-pyrido[3,4-b]indol-1-yl]-1H-indole-2-carbaldehyde (20 mg, 0.05 mmol) was dissolved in DMF (0.5 mL) and acetic acid (6 μL, 0.10 mmol) was added. Separately, 6-fluoro-2-azoniaspiro[3.3]heptane; 2,2,2-trifluoroacetate (11 mg, 0.05 mmol) was dissolved in DMF (0.5 mL) and TEA (14 μL, 0.10 mmol) was added. These solutions were combined and stirred for 30 minutes. STAB (32 mg, 0.15 mmol) was added and stirred for 2 hours. The reaction was quenched with 1 mL MeOH and directly purified by HPLC on a Kintetex 5 μm C18 100 Å column (size: 100×30.0 mm; gradient: 5-40% 0.1% formic acid in ACN in 0.1% formic acid in water) then lyophilized to give the title compound as an off-white solid (7.1 mg, 29% yield). LCMS: m/z=503.6 [M+H]⁺.

### Example 166: 2-[[5-[(1R)-2-(2-Fluoro-2-methyl-propyl)-3,3-dimethyl-4,9-dihydro-1H-pyrido[3,4-b]indol-1-yl]-1H-indol-2-yl]methyl]-2-azaspiro[3.3]heptan-6-ol

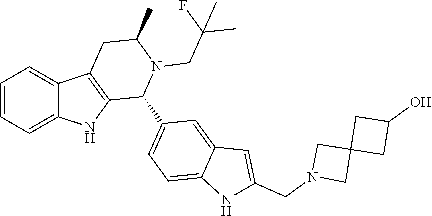 [SMILES: C[C@@H]1Cc2c([nH]c3ccccc23)[C@@H](c2ccc3[nH]c(CN4CC5(CC(O)C5)C4)cc3c2)N1CC(C)(C)F]

5-[(1R)-2-(2-Fluoro-2-methyl-propyl)-3,3-dimethyl-4,9-dihydro-1H-pyrido[3,4-b]indol-1-yl]-1H-indole-2-carbaldehyde (20 mg, 0.05 mmol) was dissolved in DMF (0.5 mL) and acetic acid (6 μL, 0.10 mmol) was added. Separately, 2-azaspiro[3.3]heptan-6-ol hydrochloride (7 mg, 0.05 mmol) was dissolved in DMF (0.5 mL) and TEA (14 μL, 0.10 mmol) was added. These solutions were combined and stirred for 30 minutes. STAB (32 mg, 0.15 mmol) was added and stirred for 2 hours. The reaction was quenched with 1 mL MeOH and directly purified by HPLC on a Kintetex 5 μm C18 100 Å column (size: 100×30.0 mm; gradient: 5-40% 0.1% formic acid in ACN in 0.1% formic acid in water) then lyophilized to give the title compound as an off-white solid (11.6 mg, 47% yield). LCMS: m/z=501.5 [M+H]⁺.

### Example 167: 6-[[5-[(JR)-2-(2-Fluoro-2-methyl-propyl)-3,3-dimethyl-4,9-dihydro-1H-pyrido[3,4-b]indol-1-yl]-1H-indol-2-yl]methyl]-2-oxa-6-azaspiro[3.3]heptane

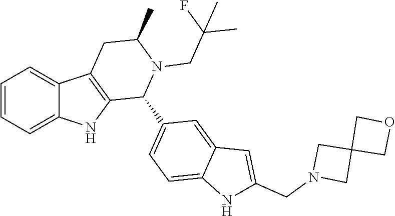 [SMILES: C[C@@H]1Cc2c([nH]c3ccccc23)[C@@H](c2ccc3[nH]c(CN4CC5(COC5)C4)cc3c2)N1CC(C)(C)F]

5-[(1R)-2-(2-Fluoro-2-methyl-propyl)-3,3-dimethyl-4,9-dihydro-1H-pyrido[3,4-b]indol-1-yl]-1H-indole-2-carbaldehyde (20 mg, 0.05 mmol) was dissolved in DMF (0.5 mL) and acetic acid (6 μL, 0.10 mmol) was added. Separately, 2-oxa-6-azaspiro[3.3]heptane; oxalic acid (9 mg, 0.05 mmol) was dissolved in DMF (0.5 mL) and TEA (14 μL, 0.10 mmol) was added. These solutions were combined and stirred for 30 minutes. STAB (32 mg, 0.15 mmol) was added and stirred for 2 hours. The reaction was quenched with 1 mL MeOH and directly purified by HPLC on a Kintetex 5 μm C18 100 Å column (size: 100×30.0 mm; gradient: 5-40% 0.1% formic acid in ACN in 0.1% formic acid in water) then lyophilized to give the title compound as an off-white solid (9.9 mg, 41% yield). LCMS: m/z=487.3 [M+H]⁺.

### Example 168: (1R,3R)-2-(2-Fluoro-2-methylpropyl)-1-(2-((3-fluoroazetidin-1-yl)methyl)-1H-indol-5-yl)-3-methyl-2,3,4,9-tetrahydro-1H-pyrido[3,4-b]indole

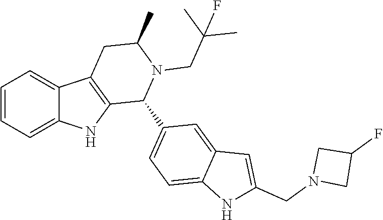 [SMILES: C[C@@H]1Cc2c([nH]c3ccccc23)[C@@H](c2ccc3[nH]c(CN4CC(F)C4)cc3c2)N1CC(C)(C)F]

A solution of 5-((1R,3R)-2-(2-fluoro-2-methylpropyl)-3-methyl-2,3,4,9-tetrahydro-1H-pyrido[3,4-b]indol-1-yl)-1H-indole-2-carbaldehyde (60 mg, 0.15 mmol, 1 equiv) in dichloromethane (1.9 mL) was added to a mixture of 3-fluoroazetidine hydrochloride salt (33 mg, 0.30 mmol, 2 equiv) and triethylamine (0.04 mL, 0.3 mmol, 2 equiv) in dichloromethane (1.9 mL). Acetic acid (0.02 mL, 0.3 mmol, 2 equiv) was added to the resulting reaction mixture which was stirred at room temperature for 1 hour. Sodium triacetoxyborohydride (0.095 g, 0.45 mmol, 3 equiv) was added and reaction was stirred at room temperature for 16 hours. The reaction mixture was diluted with dichloromethane (15 mL) and washed with saturated sodium carbonate (15 mL). The aqueous layer was extracted with dichloromethane (2×15 mL). The combined organic layers were dried over sodium sulfate, filtered and concentrated under reduced pressure onto Celite® (1 g). The residue was purified on a Biotage automated chromatography system (5.5 g, Redigold C18 column), eluting with a gradient of 0 to 100% acetonitrile in water, to give the title compound (46 mg, 65% yield) as an off-white solid. LCMS: m/z=463.3 [M+H]⁺; ¹H NMR (400 MHz, CDCl₃) δ=8.33 (br s, 1H), 7.67-7.43 (m, 2H), 7.31 (s, 1H), 7.29-7.26 (m, 1H), 7.25-7.19 (m, 2H), 7.19-7.07 (m, 2H), 6.25 (s, 1H), 5.20 (quin, J=5.2 Hz, 1H), 5.13-5.02 (m, 1H), 3.78 (s, 2H), 3.70-3.57 (m, 2H), 3.53 (br s, 1H), 3.34-3.17 (m, 2H), 2.86 (br d, J=13.0 Hz, 1H), 2.80-2.54 (m, 3H), 1.43 (br s, 1H), 1.40-1.31 (m, 3H), 1.31-1.17 (m, 2H), 1.08 (d, J=6.7 Hz, 3H).

### Example 169: (1R,3R)-2-(2-Fluoro-2-methylpropyl)-1-(2-((3-methoxyazetidin-1-yl)methyl)-1H-indol-5-yl)-3-methyl-2,3,4,9-tetrahydro-1H-pyrido[3,4-b]indole

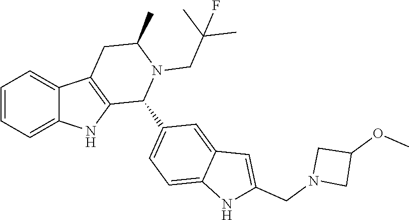 [SMILES: COC1CN(Cc2cc3cc([C@@H]4c5[nH]c6ccccc6c5C[C@@H](C)N4CC(C)(C)F)ccc3[nH]2)C1]

A solution of 5-((1R,3R)-2-(2-fluoro-2-methylpropyl)-3-methyl-2,3,4,9-tetrahydro-1H-pyrido[3,4-b]indol-1-yl)-1H-indole-2-carbaldehyde (60 mg, 0.15 mmol, 1 equiv) in dichloromethane (1.9 mL) was added to a mixture of 3-methoxyazetidine hydrochloride salt (37 mg, 0.30 mmol, 2 equiv) and triethylamine (0.04 mL, 0.3 mmol, 2 equiv) in dichloromethane (1.9 mL). Acetic acid (0.02 mL, 0.3 mmol, 2 equiv) was added to the resulting reaction mixture which was stirred at room temperature for 1 hour. Sodium triacetoxyborohydride (0.095 g, 0.45 mmol, 3 equiv) was added and reaction was stirred at room temperature for 16 hours. The reaction mixture was diluted with dichloromethane (15 mL) and washed with saturated sodium carbonate (15 mL). The aqueous layer was extracted with dichloromethane (2×15 mL). The combined organic layers were dried over sodium sulfate, filtered and concentrated under reduced pressure onto Celite® (1 g). The residue was purified on a Biotage automated chromatography system (5.5 g, Redigold C18 column), eluting with a gradient of 0 to 100% acetonitrile in water, to give the title compound (29 mg, 41% yield) as an off-white solid. LCMS: m/z=475.3 [M+H]⁺; ¹H NMR (400 MHz, CDCl₃) δ=8.44 (br s, 1H), 7.63-7.52 (m, 2H), 7.32 (d, J=4.5 Hz, 1H), 7.25-7.21 (m, 3H), 7.17-7.10 (m, 2H), 6.24 (s, 1H), 5.08 (br s, 1H), 4.05 (quin, J=5.6 Hz, 1H), 3.76 (s, 2H), 3.62-3.52 (m, 3H), 3.26 (s, 3H), 3.04 (dd, J=5.4, 7.3 Hz, 2H), 2.87 (br d, J=14.5 Hz, 1H), 2.77-2.56 (m, 3H), 1.46-1.35 (m, 3H), 1.34-1.28 (m, 3H), 1.09 (d, J=6.7 Hz, 3H).

### Example 170: (1R,3R)-1-(2-((3-Ethylazetidin-1-yl)methyl)-1H-indol-5-yl)-2-(2-fluoro-2-methylpropyl)-3-methyl-2,3,4,9-tetrahydro-1H-pyrido[3,4-b]indole

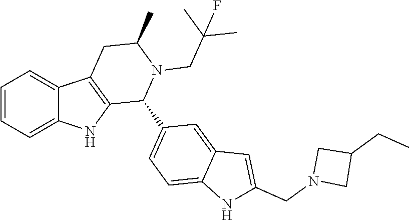 [SMILES: CCC1CN(Cc2cc3cc([C@@H]4c5[nH]c6ccccc6c5C[C@@H](C)N4CC(C)(C)F)ccc3[nH]2)C1]

A solution of 5-((1R,3R)-2-(2-fluoro-2-methylpropyl)-3-methyl-2,3,4,9-tetrahydro-1H-pyrido[3,4-b]indol-1-yl)-1H-indole-2-carbaldehyde (58 mg, 0.14 mmol, 1 equiv) in N,N-dimethylformamide (1.3 mL) was added to a mixture of 3-ethylazetidine trifluoroacetic acid salt (57 mg, 0.29 mmol, 2 equiv) and triethylamine (0.04 mL, 0.3 mmol, 2 equiv) in N,N-dimethylformamide (1.3 mL). Acetic acid (0.02 mL, 0.3 mmol, 2 equiv) was added to the resulting reaction mixture which was stirred at room temperature for 1 hour. Sodium triacetoxyborohydride (0.092 g, 0.43 mmol, 3 equiv) was added and reaction was stirred at room temperature for 3 days. The reaction mixture was diluted with ethyl acetate (15 mL) and washed with saturated sodium bicarbonate (15 mL). The organic layer was sequentially washed with water (2×15 mL) and saturated brine (15 mL). The organic layer was dried over sodium sulfate, filtered and concentrated under reduced pressure onto Celite® (0.8 g). The residue was purified on a Biotage automated chromatography system (11 g, Biotage 50 μm KP-amino-d column), eluting with a gradient of 0 to 100% ethyl acetate in hexanes to give the title compound (50 mg, ~85% purity). The resulting residue was loaded onto Celite® (1 g) purified on a Biotage automated chromatography system (5.5 g Redigold C18 column), eluting with a gradient of 0 to 100% acetonitrile in water, to give the title compound (21 mg, 31% yield) a pale-yellow solid. LCMS: m/z=473.3 [M+H]; ¹H NMR (400 MHz, CDCl₃) δ=8.53 (br s, 1H), 7.64-7.44 (m, 2H), 7.32-7.27 (m, 1H), 7.24-7.20 (m, 3H), 7.17-7.07 (m, 2H), 6.21 (s, 1H), 5.05 (br s, 1H), 3.69 (s, 2H), 3.55 (br s, 1H), 3.43 (t, J=7.5 Hz, 2H), 2.93-2.74 (m, 3H), 2.74-2.62 (m, 1H), 2.62-2.54 (m, 2H), 2.38 (spt, J=7.2 Hz, 1H), 1.53 (quin, J=7.4 Hz, 2H), 1.40 (br s, 1H), 1.38-1.30 (m, 3H), 1.27 (s, 2H), 1.07 (d, J=6.6 Hz, 3H), 0.81 (t, J=7.4 Hz, 3H).

### Example 171: 2-(1-((5-((1R,3R)-2-(2-Fluoro-2-methylpropyl)-3-methyl-2,3,4,9-tetrahydro-1H-pyrido[3,4-b]indol-1-yl)-1H-indol-2-yl methyl)azetidin-3-yl)acetonitrile

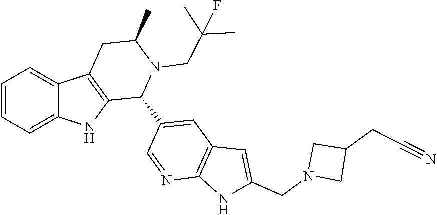 [SMILES: C[C@@H]1Cc2c([nH]c3ccccc23)[C@@H](c2cnc3[nH]c(CN4CC(CC#N)C4)cc3c2)N1CC(C)(C)F]

A solution of 5-((1R,3R)-2-(2-fluoro-2-methylpropyl)-3-methyl-2,3,4,9-tetrahydro-1H-pyrido[3,4-b]indol-1-yl)-1H-indole-2-carbaldehyde (60 mg, 0.15 mmol, 1 equiv) in dichloromethane (1.9 mL) was added to a mixture of 2-(azetidin-3-yl)acetonitrile hydrochloride salt (40. mg, 0.30 mmol, 2 equiv) and triethylamine (0.04 mL, 0.3 mmol, 2 equiv) in dichloromethane (1.9 mL). Acetic acid (0.02 mL, 0.3 mmol, 2 equiv) was added to the resulting reaction mixture which was stirred at room temperature for 1 hour. Sodium triacetoxyborohydride (0.095 g, 0.45 mmol, 3 equiv) was added and reaction was stirred at room temperature for 16 hours. The reaction mixture was diluted with dichloromethane (15 mL) and washed with saturated sodium carbonate (15 mL). The aqueous layer was extracted with dichloromethane (2×15 mL). The combined organic layers were dried over sodium sulfate, filtered and concentrated under reduced pressure onto Celite® (1 g). The residue was purified on a Biotage automated chromatography system (5.5 g, Redigold C18 column), eluting with a gradient of 0 to 100% acetonitrile in water, to give the title compound (42 mg, 58% yield) as an off-white solid. LCMS: m/z=484.3 [M+H]⁺; ¹H NMR (400 MHz, CDCl₃) δ=8.30 (br s, 1H), 7.65-7.45 (m, 2H), 7.31 (s, 1H), 7.25-7.20 (m, 3H), 7.17-7.09 (m, 2H), 6.23 (s, 1H), 5.07 (br s, 1H), 3.72 (s, 2H), 3.53 (br s, 1H), 3.44 (t, J=7.6 Hz, 2H), 3.08-3.00 (m, 2H), 2.89-2.69 (m, 3H), 2.67-2.55 (m, 4H), 1.42 (br s, 1H), 1.40-1.31 (m, 3H), 1.30-1.17 (m, 2H), 1.08 (d, J=6.6 Hz, 3H).

### Example 172: (1R,3R)-2-(2-Fluoro-2-methylpropyl)-1-(2-((3-isopropylazetidin-1-yl)methyl)-1H-indol-5-yl)-3-methyl-2,3,4,9-tetrahydro-1H-pyrido[3,4-b]indole

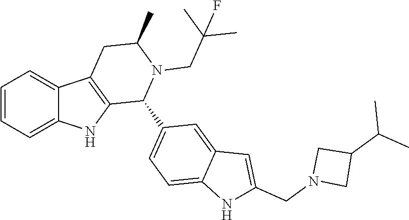 [SMILES: CC(C)C1CN(Cc2cc3cc([C@@H]4c5[nH]c6ccccc6c5C[C@@H](C)N4CC(C)(C)F)ccc3[nH]2)C1]

A solution of 5-((1R,3R)-2-(2-fluoro-2-methylpropyl)-3-methyl-2,3,4,9-tetrahydro-1H-pyrido[3,4-b]indol-1-yl)-1H-indole-2-carbaldehyde (60 mg, 0.15 mmol, 1 equiv) in dichloromethane (1.9 mL) was added to a mixture of 3-isopropylazetidine hydrochloride salt (40. mg, 0.30 mmol, 2 equiv) and triethylamine (0.04 mL, 0.3 mmol, 2 equiv) in dichloromethane (1.9 mL). Acetic acid (0.02 mL, 0.3 mmol, 2 equiv) was added to the resulting reaction mixture which was stirred at room temperature for 1 hour. Sodium triacetoxyborohydride (0.095 g, 0.45 mmol, 3 equiv) was added and reaction was stirred at room temperature for 16 hours. The reaction mixture was diluted with dichloromethane (15 mL) and washed with saturated sodium carbonate (15 mL). The aqueous layer was extracted with dichloromethane (2×15 mL). The combined organic layers were dried over sodium sulfate, filtered and concentrated under reduced pressure onto Celite® (1 g). The residue was purified on a Biotage automated chromatography system (5.5 g, Redigold C18 column), eluting with a gradient of 0 to 100% acetonitrile in water, to give the title compound (35 mg, 48% yield) as an off-white solid. LCMS: m/z=487.3 [M+H]⁺; ¹H NMR (400 MHz, CDCl₃) δ=8.61 (br s, 1H), 7.63-7.46 (m, 2H), 7.33-7.27 (m, 1H), 7.23-7.08 (m, 5H), 6.21 (s, 1H), 5.05 (br s, 1H), 3.67 (s, 2H), 3.54 (br s, 1H), 3.47-3.40 (m, 2H), 2.89-2.75 (m, 3H), 2.72-2.54 (m, 3H), 2.18-2.08 (m, 1H), 1.72-1.59 (m, 1H), 1.44-1.37 (m, 1H), 1.37-1.30 (m, 3H), 1.29-1.13 (m, 2H), 1.07 (d, J=6.7 Hz, 3H), 0.79 (d, J=6.6 Hz, 6H).

### Example 173: (1R,3R)-1-(2-((3-(Difluoromethyl)azetidin-1-yl)methyl)-1H-indol-5-yl)-2-(2-fluoro-2-methylpropyl)-3-methyl-2,3,4,9-tetrahydro-1H-pyrido[3,4-b]indole

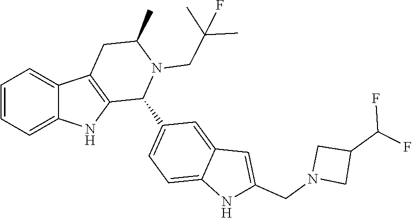 [SMILES: C[C@@H]1Cc2c([nH]c3ccccc23)[C@@H](c2ccc3[nH]c(CN4CC(C(F)F)C4)cc3c2)N1CC(C)(C)F]

A solution of 5-((1R,3R)-2-(2-fluoro-2-methylpropyl)-3-methyl-2,3,4,9-tetrahydro-1H-pyrido[3,4-b]indol-1-yl)-1H-indole-2-carbaldehyde (60 mg, 0.15 mmol, 1 equiv) in dichloromethane (1.9 mL) was added to a mixture of 3-(difluoromethyl)azetidine hydrochloride salt (43 mg, 0.30 mmol, 2 equiv) and triethylamine (0.04 mL, 0.3 mmol, 2 equiv) in dichloromethane (1.9 mL). Acetic acid (0.02 mL, 0.3 mmol, 2 equiv) was added to the resulting reaction mixture which was stirred at room temperature for 1 hour. Sodium triacetoxyborohydride (0.095 g, 0.45 mmol, 3 equiv) was added and reaction was stirred at room temperature for 16 hours. The reaction mixture was diluted with dichloromethane (15 mL) and washed with saturated sodium carbonate (15 mL). The aqueous layer was extracted with dichloromethane (2×15 mL). The combined organic layers were dried over sodium sulfate, filtered and concentrated under reduced pressure onto Celite® (1 g). The residue was purified on a Biotage automated chromatography system (5.5 g, Redigold C18 column), eluting with a gradient of 0 to 100% acetonitrile in water, to give the title compound (45 mg, 61% yield) as an off-white solid. LCMS: m/z=495.3 [M+H]⁺; ¹H NMR (400 MHz, CDCl₃) δ=8.31 (br s, 1H), 7.61-7.48 (m, 2H), 7.31 (s, 1H), 7.26-7.20 (m, 3H), 7.17-7.09 (m, 2H), 6.24 (s, 1H), 6.14-5.81 (m, 1H), 5.08 (br s, 1H), 3.71 (s, 2H), 3.54 (br s, 1H), 3.41-3.32 (m, 2H), 3.26-3.19 (m, 2H), 2.92-2.76 (m, 2H), 2.73-2.55 (m, 3H), 1.43 (br s, 1H), 1.40-1.32 (m, 3H), 1.31-1.24 (m, 2H), 1.08 (d, J=6.6 Hz, 3H).

### Example 174: 1-((5-((1R,3R)-2-(2-Fluoro-2-methylpropyl)-3-methyl-2,3,4,9-tetrahydro-1H-pyrido[3,4-b]indol-1-yl)-1H-indol-2-yl)methyl)-N,N-dimethylazetidin-3-amine

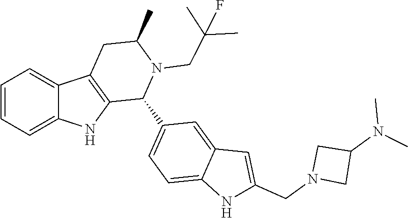 [SMILES: C[C@@H]1Cc2c([nH]c3ccccc23)[C@@H](c2ccc3[nH]c(CN4CC(N(C)C)C4)cc3c2)N1CC(C)(C)F]

A solution of 5-((1R,3R)-2-(2-fluoro-2-methylpropyl)-3-methyl-2,3,4,9-tetrahydro-1H-pyrido[3,4-b]indol-1-yl)-1H-indole-2-carbaldehyde (60 mg, 0.15 mmol, 1 equiv) in dichloromethane (1.9 mL) was added to a mixture of N,N-dimethylazetidin-3-amine dihydrochloride salt (52 mg, 0.30 mmol, 2 equiv) and triethylamine (0.08 mL, 0.6 mmol, 4 equiv) in dichloromethane (1.9 mL). Acetic acid (0.02 mL, 0.3 mmol, 2 equiv) was added to the resulting reaction mixture which was stirred at room temperature for 1 hour. Sodium triacetoxyborohydride (0.095 g, 0.45 mmol, 3 equiv) was added and reaction was stirred at room temperature for 16 hours. The reaction mixture was diluted with dichloromethane (15 mL) and washed with saturated sodium carbonate (15 mL). The aqueous layer was extracted with dichloromethane (2×15 mL). The combined organic layers were dried over sodium sulfate, filtered and concentrated under reduced pressure onto Celite® (1 g). The residue was purified on a Biotage automated chromatography system (5.5 g, Redigold C18 column), eluting with a gradient of 0 to 100% acetonitrile in water to give the title compound (50 mg, 69% yield) as an off-white solid. LCMS: m/z=488.3 [M+H]⁺; ¹H NMR (400 MHz, CDCl₃) δ=8.60 (s, 1H), 7.83 (br s, 1H), 7.54 (d, J=6.5 Hz, 1H), 7.33-7.26 (m, 1H), 7.23-7.07 (m, 5H), 6.21 (s, 1H), 5.06 (br s, 1H), 3.69 (s, 2H), 3.52 (br s, 1H), 3.48-3.37 (m, 2H), 2.98-2.79 (m, 4H), 2.75-2.53 (m, 3H), 2.08 (s, 6H), 1.43-1.22 (m, 6H), 1.06 (d, J=6.7 Hz, 3H).

### Example 175: (1R,3R)-1-(2-((3,3-Dimethylazetidin-1-yl)methyl)-1H-indol-5-yl)-2-(2-fluoro-2-methylpropyl)-3-methyl-2,3,4,9-tetrahydro-1H-pyrido[3,4-b]indole

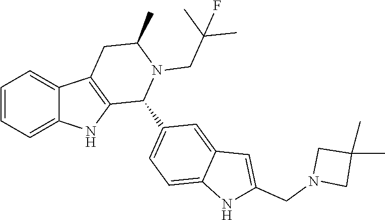 [SMILES: C[C@@H]1Cc2c([nH]c3ccccc23)[C@@H](c2ccc3[nH]c(CN4CC(C)(C)C4)cc3c2)N1CC(C)(C)F]

A solution of 3,3-dimethylazetidine hydrochloride (37 mg, 0.297 mmol, 2 equiv) and triethylamine (41 μL, 0.297 mmol, 2 equiv) in dichloromethane (2 mL) was added to a solution of 5-[(1R,3R)-2-(2-fluoro-2-methyl-propyl)-3-methyl-1,3,4,9-tetrahydropyrido[3,4-b]indol-1-yl]-1H-indole-2-carbaldehyde (60 mg, 0.149 mmol, 1 equiv) in dichloromethane (2 mL). Acetic acid (17 μL, 0.297 mmol, 2 equiv) was added and the resulting mixture was stirred at room temperature for 1 hour. Sodium triacetoxyborohydride (95 mg, 0.446 mmol, 3 equiv) was added in one portion, and the reaction was stirred at room temperature for 14 hours. The reaction was quenched with methanol (1 mL) and the volatiles were evaporated under reduced pressure. The residue was purified on an Interchim automated chromatography system (RediSep Rf GOLD 15 g HP C18 column), eluting with a gradient of 0 to 100% acetonitrile in water, to give the title compound as an off-white solid. LCMS: m/z=473.3 [M+H]⁺; ¹H NMR (400 MHz, CDCl₃) δ=8.56 (br s, 1H), 7.63-7.47 (m, 2H), 7.27-7.07 (m, 7H), 6.20 (s, 1H), 5.04 (br s, 1H), 3.69 (s, 21H), 3.53 (br s, 1H), 2.99 (s, 4H), 2.93-2.78 (m, 1H), 2.76-2.52 (m, 3H), 2.35 (s, 1H), 1.40 (br s, 1H), 1.37-1.29 (m, 3H), 1.23-1.17 (m, 6H), 1.09-1.03 (m, 3H)

### Example 176: (1R,3R)-2-(2-Fluoro-2-methylpropyl)-1-(2-((3-methoxy-3-methylazetidin-1-yl)methyl)-1H-indol-5-yl)-3-methyl-2,3,4,9-tetrahydro-1H-pyrido[3,4-b]indole

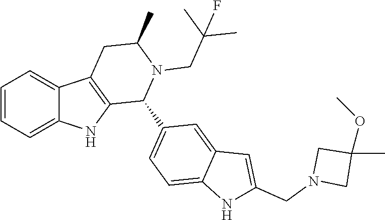 [SMILES: COC1(C)CN(Cc2cc3cc([C@@H]4c5[nH]c6ccccc6c5C[C@@H](C)N4CC(C)(C)F)ccc3[nH]2)C1]

A solution of 3-methoxy-3-methylazetidine hydrochloride (41 mg, 0.297 mmol, 2 equiv) and triethylamine (41 μL, 0.297 mmol, 2 equiv) in dichloromethane (2 mL) was added to a solution of 5-[(1R,3R)-2-(2-fluoro-2-methyl-propyl)-3-methyl-1,3,4,9-tetrahydropyrido[3,4-b]indol-1-yl]-1H-indole-2-carbaldehyde (60 mg, 0.149 mmol, 1 equiv) in dichloromethane (2 mL). Acetic acid (17 μL, 0.297 mmol, 2 equiv) was added and the resulting mixture was stirred at room temperature for 1 hour. Sodium triacetoxyborohydride (95 mg, 0.446 mmol, 3 equiv) was added in one portion and the reaction was stirred at room temperature for 4 hours. The reaction was quenched with methanol (1 mL) and the volatiles were evaporated under reduced pressure. The residue was purified on an Interchim automated chromatography system (RediSep Rf GOLD 5.5 g HP C18 column), eluting with a gradient of 0 to 100% acetonitrile in water, to give the title compound as a light yellow solid. LCMS: m/z=489.3 [M+H]⁺; ¹H NMR (400 MHz, CDCl₆) δ=8.41 (br s, 1H), 7.62-7.47 (m, 21-H), 731 (s, 1H), 7.26-7.22 (m, 4H), 7.16-7.09 (m, 2H), 6.23 (s, 1H), 5.07 (br s, 1H), 3.77 (s, 2H), 3.55 (br s, 1H), 3.22-3.12 (m, 6H), 2.86 (br d, J=13.2 Hz, 1H), 2.77-2.54 (m, 3H), 1.47 (s, 3H), 1.42 (br s, 1H), 1.39-1.24 (m, 5H), 1.08 (d, J=6.6 Hz, 3H).

### Example 177: 1R,3R)-1-(2-((3,3-Difluoroazetidin-1-yl)methyl)-1H-indol-5-yl)-2-(2-fluoro-2-methylpropyl)-3-methyl-2,3,4,9-tetrahydro-1H-pyrido[3,4-b]indole

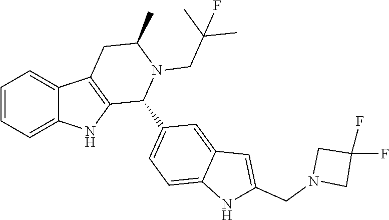 [SMILES: C[C@@H]1Cc2c([nH]c3ccccc23)[C@@H](c2ccc3[nH]c(CN4CC(F)(F)C4)cc3c2)N1CC(C)(C)F]

A solution of 3,3-difluoroazetidine hydrochloride (39 mg, 0.297 mmol, 2 equiv) and triethylamine (41 μL, 0.297 mmol, 2 equiv) in dichloromethane (2 mL) was added to a solution of 5-[(1R,3R)-2-(2-fluoro-2-methyl-propyl)-3-methyl-1,3,4,9-tetrahydropyrido[3,4-b]indol-1-yl]-1H-indole-2-carbaldehyde (60 mg, 0.149 mmol, 1 equiv) in dichloromethane (2 mL). Acetic acid (17 μL, 0.297 mmol, 2 equiv) was added and the resulting mixture was stirred at room temperature for 1 hour. Sodium triacetoxyborohydride (143 mg, 0.669 mmol, 4.5 equiv) was added in one portion and the reaction was stirred at room temperature for 48 hours. The reaction was quenched with methanol (1 mL) and the volatiles were evaporated under reduced pressure. The residue was purified on an Interchim automated chromatography system (RediSep Rf GOLD 5.5 g HP C18 column), eluting with a gradient of 0 to 100% acetonitrile in water to give the title compound as a yellow solid. LCMS: m/z=481.3 [M+H]⁺; ¹H NMR (400 MHz, CDCl₆) δ=8.34 (br s, 1H), 7.61-7.49 (m, 2H), 7.34-7.27 (m, 2H), 7.23 (br d, J=6.1 Hz, 1H), 7.18-7.10 (m, 2H), 6.27 (s, 1H), 5.10 (br s, 1H), 3.85 (s, 2H), 3.62 (t, J=12.0 Hz, 4H), 3.57-3.47 (m, 1H), 2.85 (br d, J=13.1 Hz, 1H), 2.78-2.52 (m, 3H), 1.52-1.26 (m, 7H), 1.08 (d, J=6.6 Hz, 3H).

### Example 178: (1R,3R)-2-(2-Fluoro-2-methylpropyl)-1-(2-((3-fluoro-3-methylazetidin-1-yl)methyl)-1H-indol-5-yl)-3-methyl-2,3,4,9-tetrahydro-1H-pyrido[3,4-b]indole

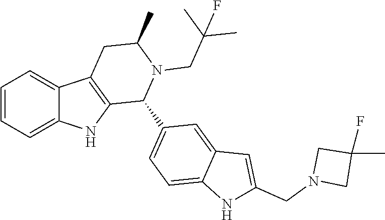 [SMILES: C[C@@H]1Cc2c([nH]c3ccccc23)[C@@H](c2ccc3[nH]c(CN4CC(C)(F)C4)cc3c2)N1CC(C)(C)F]

A solution of 3-fluoro-3-methylazetidine hydrochloride (37 mg, 0.297 mmol, 2 equiv) and triethylamine (41 μL, 0.297 mmol, 2 equiv) in dichloromethane (2 mL) was added to a solution of 5-[(1R,3R)-2-(2-fluoro-2-methyl-propyl)-3-methyl-1,3,4,9-tetrahydropyrido[3,4-b]indol-1-yl]-1H-indole-2-carbaldehyde (60 mg, 0.149 mmol, 1 equiv) in dichloromethane (2 mL). Acetic acid (17 μL, 0.297 mmol, 2 equiv) was added and the resulting mixture was stirred at room temperature for 1 hour. Sodium triacetoxyborohydride (143 mg, 0.669 mmol, 4.5 equiv) was added in one portion and the reaction was stirred at room temperature for 24 hours. The reaction was quenched with methanol (1 mL) and the volatiles were evaporated under reduced pressure. The residue was purified on an Interchim automated chromatography system (RediSep Rf GOLD 5.5 g HP C18 column), eluting with a gradient of 0 to 100% acetonitrile in water, to give the title compound as a yellow solid. LCMS: m/z=477.3 [M+H]⁺; ¹H NMR (400 MHz, CDCl₃) δ=8.35 (br s, 1H), 7.62-7.48 (m, 2H), 7.32 (s, 1H), 7.26-7.20 (m, 3H), 7.17-7.09 (m, 2H), 6.25 (s, 1H), 5.08 (br s, 1H), 3.78 (s, 2H), 3.54 (br s, 1H), 3.39-3.27 (m, 4H), 2.86 (br d, J=13.0 Hz, 1H), 2.78-2.55 (m, 3H), 1.64-1.55 (m, 3H), 1.46-1.25 (m, 6H), 1.08 (d, J=6.7 Hz, 3H).

### Example 179: (5-((1R,3R)-3-Methyl-2-(2,2,2-trifluoroethyl)-2,3,4,9-tetrahydro-1H-pyrido[3,4-b]indol-1-yl)-1H-indol-2-yl)methanol

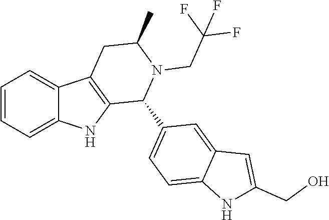 [SMILES: C[C@@H]1Cc2c([nH]c3ccccc23)[C@@H](c2ccc3[nH]c(CO)cc3c2)N1CC(F)(F)F]

A mixture of 2-(hydroxymethyl)-1H-indole-5-carbaldehyde (200 mg, 1.1 mmol, 1.0 equiv), (R)-1-(1H-indol-3-yl)-N-(2,2,2-trifluoroethyl)propan-2-amine (290 mg, 1.1 mmol, 1.0 equiv), and glacial acetic acid (0.13 mL, 2.3 mmol, 2.0 equiv) in 1,2-dichloroethane (2.5 mL) and dioxane (2.5 mL) was stirred in a microwave apparatus (110 °C, 150 W, fixed power) for 6 h. The reaction mixture was cooled to room temperature, diluted with ethyl acetate (20 mL) and washed with a saturated sodium carbonate solution (4×5 mL). The organic layer was dried over sodium sulfate (3 g), filtered and concentrated under reduced pressure. The crude product was diluted with dichloromethane (10 mL) and absorbed onto silica gel (5 g) under reduced pressure. The product was purified on a Biotage automated chromatography system (Biotage spherical silica gel column 25 g, 20 mm), eluting with a gradient of 0 to 10% methanol in dichloromethane to give the title compound (160 mg, 34% yield) as off-white solid. LCMS: m/z=414 [M+H]⁻.

### Example 180: 5-((1R,3R)-3-Methyl-2-(2,2,2-trifluoroethyl)-2,3,4,9-tetrahydro-1H-pyrido[3,4-b]indol-1-yl)-1H-indole-2-carbaldehyde

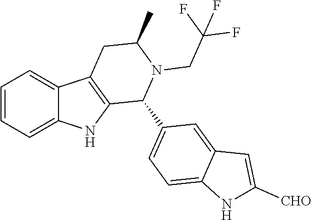 [SMILES: C[C@@H]1Cc2c([nH]c3ccccc23)[C@@H](c2ccc3[nH]c(C=O)cc3c2)N1CC(F)(F)F]

A mixture of (5-((1R,3R)-3-methyl-2-(2,2,2-trifluoroethyl)-2,3,4,9-tetrahydro-1H-pyrido[3,4-b]indol-1-yl)-1H-indol-2-yl)methanol (160 mg, 0.39 mmol, 1.0 equiv) and activated manganese dioxide (85%, 336 mg, 3.9 mmol, 10.0 equiv) in dichloromethane (3.9 mL) was stirred for 22 h. The reaction mixture was filtered through a pad of Celite® (10 g), which was washed with dichloromethane (3×10 mL). The filtrate was concentrated under reduced pressure. The crude product was diluted with dichloromethane (5 mL) and absorbed onto silica gel (2 g) under reduced pressure. The product was purified on a Biotage automated chromatography system (Biotage spherical silica gel column 5 g, 20 mm), eluting with a gradient of 0 to 50% ethyl acetate in hexanes to give the title compound (43 mg, 36% yield) as pale-yellow oil. LCMS: m/z=412 [M+H]⁺.

### Example 181: (1R,3R)-1-(2-((3-(Fluoromethyl)azetidin-1-yl)methyl)-1H-indol-5-yl)-3-methyl-2-(2,2,2-trifluoroethyl)-2,3,4,9-tetrahydro-1H-pyrido[3,4-b]indole

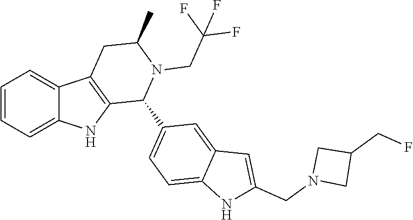 [SMILES: C[C@@H]1Cc2c([nH]c3ccccc23)[C@@H](c2ccc3[nH]c(CN4CC(CF)C4)cc3c2)N1CC(F)(F)F]

A mixture of 5-((1R,3R)-3-methyl-2-(2,2,2-trifluoroethyl)-2,3,4,9-tetrahydro-1H-pyrido[3,4-b]indol-1-yl)-1H-indole-2-carbaldehyde (40 mg, 0.1 mmol, 1.0 equiv), 3-(fluoromethyl)azetidine hydrochloride (24 mg, 0.2 mmol, 2.0 equiv), 1.0 M triethylamine in dichloromethane (0.19 mL, 0.19 mmol, 2.0 equiv), and 1.0 M acetic acid in dichloromethane (0.19 mL, 0.19 mmol, 2.0 equiv) in dichloromethane (2 mL) was stirred for 1 hour. Sodium triacetoxyborohydride (62 mg, 0.3 mmol, 3.0 equiv) was added and the reaction mixture was stirred for 18 hours. Methanol (1 mL) was added and reaction mixture was stirred for 15 minutes. The mixture was concentrated under reduced pressure, diluted with dichloromethane (2 ml) and absorbed onto Celite® (lg) under reduced pressure. The product was purified on a Biotage automated chromatography system (RediSep Gold C18 column 5.5 g), eluting with a gradient of 0 to 80% acetonitrile in water. The product was lyophilized to give the title compound (23 mg, 49% yield) as white solid. ¹H NMR (400 MHz, CDCl₃) δ=8.43 (br s, 1H), 7.65-7.44 (m, 2H), 7.32-7.27 (m, 2H), 7.26-7.05 (m, 4H), 6.27-6.21 (m, 1H), 5.05 (s, 1H), 4.57 (d, J=5.7 Hz, 1H), 4.45 (d, J=5.7 Hz, 1H), 3.72 (s, 2H), 3.51-3.33 (m, 3H), 3.24 (qd, J=9.5, 15.4 Hz, 1H), 3.15-2.98 (m, 3H), 2.95-2.74 (m, 2H), 2.58 (dd, J=8.1, 14.9 Hz, 1H), 1.14 (d, J=6.7 Hz, 3H); LCMS: m/z=485.2 [M+H]⁺.

### Example 182: 5-Bromo-2-(bromomethyl)-1,3-benzothiazole

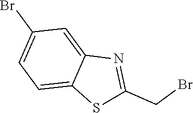 [SMILES: BrCc1nc2cc(Br)ccc2s1]

5-Bromo-2-methylbenzothiazole (1.00 equiv, 2.00 g, 8.77 mmol), NBS (1.10 equiv, 1717 mg, 9.64 mmol), and AIBN (0.250 equiv, 360 mg, 2.19 mmol) were dissolved and stirred in carbon tetrachloride (20 mL) overnight at 90 °C. The reaction was then cooled to room temperature, and the insoluble material was filtered off. The solution was then dried under reduced pressure and purified via flash chromatography on silica gel in 0-20% EtOAc in hexanes to give the title product (590 mg, 22% yield). LCMS: m/z=307.9 [M+H]⁺.

### Example 183: 5-Bromo-2-[[3-(fluoromethyl)azetidin-1-yl]methyl]-1,3-benzothiazole

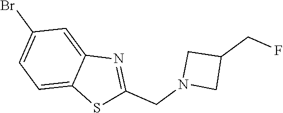 [SMILES: FCC1CN(Cc2nc3cc(Br)ccc3s2)C1]

5-Bromo-2-(bromomethyl)-1,3-benzothiazole (1.00 equiv, 590 mg, 1.92 mmol) and 3-(fluoromethyl)azetidine hydrochloride (1.20 equiv, 290 mg, 2.31 mmol) were dissolved in DMF (5 mL), and triethylamine (3.00 equiv, 0.80 mL, 5.77 mmol) was added. The solution was warmed to 50 °C and stirred for 1 h. The reaction was then cooled and poured into brine solution. The organic layers were separated and washed with saturated brine solution 5 times. They were then dried over sodium sulfate, filtered, and dried in vacuo. The crude was purified on silica gel in 0-10% MeOH in DCM to give the title product (473 mg, 78% yield). LCMS: m/z=307.9 [M+H]⁺.

### Example 184: 2-[[3-(Fluoromethyl)azetidin-1-yl]methyl]-1,3-benzothiazole-5-carbonitrile

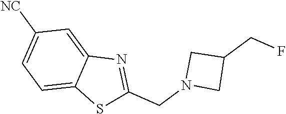 [SMILES: N#Cc1ccc2sc(CN3CC(CF)C3)nc2c1]

5-Bromo-2-[[3-(fluoromethyl)azetidin-1-yl]methyl]-1,3-benzothiazole (1.00 equiv, 454 mg, 1.44 mmol), tetrakis(triphenylphosphine)palladium(0) (0.0500 equiv, 83 mg, 0.0720 mmol), and zinc cyanide (1.00 equiv, 169 mg, 1.44 mmol) were dissolved in dry DMF (5 mL) under an atmosphere of nitrogen and stirred at 80 °C overnight. The solution was cooled, diluted with EtOAc, and poured into water. The organic layer was washed with brine 5 times, dried over sodium sulfate, filtered, and dried in vacuo. The crude was purified on a 24 g silica column in 20-60% EtOAc in DCM to give the desired product (347 mg, 92.2% yield). LCMS: m/z=262.3 [M+H]⁺.

### Example 185: 2-[[3-(Fluoromethyl)azetidin-1-yl]methyl]-1,3-benzothiazole-5-carbaldehyde

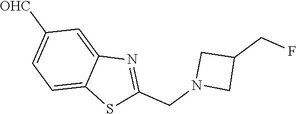 [SMILES: O=Cc1ccc2sc(CN3CC(CF)C3)nc2c1]

2-[[3-(Fluoromethyl)azetidin-1-yl]methyl]-1,3-benzothiazole-5-carbonitrile (1.00 equiv, 347 mg, 1.33 mmol) was dissolved in dry THE (4 mL) under nitrogen and cooled to 0 °C. DIBAL (1 M in toluene, 1.46 mL) was added and stirred for 1 h. The reaction was quenched with EtOAc, then Rochelle's Salt solution was added and stirred for 30 min. The organic layers were separated, dried over sodium sulfate, filtered, and dried in vacuo. The crude was purified on silica gel in 0-10% MeOH (2% TEA) in DCM to give the desired product (170 mg, 48 yield). LCMS: m/z=265.3 [M+H]⁺.

### Example 186: 2-[[3-(Fluoromethyl)azetidin-1-yl]methyl]-5-[(1R,3R)-2-(2-fluoro-2-methyl-propyl)-3-methyl-1,3,4,9-tetrahydropyrido[3,4-b]indol-1-yl]-1,3-benzothiazole

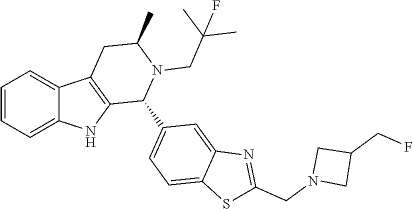 [SMILES: C[C@@H]1Cc2c([nH]c3ccccc23)[C@@H](c2ccc3sc(CN4CC(CF)C4)nc3c2)N1CC(C)(C)F]

(R)—N-(1-(1H-indol-3-yl)propan-2-yl)-2-fluoro-2-methylpropan-1-amine (1.10 equiv, 176 mg, 0.707 mmol) and 2-[[3-(fluoromethyl)azetidin-1-yl]methyl]-1,3-benzothiazole-5-carbaldehyde (1.00 equiv, 170 mg, 0.643 mmol) were dissolved in DCE. Acetic acid (5.00 equiv, 0.18 mL, 3.22 mmol) was added, and the reaction mixture stirred at 110 °C under microwave irradiation for 2 h. The solution was cooled then diluted with DCM and poured into saturated sodium bicarbonate solution. The organic layers were separated, dried over sodium sulfate, filtered, and dried in vacuo. The crude was purified on silica gel in 0-10% MeOH (2% TEA) in DCM to give the desired product at about 90% purity. 45 mg of this material was dissolved in DMF (2 mL) and purified by HPLC on a Kintetex 5 μm C18 100 Å column (size: 100×30.0 mm; gradient: 5-30% 0.1% formic acid in ACN in 0.1% formic acid in water) and lyophilized to give the title product (24.9 mg, 55% yield, >99% purity). LCMS: m/z=495.7 [M+H]⁺; ¹H NMR (400 MHz, CDCl₃) δ 8.07 (s, 1H), 7.86-7.78 (m, 2H), 7.54 (dd, J=20.7, 7.9 Hz, 2H), 7.17 (dt, J=15.6, 7.1 Hz, 2H), 4.62 (d, J=5.6 Hz, 1H), 4.50 (d, J=5.6 Hz, 1H), 4.07 (s, 2H), 3.64 (t, J=7.7 Hz, 2H), 3.34 (t, J=7.0 Hz, 2H), 2.84-2.55 (m, 4H), 1.49 (d, J=21.7 Hz, 2H), 1.37 (s, 2H), 1.30 (d, J=11.9 Hz, 3H), 1.11 (d, J=6.7 Hz, 3H).

### Example 187: 2-[[3-(Fluoromethyl)azetidin-1-yl]methyl]-5-[(1R,3R)-2-(2-fluoro-2-methyl-propyl)-3-methyl-1,3,4,9-tetrahydropyrido[3,4-b]indol-1-yl]-1,3-benzothiazole 1-oxide

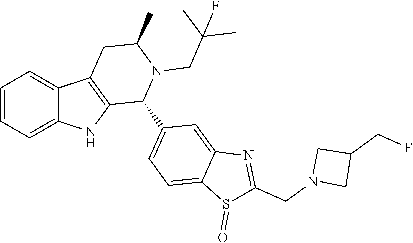 [SMILES: C[C@@H]1Cc2c([nH]c3ccccc23)[C@@H](c2ccc3c(c2)N=C(CN2CC(CF)C2)S3=O)N1CC(C)(C)F]

2-[[3-(Fluoromethyl)azetidin-1-yl]methyl]-5-[(1R,3R)-2-(2-fluoro-2-methyl-propyl)-3-methyl-1,3,4,9-tetrahydropyrido[3,4-b]indol-1-yl]-1,3-benzothiazole (33 mg, 0.07 mmol) and MCPBA (17 mg, 0.10 mmol) were dissolved in DCM and stirred at room temperature for 1 h. The reaction was poured into saturated sodium bicarbonate solution and extracted with DCM. The organic layers were separated and dried over sodium sulfate, filtered, and concentrated in vacuo. The crude was dissolved in DMF (2 mL) and purified by HPLC on a Kintetex 5 μm C18 100 Å column (size: 100×30.0 mm; gradient: 5-30% 0.1% formic acid in ACN in 0.1% formic acid in water) and lyophilized to give the title product (11.9 mg, 35% yield). LCMS: m/z=511.2 [M+H]⁺; ¹H NMR (400 MHz, CDCl₃) δ 7.90-7.81 (m, 2H), 7.66 (d, J=9.0 Hz, 1H), 7.58 (d, J=7.5 Hz, 1H), 7.33 (d, J=7.8 Hz, 1H), 7.18 (dt, J=18.9, 7.1 Hz, 2H), 5.29 (s, 1H), 5.04 (d, J=15.6 Hz, 2H), 4.76-4.65 (m, 2H), 4.65-4.47 (m, 3H), 4.36 (dd, J=18.0, 7.9 Hz, 1H), 2.85-2.54 (m, 4H), 1.47 (d, J=19.6 Hz, 3H), 1.35 (d, J=21.4 Hz, 3H), 1.28 (s, 1H), 1.11 (d, J=6.7 Hz, 3H).

### Example 188: tert-Butyl 3-(5-formylindazol-1-yl)azetidine-1-carboxylate and tert-butyl 3-(5-formylindazol-2-yl)azetidine-1-carboxylate

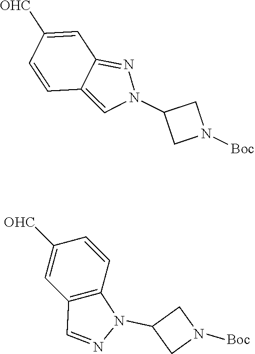 [SMILES: CC(C)(C)OC(=O)N1CC(n2cc3ccc(C=O)cc3n2)C1 ; CC(C)(C)OC(=O)N1CC(n2ncc3cc(C=O)ccc32)C1]

To a solution of 1H-indazole-5-carbaldehyde (1.00 equiv, 797 mg, 5.45 mmol) in DMF (5 mL) in a water bath was added sodium hydride (1.50 equiv, 327 mg, 8.18 mmol). After 45 min, 2-methyl-2-propanyl 3-iodo-1-azetidinecarboxylate (1.30 equiv, 2.2 mL, 7.09 mmol) was added. The reaction was stirred for 15 min in a water bath before heating at 50 °C overnight. The reaction was quenched with water (50 mL) and extracted with EtOAc (50 mL). The organic layer was washed with 5% LiCl (50 mL×2). The organic layers were dried over sodium sulfate and purified via silica gel (40 g) eluting with 25% hexanes:EtOAc to afford tert-butyl 3-(5-formylindazol-1-yl)azetidine-1-carboxylate as a beige solid (844 mg, 2.80 mmol, 51.4% yield) and with 35% hexanes:EtOAc to afford tert-butyl 3-(5-formylindazol-2-yl)azetidine-1-carboxylate as a beige solid (514 mg, 1.71 mmol, 31.3% yield).

N1: LCMS: m/z=302.2 [M+H]⁺; ¹H NMR (400 MHz, DMSO) δ 10.05 (s, 1H), 8.53-8.45 (m, 2H), 7.91 (dd, J=8.8, 1.4 Hz, 1H), 7.83 (d, J=8.9 Hz, 1H), 5.77 (td, J=8.1, 4.0 Hz, 1H), 4.41 (t, J=8.5 Hz, 2H), 4.27 (s, 2H), 1.44 (s, 9H).

N2: LCMS: m/z=302.1 [M+H]⁺; ¹H NMR (400 MHz, DMSO) δ 9.99 (d, J=0.8 Hz, 1H), 8.89 (d, J=0.9 Hz, 1H), 8.48 (dd, J=1.5, 0.9 Hz, 1H), 7.89-7.75 (m, 1H), 7.71 (dd, J=9.0, 1.5 Hz, 1H), 5.60 (tt, J=7.9, 5.1 Hz, 1H), 4.43 (t, J=8.5 Hz, 2H), 4.30 (s, 2H), 1.44 (s, 8H).

### Example 189: tert-Butyl 3-[5-[(1R,3R)-2-[3-[tert-butyl(diphenyl)silyl]oxy-2,2-difluoro-propyl]-3-methyl-1,3,4,9-tetrahydropyrido[3,4-b]indol-1-yl]indazol-1-yl]azetidine-1-carboxylate

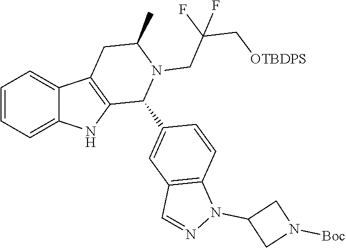

A solution of (R)—N-(1-(1H-indol-3-yl)propan-2-yl)-3-((tert-butyldiphenylsilyl)oxy)-2,2-difluoropropan-1-amine (1.20 equiv, 456 mg, 0.900 mmol), tert-butyl 3-(5-formylindazol-1-yl)azetidine-1-carboxylate (1.00 equiv, 226 mg, 0.750 mmol), and acetic acid (3.00 equiv, 0.13 mL, 2.25 mmol) in DCE (1.5 mL) was heated at 125 °C for 3 h. DCM (20 mL) was added, and the organic layer was washed with water (10 mL) and brine (5 mL). The organic layers were dried over sodium sulfate and purified via silica gel (40 g) eluting with 60% hexanes:EtOAc to afford the title compound (579 mg, 0.733 mmol, 97.7% yield) as a white solid. LCMS: m/z=790.3 [M+H]⁺.

### Example 190: tert-butyl-[2,2-difluoro-3-[(1R,3R)-1-[1-(azetidin-3-yl)indazol-S-yl]-3-methyl-1,3,4,9-tetrahydropyrido[3,4-b]indol-2-yl]propoxy]-diphenyl-silane; 2,2,2-trifluoroacetic acid

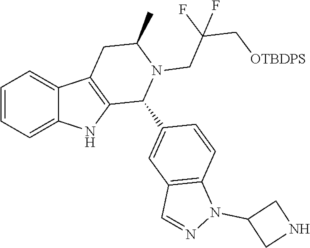

To a solution of tert-butyl 3-[5-[(1R,3R)-2-[3-[tert-butyl(diphenyl)silyl]oxy-2,2-difluoro-propyl]-3-methyl-1,3,4,9-tetrahydropyrido[3,4-b]indol-1-yl]indazol-1-yl]azetidine-1-carboxylate (1.00 equiv, 468 mg, 0.592 mmol) in DCM (4.5 mL) at RT was added trifluoroacetic acid (11.0 equiv, 0.50 mL, 6.49 mmol). After 5 h, the reaction was azeotroped with DCE (5 mL×2) to afford the title compound (639 mg, 0.696 mmol, 118% yield) as a solid. LCMS: m/z=690.4 [M+H]⁺.

### Example 191: 2,2-difluoro-3-[(1R,3R)-1-[1-(azetidin-3-yl)indazol-S-yl]-3-methyl-1,3,4,9-tetrahydropyrido[3,4-b]indol-2-yl]propan-1-ol

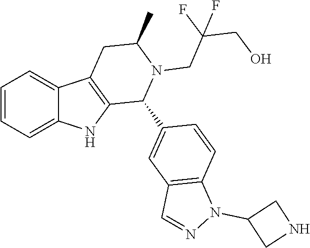 [SMILES: C[C@@H]1Cc2c([nH]c3ccccc23)[C@@H](c2ccc3c(cnn3C3CNC3)c2)N1CC(F)(F)CO]

To a solution of tert-butyl-[2,2-difluoro-3-[(1R,3R)-1-[1-(azetidin-3-yl)indazol-5-yl]-3-methyl-1,3,4,9-tetrahydropyrido[3,4-b]indol-2-yl]propoxy]-diphenyl-silane; 2,2,2-trifluoroacetic acid (1.00 equiv, 52 mg, 0.0566 mmol) in THF (0.5 mL) was added tetra-n-butylammonium fluoride (5.30 equiv, 0.30 mL, 0.300 mmol). After 1 h, the reaction was left in at −28 °C overnight. The reaction was then stirred for 3 h at RT. The solvent was removed, NaHCO₃ (aq, 10 mL) was added and the reaction was extracted with DCM (10 mL×2). The organic layers were dried over sodium sulfate, concentrated and purified via silica gel (12 g) eluting at 16% DCM/MeOH (2% NH₄OH) to the title compound as a beige solid (179 mg, 0.396 mmol, 62.0% yield). LCMS: m/z=452.2 [M+H]⁺.

### Example 192: 2,2-difluoro-3-[(1R,3R)-3-methyl-1-[1-(1-propylazetidin-3-yl)indazol-5-yl]-1,3,4,9-tetrahydropyrido[3,4-b]indol-2-yl]propan-1-ol

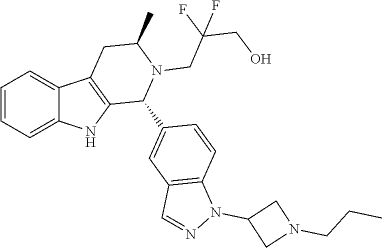 [SMILES: CCCN1CC(n2ncc3cc([C@@H]4c5[nH]c6ccccc6c5C[C@@H](C)N4CC(F)(F)CO)ccc32)C1]

A solution of 2,2-difluoro-3-[(1R,3R)-1-[1-(azetidin-3-yl)indazol-5-yl]-3-methyl-1,3,4,9-tetrahydropyrido[3,4-b]indol-2-yl]propan-1-ol (1.00 equiv, 30 mg, 0.0664 mmol), propanal (3.00 equiv, 0.015 mL, 0.199 mmol) and acetic acid (2.00 equiv, 0.0076 mL, 0.133 mmol) in methanol (1 mL) was stirred for 5 min before addition of sodium triacetoxyborohydride (4.00 equiv, 56 mg, 0.266 mmol). After stirring for 1 h, the reaction was directly purified by HPLC on a Kintetex 5 μm C18 100 Å column (size: 100×30.0 mm, gradient: 5-50% 0.1% formic acid in ACN in 0.1% formic acid in water) then lyophilized to give the title compound as a light yellow solid (11 mg, 0.0213 mmol, 32.0% yield). LCMS: m/z=494.4 [M+H]⁺; ¹H NMR (400 MHz, DMSO) δ 10.83 (s, 1H), 8.28 (s, 1H), 8.06 (d, J=0.8 Hz, 1H), 7.74 (d, J=8.8 Hz, 1H), 7.52-7.44 (m, 2H), 7.33-7.25 (m, 2H), 7.08 (ddd, J=8.1, 7.0, 1.3 Hz, 1H), 7.00 (td, J=7.5, 1.1 Hz, 1H), 5.39 (p, J=7.1 Hz, 1H), 5.15 (s, 1H), 3.79 (dq, J=9.7, 3.9 Hz, 3H), 3.45 (dt, J=20.3, 7.2 Hz, 2H), 3.32-3.04 (m, 1H), 2.79-2.60 (m, 2H), 2.60-2.51 (m, 1H), 2.48 (d, J=7.1 Hz, 2H), 1.36 (h, J=7.3 Hz, 2H), 1.06 (d, J=6.8 Hz, 3H), 0.89 (t, J=7.4 Hz, 3H).

### Example 193: 2,2-difluoro-3-[(1R,3R)-1-[1-[1-(3-fluoropropyl)azetidin-3-yl]indazol-5-yl]-3-methyl-1,3,4,9-tetrahydropyrido[3,4-b]indol-2-yl]propan-1-ol

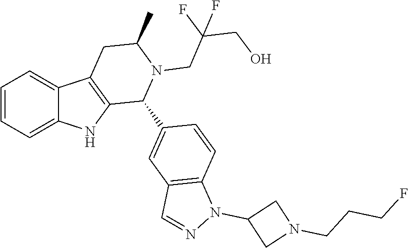 [SMILES: C[C@@H]1Cc2c([nH]c3ccccc23)[C@@H](c2ccc3c(cnn3C3CN(CCCF)C3)c2)N1CC(F)(F)CO]

To a solution of 2,2-difluoro-3-[(1R,3R)-1-[1-(azetidin-3-yl)indazol-5-yl]-3-methyl-1,3,4,9-tetrahydropyrido[3,4-b]indol-2-yl]propan-1-ol (1.00 equiv, 25 mg, 0.0554 mmol) and 1-bromo-3-fluoropropane (1.00 equiv, 0.0051 mL, 0.0554 mmol) in DMF (1 mL) was added triethylamine (3.00 equiv, 0.023 mL, 0.166 mmol). After stirring overnight, more 1-bromo-3-fluoropropane (0.2 equiv, 0.001 mL, 0.011 mmol) was added and the reaction heated at 50 °C. After 1 h, more 1-bromo-3-fluoropropane (0.2 equiv, 0.001 mL, 0.011 mmol) was added. After stirring for 1 h more at 50 °C, the reaction was directly purified by HPLC on a Kintetex 5 μm C18 100 Å column (size: 100×30.0 mm; gradient: 5-50% 0.1% formic acid in ACN in 0.1% formic acid in water) then lyophilized to give the title compound as a white solid (5.8 mg, 0.0113 mmol, 20.5% yield). LCMS: m/z=512.4 [M+H]⁺; ¹H NMR (400 MHz, DMSO) δ 10.83 (s, 1H), 8.28 (s, 1H), 8.07 (s, 1H), 7.74 (d, J=8.8 Hz, 1H), 7.52-7.41 (m, 2H), 7.33-7.25 (m, 2H), 7.12-7.04 (m, 1H), 7.04-6.94 (m, 1H), 5.40 (p, J=7.1 Hz, 1H), 5.15 (s, 1H), 4.57 (t, J=6.0 Hz, 1H), 4.45 (t, J=6.1 Hz, 1H), 3.86-3.61 (m, 3H), 3.48 (dt, J=20.2, 7.1 Hz, 2H), 3.29-3.08 (m, 1H), 1.73 (dp, J=25.7, 60.5 Hz, 2H), 1.06 (d, J=6.8 Hz, 3H).

### Example 194: tert-butyl 3-[5-[(1R,3R)-2-[3-[tert-butyl(diphenyl)silyl]oxy-2,2-difluoro-propyl]-3-methyl-1,3,4,9-tetrahydropyrido[3,4-b]indol-1-yl]indazol-2-yl]azetidine-1-carboxylate

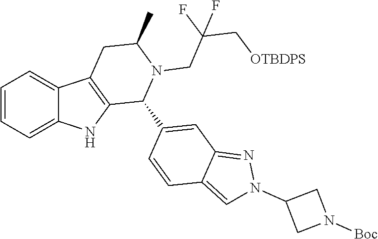

A solution of (R)—N-(1-(1H-indol-3-yl)propan-2-yl)-3-((tert-butyldiphenylsilyl)oxy)-2,2-difluoropropan-1-amine (1.50 equiv, 760 mg, 1.50 mmol), acetic acid (3.00 equiv, 0.17 mL, 3.00 mmol) and tert-butyl 3-(5-formylindazol-2-yl)azetidine-1-carboxylate (1.00 equiv, 301 mg, 1.00 mmol) in DCE (2 mL) was heated at 90 °C. After 2.5 h, the temperature was raised to 100 °C for 2 h. The reaction was further heated at 120 °C for 1.5 h. DCM (20 mL) was added, and the organic layers washed with water (10 mL) and brine (5 mL). The organic layers were dried over sodium sulfate and purified via silica gel (40 g) eluting with 60% hexanes:EtOAc to afford the title compound as a clear, thick oil (998 mg, 1.26 mmol, 126% yield). LCMS: m/z=790.5 [M+H]⁺.

### Example 195: tert-butyl-[2,2-difluoro-3-[(1R,3R)-1-[2-(azetidin-3-yl)indazol-5-yl]-3-methyl-1,3,4,9-tetrahydropyrido[3,4-b]indol-2-yl]propoxy]-diphenyl-silane

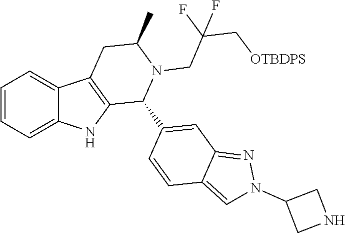

To a solution of tert-butyl 3-[5-[(1R,3R)-2-[3-[tert-butyl(diphenyl)silyl]oxy-2,2-difluoro-propyl]-3-methyl-1,3,4,9-tetrahydropyrido[3,4-b]indol-1-yl]indazol-2-yl]azetidine-1-carboxylate (1.00 equiv, 358 mg, 0.453 mmol) in DCM (3.6 mL) was added trifluoroacetic acid (11.5 equiv, 0.40 mL, 5.19 mmol). After stirring overnight, sodium carbonate (aq, 5 mL) was added, and the reaction extracted with DCM (5 mL×2) which was dried over sodium sulfate and concentrated to afford the title compound as a white solid (280 mg, 0.406 mmol, 89.6% yield). LCMS: m/z=690.7 [M+H]⁺.

### Example 196: 2,2-difluoro-3-[(1R,3R)-1-[2-(azetidin-3-yl)indazol-S-yl]-3-methyl-1,3,4,9-tetrahydropyrido[3,4-b]indol-2-yl]propan-1-ol

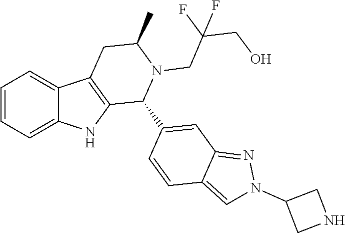 [SMILES: C[C@@H]1Cc2c([nH]c3ccccc23)[C@@H](c2ccc3cn(C4CNC4)nc3c2)N1CC(F)(F)CO]

To a solution of tert-butyl-[2,2-difluoro-3-[(1R,3R)-1-[2-(azetidin-3-yl)indazol-5-yl]-3-methyl-1,3,4,9-tetrahydropyrido[3,4-b]indol-2-yl]propoxy]-diphenyl-silane (1.00 equiv, 140 mg, 0.203 mmol) in THF (1 mL) was added tetra-n-butylammonium fluoride (1.97 equiv, 0.40 mL, 0.400 mmol) at room temperature. After 1 h, the reaction was concentrated and purified via silica gel (12 g) eluting at 16% DCM/MeOH (2% NH₄OH) to afford the title compound as a white solid (52 mg, 0.115 mmol, 56.8% yield). LCMS: m/z=452.3 [M+H]⁺.

### Example 197: 2,2-difluoro-3-[(1R,3R)-1-[2-(1-ethylazetidin-3-yl)indazol-S-yl]-3-methyl-1,3,4,9-tetrahydropyrido[3,4-b]indol-2-yl]propan-1-ol

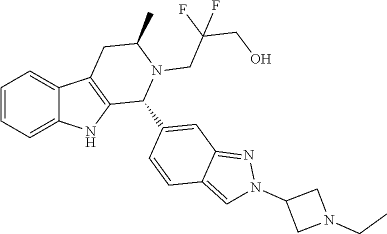 [SMILES: CCN1CC(n2cc3ccc([C@@H]4c5[nH]c6ccccc6c5C[C@@H](C)N4CC(F)(F)CO)cc3n2)C1]

To a solution of 2,2-difluoro-3-[(1R,3R)-1-[2-(azetidin-3-yl)indazol-5-yl]-3-methyl-1,3,4,9-tetrahydropyrido[3,4-b]indol-2-yl]propan-1-ol (1.00 equiv, 26 mg, 0.0576 mmol) in methanol (1 mL) was added acetaldehyde (3.00 equiv, 0.0098 mL, 0.173 mmol) and acetic acid (2.00 equiv, 0.0066 mL, 0.115 mmol). After 30 min, sodium triacetoxyborohydride (3.00 equiv, 37 mg, 0.173 mmol) was added. After stirring overnight, the reaction was directly purified by HPLC on a Kintetex 5 μm C18 100 Å column (size: 100×30.0 mm; gradient: 5-30% 0.1% formic acid in ACN in 0.1% formic acid in water) then lyophilized to give the title compound as a white solid (8.4 mg, 0.0161 mmol, 28.0% yield). LCMS: m/z=480.2 [M+H]⁺.

### Example 198: 2,2-difluoro-3-[(1R,3R)-3-methyl-1-[2-(1-propylazetidin-3-yl)indazol-5-yl]-1,3,4,9-tetrahydropyrido[3,4-b]indol-2-yl]propan-1-ol

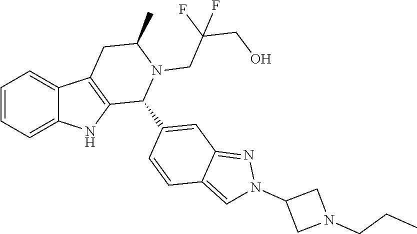 [SMILES: CCCN1CC(n2cc3ccc([C@@H]4c5[nH]c6ccccc6c5C[C@@H](C)N4CC(F)(F)CO)cc3n2)C1]

To a solution of 2,2-difluoro-3-[(1R,3R)-1-[2-(azetidin-3-yl)indazol-5-yl]-3-methyl-1,3,4,9-tetrahydropyrido[3,4-b]indol-2-yl]propan-1-ol (1.00 equiv, 26 mg, 0.0576 mmol) in methanol (1 mL) was added propionaldehyde (3.00 equiv, 0.012 mL, 0.173 mmol) and acetic acid (2.00 equiv, 0.0066 mL, 0.115 mmol). After 30 min, sodium triacetoxyborohydride (3.00 equiv, 37 mg, 0.173 mmol) was added. After stirring overnight, more propionaldehyde (3.00 equiv, 0.012 mL, 0.173 mmol) and sodium triacetoxyborohydride (3.00 equiv, 37 mg, 0.173 mmol) were added. After stirring overnight, the reaction was directly purified by HPLC on a Kintetex 5 μm C18 100 Å column (size: 100×30.0 mm; gradient: 5-30% 0.1% formic acid in ACN in 0.1% formic acid in water) then lyophilized to give the title compound as a white solid (8.1 mg, 0.0164 mmol, 28.5% yield). LCMS: m/z=494.4 [M+H]⁺; ¹H NMR (400 MHz, DMSO) δ 10.83 (s, 1H), 8.38 (d, J=1.0 Hz, 1H), 8.32 (s, 1H), 7.62 (d, J=9.0 Hz, 1H), 7.50-7.36 (m, 2H), 7.34-7.26 (m, 1H), 7.12 (s, 1H), 7.07 (ddd, J=8.1, 7.0, 1.3 Hz, 1H), 7.00 (td, J=7.4, 1.1 Hz, 1H), 5.22 (p, J=6.9 Hz, 1H), 5.09 (s, 1H), 3.77 (s, 1H), 3.74 (d, J=7.7 Hz, 2H), 3.44 (td, J=7.8, 6.4 Hz, 2H), 2.78-2.59 (m, 2H), 2.56 (d, J=9.8 Hz, 1H), 2.46 (t, J=7.1 Hz, 2H), 1.33 (h, J=7.1 Hz, 2H), 1.06 (d, J=6.9 Hz, 3H), 0.87 (t, J=7.4 Hz, 3H).

### Example 199: Ethyl 5-cyano-1-(p-tolylsulfonyl)indole-2-carboxylate

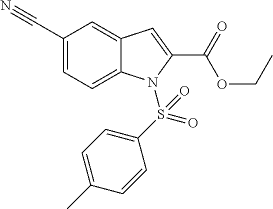 [SMILES: CCOC(=O)c1cc2cc(C#N)ccc2n1S(=O)(=O)c1ccc(C)cc1]

5-Cyanoindole-2-carboxylic acid ethyl ester (1.00 equiv, 3.41 g, 15.9 mmol) was dissolved in DMF (25 mL) and cooled to 0 °C. Sodium hydride (1.24 equiv, 458 mg, 19.1 mmol) in mineral oil (60%) was added and stirred for 30 min. p-Toluenesulfonyl chloride (1.05 equiv, 3.19 g, 16.7 mmol) was added and stirred for 2 h while warming to room temperature. The reaction was quenched with 1N HCL (40 mL) and extracted with EtOAc (2×50 mL). The combined organic fractions were washed with saturated brine solution (5×30 mL), dried over sodium sulfate, filtered, and dried in-vacuo. The crude was purified by flash chromatography in 0-22% EtOAc in hexanes to give the title product as a white solid (4.75 g, 12.89 mmol, 81% yield). LCMS: m/z=369.1 [M+H]⁺.

### Example 200: 2-(Hydroxymethyl)-1-(p-tolylsulfonyl)indole-5-carbaldehyde

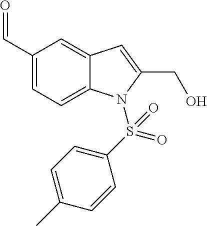 [SMILES: Cc1ccc(S(=O)(=O)n2c(CO)cc3cc(C=O)ccc32)cc1]

Ethyl 5-cyano-1-(p-tolylsulfonyl)indole-2-carboxylate (1.00 equiv, 2.50 g, 6.79 mmol) was dissolved in anhydrous THE (34 mL) and cooled to 0 °C. DIBAL (1N in THF, 4.00 equiv, 27.1 mL, 27.1 mmol) was added slowly and stirred for 1 h. The reaction was diluted and quenched with EtOAc and saturated Rochelle's salt was added and stirred for 1 h. The layers were separated and the aqueous phase was extracted with EtOAc (1×30 mL). The combined organic fractions were washed with saturated brine solution (1×30 mL), dried over sodium sulfate, filtered, and dried in vacuo. The crude was purified by flash chromatography in 0-25% EtOAc in hexanes to give the title product as an off-white solid. (1.1 g, 3.34 mmol, 49% yield). LCMS: m/z=330.1 [M+H]⁺.

### Example 201: [5-[(1R,3R)-3-Methyl-2-(2,2,3,3-tetrafluoropropyl)-1,3,4,9-tetrahydropyrido[3,4-b]indol-1-yl]-1-(p-tolylsulfonyl)indol-2-yl]methanol

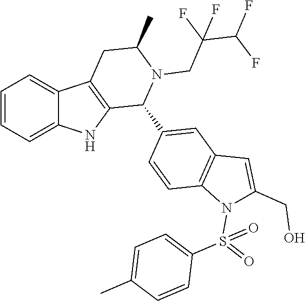 [SMILES: Cc1ccc(S(=O)(=O)n2c(CO)cc3cc([C@@H]4c5[nH]c6ccccc6c5C[C@@H](C)N4CC(F)(F)C(F)F)ccc32)cc1]

2-(Hydroxymethyl)-1-(p-tolylsulfonyl)indole-5-carbaldehyde (1.00 equiv, 227 mg, 0.69 mmol) and 2,2,3,3-tetrafluoro-N-[(1R)-2-(1H-indol-3-yl)-1-methyl-ethyl]propan-1-amine (1.10 equiv, 0.219 mg, 0.76 mmol) were dissolved in dry toluene (2.5 mL) and acetic acid (4.9 equiv, 197 μL, 3.4 mmol) was added and stirred at 100 °C for 4 h. The reaction was allowed to cool to room temperature and was quenched with saturated sodium bicarbonate solution and extracted with EtOAc (2×5 mL). The combined organic fractions were dried over sodium sulfate, filtered, and dried in-vacuo. The crude was purified by flash chromatography in 0-25% EtOAc in hexanes to give the title compound (256 mg, 0.43 mmol, 62% yield). LCMS: m/z=600.1 [M+H]⁺.

### Example 202: [5-[(1R,3R)-3-methyl-2-(2,2,3,3,3-pentafluoropropyl)-1,3,4,9-tetrahydropyrido[3,4-b]indol-1yl]-1-(p-tolylsulfonyl)indol-2-yl]methanol

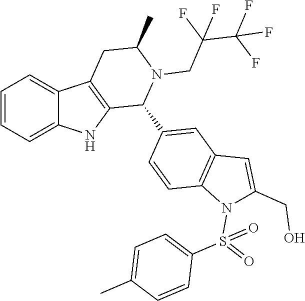 [SMILES: Cc1ccc(S(=O)(=O)n2c(CO)cc3cc([C@@H]4c5[nH]c6ccccc6c5C[C@@H](C)N4CC(F)(F)C(F)(F)F)ccc32)cc1]

2-(Hydroxymethyl)-1-(p-tolylsulfonyl)indole-5-carbaldehyde (1.00 equiv, 239 mg, 0.73 mmol) and (R)—N-(1-(1H-indol-3-yl)propan-2-yl)-2,2,3,3,3-pentafluoropropan-1-amine (1.12 mmol, 0.250 mg, 0.82 mmol) were dissolved in dry toluene (2.5 mL) and acetic acid (4.9 equiv, 207 μL, 3.6 mmol) was added and stirred at 100 °C for 18 h. The reaction was allowed to cool to room temperature and was quenched with saturated sodium bicarbonate solution and extracted with EtOAc (2×5 mL). The combined organic fractions were dried over sodium sulfate, filtered, and dried in-vacuo. The crude was purified by flash chromatography in 0-25% EtOAc in hexanes to give the title compound (261 mg, 0.42 mmol, 58% yield). LCMS: m/z=618.1 [M+H]⁺.

### Example 203: [5-[(1R,3R)-3-Methyl-2-(2,2,3,3-tetrafluoropropyl)-1,3,4,9-tetrahydropyrido[3,4-b]indol-1-yl]-1H-indol-2-yl]methanol

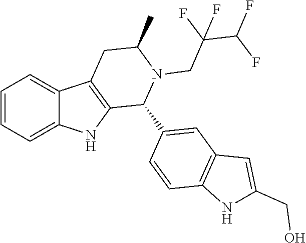 [SMILES: C[C@@H]1Cc2c([nH]c3ccccc23)[C@@H](c2ccc3[nH]c(CO)cc3c2)N1CC(F)(F)C(F)F]

[5-[(1R,3R)-3-Methyl-2-(2,2,3,3-tetrafluoropropyl)-1,3,4,9-tetrahydropyrido[3,4-b]indol-1-yl]-1-(p-tolylsulfonyl)indol-2-yl]methanol (1.00 equiv, 256 mg, 0.43 mmol) and sodium hydroxide (31 equiv, 530 mg, 13.3 mmol) were stirred in MeOH (5 mL) for 3 h at 60 °C. The reaction was allowed to cool to room temperature and was recrystallized with water. The solids were collected with a filter funnel and washed with water (3×20 mL). This was dried under vacuum to give the title product (166 mg, 0.37 mmol, 87% yield). LCMS: m/z=446.1 [M+H]⁺.

### Example 204: [5-[(1R,3R)-3-Methyl-2-(2,2,3,3,3-pentafluoropropyl)-1,3,4,9-tetrahydropyrido[3,4-b]indol-1-yl]-1H-indol-2-yl]methanol

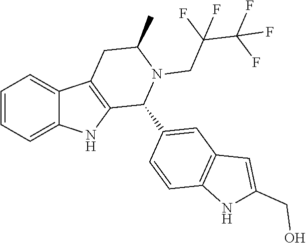 [SMILES: C[C@@H]1Cc2c([nH]c3ccccc23)[C@@H](c2ccc3[nH]c(CO)cc3c2)N1CC(F)(F)C(F)(F)F]

[5-[(1R,3R)-3-Methyl-2-(2,2,3,3,3-pentafluoropropyl)-1,3,4,9-tetrahydropyrido[3,4-b]indol-1-yl]-1-(p-tolylsulfonyl)indol-2-yl]methanol (1.00 equiv, 261 mg, 0.42 mmol) and sodium hydroxide (30 equiv, 507 mg, 12.7 mmol) were stirred in MeOH (1 mL) for 30 min at 60 °C. The reaction was allowed to cool to room temperature and was recrystallized with water. The solids were collected with a filter funnel and washed with water (3×20 mL). The collected solids were dried under vacuum to give the title product (142 mg, 0.31 mmol, 74% yield). LCMS: m/z=464.1 [M+H]⁺.

### Example 205: 5-[(1R,3R)-3-Methyl-2-(2,2,3,3-tetrafluoropropyl)-1,3,4,9-tetrahydropyrido[3,4-b]indol-1-yl]-1H-indole-2-carbaldehyde

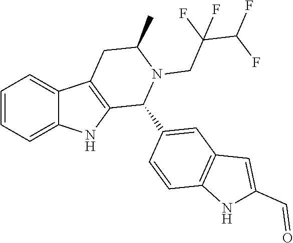 [SMILES: C[C@@H]1Cc2c([nH]c3ccccc23)[C@@H](c2ccc3[nH]c(C=O)cc3c2)N1CC(F)(F)C(F)F]

[5-[(1R,3R)-3-Methyl-2-(2,2,3,3-tetrafluoropropyl)-1,3,4,9-tetrahydropyrido[3,4-b]indol-1-yl]-1H-indol-2-yl]methanol (1.00 equiv, 166 mg, 0.37 mmol) and 45% IBX (3.00 equiv, 695 mg, 1.11 mmol) were stirred in DMSO (3.5 mL) and stirred for 1.5 h. The reaction was diluted with EtOAc and quenched with saturated sodium bicarbonate solution. This was extracted with EtOAc (10 mL) and the combined organic fractions were washed with saturated brine solution (3×10 mL). The organic phase was then dried over sodium sulfate, filtered, and dried in-vacuo. The crude was purified by flash chromatography in 0-30% EtOAc in hexanes to give the title compound (72 mg, 0.16 mmol, 44% yield). LCMS: m/z=444.1 [M+H]⁺.

### Example 206: 5-[(1R,3R)-3-Methyl-2-(2,2,3,3,3-pentafluoropropyl)-1,3,4,9-tetrahydropyrido[3,4-b]indol-1-yl]-1H-indole-2-carbaldehyde

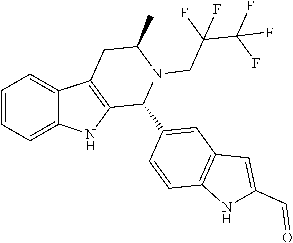 [SMILES: C[C@@H]1Cc2c([nH]c3ccccc23)[C@@H](c2ccc3[nH]c(C=O)cc3c2)N1CC(F)(F)C(F)(F)F]

[5-[(1R,3R)-3-Methyl-2-(2,2,3,3,3-pentafluoropropyl)-1,3,4,9-tetrahydropyrido[3,4-b]indol-1-yl]-1H-indol-2-yl]methanol (1.00 equiv, 142 mg, 0.31 mmol) and 45% IBX (3.00 equiv, 582 mg, 0.93 mmol) were stirred in DMSO (1.5 mL) and stirred for 1.5 h. The reaction was diluted with EtOAc and quenched with saturated sodium bicarbonate solution. The reaction mixture was extracted with EtOAc (10 mL) and the combined organic fractions were washed with saturated brine solution (3×10 mL). The organic phase was then dried over sodium sulfate, filtered, and dried in-vacuo. The crude was purified by flash chromatography in 0-40% EtOAc in hexanes to give the title compound (56 mg, 0.12 mmol, 42% yield). LCMS: m/z=462.1 [M+H]⁺.

### Example 207: (1R,3R)-1-[2-[[3-(Fluoromethyl)azetidin-1-yl]methyl]-1H-indol-5-yl]-3-methyl-2-(2,2,3,3-tetrafluoropropyl)-1,3,4,9-tetrahydropyrido[3,4-b]indole

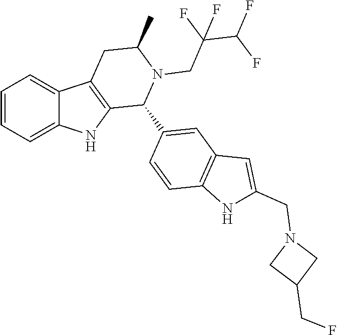 [SMILES: C[C@@H]1Cc2c([nH]c3ccccc23)[C@@H](c2ccc3[nH]c(CN4CC(CF)C4)cc3c2)N1CC(F)(F)C(F)F]

5-[(1R,3R)-3-Methyl-2-(2,2,3,3-tetrafluoropropyl)-1,3,4,9-tetrahydropyrido[3,4-b]indol-1-yl]-1H-indole-2-carbaldehyde (1.00 equiv, 72 mg, 0.16 mmol) was dissolved in DMF (1 mL) and acetic acid (2.00 equiv, 18 μL, 0.32 mmol) was added. Separately, 3-(fluoromethyl)azetidine hydrochloride (1.13 equiv, 36 mg, 0.18 mmol) was dissolved in DMF (1 mL) and TEA (2.2 equiv, 50 μL, 0.35 mmol) was added. These solutions were combined and stirred for 30 min. STAB (3.0 equiv, 104 mg, 0.48 mmol) was added and stirred for 1 h. The reaction was quenched with MeOH (1 mL) and directly purified by HPLC on a Kintetex 5 μm C18 100 Å column (size: 100×30.0 mm; gradient: 5-45% 0.1% formic acid in ACN in 0.1% formic acid in water) then lyophilized to give the title compound as a white solid (36.7 mg, 0.07 mmol, 44% yield). LCMS: m/z=517.1 [M+H]⁺¹H NMR (400 MHz, DMSO-d₆) δ 10.95 (d, J=2.2 Hz, 1H), 10.78 (s, 1H), 8.18 (s, 1H), 7.46 (d, J=7.7 Hz, 1H), 7.27 (dd, J=8.2, 4.5 Hz, 2H), 7.09-6.95 (m, 4H), 6.56 (ddd, J=58.9, 52.4, 6.2 Hz, 1H), 6.17 (d, J=1.8 Hz, 1H), 5.06 (s, 1H), 4.57 (d, J=6.1 Hz, 1H), 4.45 (d, J=6.1 Hz, 1H), 3.62 (s, 2H), 3.27 (q, J=6.2 Hz, 5H), 2.97 (t, J=6.7 Hz, 2H), 2.71 (ddd, J=22.8, 14.5, 5.5 Hz, 2H), 2.60-2.53 (m, 1H), 1.05 (d, J=6.7 Hz, 3H).

### Example 208: (1R,3R)-1-[2-[[3-(Fluoromethyl)azetidin-1-yl]methyl]-1H-indol-5-yl]-3-methyl-2-(2,2,3,3,3-pentafluoropropyl)-1,3,4,9-tetrahydropyrido[3,4-b]indole

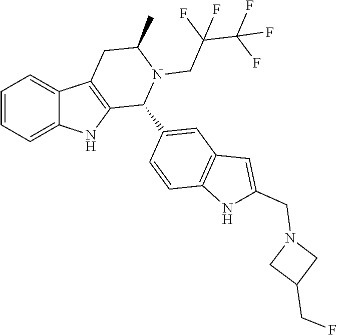 [SMILES: C[C@@H]1Cc2c([nH]c3ccccc23)[C@@H](c2ccc3[nH]c(CN4CC(CF)C4)cc3c2)N1CC(F)(F)C(F)(F)F]

5-[(1R,3R)-3-Methyl-2-(2,2,3,3,3-pentafluoropropyl)-1,3,4,9-tetrahydropyrido[3,4-b]indol-1-yl]-1H-indole-2-carbaldehyde (1.00 equiv, 56 mg, 0.12 mmol) was dissolved in DMF (1 mL) and acetic acid (2.00 equiv, 15 μL, 0.24 mmol) was added. Separately, 3-(fluoromethyl)azetidine hydrochloride (1.08 equiv, 17 mg, 0.13 mmol) was dissolved in DMF (1 mL) and TEA (2.2 equiv, 37 μL, 0.26 mmol) was added. These solutions were combined and stirred for 30 min. STAB (3.00 equiv, 77.2 mg, 0.36 mmol) was added and stirred for 1 h. The reaction was quenched with MeOH (1 mL) and directly purified by HPLC on a Kintetex 5 μm C18 100 Å column (size: 100×30.0 mm; gradient: 5-45% 0.1% formic acid in ACN in 0.1% formic acid in water) then lyophilized to give the title compound as a white solid (29.8 mg, 0.056 mmol, 46% yield). LCMS: m/z=535.1 ¹H NMR (400 MHz, DMSO-d₆) δ 10.97 (d, J=2.1 Hz, 1H), 10.79 (s, 1H), 8.18 (s, 1H), 7.46 (d, J=7.5 Hz, 1H), 7.27 (t, J=8.1 Hz, 2H), 7.10-7.03 (m, 3H), 7.02-6.97 (m, 1H), 6.17 (d, J=1.8 Hz, 1H), 5.07 (s, 1H), 4.57 (d, J=6.1 Hz, 1H), 4.45 (d, J=6.2 Hz, 1H), 3.62 (s, 2H), 3.54 (d, J=16.8 Hz, 1H), 3.27 (qd, J=7.6, 2.9 Hz, 2H), 2.97 (t, J=6.6 Hz, 2H), 2.78-2.57 (m, 3H), 1.06 (d, J=6.8 Hz, 3H).

### Example 209: Biological Evaluation

ECC-1 cells were trypsinized and resuspended in hormone-depleted media and plated at a density of 15 k cells per well into a 96-well plate for at least 4 hours. Cells were treated with test compounds in the absence of E2 (for agonist mode) or in the presence of 500 pM E2 (for antagonist mode) for 3 days and plates were subsequently frozen at −80 °C. Thawed plates were incubated with a chromogenic substrate of AP, p-nitrophenyl phosphate (Thermo Fisher Scientific), for 40 minutes at 42 °C, and absorbances were read at 405 nm. AP activity was normalized to the activity of 500 pM E2 alone. This assay was shown to correlate with the in vivo studies comparing uterine wet weight in ovariectomized rats following treatment with a number of antiestrogens.

Table 6 shows estrogen receptor modulation (e.g., agonism and antagonism) of certain compounds of the present disclosure. The compound numbers correspond to the compound numbers of Table 1 and 2. Compounds having an activity designated as “+” provided estrogen receptor agonism having (i) at least 80% increase in the E2-normalized signal in the AP assay (agonist mode) and (ii) no more than 80% reduction in the E2-normalized signal in the AP assay (antagonist mode). Compounds having an activity designated as “++” provided estrogen receptor antagonism having (i) between 10% and 80% increase in the E2-normalized signal in the AP assay (agonist mode) and (ii) between 10% and 80% reduction in the E2-normalized signal in the AP assay (antagonist mode). Compounds having an activity designated as “+++” provided estrogen receptor antagonism (i) with pIC₅₀ greater than 7.5 and at least a 10% reduction in the E2-normalized signal in the AP assay (antagonist mode); and (ii) no more than 10% increase in E2-normalized signal in the AP assay (agonist mode).

In some embodiments, particularly useful modulators of the estrogen receptor are compounds having greater than “+” activity in Table 6. In some embodiments, particularly useful modulators of the estrogen receptor are compounds having greater than “++” activity in Table 6. In some embodiments, particularly useful modulators of the estrogen receptor are compounds having “+++” activity in Table 6.

TABLE 6

| Compound No. | Activity |
|---|---|
| I-1 | + |
| I-2 | + |
| I-3 | + |
| I-4 | + |
| I-5 | ++ |
| I-6 | ++ |
| I-7 | + |
| I-8 | + |
| I-9 | + |
| I-10 | +++ |
| I-11 | ++ |
| I-12 | + |
| I-13 | + |
| I-14 | + |
| I-15 | ++ |
| I-16 | ++ |
| I-17 | +++ |
| I-18 | + |
| I-19 | + |
| I-20 | + |
| I-21 | + |
| I-22 | + |
| I-23 | ++ |
| I-24 | + |
| I-25 | + |
| I-26 | +++ |
| I-27 | ++ |
| I-28 | ++ |
| I-29 | ++ |
| I-30 | + |
| I-31 | + |
| I-32 | + |
| I-33 | +++ |
| I-34 | + |
| I-35 | + |
| I-36 | ++ |
| I-37 | ++ |
| I-38 | ++ |
| I-39 | ++ |
| I-40 | +++ |
| I-41 | ++ |
| I-42 | ++ |
| I-43 | + |
| I-44 | ++ |
| I-45 | +++ |
| I-46 | +++ |
| I-47 | + |
| I-48 | + |
| I-49 | + |
| I-50 | + |
| I-51 | + |
| I-52 | ++ |
| I-53 | + |
| I-54 | + |
| I-55 | + |
| I-56 | + |
| I-57 | +++ |
| I-58 | + |
| I-59 | + |
| I-60 | + |
| I-61 | + |
| I-62 | ++ |
| I-63 | ++ |
| I-64 | ++ |
| I-65 | ++ |
| I-66 | + |
| I-67 | + |
| I-68 | + |
| I-69 | ++ |
| I-70 | + |
| I-71 | ++ |
| I-72 | + |
| I-73 | + |
| I-74 | +++ |
| I-75 | +++ |
| I-76 | +++ |
| I-77 | +++ |
| I-78 | + |
| I-79 | + |
| I-80 | +++ |
| I-81 | + |
| I-82 | + |
| I-83 | + |
| I-84 | + |
| I-85 | + |
| I-86 | + |
| I-87 | + |
| I-88 | + |
| I-89 | + |
| I-90 | + |
| I-91 | + |
| I-92 | + |
| I-93 | + |
| I-94 | + |
| I-95 | + |
| I-96 | + |
| I-97 | + |
| I-98 | ++ |
| I-99 | ++ |
| I-100 | ++ |
| I-101 | ++ |
| I-102 | ++ |
| I-103 | + |
| I-104 | + |
| I-105 | ++ |
| I-106 | ++ |
| I-107 | + |
| I-108 | + |

Compound I-109 and Compound I-110 provided estrogen receptor antagonism with (i) pIC₅₀ greater than 6.4 and at least a 10% reduction in the E2-normalized signal in the AP assay (antagonist mode); and (ii) no more than 10% increase in E2-normalized signal in the AP assay (agonist mode).

The embodiments of the disclosure described above are intended to be merely exemplary, numerous variations and modifications will be apparent to those skilled in the art. All such variations and modifications are intended to be within the scope of the present invention as defined in any appended claims.

1. A compound of Formula I: 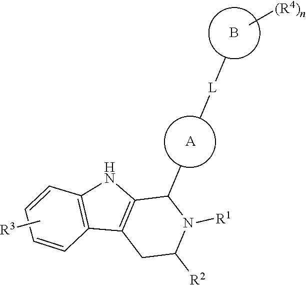
or a pharmaceutically acceptable salt thereof, wherein:
A is an optionally substituted 7- to 10-membered bicyclic heteroaryl comprising 1-4 heteroatoms selected from N, O, and S or an optionally substituted 3- to 6-membered heterocyclyl comprising 1-3 heteroatoms selected from N, O, and S;
L is a covalent bond or an optionally substituted bivalent group selected from —C₁-C₆ aliphatic-, -L^{a}-C₀-C₅ aliphatic-, and —C₁-C₅ aliphatic-L^{a}-, wherein L^{a} is selected from —S—, —SO—, —SO₂—, and —N(R^{a})—;
B is selected from —OH, —CO₂H, C₁-C₆ aliphatic, 3- to 12-membered heterocyclyl comprising 1-3 heteroatoms selected from N, O, and S, and C₃-C₆ cycloaliphatic;
R¹ is selected from hydrogen and optionally substituted C₁-C₆ aliphatic;
R² is selected from hydrogen and optionally substituted C₁-C₆ aliphatic;
R³ is selected from hydrogen, halogen, —CN, —OR^{a}, —C(O)R^{a}, —C(O)₂R^{a}, —OC(O)R^{a}, —C(O)N(R^{a})₂, —OC(O)N(R^{a})₂, —NO₂, —N(R^{a})₂, —N(R^{a})C(O)R^{a}, —N(R^{a})C(O)₂R^{a}, —N(R^{a})S(O)₂R^{a}, —SR^{a}, —S(O)₂R^{a}, —S(O)N(R^{a})₂, —S(O)₂N(R^{a})₂, and an optionally substituted C₁₋₆ aliphatic group;
each R⁴ is independently oxo, halogen, —CN, —OR^{a}, —N(R^{a})₂, —C(O)R^{a}, —OC(O)R^{a}, —C(O)₂R^{a}, —C(O)N(R^{a})₂, —N(R^{a})C(O)R^{a}, or an optionally substituted group selected from C₁-C₆ aliphatic and 3- to 12-membered heterocyclyl comprising 1 to 3 heteroatoms selected from N, O, and S;
each R^{a} is independently selected from hydrogen and optionally substituted C₁-C₆ aliphatic; and
n is 0 to 5.

2. The compound of claim 1, wherein R¹ is optionally substituted C₁-C₆ aliphatic.

3. The compound of claim 1 or 2, wherein R¹ is C₁-C₆ aliphatic optionally substituted with one or more halogen or —OH.

4. The compound of claim 3, wherein R¹ is C₁-C₆ aliphatic optionally substituted with one or more fluoro or —OH.

5. The compound of any one of claims 1-4, wherein R¹ is

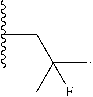 [SMILES: *CC(C)(C)F]

6. The compound of any one of claims 1-4, wherein R¹ is

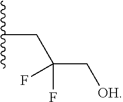 [SMILES: *CC(F)(F)CO]

7. The compound of any one of claims 1-6, wherein R² is optionally substituted C₁-C₆ aliphatic.

8. The compound of any one of claims 1-7, wherein R² is methyl.

9. The compound of any one of claims 1-8, wherein R³ is hydrogen.

10. The compound of any one of claims 1-9, wherein A is an optionally substituted 7- to 10-membered bicyclic heteroaryl comprising 1-4 heteroatoms selected from N, O, and S.

11. The compound of any one of claims 1-10, wherein A is: 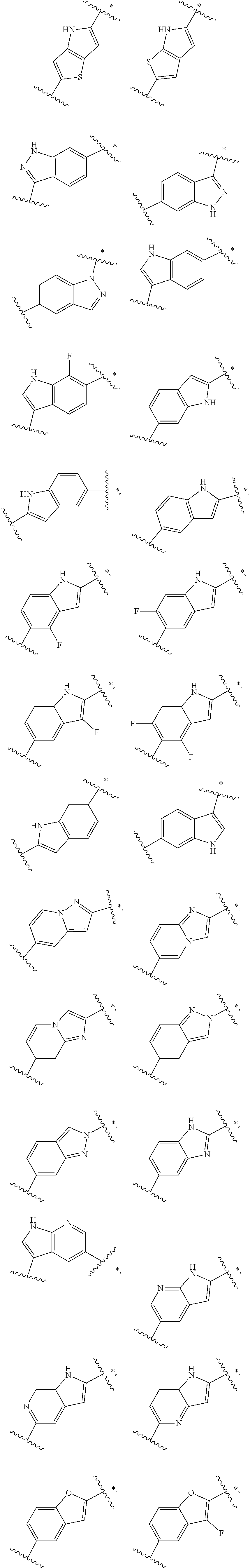 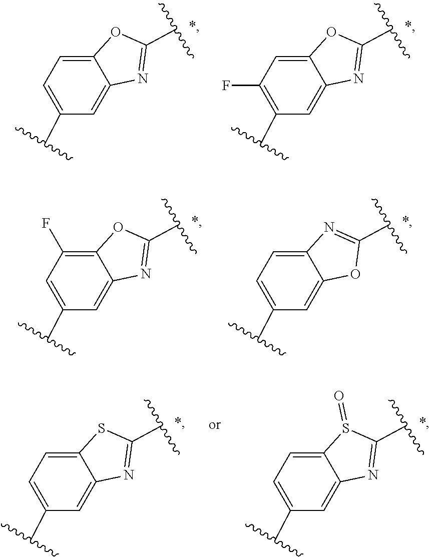 [SMILES: *c1ccc2oc(*)nc2c1 ; *c1nc2cc(*)c(F)cc2o1 ; *c1cc(F)c2oc(*)nc2c1 ; *c1ccc2nc(*)oc2c1 ; *c1ccc2sc(*)nc2c1 ; *C1=Nc2cc(*)ccc2S1=O]
wherein *represents a point of attachment to moiety L.

12. The compound of any one of claims 1-9, wherein A is an optionally substituted 3- to 6-membered heterocyclyl comprising 1-3 heteroatoms selected from N, O, and S.

13. The compound of claim 12, wherein A is: 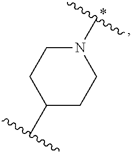 [SMILES: *C1CCN(*)CC1]
wherein * represents a point of attachment to moiety L.

14. The compound of any one of claims 1-13, wherein B is 3- to 6-membered heterocyclyl comprising 1-3 heteroatoms selected from N, O, and S.

15. The compound of any one of claims 1-14, wherein B is 4- to 5-membered heterocyclyl comprising 1-3 heteroatoms selected from N, O, and S.

16. The compound of any one of claims 1-15, wherein B is azetidinyl or pyrrolidinyl.

17. The compound of any one of claims 1-16, wherein B is azetidinyl.

18. The compound of any one of claims 1-13, wherein B is 6- to 12-membered bicyclic fused or spirocyclic heterocyclyl.

19. The compound of any one of claims 1-18,
wherein: 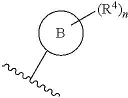
is a moiety selected from:

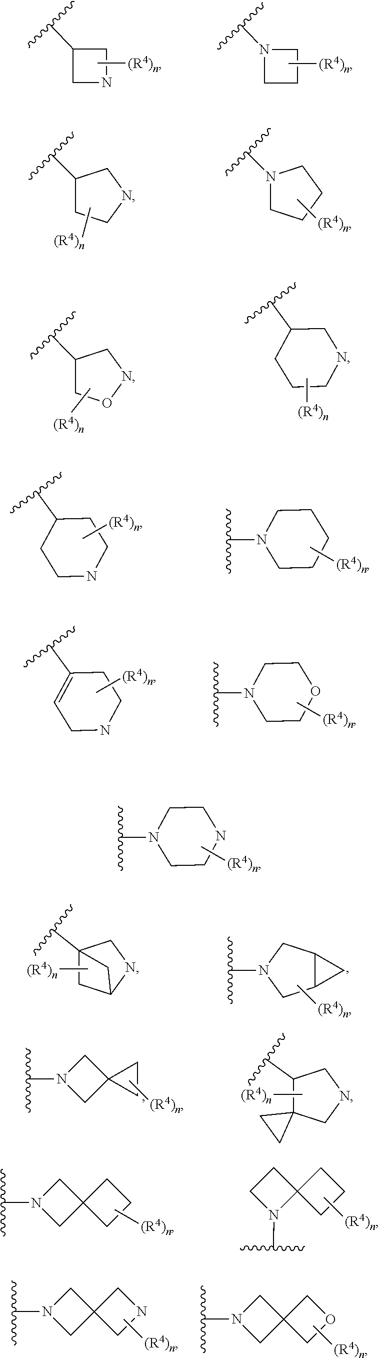

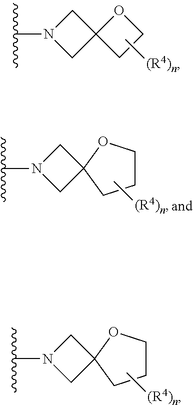

20. The compound of any one of claims 1-19, wherein 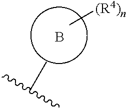
is a moiety selected from:

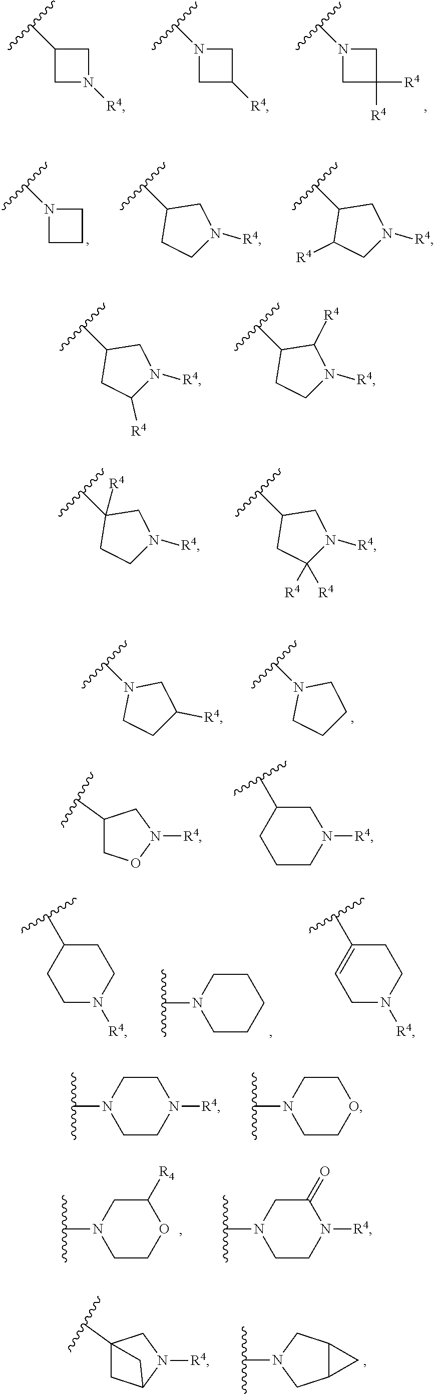

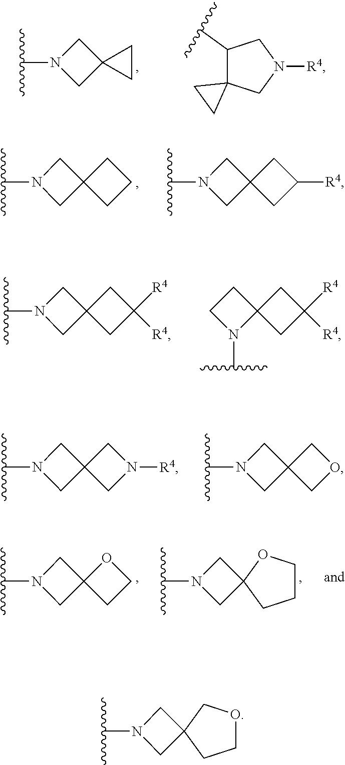

21. The compound of any one of claims 1-13, wherein B is C₃-C₆ cycloaliphatic.

22. The compound of claim 21, wherein B is selected from cyclopropyl, cyclobutyl, cyclopentyl and cyclohexyl.

23. The compound of any one of claims 1-22, wherein L is selected from optionally substituted C₁-C₆ aliphatic, —S—C₀-C₅ aliphatic-, and —SO₂—C₀-C₅ aliphatic.

24. The compound of any one of claims 1-22, wherein L is optionally substituted C₁-C₆ aliphatic.

25. The compound of any one of claims 1-24, wherein L is —CH₂—.

26. The compound of any one of claims 1-19 or 21-25, wherein n is 1.

27. The compound of any one of claims 1-19 or 21-25, wherein n is 0.

28. The compound of any one of claims 1-26, wherein each R⁴ is independently selected from halogen and optionally substituted C₁-C₆ aliphatic.

29. The compound of any one of claims 1-26, wherein each R⁴ is an independently selected optionally substituted C₁-C₆ aliphatic group.

30. The compound of any one of claims 1-26, wherein each R⁴ is independently selected from fluoro, —CH₃ and —CH₂F.

31. The compound of any one of claims 1-30, wherein the compound is of Formula II: 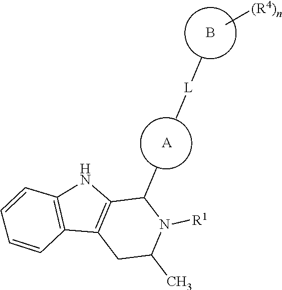
or a pharmaceutically acceptable salt thereof.

32. The compound of claim 31, wherein the compound is of Formula II-a: 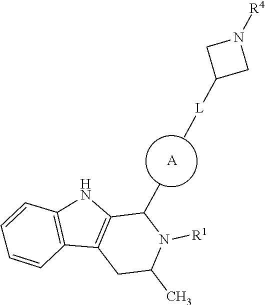
or a pharmaceutically acceptable salt thereof.

33. The compound of claim 31, wherein the compound is of Formula II-b: 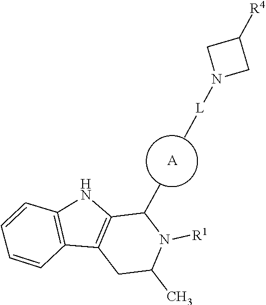
or a pharmaceutically acceptable salt thereof.

34. The compound of claim 31, wherein the compound is of Formula II-c: 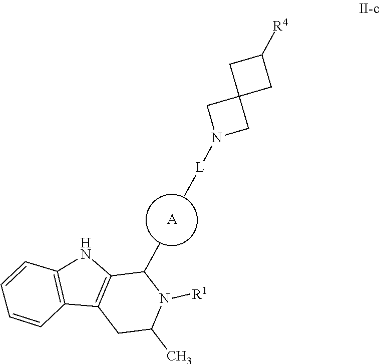
or a pharmaceutically acceptable salt thereof.

35. The compound of claim 31, wherein the compound is of Formula II-d: 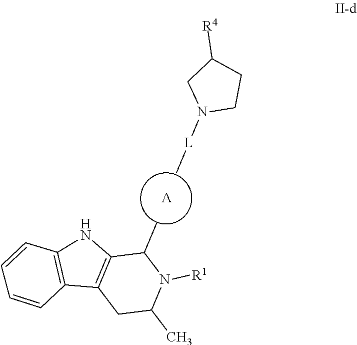
or a pharmaceutically acceptable salt thereof.

36. The compound of claim 31, wherein the compound is of Formula II-e: 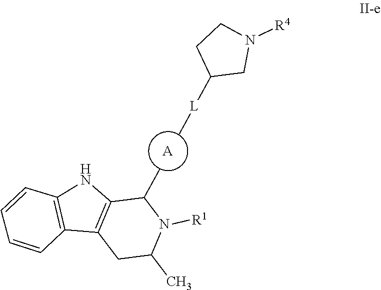
or a pharmaceutically acceptable salt thereof.

37. The compound of claim 31, wherein the compound is of Formula II-f: 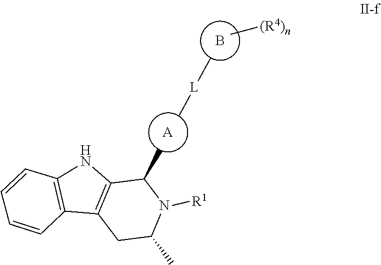
or a pharmaceutically acceptable salt thereof.

38. The compound of any one of claims 1-11 or 14-31, wherein the compound is of Formula III: 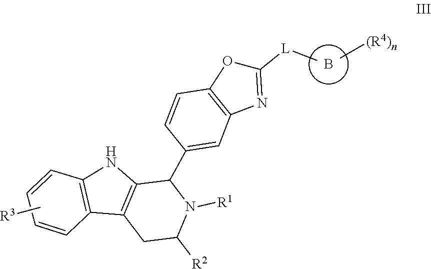
or a pharmaceutically acceptable salt thereof.

39. The compound of any one of claims 1-11 or 14-31, wherein the compound is of Formula IV: 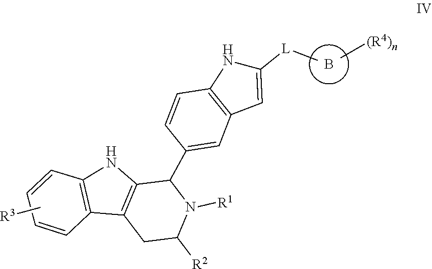
or a pharmaceutically acceptable salt thereof.

40. The compound of any one of claims 1-11 or 14-31, wherein the compound is of Formula 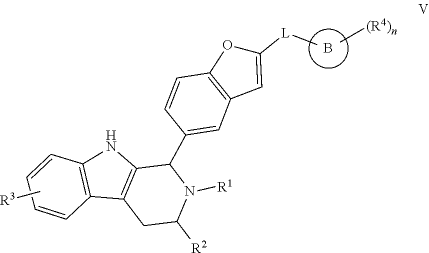
or a pharmaceutically acceptable salt thereof.

41. The compound of any one of claims 1-11 or 14-31, wherein the compound is of Formula VI: 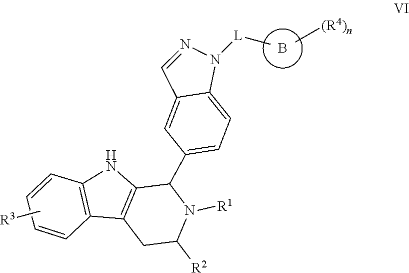
or a pharmaceutically acceptable salt thereof.

42. A compound selected from Table 1, or a pharmaceutically acceptable salt thereof.

43. A compound selected from Table 3, or a pharmaceutically acceptable salt thereof.

44. A compound selected from Table 4, or a pharmaceutically acceptable salt thereof.

45. A compound selected from Table 5, or a pharmaceutically acceptable salt thereof.

46. A pharmaceutical composition comprising a compound of any one of claims 1-45, or a pharmaceutically acceptable salt thereof, and a pharmaceutically acceptable carrier or excipient.

47. A method for treating a disorder mediated by an estrogen receptor in a subject, comprising administering to the subject a therapeutically effective amount of the compound of any one of claims 1-45, or a pharmaceutically acceptable salt thereof, or the composition of claim 46.

48. The method of claim 47, wherein the disorder is selected from the group consisting of breast cancer, ovarian cancer, endometrial cancer, vaginal cancer, lung cancer, bone cancer, uterine cancer, and endometriosis.

49. The method of claim 47 or 48, further comprising administering the compound or composition in combination or alternation with an anti-cancer agent.

50. The method of claim 49, wherein the anti-cancer agent is selected from an mTOR inhibitor, a CDK4/6 inhibitor, a PI3 kinase inhibitor, an aromatase inhibitor, an antibody to or inhibitor of PD-1, PD-L1 or CTLA-4, or an antibody to or inhibitor of EGFR, PGFR, or IGFR.

51. The method of claim 47 or 48, further comprising administering the compound or composition in combination or alternation with an estrogen receptor antagonist or a partial estrogen receptor antagonist.

52. The method of any one of claims 47-51, wherein the disorder is breast cancer.

53. A method of treating a subject suffering from a cancer comprising administering the compound of any one of claims 1-45, or a pharmaceutically acceptable salt thereof, or the composition of claim 46, in combination with an anti-cancer agent.

54. The method of claim 53, wherein the anti-cancer agent is selected from an mTOR inhibitor, a CDK4/6 inhibitor, a PI3 kinase inhibitor, an aromatase inhibitor, an antibody to or inhibitor of PD-1, PD-L1 or CTLA-4, or an antibody to or inhibitor of EGFR, PGFR, or IGFR.

55. The method of claim 54, wherein the anti-cancer agent is an mTOR inhibitor.

56. The method of claim 55, wherein the mTOR inhibitor is selected from everolimus, sirolimus, temsirolimus, and LY3023414.

57. The method of claim 54, wherein the anti-cancer agent is a CDK4/6 inhibitor.

58. The method of claim 57, wherein the CDK4/6 inhibitor is selected from palbociclib, abemaciclib, ribociclib, lerociclib, trilaciclib, and SHR6390.

59. The method of claim 54, wherein the anti-cancer agent is an antibody to or inhibitor of PD-1, PD-L1 or CTLA-4.

60. The method of claim 54, wherein the anti-cancer agent is an antibody to or inhibitor of EGFR, PGFR, or IGFR.

61. The method of claim 53, wherein the anti-cancer agent is a HER2 inhibitor.

62. The method of claim 61, wherein the HER2 inhibitor is selected from tucatinib, trastuzumab, pertuzumab, ado-trastuzumab, trastuzumab emtansine, ado-trastuzumab emtansine, trastuzumab deruxtecan pertuzumab, lapatinib, and neratinib.

63. The method of claim 54, wherein the anti-cancer agent is a PI3 kinase inhibitor.

64. The method of claim 63, wherein the PI3 kinase inhibitor is selected from perifosine, CAL101, BEZ235, XL147, XL765, GDC-0941, and IPI-145.

65. The method of claim 54, wherein the anti-cancer agent is a PIK3CA inhibitor.

66. The method of claim 65, wherein the PIK3CA inhibitor is selected from alpelisib, taselisib, and LY3023414.

67. The method of claim 54, wherein the anti-cancer agent is an aromatase inhibitor.

68. The method of claim 67, wherein the aromatase inhibitor is selected from aminoglutethimide, testolactone, anastrozole, letrozole, exemestane, vorozole, formestane, fadrozole, 4-hydroxyandrostenedione, 1,4,6-androstatrien-3, 17-dione, and 4-androstene-3,6, 17-trione.

69. The method of claim 68, wherein the aromatase inhibitor is selected from anastrozole, letrozole, and exemestane.

70. A method of preventing recurrence of a cancer in a subject comprising administering to the subject the compound of any one of claims 1-45, or a pharmaceutically acceptable salt thereof, or the composition of claim 46.

71. The method of claim 70, wherein the cancer is selected from breast cancer, ovarian cancer, endometrial cancer, vaginal cancer, lung cancer, bone cancer, and uterine cancer.

72. The method of claim 70 or 71, wherein the compound or composition is administered as an adjunctive therapy after or instead of chemotherapy, radiation, or surgery.

73. The method of any one of claims 70-72, wherein the compound or composition is administered after surgery.

74. The method of any one of claims 70-73, wherein the compound or composition is administered prior to surgery.

75. The method of any one of claims 70-74, wherein the cancer is breast cancer.

76. The method of claim 75, wherein the breast cancer has progressed in the presence of endocrine or aromatase therapy.
